SEQUENCE LISTING (404 sequences)

SEQ ID NO: 1 - protein, 1024 aa, synthetic construct
MYTCSECHRSFTTKNGLGVHRRRQHLAAANAEIVTERHRARWTEEEVLSLAKAEAELFLERDARFFFVNQELIRMFPDRTLEAIKCRRRQAAHKQLVRQFMEALEIGRGEEPASRRGAASPLPDAGEAAAPPVDAAEDFAADTTGPPPEGPTDAAIWEHLAGLPASAQRFSALDRVIGLGRGTPPDVILGMLPDALASVGSRGERSITRTQRPRQPSKRPPAAPPTQKRKRRRWEYARTQDAFRRSRARCVRGLLDGTLLQPPPAIPGLLDFWADLFTKKPISTAGFIRDRLLPHSEPVALECIWGPVTHEEVAAALPPRGSAAGPDGLTPAELRRLPHEVLVKVMNLFLLARALPERLLRARTSLLPKTAAPTSPADFRPITVCSVLARTFHKVLASRLMRACAVDERQRAFIPRDGMLENTFILDTALTDAVRSCRSVFVASIDVSKAFDSVDHAALRPVLKAHGLPDCFVEYVERCYEGSTTVIADGAGVGVSVQPARGVRQGDPLSPLLFNFAVDYVLGQLPSHIGARILGRRVNAAAFADDVLLFAATPRGLQSLIDAATAALAHLGLQINARKCFTLALVASGREKKVKVDSNVTFTAGNTTMPALRVGETFRYLGLQFSTAGRCVFNPRSHLVEQLDVISRAPLKPQQRLHALTNVLLPGLYHGLALSRTRVGALKSADVTIRAAVRRWFRLPADTPLGYFHAPVAQGGLGIPSCRWMGPTLRRSRLLALKKIGPACDGAGMDEVQREIEVLERHLMWEGHLLKSSTQVGEMWAARLHIAIDGAALSSSAAVSGQHQWVADTSRLLSGREYIDALRARINAFPTKARRSRGREADTRCRAGCQAVETANHVLQACFRTHGSRVKRHDAVVRYVARGLAQRGFNVSVEPHLRTPEGIRKPDVVAVKDGIARVVDAQIVGDHLRLDWCHSQKAAYYDTPSIRRAISNLHRDVEEVIVSTATLNWRGVWSPASARDLAALGFRPRELAVLSTRTLQSCCKSYKIFERMTAPSPKQRVGVG

SEQ ID NO: 2 - protein, 1042 aa, synthetic construct
MSHVGESMNSAPRITALTATNSTPTANSLGAQHEPGTAVNPASQVPCMVCGRLFKTSRGLGVHMSHRHKDELDTARRRVDVNLRWSEEEAYLLARKELELEASGNLRYPNKKLAEFFNHRSSEAIKCYRKRGEYKAKLEQLRGQVAHAPEALEPNTTQRRPSSIERRDRVSTTDAPPIMIPQQSNCEILRILQGYRPVVCNPRWRAEVLQSIIDSAPDLGRETTLQRLSIYLLEIFPKPKQVELSRPPRRPRNRRQLRRQQYAAVQRNWDKHNGRCIKSLLDGADESVLPNREIMEPYWREVMSQPSTCSCDNAMFHLEHSLEKVWSAITAHDLRVNKVKLSSSPGPDGISPKTARSIPTGIMLRIMNLILWCGNIPQPIRLARTVFIPKTVTANRPQDFRPITVSSVLVRQLNAILASRLTSSVNWDPRQRGFLPTDGCADNATMVDLILREHHKRWKSCYIATLDVSKAFDSVSHQAVYNTLTAYGAPKDFIDYLRKLYEGGGTTLNGEGWRSEEFVPARGVKQGDPLSPILFNLIIDRLLRSFPDRVGAKVGNAITNAAAFADDLVLFAETPMGLQKLLDITVDFLSSVGLTLNADKCFTVSIKGQAKQKCTVVERRSFCVGSRECPSLKRTEEWKYLGIRFNADGRARFDPADDFGPKLLRLSRAPLKPQQKLFALRTVLIPQLYHKLTLGSVTIGVLRKLDRLVRSTVRRWLDLPLDVSVAFFHAPHTCGGLGIPSLRWTAPMLRLKRLSNIKWPHLEQSEVASSFIDDEMQRARDRLLAGIGRLLTRAEIDSYWANRLHMSVDGSGLREAGHFRQQHGWVCQPTRLVTGKEYLNGIKLRINALPSKSRTTRGRHELGRRCRAGCDAPETTNHILQQCYRTHGRRIVRHNGVVNALKRGLERKGYVVHVEPSLQCETELNKPDLVAIRQNHAYVIDAQVVTDGLSLDDAHQRKVERYDRQDVRTELRRIFGVTGNIEFHSATLNWRGIWSGQSVKRLIAKDLLITGDSNIISVRVVKGGLLCFKQFMHYCGYSRAWT

SEQ ID NO: 3 - protein, 1374 aa, synthetic construct
MDTTLTAESDKRKNSAHNKRDQSAHYGDSELCIINEQSEIIKINEESENNERWKNNEHSDRYNEDSEFYSEQTTHCGYSEHCEVNKQSEFNTINEHSERNDRSESVTSLSSVTFNERQSNANSNGRRQSMTGSRTSAGQPTADDQRGSQDTQLNNELDIAQVAESDRNEESAQNSRFENHLHLEESENSGQNEHYEHSEQSEHNEQSVINEINEHSELNELRETHEIREVTVSVPLVNIDYCPVCQYFNKSVAFLNMNDLKKHLSEKHTTVKVMWQCKKCLKKFPGLRNCQCHIPKCKEIVETQQLEHICQICSQSFSTVRGLSTHERHRHPTERNKKRIELCERPKGRPGRRNHVWTEEEIEELKKLNEIYQNVRFPNVQIQKHLPHKTTKQISDKRSEIRKANHTNNAPQIASGNINDNSTASEHTGLHMNASFESRMNELTNAEDIPPMDIRENSSNENTEEGPRNADRRMSIEDTDILDLNLGSLDLPEEEEEEGWKESFRTAIRANPLSEDHELKSIEETIMQLATKDSLDPLDTEGIIEALVEKIGTGEEKNQGNPRKKKEHGRAEKNKNKNSDRTRFKKYKYSRFQEMYKKCPKKLIDMAVAGESLEKREEVELPERTSIAKLYGDLWGVEGPTTGIPNFANARTVSPIESVWKPIDIGDIRIKMKKIKKDTAAGLDGVTKAHLKKPGTLYTLSKFYNLLMLTKSYPAQWRLNRTTLIPKPGKDAREAKNWRPITIGSLLSRIYSSMLDNRIRQVVKNNKRQKGFTKEDGCKQNIAILDRIITEMKERTGGIVTVVDISKAFDTVPHKSIGDGLKRKGIPETITTYIEKMYDGCRTEIRTKEESIKIMLKRGVKQGDPLSPLLFNTIIDPIIENIDETTEGIEVGEENVSILAFADDLVLIAKTTEEAAKQAQKLNQYLKELGMGMQAEKCSTFQIIQKGKTWHIADPEIKIGEQKVPASDPDDVISYLGLTIGPWKGVHRAQIEDIIKAAGNVRKMSLKPDQKIELIRTYLLPRYIHNLVANPPALSTLRSLDHEIKQIVKAILHLHPSTTDGLIYTEKKHGGLGIQKIADIVRIAKLRNSLRMMGNEDHAVREAYKKQEATTIKYARSVGLRWPASMREIEGARCKLKESNRETWEQLASQGQGVHEFANDKTGNAWLYTPSLLKPSRYLDALKLRSNTFGTRVVLRRANKDVDPICRRCKGQIETLGHVLGSCTHTKSARMNRHNEIVRLIKEKTSNKCAVFEEPTIMVLGELKKPDLVIKDQERLFVVDVTVRYEDKDNLRKAKKDKIDKYTETAKLLKLKLKCQTAQVLPIVVGCRGAMPEATIDNLKTLGLTKGERLTVAMIAIRSSIEMANQFIDYDRIV

SEQ ID NO: 4 - protein, 1046 aa, synthetic construct
MNSIREILSGHPDLPQESGSLAHSPATRTGASASGPVVDWTCPVCERKFSTKTGLGVHKRRAHPLEMNAEAAPASVKRRWREEEVLLMARTEARLLEETGRCTNVDLLNCLTGFDRTLEAIKGKRRGGDYRSLVQRCRDEFRPPSWGGRQSRDSLQPVMETESAESSVQPQASPTRTPPAVNPGIPGTPCASRLIEDLLSVAAARKARYGLGAKSAKRFRININLDYDTNNASARLSSRKRRRIEYARVQELYRKSKSRAAAEVIDGAHRRVGHTLEQLESYWRPVLERVSDATGPTPGALRALRRAERDGGERDYSALWRPFTSDELKASCFDWQTAPGPDGIRPEEWRQVPWDLKAEIFNSWLSAGEIPEPLRQCRTVFVPKTDAPAGPGEYRPISIASIPLRHMHSVLAKRLLDCCPPDVRQRGFVRADGTLENSAVLDAVLGDCRKKLRECHVAVLDFSKAFDTVSHKALVGLLRARGLPVPFCDYICRLYETATTTLVVDGKSSTPAHVGRGVRQGDPLSPILFNMAMDVILASLPSEVGYRLETERVSALAYADDLVLLAGSKVGLQESIDRVFEVASTMGLMINHKKSSVLSMVPDGKRKKHHYVTEKTFKVGRMWLPQISCTERWRYLGVDFQVSGCVTLEHDIKNALNNITKAPLKPQQRLEIVRGHLIPRFLHGLVLGNISDDRLSMLDVQIRGAVRKWLRLPKDVPVGYFHADTKDGGLAIPSLRAVVPDLIIKRFGRLDSSSWSVARAAARSDRISKKLSWAHRQVRKFTIEDPVTGLRSVARFWRNKLWSSVDGFELRESARTPASTKWIREECTRFTGRDFVQFVHTHINALPSRVRTSRGRGLVRESASKCRAGCAANETTAHTIQQCWRTHGGRIERHNRVVRLVAEAMTSNQWSVQLEPQVKTARGLRKPDIIAAKGNVGVIVDAQVVSGQRPLDDAHREKRSKYGTHDELVEKVAGILGLPGKENVRATSCTISWRGVWSHSSYKEMKSLLGLKESLLRLIPSLVLRGSHINWTRFNQMTGTWAGTPT

SEQ ID NO: 5 - protein, 1031 aa, synthetic construct
MDDAPMLGGHQNSGASTGDAAPVPEGNSESARSLGIGYACECPGCDRIFPSKSGRGVHQQRAHKDWYDAQQDTTRVKARWSPEDIAILARQEARLTLQGVRFINQELLKIIPDRTLEAIKGQRKRADYKAIVEQLITSMRSLEVAASPPTMPPMVDDTSDQHPPDEVPNGISDQTTRNLVIEYLQSLPPCENRSFNNIRMNNIIGSLNIWNPAQIYEEICIYLRETFPSRRKVARHRIPATSPPKSRRKLRRAEYARVQEAWKRNRTSCMRDLLRDKRTSSAPPKEVMVPFWEKIMTSSSSNTPGAAYGKEVLMELWAPVIPNEVKAALPPLGTAPGPDGFSAKQFRDVPIGIWTRIFNIFVLCGKLPQHLLESRTTLIPKKDGASEPGDFRPITVSSTITRAFHKVLANRMSRYIRLDPRQKAFRPIDGCSENIFLMDFILKYSRQNHQPLFMASLDVAKAFDSVTHQTILDTLRSSGVPNMMIEYIAEAYGRSVTRLQCEEWESHAIHPTCGVKQGDPLSPIIFNLVIDRLFSKLPKEIGVRVGNSIVNSMGYADDLVLFALSANGLQQLLDISADYLQECGLNVNASKCFTVSLKSVPKQKKSVVDASQSFSCLGRPMPALKRSDEWRYLGVPFTPEGRLMNNPLNRVKQEINILATAPLKPQQRLFTLRTVVIPALYHLLVLGSTNISLLNKLDVMIRKAVRKWLDLPHDVPNAYFHADVKDGGLSLPSLRWIIPLQRYNRLRALKIMENDEVPRLMRLFVESEIKRAQLRLNDHGQFIGSLTQCKERFAKLLHASNDGCPLEKSRDVMQQHAWLTDGTLFVSGHDFIHMNKVRINAVPLRSRMARGKVRDRHCRAGCNATETLQHVLQQCHRTHDARIKRHNACLDHLLRRQKAPRLVEKEPNFKTPNGLLKPDAIIKMDTNAIVVDAQIVGERVNLEQAHQAKIDKYTVLEGAIKARYAVEQVIFTSLTLSSRGVWSRVSFEHLIQLGLLKKSDAKIMSTRALIGGLHALNIFNRRTTVRRRLAA

SEQ ID NO: 6 - protein, 1084 aa, synthetic construct
MDKNFEITVPIVGTQVACPVCQEGGINLFFLSLTDLGKHLEQHHLTARIQWRCQYCSKSFSKLHGAHCHQPHCGGGPIEISEGPYKCEACPRSFGSQRGLSTHERHAHPAVRNVKRRGTDPPGKKWTFQELALLKELCETYKGHRNINIKINEILTSKTIDQIKYQRKKLEELEEEIQEATQVTEGGCDPVDPGNAYLEEPGLSGDFEEWKLQLEKAITTHTEVPPVLRWVYTRLMVVWDTLKGNREALEGGINDFLQTLYGAIEHYNKEHGKVIERIKTKRIVHQNKKSRRNAKKRYSFARCQDLFLENPKRLAEVVIDNDQAFLQPARDPPDAAEVRRLYEDLWGQTGPINAPVPGSGAPELSLCEVFPPIAVEDVEERIKRIRKKAAAGPDGFQKEHLLIPGLPIILAKLYNILCYISYFPSAWKENRTTLIPKVNKPSSQVENWRPITIGPILGRIFSSIIDGRIRRGVVLNPRQKGFTAESGCKINIDLLSAALDHSKKGKGGIFTIVDISKAFDTVPHSAIEPCLARKGVPAPIVQLIKNMYKGMSTTIKTKKNMGVKINILRGVKQGDPLSPLLFNLCLEPLLELILEKTSGINVNNNRKIPVLAFADDVVLLGANQGEAQYQVDLLDQYLSGLGMNISREKSQIFEIVAKKDTWFVKEPEVKLGENIIPTVDPGEAFKYLGAKIGPWKGVHCGIIVPELLSVVRRVRKLSLKPCQKLELITKYIFPKYIYHLLVSPPGDSVLRLLDSEVRQEVKAIFHLMPSTATGFFYAPKSWGGLGLPRFEHIVKLGTLKNAIKMKNSKDPAVSGLIDEHGELKLKKIANSLRINWPASLEDIEKARKTLKAAHVKQWAELRSQGQGVLDFSRSKIGNVWLREYELLKPSRFIDALKLRTNTFGTRTVLARADKNIEVACRKCRAQPETLGHILGLCQYTKGLRIKRHDEAKTTLADNLRSNNEVFIEPTLRVEGNLYKPDLVIKNEERVLVVDVTVRYENKDYLSKAEKEKINKYKPCLEHLKEKFNVGRGEVIPVVLGSRGAITPSTEKSLKQMGISNKVIKTLVVNVLRSSIELCNIFLDE

SEQ ID NO: 7 - protein, 1214 aa, synthetic construct
MADAHNTQASDTELPGPGSSVTDSSVLEGVIYDDSEMIPVMPLVSVKGPYSIEVPLSTEQTECPQCKEKGACQFFVSLTDLEKHLKAKHLRTVVHWKCQKCHKPYTKLHAARCHFSKCKGAPNEDNKTIQCSGCSMRFETKRGLSTHERHMHPEIRNRKRQAEEARKNVDPGPGRKNMVWTREEENLLRELEPQYKSCRQPNKEIQKHIPYRSCKQISDKRVTLFSKQQVAININEVAATGTNAGEIIINQVQGPKDDSFSAGEGNNVESSAHPNEDDSNADPYEDDQTKWLEALFQEANNTDLTSTPKEVRELYCDIRATFQIYYDNHEEPTNKIEEFLKKLMLFFKNSVATDNKKGTGKHAQKSKDKQSDKNNKFNNNNKFNKSDRNNDNHASKTNKNNNNNNHANKKNRKSNNKNKTSRNQLRKYMYARCQELYKECPKKLADLIVSHDEFTIKPPIPPPQAQAIKDQYEDIWGISGPANIPLKFNNPSPFKLMDFFFPVTAQEIALKIKKIKTNTAAGVDGIKKHHLLMDGVPIILASLFNMLLYTNHYPESWKINRTTLIPKPNKDLNNVGNWRPITISSLISRIFSSLVDARMRTVIVQNMRQKGFTSENGCAQNTAMMDNALSTCKLNLGGVFTILDISKAFDTIPHAMIEIGLNKRGIPPRIIELIKNMYKQCFTSIKACNNESVEIEIKRGVKQGDSLSPLLFNLSLEPLLENLEEHTTGLSINNNVHIPVLAFADDLVLLSKDKEEAQRQLNFVTSYLNKLGMNISASKCNTFQIMTKKDTWFVIDPKIKTQDGKLIPYEDPQEVLRYLGAKIGPWKGMQCGIVVPEIIDLVKKIRTLPLKPQQKIDLLTRYILPRFIYQLLISPPSDGVLKLLDSEVRQQVKEILHLMTSTASGFFYTPRNKGGLGIPRFEHIIKLAVLKNAINLKNTMDPAVLGLINDKVENKLKAIANSLRINWPASLDDIEKAKARLKNEHLSQWMSLKSQGQGVKEFADDKIGNSWLASNALKPSRFIDAIRLRTNTFGNKTTLARAKKDIDVTCRRCHNQPETLGHILGLCQHTKSLRILRHDEIKKFIAEKNEPNNQTFIEPSFQMENNTYKPDLVIKNEKSIFIVDVTVRYENKDYLQKAHLEKISKYSACLGYLKNRFKIKEGEVLPIVVGCRGALPLTTQINLKKLGLNKHHMKTISLSALRCSIEIANIFIDD

SEQ ID NO: 8 - protein, 1226 aa, synthetic construct
MDNTSNNNDASDTQLPGPGSCVADSSALEGTTGEDMDLNTDNETVISAAITIRVPLQENGMLCPICIKDNLQISCEKLRDLTLHFDKHHLVQTFIWECRDCGKSFDKPHGWSCHYCKCKGAVRVEAKPFQCEGCDMSFSSIRGLSTHERLVHPAIRNEKRKQALVRPLECPAVADGRSDRGVWSAEETNKLIELDARFRLHASTNKEISKFLTSKTAKQISDKRGSLRKKGVIKDPESIPREDMVVELDEVESAETNPNGLVEEFQDISDQPESVNPQAIPRQEDLNSEAHDPNAEDTDWRKTMTTAIESISINDAPPSIKNLHKKLKNIWLKHKDDIDKLKLEIDSYVSDTLVTFLLGSDEATDSNMPAGKNARNNNKKKKNNNETCNNNQNNNNNNNNISNTNNNNIRNNNNNNNQNRRFRNKTRINNSNNRKKFRYARCQDLYKECPKKLADIIVKGDHTFIDPPRPPPIASEVKELYTNLWETNGPVLQDKTLMRQPISISNILYPILPAEVDIRIKKMNNKTAGGPDNIQKKHLKTQGITTVLAMLYNILCFVNHYPTAWNQNRTTLIPKVGSDLEKVENWRPITISSALSRIFSSIIDGRLRGVIVQNLRQKGFTTENGCKYNTMILNEAIKRSKYLDGGVFTILDVSKAFDTLPHSVLEPSLQRKGIPDQVINLIRSMYANCVTSIKARDKISVQVRLLRGVKQGDPLSPLLFNLGMEHLLEMIEEETEGIPINEDTKVPILAFADDIVILGNDQKEAQKQLNMVTQYLSELGMKLSAKKCQTFQIVAAKDTWYVKDPELKAGIETVPKVDPDEVFKYLGAKMGPWKGLHQGIIIPDIVAIIKRVRKLALKPQQKIELIITYILPRFIYGLLIHPPADCTLKLLDSEIRQQIKDILHLVPSTATGFFYAPKKSGGLGLPRFEHIVKLAILKNAVKMKIAVDPAVTGVINEKTEASFKKMANSLRINWPATLEDIEKAKMRLKVDHIKQWSDLPSQGQGVQDFAKNRIGNCWLREGYLLKPSRFIDAIRLRTNTFGTRVVLARTKKDIDTTCRRCKCQPETLGHILGLCIHTKSLRIKRHDEIKILLADILAHNNEVSIEPQMTVGGNSYKPDLIIKNEERTLIVDVTVRYEHREYLQAAHDEKVEKYKACAEDIKRRYTNHTCKVLPIVVGSRGAIPEHTLQNFKTLGIKKNDIKTIALIALRSSIEMANIFIDYDEVT

SEQ ID NO: 9 - protein, 1072 aa, synthetic construct
MNWSSPFSYPGRSGGIHNLPVGRMYTRLLDFRVGTPVVFFQSTPRNVKKPRMREEPTGSSLSNNILLEVNDPPDHKCEHCGREFTTKTGLGVHKRRAHPLSHDTDNVRVEVKARWSREELDIMAKKEAFFLKERGVDRKINQWLCESLYSDNRRTVEAVKKQRQKTAYRARVEQFSTELSSAPTLLTSRGAMMESLDIDHSRTELLEQRQFLLYCLDLPDVANASKFHYSDLLDIVEKAESAGINSTLASLSVYLDKILPGSAPKNNRESVARAPPKNSRGRRRMEYARVQSLWKKHNARCIGEILDGPNQSVMPQRRVMEDYWQNVMAPQVSDCSEVWAPLEQESYLNPKIDVWKPISEDDIKVAYPGYSTAPGVDGISCRQLRRVPPGILARVLNMILWCGNLPVRLRSARTIFIPKSSDGSGPGDFRPITITSVVVRILHNVLAKRLSTMASLDPRQRGFRQTDGCGDNTTLVDLILGHSRQNFKSCYVAVLDVRKAFDSVTIGSVLRAAANSGVHYGMVRYLANYYRYSDTVLTGKNWQSPPITTTVGVKQGDPLSPILFNLIIDRLLRALPEEIGYDLGPGQRVNAAAFADDLILFASTSHGLQELLDSVVEVLGECGLLLNASKSLTMSIKGMPKQKKSIIVPDRFVAYGQVLPSLKRGETWKYLGVTYTADGRTPFTPHTELGPKLDRVTRAPLKPQQRMFILRTYLLPQLYHRLALGCVRVGVLNRVDRLIRAYIRRWLQLPSDVPISFFHAPLAVGGLGVDSLRWLAPLLRKGRIEGLGLAKDGIAEAELGIHLANEFVRIEHRLSTPSGVLTSRASMEKMWSDRLHSSVDGWGLRDANEHTKQHHRWLREPTRLLTGRDYVTCVRVRANALPSRSRTSRGRAKINRMCRRGCPKKETTNHIVQQCDGAHAARIARHDSVVTYVSRGLKARKFVVVVEPKIRTGLGLHKPDMLVIKDRTLTVLDAQVVTDGRPADEAHFRKIRKYNLPDLKAQLTNDYGDMDVHFASVTLNWRGIWSRKSVEDLLAKGFISQKDTAIISTRVMIGTAICFRKFNSSTFRAGVG

SEQ ID NO: 10 - protein, 1035 aa, synthetic construct
MYSKRKTGQRVSGLPLESESALNPFPSACAGTSQGGPTCCTCDICGRSFGTKTGLGVHKRRAHPELANAEAAPKQLKRRWRDEELALLARAEARRLVEHGVCSNVALVKDLPGFERTLEAIKGVRRKDTYKNLVQKYKEELSNDTTNEPVTTVQPKSPTTSPRMVMPGPVGYLNAASASKSNARAEDASESLKIVDELLMRASARMTANRSHSSRNRRFIPPQAVRLNARQRRRVEYARVQDLYRKNQSRAAAEIIDGYCRKATHSLEELEAYWRPVLEGVDTVSGPSREALKALRVEESLMERRDYSQLWAPITEDEVKASSTDLKTAPGPDGITAQDWRGVPARRKAEIFNRWMSEGGVPERLQQCRTIFVPKKDEPAGPSDYRPITIASIPLRHMHSILAKRLEACCPPDIRQRGFIRADGTLENSAVLDAVLGDCRKKLKECHVAVLDFSKAFDTVSHAALVDLLRRRGLPPAFCDYIAELYNNASTILAANGQLSETPVRVGRGVRQGDPLSPLLFNMAMDIVLASIPKMIGYRLEGELVSALAYADDLVLVSGSRVGLQESIDVVRRQGSSMGLNINVGKSSVLSMVPDGKRKKVHFLTERTFKVGQKWLKQTSCVERWRYLGVDFNSAGCVTLEHDIKIALNNIKRAPLKPQQRLEVVRVYLIPRFIHGLVLGNISDDRLRLFDVQIRTAVRTWLRLPSDTPKAYFHASIKDGGLAIPSLRTWVPDLIIKRFGTLGSSSWSAAKAASKSERICKKLRWATKLFTKLSREDPVTGRRSVACYWREGLHSSVDGFELREITKTPESTKWVRERCSQISGRDFVQFVHTHINALPSRIRNSRGRRTDERELNCRAGCMVRETTAHTIQQCHRTHGGRILRHNNVARQIAAELRREGWSVEEEPKFLTKAGLRKPDLVVSRGQSGVILDVQVVSGQRPLDDAHREKRNKYAHHDELVEKVAERLGLPRNNVKVTTCTISWRGVWSRKSLCDMKDYFGLKRYIFWDLPIAVIRGSHLNWSRFNSLVSQVGFSD

SEQ ID NO: 11 - protein, 1224 aa, synthetic construct
MIAPKPGEAGMRPPAQKKILSRAKKDSGPLQGATTVTTAGPSRFCCETCGVSYTSKANLARHAKDCGKRDRTKCTYCPLTFDSFAAVRQHERRAHQEQYREELESRLGAPEAMLMEKLAKIEARAIKGAPFLKEMTVATGLTMHQVRSRRERPEYARYLDRARREVKESSLRTFKKTAAPLSTRIVPAPKITSAATPPSAPSSAPSPLSSAPPPIELSDTDSEVEIVQVDKPATPKPQKPPIRTLGKPGGGETSAVSEKRHRLSSPLKPPAKRRDIRPEGDRGTLEASTKTPLGRGPAEKRKRSTPTSDDQPPIRPRLATHHGSPRRADDPPPSPPPRVPRRLSISATELRSIGGPSSPTVPIEILDEAFLSCLVTSRSSVDDATKALIGVGLSGQKEELDEAITNWLMTAFPVDRRKRPTTSTPNVRPPPPTTSRQRRMSAYKKAQDLFQKDRTALAERVLTGCPLLAEEVYPKIKDVEDFYAGIYERPAPTDPRPVTISGESESVFKPVTSEEITASIKTWANSAPGPDGITIALIKRCPTNLLEVLFNIILYRRMTPTAWKTSRTILIPKEGDRRDPANWRPITIGPALQRLLHRVLAKRLGKAVPLHPAQRGFRSIDGTLANLVLLEHYVKARRLKGKSYNVVSLDVRKAFDTVSHAAILRALRRLNLDLGLQEYIKSTLADAHTTISVGGSSTRSLQINNGVKQGDPLSPFLFNAVLDELIGILNERQPGGTLTPECRVSAMAFADDILLLEDRDLDVPISLGTTMDFLSASGMELNPSKCSSISASVVSGKSVPRSRPVFKIDGQWIRVLAGPSSFRYLGLLFGSQGALKPSLHNLIGWLNNLERCPLKPHQKFCLLKTYIVPRLFYGLQSPALTGGILQEGDRLIRRTARRILHLNVHTGSQFLHARIRDGGLGLVQLRYKIPVILLRRLDNLRQDGKDITKWETVFNIDGPAQSLYYRVKGLASRGDPDPYWREQIATRPYSAGLQDAADDGASRRWIDSIPRGWAPRDYISAVQLRTGNLPTQALPYRAAGQRGCRHGCPRVESLSHVLQGCPATHNERIRRHDEVVAKLARHTRGKGWRTEVEPYVYHQDGQLFKPDLAIHQPNDTLVICDAQVCWEGPRTLATSWDNKRLVYDNVRFREAAARHWGKKTLVFSPILLGARGVWPRCNKPTTDILGIPPILRSSCVCSCLKWGPTLHRRFMAGVWRRRPPDRRP

SEQ ID NO: 12 - protein, 1129 aa, synthetic construct
MNRNIKASDAAPLSPGGVVADLSAPEGTQGDNTGASPDMILVPLMGNEIACPICERREIHLLFMNLSDLDRHFNEHHVEVPIKWVCRECRRCFHKLHGVRCHIPKCSGSTQASEGRFGCGVCSMRFESQRGLSTHERHAHPAVRNIKRRGTDPPSVASGRAWKAEEVALLKELDAIYRDHRFPNIEISKILTTKTAEQIRHKRKTLRNIETEGNLQEEASTTEGGCDLVEPFGVTESQERIVNSDEMVREWHQSLINEIEKPAEVPPVLREVHALLCKIWEDRNSNREALKTELEKFLVEDLNRALRDKTEKPQGNKVSSQNRKRTRPAKRSKKVLRNYKKKYTYARCQELYNECPRKLAEAIINNDTSCLEPAKQPPEANEIRRLYNGLWGRTGPTNIEIFEKSESELSLSDYFGPITVGEVEDKIKKLRKKSAAGLDGLTKENLLIPGLSAILAKFFNMLWFSSYFPTSWKENRTTLIPKPNKSSSYVENWRPITISSILSRMFSSILDGRLRSGITQNVRQKGFTSESGCKLNIELMNAALDHCKRNQGGVFTIVDISKAFDTIPHAALRPCLARKGVPAPIIDMIVGMYVKVKTIIKGKDDMGVEIDILRGVKQGDPLSPLLFNLCLEPLIESISEKTGGININDRNKIPVLAFADDIVLLGGDRREAQRQMDELHRYLETLGMNISIDKSQTFQIVTKKDTWFVDDPRIKVGSRSIPQVNPEEAFRYLGAKIGPWKGVHCGIAVPEILSMVRRVRKLALKPCQKIELLTKFIFPRFIYQLLVNPPGDGVLKLMDSEVRQEIKAILHLVPSTATGFFYAPKNFGGLGLPRFEHIIKLGTLRSALKMKGSADPAAASLIDEGADKKLKRIANSLRINWPATFEDIEKASKRLKGEHVRQWAELRSQGQGVADFAKEKLGNIWLKEHNLLKPSRFIDALRFRTNTFGTRTTLARTKRDMDVTCRRCQAQPETLGHILGLCQFTKGLRIKRHDEAKTFLANKLRESNEVFVEPSINVRGSLYKPDLVIKNEERILIVDVTVRYENKDYLQRAFKEKVDKYRSCLSLLKSTYGRAEGSILPVVLGSRGAIPTRTREILEELGLNKSEIKTIVMNVLRSSIEMANIFLDG

SEQ ID NO: 13 - protein, 1045 aa, synthetic construct
MRLSPGWPRTASANVRVLGAATPLDRLLVGPNSINIASDGLNRVGGSTDPSGPTKSSNQFPCGVENCEKTFATVRGKSVHQQRAHKNWYDEMTANSINNSKAPWSTEETSLLARQEAVLVLGGAKYLNQALLPYFPNRTLEAIKGQRRYVKHKEAVQQFLAKLQEGPHKEPPKSESETEGVDSMNALKKAIINVMRGLSPLKGSEFRADKLGRILSNVLTSSNDEIAAELEAYLLEVFPPKPDNADPQPARSKERPLTRRQMRRVQYSKTQKAWRKSPNKCLKFLLKTKSSSQVPCKEQMVPYWRTTMTNKSDLSPWSGQTPERELEILSPIIPAEVTMAFPARSAAPGPDGLTPRQLRKVPLNILVGIMNLLLIGGKSPKFLLESKTTLIPKKEEASDPGDFRPITVSPVIIRTFHKIIANRVAKAITLDDRQRAFLPRDGTARNIFDLDVILHYHRQNFKPLYLASLDIAKAFDSVSKRTINDTLSIYGVPSQIVNYLSDVYKRSTTRLSCDGWTSDTITPECGVKQGDPLSPLIFNLVIDGLLRRIPQEIGVKIDDVHYNALAFADDLVFVASTPQGLQRTLDVAAEYLTKCGLAINAGKSFTVAIRSVPHMKKTVVDGGVAFKCAGSQLPALKRADEWKYLGVPFTPEGRSKGHEIELLKLGLQQLTQAPLKPQQRLFALRVMVLPGLYHLLTLGNTTLSKLKKIDLMVRSSVRKWLDLPHDTNNAYFHANFKDGGLSIPSMRWLMPLHRWRRLRSLTQENTASSFLNLELCRARRRLRDGHQTIETLEQLDKRWAEALHTSNDGHGLRESRGTPQQHRWILEGTRLLSGKDFLNANKLRINALPTRSRTARGRIKDRSCRAGCNTIETADHILQKCYRTHKARIERHNAVVSYIKRALEKSYAVEEEPRFDSVDGLKKPDLIAIKGETAEVLDAQVVGANICLDLAHDTKTAKYRCLAKSIKEKYGVKEVCFSSVTLSYRGIWSKRSAQYLLNKKILKSGELMVISTRVLVGGLMEFRHFNKATTTSAFLTRSKQRTGIG

SEQ ID NO: 14 - protein, 1123 aa, synthetic construct
MNKAKNDSDVPGSRMARGLRASARLSERARGAEDPDGVAAGPSRITIRTSQAEVDSLEEEMVSCEHCSARFRNMRGLGVHKVRAHPLQANSAINVDRVKARWSVEETRLMARAEAEGTLAGEPFINQYLETRFPNRSLEAIKGKRRQEPYRVMVRDFCQEIQSRRAIVIDEGDRDGVVMTRADTIPAAIEASTDDQSAHQPPPEQSSQRLRDKIQSLVEQSAAFRSYEADTLAGTARDALTAGRVDVLAIERWLGMIFPRPLQVRPPQRVSAKKHCPPVKQESKEQKRKREYAIVQSLYRKNKKSCLSRILEGDSVRERPSAVEFLNCWRPIMEASSGADGDMTDLRELFGEAAQAASAGRGRTSGPRGIHLEGLGDGAATDPVPVADKTLLWDPVTPAEVNRISVRTGTAPGLDQITPRMWNAVPAILRALLFNLLLLAETIPSSIATTRTVFLEKGGMSDRPGPTEYRPLSIGSVIIRHFHKILAKRLAALDIFDRRQRGFRPVDGVCENVTVLSSVLWDARRRCRSLHVACVDLSKAFDTVAHAAIHTTLDELGLPREFREYVRAVYSNALTELQGVSAKEVSAIKIGRGVRQGDPLSPLLFNLVVDRALGILSEDVGYRLGGRLINALGYADDIVLLASTRDGLQENLTRLHDAFMRNGLEINARKTGVLSMVASGKDKKVKVDTTPYFTVGGAMIPQRSLVDVWTYLACEYTGARELASDPPLAQAIEQLTRAPLKPQQRLRLLRDCLLPRYYHRWTVGTVSAKTLKGKDIIVRTAVRRWLRFPHDVPAGYLHAPIQSGGLGMPLLRTLIPILKHNRLQRLCRSTLPAACAAAETTYVARQLVWCENQMRVRGHRVTTTAELRQQCAAWLRESCDGSGLREVAASNLSSYWVSAGADAIPGADYVHYHHIRANCIPSRARCSRGRDGRDNRCRAGCPETETPAHCVQRCFRTHGGRILRHDDLCRQVGGNLQQKGWRVDAEQAYSTSAGRRRPDLTIAKGGEAVVVDAQVVSSETALEMSHERKVEKYRSCNDLAAAVAERTGVPRNNVRFTAITISWRGVWSSRSESELRELGLTSAQLRTLTTRVLWGSWLNWKRFNAMTSRYNEGRRNVASSGGA

SEQ ID NO: 15 - protein, 1108 aa, synthetic construct
MLLIMEKANCKSTTQAGESRGAPASSPRSYSGGGNNCYTMRRRGKPASRVLPDLPQEAGDAFIDGADGAQTGEPVVRAVGARRGGAEPGEPQAGISCSVCGRTFASKIGMGVHKRRAHPLEANEEAAPAMVKRRWRDEEVALLARTEARLLMTEGGCGNLELQRELPDLGRTLEAIKGKRRVESYKALVRNFLAELETPVSQLSDSGPPELAVSDVSHQLTIVSDQPRPPPESSLADVETDRLASPARVELFAVPLSSGRRALEQVVEGLLKKGAAQRRKQGGRRSLTNQPVAGLNSRNKRRMEYARVQELYRKCPSRAAREVIDGQHRGVKHSLQEMTSYWKPILEAVSGAPGPTAEALSALVREECSGGSRDYSQLWTPITEDEVKASCVDLHSAPGPDGIRPGDWHAVPVETKAQLFTEWLARGEIPLQLRQCRTIFVPKKDDPAVPSDYRPISIASVPLRHMHTVLAKRLLACCPPDARQRGFIRADGTLENSAVLDAVLGDSRTRLRECHVAVLDFSKAFDTVSHAALVELLRKRGLPVAFCDYIARLYDTASTTLAVGGQTSDLVTVGRGVRQGDPLSPLLFNIAMDLVLANLPKDVGYELESERVSALAYADDLVLLAGSKVGMQASINAVSSFGQMMGLKLNHTKCSVLSMVPDGKRKKVHFLSTRSFRIGKRWFKQVLCVEHWRYLGVDFRSSGSTTLEQDVQQALNNIRSAPLKPQQRLEVLRGHLLPRFLHGLVLGSITEGRLRMLDVVVRAAVRKWLRLPADVPKAYFHAAIKDGGLAIPSLKTCVPDLVIRRFGALDSSEWSAARAAARSERIRRKLRWARGQFAGLSREDPRTGVRKASLYWREGLHTSVDGIELQESHRCPATTKWVRELSNNYTGRDYVQFVHTHINALPSRVRNSRGRRTSDESALNCRAGCMVRETTAHTIQQCFRTHGGRILRHNKVASILAAAMVEVGWKVNEEPRIRTREGLRKPDIIAARGGEGVIVDAQVVSGQRPLDEAHRAKRTKYGNHVELVEKVAGILGLPARRVNATSCTLSWRGVWSFGSYKELKKLLGLKEVFFQNLPSIVLRGSHTNWTRFNQTTFRADSGYDLSPV

SEQ ID NO: 16 - protein, 1224 aa, synthetic construct
MASTTNSDASDTQLPGPGSSVADLSALQGTTDDDLALNTDNETDVSSAITIRVPLQGNGTLCPICLKKNLQTSYEKLRDLTTHFINHHLTQEIIWECRDCGKCFDRAHGWSCHYAKCVGPNVLDAKPFKCEACAMSFSSIRGRSTHERHEHPALRNEKRKQASVRPQGGLAAADGRSDKGIWSVEETNKLIELDARFRLHTFPNKEIMKFLTNKSAKQISDKRRNLRLKGVIKDSESTQREVAVAELEEVEIAGTDSDSSSAEDFQDAIDQPESVNLQVDLMQEGLNSEVHDPNAENAEWRNAMAAAIENIDINNVPPRVRNLYEKLRNIWLNCKEDINELKLEIDSYVSDTLVTFLLGSDEATNGNIPAGRNARNNKKKNNDKNNDACNNNQNNNYSSSNKRNNNDNNKFDYNQNRRVRNKTRINNSNNRKKFRYARCQDLYRECPKKLADIIIKGDPSFIDPPRPPPPASEVRELYTKLWETNGPVLKEKIAARRLPLPISDILYPILADEVDKRIRKMSNKTAGGPDNIQKRHLQTQGIIIILAMLYNILCLINHYPTAWYQNRTTLIPKVGGDLEKVENWRPITISSALSRIFSSIMDSRLRGVIVQNLRQKGFTTENGCKYNTMLLNEAIKRSKYLNGGVFTILDVNKAFDTLPHSLLEPSLRSKGIPDQVINLIQNMYANCVTSIRARNKISVQVRLLRGVKQGDPLSPLLFNLGMEHLLEIIEEKTEGVPISENIKISILAFADDIVSLGKDKEEAQKQLNMITQYLDELGMKLSARKCQTFQITAKKDTWYVEDPELKIGNENVPNVDPDEAFKYLGSKMGPWKGLHQGIIIPDVVAIIKRVRKLALKPQQKIDLIITFILPRLIYGLLIHPPADCTLKLLDSEIRQQIKDILHLVPSTATGFFYAPKNSGGLGLPRFEHIIKLAILKNAVKMKNAVDPAVTGVINEKEEASFKKVANSLRINWPATLEDVERAKIRLKRDHIKQWSDLHSQGQGVQDFAKDPIGNCWLKEGYLLKPSRFIDAIRLRTNTFGTRVVLARAKKDIDTTCRRCKCQPETLGHILGLCTHTKPLRIKRHDEIKKFLADTLARNNEVSIEPQMTVGENSYKPDLIIKNEERSFIVDVTVRYEHREYLQAAHDEKVAKYKACADEIKRRNASNNCKVLPIVVGSRGTVPPHTTQNLRILGFKKKDIKTVALIALRSSIEIANIFIDYDDVT

SEQ ID NO: 17 - protein, 1126 aa, synthetic construct
MQGDRNEASDAASPGPGGAVADSSALEGTIGDDLAMSLDTIFDISIPIVGNQVACPVCEEREVHLFFMNLTDLGKHLDQHHTTARIQWSCKECGKGFSKLHGARCHLPHCSGGPSQRSEGAFKCEACPMSFGTQRGLSTHERHAHPAVRNVKRRGTDPPEKKWTFQELALLRELCETYKDHRHPNKKISEILTSKTIDQIKYQRRKLRMLGEDPQEAAPATEGGCDPVDPGNACFEEPEISGNNQIESWKLQLELAIMTQTEVPPVLRWVHTRLINTWVTLKGNREALESGINEFIKVTLYGAIKAFNKDKIKENKYKEKFFKRIGHQKKSKKRNAKKRYSYARCQELLVENPRRLADAIVNNDQAFLQPAREPPDAEEVRRLYEDLWGKTGPVDAPVPGSRASELSLSEFFPPIAVEDVAERIKKIRMKAAAGPDGFQKEHLLIPGLPIILAKIYNILCYISYFPPIWKENRTTLIPKANKDSSLVENWRPITIGPILGRIFSAIIDGRIRRGIVQNPRQKGFTAESGCKINIDLLSAALDYSKREKGGVFTIVDISKAFDTVPHSAIKPCLVRKGVPAPITELISKMYVNIKTRIRAGNNMGVDIAILRGVKQGDPLSPLLFNLCLEPLLELMETQTSGININETRKIPVLAFADDVVLLGADVGEAQRQVDMLDKYLEDFGMNISRGKSQTFQVVSKRDTWFIEEPKIRLRENTIPTVDPGEVFRYLGAKMGPWKGVHCGIIVPELLSVMRRVRKLSLKPCQKFDLITKYILPRYIYHLLVNPPGDCVLKLLDSEIRQEIKAIFHLMPSTATGFFYAPKSWGGLGLPRFEHIIKLGTLKSAIKIKNSVDPAVSSLVDDQTEMKLKKTANSLRINWPASIEDIEKARKRLKAAHIRQWADLRSQGQGVFDFSRNRIGNAWLVDNKLLKPSRFIDALKLRTNTFGTRTVLARADKNIDVTCRKCRAQPETLGHILGLCQYTKGLRIKRHDEAKMTLAEALRKKNEVFIEPTLKVGGILYKPDLVVKNEERVLVVDVTVRYENKDYLSKAEKEKSEKYRPCLEHLKAKFNVSRGQVIPVVLGSRGAVPLFTERCLKLLGIGDQTIKTMALNVLRSSIELCNIFLDE

SEQ ID NO: 18 - protein, 1291 aa, synthetic construct
MVKAIVKNMSALNLPLVVQSGWEASRINNLPAAAVVTTRSVKYQTRSYRDTSRSTSTLSEEGSAIPEAYLAPCGDSGVASRITVPEGHKEADLTTEVESAGKTANPPAEGSIKTLTFYNKSFHKTNKGTVTSHPNENSLLGEKDHINVLDNNTETIVVRPSHDRTTIKEVVVQYPVDGLLCKPCDTLFPKIGLFAQHLRRTHQTKIIRTQCSRCHKTGEFHSIACHYSKCKGAVQTVMGEFQCEVCEANFTTKTGLSQHERHMHPQLRNNKRIDANKPSAKRRKGVWSEEEETLLEQLEINLAGARFINVEIAKVLTSKTSKQISDKRRLLQQKRLAKAAAPTSPQTVEREEQEPIQEEENGTDIPVEQQFPYKIPAKLPKHREVDEQIRQAEELLKKANDPLCMAQARGLVDCITDEINGGHAGGHSRRRQKECKRKAEKKGNNNGARKRFATKKAVFKDTQKLYQTNRRQLARKIMGAPDTSKCPISREELEKKFREKLSVPNNKADLRKFAGYAGPVDETVLLGPIREEEVEEALSGIDKHSAPGPDGKKLKDLEAIQEADASRIPRLFNLWLKTKAVPETMKKSRTVLIPKCEDPDRLKNLDNWRPITIGPMLLRLFTKILAKRLSKAVQINPRQKGFLAATPGCNENIAILQNIIKGSKKNRKELAIVFVDLAKAFDSVGHKLLISSLKRMKLPNAFVDLIRDLYTNNTTVIEGKGKPTEQIKIERGVKQGDPLSPLLFNIAIDPLISTLEQTGQGITMPYGDRMFNCTALAFADDIALLSNSHAGMQENLKILQSYCDKTGLDVNVNKTKGFHFTCKRKTFIYNHVAKWKIRNEAIKYIPPGDTEKYLGARIDPWAGVKESEWEEKLKAWVEGLRSAALKPTQRLELLKVHVIPRLYFHLILTEASQNTLVKLDQIIRNAVKEFLHLPQHVTDGLLYSRNRDGGLAIPKLEVQIPSAIIRKRESLERSTDPIIRASFQFREESNKETIEEYRSLNVLQEIKGFLVEKNIPVVPDPEVLTMMLEGGQLSEKQKQRLLKPPNEYGAWREWEFEKWSNMECQGTGIKYFHGDRISNAWLKPHAKNRSRYVISSLLLRSNLYPTRTTLSRGNPFANKSCRRCNDRQETLAHISGYCPFVKNKRIKRHNKVLDHLRQYVGKYGWTSYLEPRLVAKDGSLWKPDIIFKKDDSSKVAVVDVTVRFENNKQSLEDAWTEKTQKYKHLSEEVERLTGGTKPQFFGFVMGARGKWFGKNDSLMKFLGIKRYKTFASKVSRETLSMTLQLMQIFND

SEQ ID NO: 19 - protein, 1140 aa, synthetic construct
MDAIGYVCVGFITKGKMDENDNASDAASLGPGDAVADSSAREGTSGDDLAMSPVKILVPLVGDQVACPVCERREIHLFFMTLGDLNKHLDQHHVDARIQWGCNFCEKSFPKLHGAQCHVPKCSGTNQSKNLPHKCEACPMSFGTQRGLSTHERHAHPAVRNAKRRGADPPNRMWKVEEVALLRELEEIYKNHRFPNVEISKILTSKTVDQIKYKRKQLKQASEDTSPQRATQVTEGGCDPVNSGNARNIAPESSGISNDSESTQQWRLDLLNAIDQPIEVSPVLREVYDRLRNILMDYRGNRETLGFKINGLISTTLYGAIKENSKDAALTKPLEKPRVQANKKNKINRNSRKRYSYARCQELFLECPKKLADVVVNNDRAYLEPARQPPSEETVRRFYEDLWGQTGPTNPPIPSSGTSELSLDRFSPITAEEVGERISKIRKKAAAGPDGITKEQLLIPGLPALLAKIFNICLYCSYYPTAWKDNRTTLIPKPNKSSSLIENWRPITIGPILARIFSSILDGRIRRGIVLSLRQKGFASENGCKINIDLFNSALTHCKRSKGGIFTIVDISKAFDTIPHSAIRPSLERKGVPAPISELIENMYLSTKTTIRAKDGMGVEVSILRGVKQGDPLSPLLFNLCLEPLIEKIEEQTSGVHINDNRSIPILAFADDIVLLGKDKGEAQHQVDILHGYLRDLGMKVSAEKSQVFQVVAKRDTWFLDDPKIGLDNRDIPAVDPNEAFKYLGAKIGPWKGAHCGVIVPEILSMARRVRKLSLKPCQKIELLTSYIFPRYIYHLLVNPPSDSVLKLLDSEIRQEVKAILHLMPSTATGFIYAPKNCGGLGIPRFEHIIKLGILKSAINMEKSIDPAVSGLIDEEANKRLKKIANSLRINWPATLSDIEKARKRLKYDHVRQWAELRSQGQGAYDFARNKAGNVWMEQYNLLKPSRFIDALRLRTNTFGTKTVLARADKKMDVTCRRCRAQPETLGHILGLCQYTKGLRIKRHDEVKSILANQLRKKNEVSIEPTLRVGSNLYKPDLVIKNEERILVVDVTVRYENKDYLRKAEKEKIEKYASLLQHLKNKFNIGDGEVLPVVLGSRGAITPNTLNNLKKLGIDPKHIKTIIMNVLRSSIEMSNIFLDH

SEQ ID NO: 20 - protein, 1017 aa, synthetic construct
MTTTVGGPTGLSGPTLTPSDDSNEDTEPEPAVLFPCRMQGCGRAFQTSRGRGVHEQKVHKNWYDEQQALSITNRKAPWSDEEAALLARHEAHLTIMGERFLNQALAPYFPNRTLEGIKGQRKYARHKQKVLDLIRELREAPGSPQDRGIADEHQSEAVDSLRDKIRDGIEALEPLSGTEFIAEQLDLICKHIKQWSDERIFEELEIYLQGLFSAPPEVRRKNEVFAPDRTPTNRQKRRAEYAQTQRAWQRNPCDCLRTILKDKVAANVPNKETMVSFWRAVMTDTSDVTPGVGVRQPIIEAIWEPIGGDEIKKAFPEVSTAPGPDGITARQLRAMPLEILARIFNILLICGKLPERLLKSRTTLIPKKDNAVEPGDFRPITVSSVITRTFHKILAKRLMRHIALDQRQKAFIPADGCAENTFKFDLLLRYHRQNFKPLYLASIDIAKAFDSVTHQSIIDTLGTMGLPSPMISYVEYVYRNSKTKLSCNGWVSDEIHPTCGVKQGDPLSPILFNMVMDRLLKRVPREIGVDIAGEHYNAFAFADDLVLIASTPQGLQMTLDIVTGFLAECGLAINTGKSFTVAIRNVPHMKKSVVDSNIRFRCDGRDLPSMKREDEWKYLGVPFTPEGRTLVSPEGQLREALTKLTKAPLKPQQRLFALRVMVLPGLYHVLTLGGTNLSRLKKVDTLIRGAVRKWLALPHDTVNAYFHANAKDGGLSIPSMRWLMPLRRRERLEGLVRRGEEPGPFISQEISVSRRRLKEGQTDLNCKAKIDKRWADLLHASSDGKALMLSREVPQQHQWVTEGNRFLSGKDFINSLKLRINALPTNSRSKRGRIASRGCRAGCNEIETLNHIMQRCHRTHAARVARHDAVVSYLARALEKKYERVDVEPHFQTPEGLRKPDLVAKLGQTALVVDAQVVGEQVDLATAHERKINYYQELRNDVEARYEVTSVVFTSATMTCRGLWSHTSVEDLIRMDVIKKKEIKVLSSRVLIGGMNAYRIFSKSTTTIRRRPRTGIG

SEQ ID NO: 21 - protein, 1118 aa, synthetic construct
MLSVPSSGDSDGGNTNQGVFNPLVLSITSNSRSRQKAEVPDRCPSCCIQLTDRTRLSAAQHVRRCKAPPAQIDGYACEICASKFKTYPGLRQHIRRTHTQQYNNMDLELTHERQKTARPQYSDGEIKAIACFEATIPNRSNLLSKSINEIISHETGRTVDGIKKLRQKQVYKTYLSEEIDNLRRSKLRELCPLTPRVLLSRVDDTPSVSQRSPPHSTELPITEPQDTPHVHPESADDMCALISSREICSALNGIRGDTSPEISAIIDSIINNKHATTVIDLLDRYISTIATPNRLKSKRRRRSTFKNSEAKIKGNRAKRRASQYATMQKLYQNNPTQAAEIILSGSTLDAPETPAMSEFEDYYSNIFAYKDLNWKSQASQPKCTVDISHPINLVEIHSNLLKLKESSPGLDGIKREHLRSMPRSDLLALLNIIWGMKCLPPILRSNRTSLIPKSSNTKELKQWRPITISSRVLRLLNKIIVSRLENEIQLSYSQRGFTKTDGIIANNSILQTIIRTMRNSSKPFVILSIDLAKAFDSISVVSIVDALRKRGVDEHTIQYIEDNYHDNTSVLECHGKRSKPIKINRGVKQGDPMSGFLFNTVVDGLLQKLHNRHGIKIGDINIVSLAFADDIVITAPSPLIMQSHIKIIESYFKRHGLAVNVDKCAAFQYISVPGTKRLVVETKSLLSINGTAVPTLGVSSQLKYLGLQYTFRGVASPTPSKLEEMLDRLKKCPLKPWQKLNILQRYLIPRLHHGMQTMDVTKKKLAYLDSCISKFVKATLHLPITTPSAYLHAPLKSGGLGIPSLQLHIASIYLRRLERLSLRGDSETRVIMRTPVIQQLCVKLRRILSGVNVATKSAVQDHWAEVLHSSALGAGLKSMSPGVSSWIYQPPIIWKGRDYIGAVKLRIGLLPTKGAPYMSDTDCRNPSCAGTRESLYHVLQRCPVTHYSRINRHDSINKTLKTSILKTGLMCEEVPRLDTKADRYVPDLMCVKEDTAYVIETTVAYESKPTSTANAYQIKKDKYSSPDLIDKIKQIYRVSDVQVMPFVVGARGSWIASNDAVINTFKLPRGLRPIICNLALQWGVSIHRQFMSTVWRVAKQTTLPGAQRVNYKRRKKKV

SEQ ID NO: 22 - protein, 1080 aa, synthetic construct
MNDVPAARPPPPQRFCPYRRPSTVPPTPMSPPTISGTDPGPSGVRSPRLGLPCPYCDRSFPTAIGLGVHRRHAHPDQANAEVNVERAKKRWCAEELRLMAREEALAVVQNVQFINQHLMNALPMKRTLEAIKGARRKAEYKDLVVHALHDLQTSEGADDGQNNSDPSLRSATDELGDNPPRSDGASVLTPSTSPARPPSPRSTWGSDTRCNGFTLAMQELIPVARCIRGFGSLTLVRIADNFIRGSDVTTQLCAWVREVFPVPPPRRPRPLLEPHATIKVPKKRLRRREYARVQQLMKTHMSRAAKEILDGRPEARTPDVATMAAHWGPFVSQESFPVAEIRRTPPSPSLEYMWHPVTCDEVMEVTMAISSAPGIDGITTRQWKAVPASIKALFFNIVLASGGFPPEMLTSRTVFVPKRDGAGEPSDFRPISIASVTVRHLHKILAKRLREAGLVDVRQRCLDDGCAENISMMATLLEDARTHLKEIHVVSLDVAKAFDSVSHQAISAVLGRTGLPEEFSNYTARLYANSTTMFEVEGVRSEPFHVRRGVRQGDPLSSLLFCLVVDEVLRSVPRDVGYEIGGCRINAFAYADDIILISSTGLGMAAALREVETRAGEQGLSFNARKSVCLSIVPAGKQKKYKVLVDPQYRLADGEPVKQLSPSEQWRYLGVDFRPVGLKRAGSVLNTEMDRLTRAPLKPQQRLKILRSFLIPRYYHALVLSGTTLGKLRALDRQVRVAIRKWLRLPHDTAVGYFHTPCRMGGLGIPSFTTTIPGLMLTRLSNLATSSSPAVRVIAASGWVDRRLSWAKRALTLDGEYLPSKERRDEWWARRLYRTVDGYELRECGVSNLSNWWVDAGSYGVPGRDYVQYHHVRVNSLPTRIRTSRGIRRETRELRCRAGCRVTETAAHIIQACHRTHGGRIKRHDAVCKVLASGLRDAGWTVTVEPRFHTSAGLRKPDLVAKRGERISVIDAQVVSGARSLDDQHERKRSYYADNPDITRTLAEESRVSPRDISFSSCTLSWRGVWSSKSADYLQGLGLPKRLLCGITTRVLQGSHTNWVRWNRMVDMGTREGHPRVGVG

SEQ ID NO: 23 - protein, 1248 aa, synthetic construct
MVQSLALRYVSTATQTDPLDLRVYDVIVPSVPSLPREEGFDLVSLEFVNSSIVKGDGIHHADVWEFLGQLPEPYEISPMGLAQDEVLSAEGSIEEPRTPEAESRRGSTEEPKTPEAPDGSNAIGTTPKTPVVSMPDKQPSCPRCETRFTRMLGLIDHLKRVHGQRKILFRCAKCGKQNLKYHSISCHIPHCKGMVCVAPTGDWVCEVCQRGFTTKIGLGQHKRIRHPLVRNEERIVASHPKESSARGAHKRVWTEEEEALLIQLVDKYTGKRNINQLIAEHIPTKTAKQISDKRRRLVRDPSAEDHESLVGAVGIEHPPESVKLPKEGQLKLQYREKIKAGLSAGKFTVYREAFEKVVDGQDPGCVVNDVYNEFLGILGEPSQSKSICKGRKKQAPRGEKTSKTRPNWMCKRAIKRGKFLRFQLLFKSHRRRLARIILDSTEAIQCQIPSGVVHDHFKERWDTSGLFGGLLGFPPYKEANNAVFEALITEKEVDKNLKEMCKTSAPGPDGITLGQIIAKDPRFSRLTEIFNLWLVTGTIPDALRDCRTVLIPKSSDVEKLGDINNWRPITIGSVVIRLFSRIATARLSRACPINPRQRGFICASGCAENLKLLQLVVKTAKRERKHLGVVFVDIAKAFDTVCHQHVFEGLVQRGVDPHVIELVREMYRDVRTYISVGKGKTDPIYIRSGVKQGDPMSPLLFNLAMDPLLCKLEREGEGFHHGGWKTTAMAFADDLVLLSDSWEGMRRNIKILEAFCNLTGLSTKGEKCHGFYIKPTKDSYTINDCPAWIINGSPLNMIDPGSSERYLGTWVDPWTGFADPRLSEKLTDWLHRIGRSPLKPLQKVDILRTYTIPRLIYLADHTEVKVGLLESLDQMIRNTVKEWLHLPQSTCDAILYSSFKDGGLGLIRLVALIPSIQARRLHRLAQSSDETLVSYLREIHLERLYERLWLKAGGTQESVPSIWEPLPEVELGGGDEARLEWEAPAPKVSYPKPCNWRKLEFQKWTHLVSQGHGIENFENDEISNAWIRRFGGIPHRKLITALQLRANVYPTREFLARGRQENVVRSCRHCGARYETVAHIVGNCAITQEARIKRHNTICETLSEEAKRECWTVYEEPHIRDEANELFKPDLIFVKGLKAVVVDVTVRYEHSDISLSEAASEKAKKYQRLHKQIEDLTNAKEIEYVGFPVGARGKWYGKNTELLTALGLTKTRLERTARALASKVLFASVDIVHIFVSKARSIVPVNIL

SEQ ID NO: 24 - protein, 1176 aa, synthetic construct
MESSENRNINLVHQEVSDPPVPADGRGVRAGPHASSSCDRNRQSESEERVVVPFGGPNPCPVCRKRGRIFIFIRPTELNKHIQEEHLSSRITWSCRRCGKTFDKLHPLSCHLPRCKGEQVIHEHRCSSCEMSFPTKIGLSQHERHRHPTVRNETRAAVANRTPKPKGAPSSVWSEEEVRELVALNETYAGMRNINVLIAQHMPNKTNKQISDKRRQLSINRPRSETHDPTPTGSDSEEQPEPAEETDTSIEPSPSPPSVYRGSNSATPAAHESSEISTGPSEDHGTWREKLKEAVLLRNDVPLAWTEWCRKMDEIASMWPVDTAQIEHLYEAMEKQLCSLEKDENVPRVRDEKPNGGNVTNNRRNHGRKSKQFKQPSRSERRRFGYARCQELFNNEPKKLADIIASNDMSLLQQHSLPEKAQTVELYKRLWGVEGPTEALPFDGRSSHVTDIEDVLIPISACEIEDKIKHVNNKSAAGPDGITKRDLKSDGVSVVLAKFFNILLMCKYYPNQWRKNRTTLIPKVGKDPSDVKNWRPITISSMLSRLFSALVDRRLRRIIDQSDRQKGFKHENGCFANVRILSAALRILKETGGVLGIEDVAKAFDTVPHAAIRPALERKGVHPEIAAYVASMYNNCETYIKTKEGNVPITLKRGVKQGDPLSPLIFNLCIEPIIERLQADTDGIKIDGLNVAVMGFADDLVLMGANKEMAEKQMRMLVEYLEAMGMELSVNKCSAMEFVRASKSWFIRDPGININGVKVPSAQADEAIRYLGTKITPWKGLIENFECDAFQELMVRLKRVSLKPMQKLELLTTYLLPRYTYGLIMNPPSQNTLRKIDGLVREEVKQMLHISHKTNTPFMYVPKRLGGLGLVNIESMVKIAVLKNAEKAKESNDPITGLAIQYEKIEPRLKEYAKALGVSWPTTIEELDLCKNRIKNDFVKQWAQLKVQGQGASEYLQKESNAWLIRNDMLKHSRTADAIKLRTNTYPTRVCMKQFVANGQPNFDTTCRRCRGPQETLGHILGECCATKNKRIQRHNEIRDLIANKLAANHAVCLEQSFQSEAGTLKPDLVVKMNENQAYVVDITVRFERGDYLNIAANEKKVKYLRLIPQVVRQLGVTTASVLPIVVGSRGAISTATLRELKKLGINRNERITISMIALRSSIEMANEFMDYGRNI

SEQ ID NO: 25 - protein, 1232 aa, synthetic construct
MVQSLGLRFISTGTQTDPLDLRVESAKVVSPPASQSEDVGFHLVNLEFVNSRFGYDPGIILESVREELAKFPEPFEALPSRPPISVGSADAQGDGEEPKTPEAPPGSKEGVTPVATPVVSVPDKTPSCPLCATRFTRMLGLIDHLQRAHGQKKIWFKCSKCGRQNLKYHRIVCHVPHCKGEVCVVPTGAWVCEVCRKGFPTKIGLGQHKRLMHPLVRNLERIAASHPKESSLKGAHKKVWTEEELALLKRLMVTYAGNRNINKLIAEHIPSKTAKQISDKRRLLMKSPSDMVTTGEEGVLQEGTEDQAQVVSLPKEGHLKLHYWDSIKKGLSVGRFTRCKRAFERILEGEDPQKVVNDCYEECFGDLGSLSQIKSKGSRSQRPQSQKASRKTANWMRKRAKKRGKYLRYQRLFDLHRGRLARVILDNSDALQCDIPSDSVYLNFKARWESSAEFKGLEPFLSYKEADNTKFRALITEKEVEKNLSEVKRTSAPGPDGFTVGHLKKWDPKYSLLSNSFNLWLATGTIPDALRACRTVLIPKSSDPERLRDVNNWRPITIASMVLRLFSRVLTARLDRACPIHPRQRGFIRASGCSENLKLLQLVVKYAKSEHKYLGVVFVDIAKAFDTVCHRHIIKGLEQRGLDPHLIHLIGEMYETITTCITTKKGQTDPIKILTGVKQGDPMSPLLFNLAIDPLLCKLEQIGKGFHHGGMNITAMAFADDLVLLSDSWDGMRDNIKILEAFCDRTGLKTQGEKCHGFYIKATRDAYTINDCPIWTIGGTPLNMIDPGHSEKYLGTWVDPWTGFADPGLSEKLEKWLHQIGDSPLKPLQKVDILRTYTIPRLVYLADHTEVTAGLLESLDLQIRAAVKGWLHLPPSTCDGLFYASCKDGGLGILKLVALIPSIQARRLHRLAQSSDETLKVFLKDSHLDALYEKLWVRAGGVKESIPRIWEPIPGLENVVVGDEELSEWEAPVQKFRYPRPCNWRKSEFQKWKQLAAQGHGVENFENDTISNGWITRYKGIPHRKLITALQLRANVYPTREFLARGRHGDCVRSCRHCGAEFETTAHIVGNCAITQDARIKRHNNICEILSHEAKKENWTVFQEPHIRDEDNELFKPDLIFVKEEKAIVVDVTVRYEHSNSSLKDAAAEKVRKYQPLLNQIEELTNAIVVDFVGFPLGARGKWFAKNSELLSALGLSKTRLEKTARTLASRALLSSVDSVHIFSSQARSIVP

SEQ ID NO: 26 - protein, 1278 aa, synthetic construct
MVSVATQTCKEDFITSRPTKLKGNDSELGLLVNFSFEVYRSDLKGDVQFEGVHFPDNWGVLDGFPEVYEQLAPHTSEGDDLDPSLPGDSEGDVLEKGSSEKEKEVTPETLPPKQKAHSEQSQDNIVKVTVPDKNPPCPCCGVRLNSVLTLIEHLKSSHGKRRVCFRCAKCGRENFNHHSTVCHFAKCKGPQTERPPVGEWICEVCGRDFTTKIGLGQHKRHMHAMERNQERIDASQPKETSNRGAHKRCWTKEEEELLIKLEVQFESHKNINKLIAEHLTTKTAKQISDKRRMLLKKGRVTTGHLESEPGVSHQSQAEVKDIELGGGPLSGGLAVDKGTIGKLGQHLEKGNGHQETADQKKERGLQARYKRRIMKRLAAGTINNFPKVFKELLNCQEARPLINQTTEECFGLLDSACQIRTALRDKGRPQEERPRKQYQKWMKARAIKRGNYLRFQRLFHLDRGKLARIILDDNESLSCDISPSEIYSVFKARWETPGHFNGLGDFEIKGKANNKAFRDLITAKEIEKNVREMSKGSAPGPDGIALGDIRKMDPGYSRTAELFNLWLTSGDIPDMVRGCRTVLIPKSTAPERLKDINNWRPITIGSILLRLFSRIITARMTKACPLNPRQRGFISAPGCSENLKLLQSIIRTAKSEHRPLGVIFVDIAKAFDTVSHQHIIHVLQQRRVDPHIVGLVNNMYNDISTRITTKKDTHTDKIQIRVGVKQGDPLSPLLFNLAMDPLLCKLEQSGKGFHRGQSSITAMAFADDLVLLSDSWENMKENIRILETFCDLTGLKTQGQKCHGFYIKPTKDSYTVNNCPAWTINGTPLNMINPGESEKYLGLQIDPWTGIAKYDLSTKLKSWLERIDRAPLKPLQKLDILKTYTIPRLTYLADHSEMKAGALEALDQQIRTAVKDWLHLPSCTCDAILYVSTRDGGLGVTKLAGLIPSVQARRLHRIAQSPDETMKDFLEKAQMEKMYEKLWIQAGGKKKGMPSIWEALPATVPPVNAGTLSEWEAPNPKSKYPKPCDWRKKELKKWTKLESQGRGVKNFRNDSISNDWIQYYRRIPHRKLLTAIQLRANVYPTREFLGRGRGDDCVKFCRHCEMDLESCGHIIGFCPVTKDARIKRHNRICDRLCEEATKRDWVVYKEPHLKDATRELFKPDLIFVKEGRALVVDVTVRYEAAKTTLEAAAAEKVDKYKHLGPEVRELTNATDVVFMGFPLGARGKFYKGNFDLLETLGLPKTRQIMVAKTLSTYALMSSVDIVHMFASKGRKSNV

SEQ ID NO: 27 - protein, 1183 aa, synthetic construct
MAEVNNLSLGGGEPIGPISPTVVGSTNQGTSDDEEGANRPIDRIMELVEVSVPIDPANTLCEECLQHGQRRGFISLKNLDTHFKASHPRIIVIYICRVCDKRCPSKHSWDCHRPKCPGGQNERRGILKCDHCERYFDTQRGKSQHERHAHPVVRNRAREAEANREWAPQGRPPTRWTPEEFALLKQLNLRFKNSRFPNVEIRAFLPGKTLKQISDKRRDIPNEESEHEDTERPSSSDNEEADDSNNIDTDSTNDLEDQTDLAEAENNDQNENDIDESHVRWLERMEEKIRSIELPEANRFYNEHKQLIEIWDTYKNEQQELELKVNEFISRMTTLLVGDGAQGLPSGTRPSKNKEANERHARTRPMNGNQRRKHKYARCQDMFKNCPKRLAEIAISGDFSFVEARKEPPPVEVIHGLYDQLWGSEGPLDTTFMLEEGQYEQQRINDVLDPINLKEVKDRIARMKSGSAPGIDGIKKGHIAKTGVSELLSVLYNILLMHNIFPEEWSKNRTTLIPKDGKDHKDIKNWRPITVGSMLGRVFAGLIDQKMRKFVKQSIRQKGFTAEDGCRSNTLILDKAIEQMKGVHGGAVMIADVSKAFDTVPHSAITTALKRQRVPRHLIKLVEKMYDNCSTSIKGPNGGRVEIVLKRGVKQGDPLSPLLFNLIMDPIINVLDQEGQGINLEGENLAILAFADDIVLIGRDTERAQAQTRKLDEYLSKLGMSLSAPKCSAFSIVASSKTWYTRDPKLTIRGQAIPAIDADEICRYLGTKFSPWTGLQRGREIPKIMEAIRNTRNLSLKPYQKVALITTYILPHFMYELTASPPAASILKVLDTDIRNHIKEILKLPISTANGFLYTARRDGGLGLIRITRAVQLLTLKSGITTAASNDPILRAIRTNPKFRRKLERSAEVLHIARPEGMEEYEKAKNDTRQQERLSWKNLKSQSHGVNDYMGDKIGNCWLKNPALLKSSRYLDALRLRTNTFGTRVVLSRTKANTETVCRRCGLMPETLGHILGQCVSTKPARIKRHDEIKNLIAERTGRRHVVMVEPTIDEGGTLKKPDLIIKTNNEVFIVDVTVRYEKDSYLADAHKEKVEKYKDTALLMAQRLGTETGRVLPVVVGSRGTLPRRTITALEKLGISKKDRMTIALTALRSSIEIANAFIDYDNGRNHRPANFNIDNAQRMNF

SEQ ID NO: 28 - protein, 1097 aa, synthetic construct
MGVFGPQTEQLLRKGFTTAVQCPSGRASHPTHIWFKLVANDDNKETYGRSSGNPQEPEVPTLKPMCPVARSSPQSGRGECDWNCPECSRSFRTKTGLGVHRRRAHPVTANAAAAPPQVKRRWREEEGELLAQAEARLVRAGGGAMNINQDLMRELPQLGRTLEAIKGYRRKESYKSRVQECLADLPRTPSPGTSGEASLPSRGTPIAGTTDSLTPERPHVRAESPLEDVPVSVNMQSPTLPDTLDLARIVEDLLASSEARMARGARGGGRRGRPRRKGQSPEETLTANARLSARKRRRMEYARVQELYKTCRSRCAAEVIDGQTRGVRHSLAELEAYWRPVMEAVSDAPGLTPEVLNALKRSELSGGSRDYSQLWTPFTPEEVKASRIDNKSGPGPEGINPGAWNTLASATQAEIFNAWMIAGEVPDRLRGCRTVFVPKTETPACPGEYRPISIASVPLRHMHSVLAKRLEACCPPDARQRGFICADGTLENSAVLDAVLGDCGKKLRECHVAVLDFAKAFDTVSHAALVDLLRKRGLPVGFCDYVARLYATSETVLVVNGTRSGPARVGQGVRQGDPLSPLLFNMAMDAILAALPREVGYKLEGERVSALAYADDLVLLAGSKVGMQSSIDCVWRTGKMMGLFVSHAKSAMLSMVPDGKRKRVHYLTDRTFKVGSRWLRQVSNVERWRYLGVDFQASGCVTLEHDVKEALMNITKAPLKPQQRLEMVRVHLIPRFLHGLVLGVITDDRLRMLDVQIRKAVRTWLRLPKDVPVGYFHAATTDGGLAIPSLRTCVPDLLRKRFGRLDSSRWRVAKAAARSDRIKRKLLWADKQLRKFTAENPKSGERTTAMYWRETLHAAVDGFELRDSPRVPASTKWLRERSVQYTGRDFVQFVHTHVNALPSRVRNTRGRRTGVASELNCRAGCMVRETTAHIIQQCHRTHGGRIKRHNCVADVVGSAMMDKGWTVVKEPKVRTALGLRKPDIIASRGGIGVIVDAQVVSGQRPLDELHRVKRNKYGKHEELVEKVAGLLGLPCKDSVHTTSCTISWRGVWSLASYRELKRLVGLDEGVLAGIPSLVLRGSHMNWTRFNQMTTRID

SEQ ID NO: 29 - protein, 1133 aa, synthetic construct
MFEENNRINIMVNKDIESASDAASLGPGDVAADSSALEGTSVDDLASSPDTIDIMVPIVGDQVACPVCQKREIDLFFLSLSDLGRHLEQHHVTARIQWRCKDCGKGFPKLHGARCHLPHCRGTSQGPSGSYRCETCPMSFGTLRGLSTHERHAHPAVRNVKRRGTDPPEKKWTAEELAQLRELWEIYKDHKFPNKMISEILTSKTIEQIRYQRKKLRLIGEETSSQEPTQVTEGGCDPVDPGNACFEEPGVSGLEYEQWRLQLESAIMTQVEVPPVLRWVHTRLMTIWVSLKGNREALESAINEFLEFTLYGAIKEINSKKRASSVKNKRIGHQGKKKGNRNARKRYSYARCQELFTENPRRLADAVINDDRAFLQPARDLPDAEEVRRLYEDLWGQTGPVEAPVPGSRASELSLCEVFPPIAVEDVAEKLSRIRMKVAAGPDGFQKEHLLIPGLPTILTILYNILLYCSYFPPIWKENRTTLIPKPNTSSSLVENWRPITIGPILGRIFSSIIDGRIRRGIVLNPRQKGFTAESGCKINIDLLSAALDYSKKKKGGVFTIVDISKAFDTVPHSAIVPCLRRKGVPIPITDLIRNMYSEGKTTIKSKNSTGVEIKILRGVKQGDPLSPLLFNLCLEPLLDLIEQQTSGISVNETRKIPVLAFADDVVLLGADIGEAQRQVDVLDEYLNGLGMTISRDKCQAFQVVAERRTWFIKEPKIKHGNNTIPTVDPDEAFRYLGAKMGPWKGIHCGIIVPELLSVVRRVRKLSLKPCQKLELITKHIFPRYIYNLLVSPPSDSVLRLLDSEVRQEIKAIFHLTPSTATGFFYAPKSWGGLGLPRFEHIVKLGTLKSAIKIKDSIDPAVSSLVDEKYNEKLKKMANSLRINWPASVEDIERARKRLKAGHVKQWAELRSQGQGVPDFSRSKAGNVWLKDCELLKPSRVIDALKLRTNTVGTRTVLARVDKNLDINCRKCRAQPETLGHILGLCQYTKGLRIKRHDEAKMTLAESLRKNNEVFVEPTLRVGGALFKPDLVVKNEERVLVVDVTVRYENKDYLSRAEKEKVNKYRPCLEHLKARFNVGKGEVIPVVLGSRGAITPSTESNLKRMGIGGQIIKTLVLNVLRSSIELCNIFLDT

SEQ ID NO: 30 - protein, 993 aa, synthetic construct
MTRQESEQFLCRMPGCNRVFPSLRGRGVHEQRAHKNWHDERQVEMLDSRKAPWSAEERALMARQEAKLTIEGVKYINQALIPLFPNRTLEAIKGQRKRADYKDKVLELIRDLEDTDSADQLQQQLQDTSDDLRLRTNDMTAAIIEAHQGMSPLEGNDFHVDELGRIYDNIGTWPLERTFEELQLYLLKVFPIPPRKKRKSPREYSDQAVTKRKARRIEYARTQRAWKRHPCKCLRTIIKDKVSAHLPPKDVMLPFWNAVMTESDSQSPGIGNRSPVKAELWRPIVPEEIRRSFPELTTAPGPDGLKTRQFRAMPLDVIARIFNLFMLCGKMPKHLTASRTVLIPKKDNALEPGDYRPITVSSIIARTFHKVLGKRIMCTLELDPRQKAFVPVDGCNENIFDLDLVLRYHRQNFKPLYMASMDIAKAFDSVTHNTIRDTLMNVGLPDPMVRYIVGTYEQSTTRLVCGDWESDDIHPTCGVKQGDPLSPIIFNMVMDSLLKRIPPEVGVDIGGVHYNALLFADDMVFMASTPNGLQKSIDIAAEYLAQCGLNINASKSFTMSIRNVPHVKKSIVDAKMKFRCLGKTLPSMSREDEWKYLGVPFTPEGRQGVNEVARLKEALDKLTKAPLKPQQRMFALRVMVLPGLYHLLTLGHTTLSKLKKVDTTVRGAVRRWLNLPHDTPNAYFHANAKDGGLSIPSMRWLMPLRRKERLRAMVGDGEPSSYLDLEIQRATRRLSEGREEINSWQSLEQRWTKLLHSSSDGKALKDARKVPQQHSWVTDGSRFLSGRDYINIVKLRINALPTRSRTARGRRKEKICRAGCNETETLNHVLQRCHRTHGARIERHNAIAKYLRRALGNKYENVEEEPHFLTPEGLRKPDLIAVKGHEAVIIDVQIVSEQSDLRKAHQNKIDRYCQLKDSIISRYKVREVKFTSATLSCRGIWSQKSAEDLTTLHVIRKSELKILSTRALIGGLNAFWRFNKMTACRTHQRTGIG

SEQ ID NO: 31 - protein, 1035 aa, synthetic construct
MESESGESTDSADPTNLNLPSTSKDKGHRNVSENDGACRFPGCYKTFKTTRGRGVHECKAHPDWYDTKKLEVENVKIRWTDEEKLLMARREAKILFESESKVMNINQRIQEIFGAGRTIESIKGHRRRPSHKRRVQKFLTELTEQARAEAEENSESDDVSAPDPSSGRPANTSLDELARCLKDMRPLNYESYQTQRLNEICWAVPNVPKESTLINTALYIREILPPPKDHQPKASREGDRAVNRRQARRAEYAKVQDLWRKNPSRCVKTILSDQIGDEPFIDKDIMEPYWRAVMNKNSGLAPTVTAPIRTEDSLWKPISAKEVKAAYPPTRTAVGLDGISTRALKAIPVEILVRLFNIFMWCGKLPEHLYLSKTVLIPKKKDAQEPGEFRPITISSVIVRTFHKILANRLKIIQLDERQRAFRDTDGCSDNIFLMDMVLRYHRAKHRKLFLASLDIAKAFDSVSFEALVAIMRSKGLPEAMIDYIEEVYENSSTSLIHGGWTSRPIHPTCGVKQGDPLSPVVFNFVIDGLLQIIPEEIGVNLDGFKVNVAAFADDMYLMASSEVGLQELLDLVSKYLYSCGLEVNASKCLTVSLKTVPKQKKTAIDARCKFKINGRDLPALKRTDEWRYLGVPFTPEGRVHNSGLERLKPNLERMSKAPLKPQQRLWALRTVLIPKILYELTMGSAQIGYLRKTDRHIRAAVRKWLNLPNDCPNAYIHASIKSGGLGVQSVRWTAPILRRARLTALKYSMHISGQVPGAFIEKEYQAVERRLTEANNVIASQKQMDRRWADLLYKSVDGSALRNSELVDHQHDWVKGATRFISGKDFVTTCKARINALPTRSRTTRGRHRDRACRGGCGMSETLNHVLQHCHRTHGVRVKRHDAVVNYVARGLANQEWWVQKEPIFASSEGQRKPDIVAARGKTALIVDAQVMSEQTDLDRANGNKVNYYANNKSLIEAIRTTCKAETVLVLAVTLSWRGLWSRKSATELIKRGILRPKDLTIISSGVVIGGVAAWNQFNRTTSMPRTHRRTGIG

SEQ ID NO: 32 - protein, 998 aa, synthetic construct
MCVVCGRSFSTKTGLGVHMSRMHKDRFDELRLRVDVKARWNEEEIAMMARKELELIAKGERFINKKLAEVYTHRSVESIKKTRQRDTYKAKIEQFRREAARIPEANNEPSPTPRPSNNEPGPSTRSRLSPIDPLVHSDMVLLRILQDLPPVRCHQSWRVEVLQSIVDRAQVSGKVATLQCLSNYLLDIFPNRNPTPVPARARVRPQPRNRRQDRRQQYASVQRNWDKHKGRCIKSILEGPDESVMPNKEVMEPYWRQVMTQPSTSSYSPETLPWGHLLEDVWSPITIGDLKAHKVSLTTSPGPDGISAKTARSIPNGIMLRIMNLILWCGELPKLFRMARTVFIPKTGKASRPQDFRPITVPSIVVRQLNAILASRLSSSVSWDPRQRGFLPTDGCADNTTIIDLVLRYHHKRFASCYIGTLDVSKAFDAISHQAVITCLNTCGAPKGFVNYIQNMYKGGGTSLKGRGWSSEEFIPARGVKQGDPLSPILFNLVIDRLLRSLPDEIGVKVGNATTNAAAFADDLVLFASTPKGLQKLLDTTVDFLSSVGLTLNADKCFTVSIKGQPKQKCTVVEHRSFSVGSRMCPALKRSEEWKYLGVHFTAEGRARYDPAADLGPKLDRLTKSPLKPQQKMFALRTVLIPQLYHKLTLGSVALGVLRKCDKLVRSTIRKWLDLPMDVSVGFFHAPHISGGLGIPSLRWIAPMLRMKRLENIKWPHLEQSVEANSFLSAELQRARDRCQAGIELLISRTTVDTYWANRLFSSVDGSGLREANRFGAQHGWVSQPTRLLTGKAYLNGIKLRINALPTRSRTTRGRQELARQCRAGCDAPESLNHVLQQCHRTHGRRVARHNNVVTRIKQGLVRRGYVVIAEPSLQGENRIYKPDLVALRHDRTLVLDAQVVTDGLELDRAHQSKIDKYSGQDVQTALRRDHPVHGDIEVVSATLNWRGIWSYRSYTRLRALDVITASDSNIISARVVTDGICGFKMFMHHAGYHRGVT

SEQ ID NO: 33 - protein, 1027 aa, synthetic construct
MVSSLGNQQRGSIMEVLSNQTSFVCEICGQICRSKGGLGVHMARKHKDELDLARLRVDVKLRWSDEELLMMARKEIELTEHGNIRNINQRLAEAFAPRTVDAIKKCRQRDNYKSKIEQLRGQAALIPIPNVEVAIVRPSESEQDRQVSIPSTVRVNPLERSNSEILRRLQGLGPVECARRWKVDVLQTIIDRAHTLGKSGTLQCLSNYLREIFPVQNRVRTVLSRRSPNRPRNRRQLRRQQYARVQRNWDKHKSRCIKSLLDGPDESVMPNQEIMAPYWREVMTHPNPSNLTSTVVVPMNHLLEGVWDSITPAELKKFSVSLTSSPGPDGITPRVAKSVPSGVMVRIMNLILWCGNIPKAVRPARTIFIPKTVRASLPQDFRPISVPSVLVRQLHAILASRLNSRIQWDPRQRGFLPTDGCADNTTMVDLILRDHHKRHASCYIATLDVSKAFDSVSHAAIITILKSYGVPDELVNYVRTLYKEGSTTLNGVDWCSEEFVPAHGVKQGDPLSPILFNLVIDQLLRTLPREIGVKVGSAMTNAAAFADDLVLFASTPKGLQKLLDTTVDFLSTVGLTLNANKCFTVSIKGQPKQKCTVVVPQTFCVGSRTCPALKRSEEWKYLGIHFTADGRTRYYPSEALKPKLESISKSPLKPQQKLFALRTVLMPQLNHQLTLGSVSIGVLRKCDKLIRFYIRRWLDLPMDVPVAFFHAPHSHGGLGIPSVRYAAPMLRVRRLSNINWPHLEQSGVANAFISAELERARGRCQAEGNELSSRAIINSYWANRLYMSVDGSGLREASHFGPQHGWVSQPTRLLTGKDYLNGIKLRINALPTRSRTTRGRHELGRRCRAGCDAPETTNHILQKCYRTHGRRVARHNSIVKFIKGNLETRGYVVHAEPSLQGDSGLNKPDLVALRHGHAYVIDAQIVTDGHALDQAHQRKVEKYDQRDIKEAVRRDFQVEGNVEVVSATLNWRGIWSGQSVKRLIALGILNASDSNVMSARTVTGGISCYRAFMFYSGYSRFRQYENG

SEQ ID NO: 34 - protein, 1234 aa, synthetic construct
MRLWLGIRTSSWTRFRNFCIRKHNETFIFMENTTNPDASDAESPVPGTSVTDLSVLQGDQYDDMASPTDTNNTTVAVPFTIQVPLLMNQIACPVCERINANHNFLSLGDLDKHLKLKHLRATIRWVCNNCKKPHNGLHSARCHFAKCKDVEDENPDTMLFQCQACPAKFDSRRGLSTHERHRHPDIRNINRQTAPVRPPVQPTRRGNAVWSENEIDLLRQLELEYGNYRYPNKEIHKHLPSKTPKQIGDKRRDLRLASIRTASRNAQLSSTEDSSSNPDRTETIPTEEESSPDEPGEAIGGNNLTSVEDDESEWQDSLRRQVINAGTGNESEQIANLLHALKQIAENANSNQNAQNDNIESFITEQLIPFLQNNETIPNDSSNTRANKSNRKNNHNNNYNNNRNNSRRKFNNNNGINNQHRNRNNKNPHNNRNQTRRTNKNRQRKYMYARCQDLFNECPKKLADLIVNHDEFIIKPPLQPPTEEDIKELYVDLWETPGPSQINIGIKNPPKLKLQEFFTPFTGEEIIRKISKIRTKTAAGLDGIQKRHLQIPGLPIILASLFNILCIANYYPEIWKINRTTLIPKPNKDTTKIGNWRPITISSIIGRIFSSLIDIRLRSVITQNLRQKGFTAENGCVHNTSLLNNILAHCKLSEGGVFTIIDISKAFDTVPHEMIRAGLNSKGIPDKIIDLINKMYDNSSTKIRARYGKSVSVKLKRGVKQGDSLSPLLFNLSIEHLLDELEEKSTGLSTKEGPNVSVLTFADDMVLLAKNAIEAQLQLNMVVNFLKKLNMEISAIKSKSFEIVTKKDTWFVKDPKLRISDREEIPSTKPEEVLRYLGAHIGPWKGMECGTLVPEIVNLITRTRVLPLKPQQKIDLIVNYIIPRFIYQLTICPPSDSVLKLLDNEIRQQVKEILHLTISTATGFFYTPRNNGGLGLPRLEHITKLAVLKSALKKKGSLDPAISGLINEAYEKKLKTIANSLRINWPATLEDIDKAKKRLKKDYMSQWETLRCQSQGVKEYYQDKIGNSWLTEYNLLKPSRFIDAIHLRTNTFGNRTTLRRANKNVDILCRKCKAQAETLGHILGLCQSTKGLRIKRHDEIKNFVAKITSSDNEVFIEPSISAENKSYRPDLIIKNEKRFFIVDITVRYENKDYLHKAREEKINKYKPCLNYLRNKFKIKEAEVLPIVVGSRGIIPGDTKSDLLKKLKIKKHQIKTIALIALRSSIELANIFIDS

SEQ ID NO: 35 - protein, 1123 aa, synthetic construct
MSRVLRVSSPKSPGACSTLIATNTKSRPRNLDCFLVDDSAMSLDNPVDTNLDSVCPLCPSCRSDLSSRPIAQQKQHIRRCNQPPANPDGKYSCELCSSSFKTYAGLRQHVKRTHPVDYNTHDVQLTVERAASTRAKWTPAEEKQLARLELQLPTGLTQKDINIRLSNLHPGRTAEGIKKLRQKESYKTIFNEISNSNQTSSSTFSRPSIIPLSPRICLQRVDDATAIESIDHQPPITDSSLTENDGVSCTLIGHTEICAALAVLQPDCSPEMQHIIAELFDNKDFKSIDTLICAIIPKFIKSKVENTHAEPKSKKSKIMKKKRTNKHTKKHNRASIRASKFCTYQRLFAKNPTQLAHTILDDKDLQVSTFPSMAEIEREFRPVFEERPEYTCSISPYAGEIIHLDYPITFSETEINLRRLKQSACGPEGLTRDDLRSIRKADLVALLNIIFGLCNTPSLLRHNRTVLIPKKGDPSKVSNWRPITVSSILTRLLHKILAARLNQNIKLHHAQRGFTSGDGILSSSTILDTVLREHRNNGRQLYALSIDLTKAFDKVHPYAIENALISKGVDSHTVKYLMSTYDNVDTIIECHGQRSSSINMRRGVRQGDTDSPILFNITMDQFVASIDPSDGITLGDTRIGCLLFADDIVLLSETPHGMREHLHKLHDFLGKTCMEVNPSKCRALQLARVPGAKRVAVDTSPCFAIGGRDVPTLGVVEQFKYLGHNYSQRGMIAPSASNLEIMLERLRRASLRPWQKLFILQRYLVPRLIHCSQSSDITAGNLSHIDRLIRRFVKATLHLPATTPTPYFYARIRDGGLSIPCLRHQIGVIYRRRLQRISLQCDPDFRAAFQTPTIKRLLRKLDVICEGITSASSAVANYWANKLHTCALGNGLQHNTRESSSWILNPPPFWTGRDYIRAIQLRIGLLPTGGAPYINGEAAQCRYPTCVGRRETLSHILQWCPVTHFQRIQRHDTATRDLSNFLTKLGYSVQDVPRINTRDNRYIPDLLVIDTKNRTGGIIETTVVWERQSSLLEAAEIKRKKYDIPEILHSTKLKYNLDSNPQVFPFVIGARGSWPPSNDKIWNYYNLPQYLQRIIVCNVLRFGSSIHKYFMAHTWRSRKKKKK

SEQ ID NO: 36 - protein, 1015 aa, synthetic construct
MNIHNLAGEPTGSPGPTSQTGDEVVNSNSVKEIFKCEAPGCDRTFPNARGRGVHQQRAHKDWYDERQLETHQSKKAPWTTEEAALLARQEARITLEGKERFMNQALLPLFPNRTLEAIKGQRRSARHKARVLELIQEMGMEQQGNEPSPTTPTEVNDEGTNLVKKIADYFGKLKPIVGTDFRVEHLNDICGNIQTWTVDRIFEELKIYLLQVFPLPTEKQRPVRQENSTGKRLTKRQERRADYARTQRAWKKNPCKCLNVILDTKNGAEVPGKESMTSFWDTVMTSEDKSSPGIGQKAEEVLAWEPIVAEELRESYPAMTTSPGPDGLTARQIKAIPPGILLRILNLFLICGKLPQHLLESRTTLIPKKDEAKEPGDFRPITVSSVLTRTFHKVLAKRLGRLVVLDERQRAFMPIDGCSKNIFELDMLLRYHRQHFRPMFMASVDIAKAFDSVTHNTIRDTLVNKGIPDPMIRYIIYTYEQGITRLKCDGWVSEPIHPTCGVKQGDPLSPIIFNMVIDRLMQLLPAEVGLEIAGRRVNAFAFADDLVLMAQTQQGLQQIIDAATDYLSKCGLMINTGKSFTVALRNVPHMKKSVVDKRTVFTCRGRQLPAIKRDDQWTYLGVPFTPEGRLLCKPEVQLQEAIRKLTKAPLKPQQRLFALRVVVLPGLYHQLNLGSTNLSRLKKVDTLVRAAVKKWLDLPHDVVNAYIHANFRDGGLSIPSMRWLMPLQRRQRLKSLLKEGVEPAPYHAQEIAIAERRLNDSGVQITSNVELYKRWAKHLHSSSDGMALRESDNIPQQHQWVIEGTRFLSGRDYLNAIKLRINALPTRSRTTRGRIQDRMCRAGCNEIETLNHVLQRCHRTHAARVERHNAVAAYIKRALQKKHDLVEEEPHIMTTDGLRKPDLIAVKGKAATILDAQVVSEQTGLDIAHDKKVKYYENIKEAVKTKYKVDEVSLSSITLSCRGIWSGKSAQDLTDRRIIKKQELKILSTRAIIGGLNAFWKFNKSTSMRRRTGIG

SEQ ID NO: 37 - protein, 1031 aa, synthetic construct
MPRIAESYSVNSPTASVGAHQGRNVDANPVVGHLCVECGRSFGTKTGLGVHMSRQHKDRLDELRTRVDVKTRWCEEELAMMARKELELIANGERFINKKLALHFPERSVDAIKKCRQRDNYKAKIEQLRGQAALIPEISEPQTITRRPSISGLESSSSMSESVPITPSIHSDDVLMRILRGFGPVGCNRSWRVEVLQSIIDGAQTSGKEATLQCLSNYLLEIFPNRNEGPVSARVFPEPRNRRQNRRQQFARVQRNWDKHKGRCIGSLLAGPDESVMPNQEIMEPYWREVMTQQSLSSCNSTVPHMGHLLEGVWSPITSRDLQLHQVPLTSSPGPDGISPRTARSIPSGVMLRIMNLILWCGKLPTLFRMARTVFIPKTVRASRPEDFRPISVPSVIVRQLNAILASRLASSVDWDPRQRGFLPTDGCADNATIVDLILRDHHRRHASCYIATLDVSKAFDSVSHAAVIDTLSAYGAPTGFVDYVRSLYLGGGTSLNGKGWRSEEFVPARGVKQGDPLSPHMFNLVIDRLLRSLPDEIGVSVGNAKTNAAAFADDLVLFASTPKGLQALLDTTVSFLASVGLTLNANKCFTVSVKGQPKQKCTVIESRSFCVGSRTCPSLKRSEEWRYLGIQFTADGRARYNPTEDIGPRLDIISRSPLKPQQKLFALRTVLIPQLYHKLTLGSVAIGVLRKCDRLVRSVVRKWLDLPMDVSVAFFHAPHTSGGLGVPSVRRTAPMLRIQRLTNIKWPHLEQSETASSFLSAEIRRARDRCKASGDAALDTRLLIDSYWADRLHSSVDGSGLREAGRYAPQHGWVVAPTRLLTGKAYLNGIKLRINALPTRSRTTRGRHELERQCRAGCDAPESLNHILQKCHRTHGRRVARHNSVANYLKRGLETRGYSVIAEPSLQGENRIYKPDLVGFRPDHTIVIDAQVVTDGLDLDQAHQSKVEIYDRQDVRTALVRDYRAHEDIKIVSATLNWRGIWSYRSVKRLRALGVLNAGDSNIISTRVVDGGVCGFKTFMFHTGYRQGVG

SEQ ID NO: 38 - protein, 1032 aa, synthetic construct
MPRINLIDPLTTSSPTVSSLGGQHEPDAAATSVPSGLCTICGRSFRGERGLGVHMSRQHKDAFDEMRLRVDVKLRWSEEETWMMARKEVELAANGRRNINQQLAEVFTHRSVEAIKKARQRGDYRQKVEQIRGQSAPTPEVAEITIMRRPSLCELDPPVSTSEASPIIPFEQPNMEILQVLRSYRRVECNPRWRAQTLQSIIDSAQEEGKETTLHRLSLYLLEVFPAVTRPVNARPPRRPRNRRESRRQQFASTQRNWDKHKGRCIKTLLNGNDESVMPNQEYMVPYWREVMTQPSPSYVVNNAVLQPEHSLARVWSAISVQDLRATKVSLSSSPGPDGISPRTARSIPTGIILRIYNLILWCGNLPQSIRLARTIFIPKTVTANRPQDYRPITVSSVLVRQLNAILASRLTSSINWDARQRGFLPTDGCADNATMVDLILRNSHKSSLSTYIATLDVSKAFDSVSHAAVYNTLTAYGAPKEMVDYIQKTYEGGGTTLNGEGWRSEVFVPARGVKQGDPLSPVLFNMIIDQLLRALPNEIGVKIGNAMTNAAAFADDLVLFAETPMGLQKLLDLTVSFLGTVGLKLNADKCFTVSIKGQPKQKKTVVIQQSFRIGQIACPALKRTDEWKYLGIRFNADGRVRYNPAEDLGPKLLRLTRAPLKPQQKLFALRNVLIPQFFHKLTLGSVTIGVLRKFDKLVRFYVRRWLGLPLDVCVAFFHAPPKSGGLGIPSVRWIAPLQRLRRLSNLKWPHLEQSEVASSFVEEELRRVRVRLLAGNTQLHTRTEVETYWANRLHMSVDGSGLREAGQFGPQHGWVSQPTRLLTGKEYRSGIQLRINALPSKSRTTRGRHELDRQCRAGCRASETTNHILQQCYRTHGRRVARHNSVVNFLKRGLEEKGCVVIAEPSLQCESGLSKPDLVALRNNIIHVIDAQVVTDGHSIDEAHQRKIDRYDRLDIRTELRRRFGAACGIEFHSATLNWRGIWSGQSVKGLIAKNLLSKGDSNIISVRVLSGGTYCFRQFMYLCGYTRDWT

SEQ ID NO: 39 - protein, 1323 aa, synthetic construct
MEALASAQLPLSRRANSTILDTEIVPWGRCISRHFIPPVRYRVTVSTVATQTDSPGTTDGVTPLTREFTQTLTTAGEMDPISGPSQTSSTEGVISGNTIAQTEDWEPLPPLPLEFLNSVFGETVRPDTSPEPCAPTTPASEKEGDAEGSTTLADEIDNNYVSTDASTVPTTEGDGETRAQEKVLVQLPHDNPACPFCGDVVGKATALHVHLKRHHGGKDVAFQCALCGKTDPKAHSILCHIPKCKGLTLQVPAGDWPCEVCAKQFSTKSGLSQHKRLAHPMVRNAERIAASQPKQNSQRGKHKSCWSAEEELLLLDFDKMYTGEKYINKLIAEAMKTKTAKQVSDKRRLLKLYPRASSETADPGPGATPHSLEARSEAPFPTRGLHTNYMRKIRQCLSKQKQGPDHQNLAQFETEILSAWVAGDPNIRSLIEAASIDVLSSFLAPESEPKKGKRRATVPKAKSGKERSWMKKRAAKRHTYLRYQHLYEHDRCKLASIILDGTERLQCQIPLEEVFQAYKSKWETQTPFQGLGQFKPKTRADNTAFDSLISAKEVMKNIKEMNKNSAPGPDRVSLKDLVRVDPEGDILAGLFNTWLVTGIIPDGIKECRSLLIPKTMDPDALNDLGNWRPLTIGSIVLRLFSRILTNRLAKACPLNERQRGFIAAPGCSENLKVLHTIMKQAKREKKPLGVVFVDIAKAFDSVSHQHILWVLQQRGLDQHVIDIICDSYRNVHTRMEVGKELTAPIAIDVGVKQGDPMSPLLFNLALDPLISTLEMAGKGFSVGTQKVTALAFADDLVMLSDSWEGMNANIQILESFCTLSGLKVQARKCHGFQLTPTHDSFTINNCEAWKIGADTLNMINPGESEKYLGVKVDPWIGFSKPALSDKLDTWFERINRAPLKPSQKLAMLNTFTIPRLIYLADHTDCKIALLSTLDDKIRRAVKEWLHLPANTCNGFLYARSRDGGLGVTRLASLIPSIQARRLHRIAHSEDDTIRCIALANDIESEYRKLWLAAGGTQDTMPAITDPVIMDHQLPQHVLEQLTEWEKPAPRAIYPIPCNWRNAEFSQWKDLPCQGSGVSHFENDPTSNDWLTHHKGFSQRQFLTALKLRANVYPTREYLGRGKNNSIVRCRHCSASYESLSHILGQCPAVQGARIRRHNKLCDILSREARKLQWKIYQEPHLRTANNELRKPDLIFVKEGKALVVDVTVRFEYKEKTFSDAAAEKVRHYQPLTEAIKKLTGANKVSYFGFPLGARGKWPTINESVLSSLGLSESAIKRAARLLSRRALLYSTDILSTFENIGKGLSNPADAKPEGTPKRQMGIL

SEQ ID NO: 40 - protein, 1326 aa, synthetic construct
MMEALASAQLCPRRRANSTHLDADVVPWGRCTFRTFEPLARYRVKVSTVATQTDSHETIDGEAYPTLKSSQATVTEGEMYPIPGTSQTPTAAGAISCETVAQTEAEEILPALPLEILNSAFGETVRPDTSPEPCVPTTPVSEKERDADGSETLADGIDGTIGVPSNAPTVVVTEGECENRVQGKVLVKLPHGNPACPFCGDHVGKPTALSVHLKRHHGGKDVEFQCSLCYKVDPNVHSILCHIPKCKGLTSQEPAGDWACEVCARRFGTKPGLSQHKRRAHPVVRNTERILASQPKQNSQRGKHKSCWSAEEELLLIDFDTMYSGEKYINKLIAEAIKTKTAKQVSEKRRLLNLYKKGTSGVADPGPGAQRHNLEVRSDTPTSTAGLENNYRRTVTNRLRDHNQGPLRQGLENFEAEVLSAWVAGDPNIRSLVENTSIDVLSSFLAPEHEHKHKEKGNTKGRRPKSGKDTSWRQKRATKRGTYLRFQHLFENDRCKLASIILDGTERLQCQIPLEEVFKTYKDKWESRTPFQGLGQFKSYALADNSAFDILISAKEVLKNIKEMNTNSAPGPDKVSLRDLVRVDPDGDTLAGLFNTWLVTGIIPDGIKECRSLLIPKTMDPEALQDLGNWRPLTIGSIVLRLFSRILTNRLAKACPLNARQRGFIAAPGCSENLKVLHTITKQAKKEKKPLGVVFVDIAKAFDSVSHDHIMWVLQERGLDQHIINIIGDSYRNVHTRMEVGKELTSPIAIDVGVKQGDPMSPLLFNLALDPLISTLEMAGKGFSFGAQKVTALAFADDLVMLSDSWEGMHSNIQILESFCHLSGLRVQAKKCHGFMLNPTHDSFTVNNCEAWKIGSDGLNMIAPGESEKYLGVKVDPWIGFSKPALSDKLNTWLERINRAPLKPSQKLEMLNTFTIPRVIYLADHTDCKKTLLSSLDDNIRRAVKEWLHLPADTCNSFLYARSRDGGLGVTRLASLIPSIQARRLHRIAHSEDATIRFIALANDIESEYQSLWLAAGGTRDTMPAITDPVTIDHRLPQRALELLNEWERPAPRAIYPIPSNWRDSEFSQWKNLPCQGSGVSHFGNDSTSNNWLKHLQGFSQRQFLTALKLRANVYPTREYLGRGKNEYIVRCRHCDAPYESLSHILGQCPAVQGARIRRHNKLCEILTREARKLEWTVYQEPHLRTSTNELRKPDLVFVKDGTALVVDVTVRYEYKDQTFSDAAAEKVRHYQSLTGEVSRLTGANKVRYFGFPLGARGKWPTINDRVLSALGLSESAQGRAAKLLSRRALLYSIDVLNTFSGIGKGLLNPADPKPVGTHREQMGIS

SEQ ID NO: 41 - protein, 1042 aa, synthetic construct
MNSLLGLPLEPSSRTLNSPDAERGDSDRRPEPSGWACLVCARTFTTKNGLGVHKRRAHPVLANTEAAPNMVKRRWSDEELALLASTEAQLVTEKGKCTNIDLVARLPHLNRTVEAIKGQRRSQKYRSLVQMYLTNVPEASPNPEIPVAGCSTPTRNVAARPADSPGVAGESMARPADDPSVAVVVPPGVAETVSELLARAARRKSRSGPSAKAARRPHKTGIDESAHSARVSKRALRRMEYARVQELYKKNRGRAAAEVIDGQCRVVEHSLEDLEAYWRPIFEAVSGARGPTPEVLEAAKRKGQYGGEEARSHLWDPITVDEVRDTRLDLHTAPGPDGIRAEEWNAVPADRKAEIFNAWMSTGETPEPLRQCRTIFVPKKDSPAGPGDYRPISIASIPLRHMHAILAKRLLACCPPDPRQRGFIRADGTLENTAVLDAVMGDCRKRLKECHVATLDFSKAFDTVSHKALLELLRARGLPKAFVDYIARLYETATTILAANGKTSDPVRVGKGVRQGDPLSPILFNIVMDSILDSLPAYVGYRLEGALISALAYADDVVLITGSQVGMTEVLKLVSRCAEQLGLRLNHSKSSVLSLVPDGHRKKLHYLTEKSFGVGRRKLRQATCVERWRYLGVDYSAAGCVTLEHDITEALNNITRAPLKPQQRLEMVRGHLVPRFMHGFVLGKIADDRLRMLDVQIRGAVRKWLRLHKDVPTAYFHAKVQDGGLGLPSLRAVVPELIVKRYGALDRSTWEAARAAARSDKIRKKLSWAKRQMRKYLHDTGGGSSTSEATHTADYWRESLYRSVDGFELRESSKTAASTKWLRERSGRYTGRDFVQFVHTHINALPSRVRTTRGRRNGTGSELNCRAGCMVRETTAHAIQQCHRTHGGRIERHNLVVRVFSEELARQGWHVEVEPKVVTSLGIRKPDIIAVRGEVGVIWDAQIVSGQRPLDDAHRTKRTKYGSDVELVEKVADKLGLPIGCVQATSCTLSWRGIWSRASVNEQRRLLGLGSSLFEWIPELVLRGSHTNWTRFNQLTTVKGRG

SEQ ID NO: 42 - protein, 1060 aa, synthetic construct
MRIHSRQQAQQTSSAESQPLTPAVDPVASPSTSQGRFVCEDCAPPVTFSTKIGLGQHRRHCHPDVTNAEINIERVRKGYSSEELRVAASEEAYLVKHGKDRLGNKVRFINVYMAQFFPNRNTDGVKNARKTKIYRDLVAQYVSQLDEGETPESSTPDSSPGRTPPESPARTSAAPIPEEPSPLDPRDRYKEAISSSLVKMAEDESLQDHPLVALGLDLINGRDISLEMISWLRTLLRPKVRAKASRKGRRRSKRPFNFNAKPSAWRSRRREYARMQDLFKKNMSRAAKAVLDGEDEATLPSLEEMSDFWKPVFEHPSIPVQQHPVLPEHQQKTSLADPITIREVNSINVPSCSAPGIDGITPAVWRQCPALCRALLYNMVLYSGSFPPDVLTSRTVFVPKKVKATKPSDVRPISIASVVVRQLHKILASRMMAAGLIDERQRCLEDGCAENISILASTINHARSNRRELHMVSLDVAKAFDSVSHQAITSALTEAGVPQVMVDYIRTSYEKSTTILEVGEQKSGRIVVSRGVRQGDPMSTVIFSLVNDRILRSIPTTVGYELAPNHIVNGVAFADDIVLLTGSVSGMTATLNAVEETARKQGLAFNPSKCVATSIVPSGRDKKVKVLTEPQFALQDRSLIRQLGPVDEWRYLGVNFSPVGIKKASPSLEVELQRITRAPLKPQQRLKILRCYLVTRFYHLLVLSGCTLKVLRHFDFQVRAAVRRWLRLPKDIPIAYVHASCRDGGLGIPSFATTIPGLLLNRLEQMKDSSFKSARTAYRSVWVQKKIEWARGVLNCNGERLNSSEHRAKYWARCLYERVDGMELRDCGRTSASTKWIDAGALGVPGRDYIQYHHVRINSLPTKVRTSRGQRHVYTNIKCRAGCDSTETAAHVIQSCFRTHGGRIKRHDAVCKTLADHMRSRKMRVHVEPHLRTLVGLRKPDILVADDDKGFIVDVQIVSGAVPLDDSHKRKVEKYDSLPIRSEAAKVLRVKPSAVTVLTCTLSWRGVWSGTSADTLRSDLRIPLGLLCGITTRVIQGSHTNWTRWNQMTSRIPAERAGIG

SEQ ID NO: 43 - protein, 1125 aa, synthetic construct
MRNVFRSGRNPSGVRGGSTSNSPVGRRACSDKSIRFLRLGHYKSEGRTSVAGQHPSTTEDYLNAKGNDKGNKLNRCLSDLPQESEAASNPLPDAQAGAQAGPSTASEWTCDICARSFSTKTGLGVHKQRAHPVEANEQSAPPQVKRRWREEELELLARTEALLIRGGSACANVDLLNALPNLGRTIEAIKGVRRKETYKQRVQTLLLGSPPTQERSYEVSTQEQHNSSCQNDEMERVDERAGGESSEEDVVEVRPVSSGEGAPLVADLRQAETGRPTCKPIVEELLARASARRLQTAKRSMTARGNKSHTEVKLTSRKRRRMEYARVQELFRKFPGRAAAEILDGQCRGVRHGLDVLEAYWRPVLEKVSGAPGPTPEALRAVRSNGVDGGALDVQMLWAPFTSEEVKESGIDLRSAPGPDGIRPDEWRAVPAETQAEIFNEWMSRGEIPEPLRRCRTVFVPKREEPAGPGDYRPISIASVPLRHMHALLARRLEACRPPDVRQRGFIRADGTLENSAVLDAVLGDCRKKLRECHIAVLDFSKAFDTVSHAALISVLRGKGLPVGFCDYVARLYDSATTTLAALNEQSEPVVVGRGVRQGDPLSPLLFNMVMDVVLASLPKGVGYRLEGELVSALAYADDLVLLSGSTVGMREALGAVERVGEMMGLQLNQEKSSVLSMVPDGKRKKPHYVTSRTFKVRGRWLRQVDNVEHWRYLGVEMSASGVCTIEHDIKEALKNVTKAPLKPQQRLEIVRTHLVPRFLHGLVLGNITDDRLRMLDIQIRHSVRQWLRLPPDVPVAYFHASVQDGGLAIPSLRTCVPDLVVRRFGKLDSSRWSVARAASLSERIRRKLKWANKQLSKFSIEHPRTGEKSVKQFWRKSLHDSVDGVELRESTKTPASTKWIREKCMSITGRDYVQFVHTHINALPSRIRNSRGRRTGRESELNCRAGCMVRETTAHTIQQCHRTHGGRIERHNCVANYLAKELGNRGWRVESEPRIKTAVGLRKPDIIASRDDGVGVIVDVQVVSGQRPLDEWHRNKRSKYGSHDELVEKVADRLGLSKEHVRVTSCTVSWRGVWSSGSYKEMKELLGLKEYVYETIATLVLRGSHMNWSRFNRMTTMSHGRDSE

SEQ ID NO: 44 - protein, 1169 aa, synthetic construct
MPCGSLGLATRLNHAYGQPQTAPNNSKTSLGRCRSRTVAATQVDSTNQGGNIPLANNSNNDSSDGPQRNIPDNCPSCGVRLTDRTLASAKQHLRRCNAPPKEELGYTCSICQNIFKSYAGLRQHQRRAHPHEYNRADVELIQKRAPTTRSQYTDGEIQAIANFEARIENREKLKVKEINSIISDETGRPIEGIRKLRLKEIYKLYLNSEIENIKKQKYRDLCPLSPKIILSRVNYDDETAIDNIEPDLNSNTNTSTTTCKLIALPAIYQLLHELKSLTTSDMTDIIDKIINGNPETEILGDIDQYIKSIVNKICVKTIKNKNRICNIKITSKSKTYNKNCTQKQNDACNKNYIQSQNNTCNKTRMQNKNLTGNNIKKPIKGNRSKRRATEYMRIQKLYNKNPTQAAEHILSGTELNAQEYPSIETFAEYYTNLFAENKLNWNPIPHTNNSVQISHPINPTEVHKELRSLKESAAGIDGIKREHLREMPRHDLVALLNIIWGMKKLPSSLRTNRTSLIPKSGNLKDPKQWRPITISSRILRLLNKILTNRLENEIKLEHSQRGFVKTDGIMANSTTVQTIIRSMRNKAKPFIMLAIDLAKAFDRVSINSVIDALRRRGVDEHTIEYINDIYRNNITELECHGQKSLPINIKRGVKQGDPMSGFLFNLIIDDLILKLKPREGIQIGNSNITSLAFADDLLILAPSPTVMKNHIRIIESFFSKHGFEVNVEKCVAFQYMSVPGTKRIAVVTEPKLKIKNTKIPTISTASQLKYLGHQYKYTGISSPSPSKIQDMLDRLQSSPLKPWQKINVLQRYLIPRLHHGMQTMDVTRKKLEFVDSKIIKFVKNTLHLPITTPTCFIHAPMRCGGLGIPSMRLHISSVYLRRLERLAVRGDEEIQEIIKTTVVQQLINKLKRITKDLYNNKKETVHNYWSDQLHSSALGAGLKSITPGVSSWIYNPPKYWKGRDYIGAINLRIGLLPTKGAPYMTNTDCRNPSCEGTRESLYHVLQRCPVSHHTRINRHDNINKIIKQTIIKNKVKCEDVPKITTMNNTYIPDLIAIKDQVAYIIETTVAYESKATSLEKSYLIKKDKYNRLDLKHKIKQMYMVNDVKIMPFVVGARGGWHTGNDEIISSLNLPRHLKEILVTNSLQWGVSIHRSFMRTAWRITRHDPG

SEQ ID NO: 45 - protein, 1042 aa, synthetic construct
MENRCSPSFPREAGSTSNLVPDGRPGASDGPGMASGYGCDLCGRIFKTKIGLGVHVRRAHPADANAQSAPPQVKRRWRDEELELLASTEARLMGEGSANVNIDLLKALPNLGRTLEAIKGVRRKEQYKTMVANLLMGSPSTSNDDTERVVADVEREVPEEAVAEVRPVSGSEDAPLVADLRVDDRSACVSVVEDLLERATQRMMRGSVKSKRNKRHKICLKTRTEVKLNSKRRRRMEYARVQELFRKFPGRAAAEVLDGQCRGVRHRLDELEAYWRPVLEKVSDAPGPTPEALRAVRRNGQYGGTLDRLQMLKLWSPFTEEEVKEAKLDLRSAPGPDGIRPEDWRSVPARTRAEIFNCWMSQGEIPLPLRQCRTVFVPKRDEPAGPGDYRPISIASVPLRQMHALLARRLEACCPPDTRQRGFIRADGTLENSAVLDAVLGDCRKKLRECHIAVLDFAKAFDTVSHAALVSVLREKGLPVGFCDYIARMYDTAQTTLAVGNDRSEPVVVGRGVRQGDPLSPLLFNMALDVVLASLPKEVGYRLEGEQVSALAYADDLVLLAGSKAGMQAAIDSVAETGASLGLQLSREKSSVLSMVPDGKRKKPHYVTTRSFKLGRRWLRQVESEERWRYLGVELSVSGTVTLEQDVKRALENIKKAPLKPQQRLEVVRTYLIPRFLHGLVLGNITDDRLRMLDLQIRHSVRQWLRLPKDVPIGYFHASTRDGGLAIPSLRTCVPDLIQRRFGKLDSSRWSVARAASKSERIRRKVKWAERQLRKFSADAPRTGERTVVQYWREKLHQSVDGFELRESTKVPASTKWITKKMGDLKGRDYIQCVQTHINALPSRVRNTRGRRVGRESELNCRAGCMVRETTAHTIQQCHRTHGGRILRHNCIADFIAKELGQAGWSVEQEPRIETAVGLRKPDIIASKGDDVGVIVDAQVVSGQRPLDDAHRDKRSKYGGHGELVELVADRLGLSKDRVSATSCTVSWRGVWSLGSYREMKKLLGLKEQVFERIAALALKGSHMNWSRFNRMTTRYLDEAPD

SEQ ID NO: 46 - protein, 1280 aa, synthetic construct
MGWVLPANPGISSVKTNMLVPLCSIGQDHSSGLSKRVPVRLEMGSSPRQRICLTHVFEVGCVWYRRINLPSGRWDQPLGGLSRVPTGTVAAASSPNLSRVKSISIGTQVQESDWQMLPGVSSVEAELMTETASAIRVEAAEVVLAPQLEQVVGAPHTLPEVGRTESPRARADALTEVRVLLPWLASDCPLCGVQVKGIVALGKHLAIRHGGVTAMYVCSKCGRSSSSRHSISCHVPKCGGGRAEEPASGFACELCDARFPTLVGVSQHKRHVHAAAWNEVRLARRKVPAGDIRATKLWSVGEVRRLIQLIREHGDSVATYQVIADDLGNGKTAEQVRSKRRNLRIDMAGDCSNIAEVEGGEMVADPEPQAEQSSAPSPQVLSSDRIREALVGWGDREGGEEVRAIADLVRDVDQDPFLIESSASEIISRLGRGVERNGRARPPVREQNQVRGWERRLSQRRRAYRECQFWYSRNQARLAARILDGAEGQECALPADQIYEAFRERWEVGRQFHGLGEFRTGLVSDNREFCTPILAAEVMENLAKMSNGTAPGPDRISKKGLLRWDPRGEQLARLYTAWLVRGVIPRAFKECRTTLLPKSTDPGELQDVNGWRPVTIGSVVLRLFSRILTMRMARACPLNPRQKGFLVSSSGCAENLLMLDEIIRRSRVEGSPLAVVFVDFAKAFDSIAHEHILCALGDRRIDKHVIELIRNSYVDCVTRVNCVDGPTQPIHMKVGVKQGDPMSPLLFNLAMDPLIHRLEQTRLGLRWGDQSISTLAFADDLVLVAGSVEAMNGSLGILEEFCQRTGLSVQPKKCHGFYLDKGTVNGCEAWRIGGVPIHMIPPGETVRYLGVQVGPGQGIVPPDLIRTVSTWVTRISEAPLKPSQRLRLLNTFALPRLMYQADLGGVSTTKLARVDGLVRKAVKQWLHLPPSAGNGLLYSRVQDGGLGVIRLERLIPSIQVRRLYRMSRSTDVWTRNLTKQAVSQSDWERLWIKAGGDAGSVPVMGTGEATPDAGGPPVYPDWRRGESLAWAALEVQGVGVAQFRGDRVSNSWIADPTAVGFRQRHWIAALALRTGVYPTREFLARGRDKSGAACRRCSARLESCSHILGQCAWVKGSRIRRHNKICELLATEAEGCGWTVTREFRVDGPEGGLRIPDLVCKKADTVFVIDVTIRYELDGGTLARAASEKVARYLPVAAEIARRMGGRCVEVRGFPVGARGKWPTCNNTVLMELGVPIGRRRAFARLVGRRTLLYSLDVLRDFLYEPEGRGLCAPPMAAVP

SEQ ID NO: 47 - protein, 1043 aa, synthetic construct
MERCPSGLPQESEEASNPLPDARAGAAAGPGGSSEWTCDVCARAFSTKTGLGVHKRRTHPVEANAQSAPPQVKRRWREEELELLASAEARLTREGTSNMNIDLMKALPTIGRTLEAIKGVRRKESYKTLVMTLLQGSTPTRSETKITERVDGSAGGRSPVEVVSEARPGGSRENTPLVADRRQAEIGDVPKCMSVVEELLERAKTRGQRGGRRSMRAGGKARPCSTPTEVKMTSRRRRRMEYARVQELFKKCPSRAAAEVLDGQCRGVRHGIDALEAYWRPVLETVSSATGPTPEALRAMRRKGQYGGQLDARRLWLPFTPEEVKAVGVDSKSAPGPDGIRPGEWRQVPAETKAEIFNYWMVRGEIHETLRQCRTVFVPKKEVPEGPGDYRPISIASIPLRHMHALLARRLEACCPPDIRQRGFIRADGTLENSAVLDAVLGDCRKKLRECHVAVLDFSKAFDTVSHAALVSVLREKGLPVGFCDYVARLYDTAHTTLAVNEKRSEPVAVGRGVRQGDPLSPLLFNMAMDVVLASLPKGVGYRLEGELVSALAYADDLILLAGSTVGMREALAAVERTGAMVGLQINREKSSVLSMVPDGKRKKPHYVTTNLFKMRGRWLRQVDNVEHWRYLGVEMSSSGTVTLEHDIKQALMNITKAPLKPQQRLEVVRTYLIPRFLHGLVLGNITDDRLRMLDVQIRHSVRQWLRLPPDVPVAYFHASVQDGGLAIPSLRTCVPDLVSRRFGKLDSSQWSVARAASKSERIRRKIKWANRQLQKFSVENPRNGERTVVRYWRDNLHASVDGIELRESTKTPASTKWIREQSVQYSGRDYVQFVHTHINALPSRVRNSRGRRTGRESELNCRAGCMVRETTAHTIQQCHRTHGGRIQRHDCIAKYLAKEMGDRGWTVALEPRIKTAVGLRKPDIIASNGDGAGVIVDVQVVSGQRPLDECHRDKRRKYGSHGELVEKVADILGLTKECIKTTSCTISWRGVWSSGSYKELKALLGLKEQVFEVIVGLALRGSHMNWSRFNRMTTMGHDGAPE

SEQ ID NO: 48 - protein, 1040 aa, synthetic construct
MDHPDLPQESGSGQITSSSARPDDRDLNVGESSDFDCPLCGRSFKTKTGLGVHKRRAHPAEPNVEAAPKGVKRRWRDEEVLLMARTEAQLMKESGKCSGSDLLAKLPQLQRTLEAIKGKRRGEDYKRLVKTCLEEFSSPAPVPSASGCRSADHSGVAQPPGAQEECTSEDVPPAGEPHTATRASISPNAEQALSKLLQVAERRRKRCGLGARAARRLHLNGIDGCTLESLSRKKLRRAEYARAQELFRKCPGRLAAEIITGQSREVKNSLEEMEAFWRPILEEVSEAPGPTEVSMRAMRRARDAEGANVFDRLWQPISVDEVKATNVDLQTAPGPDGIRSDEWRSVPNQLKAEMFNSWMANGETPDQLRQCRTIFVAKTDTPKRPSDYRPISVASVPLRHLHSILARRLLDCCPPDVRQRGFIHADGTLENTAVLDAVLGDSRRKLRECHVAVLDFSRAFDTVSHKALLELLRARGLPKGFCDYLARLYETATTTLAVKGKQSQPVKVGRGVRQGDPLSPVLFNMAMDGILASLPKGVGYGLEGERVSALAYADDLILLAGSKAGMQESIDCVVRSGEAVGLFVNRSKSSVLSMVPDGHRKKHHYPTENLFSIGRKKLRQVTCVERWRYLGVDFDSSGTATFEQEVCKALDNISRAPLKPQQRLELVRGHLIPRFMHGLVLGKISDDRLRLLDVRIRGAVRKWLRLPADVPVGYYHASVKDGGLGVPSFRVVVPATIVRRYDGLAQSSWPAASAAARTDRIRKKLRWARRLLRKFGMTSAEDVTSVAKYWRERLHTSTDGFELRESSRVPASTEWIRKRSGQLTGRDYVQFVHCHINALPSRIRASRGRRDGMDTRCRAGCVEKETTAHIIQQCFRTHGGRIKRHDFVTRVIAEAMTQRGWSVELEPKIMTSLGHRKPDVIAVRGGVGVILDTQVVSGQRPLDEAHRAKRKKYGKHEELVKKVAGRLGLPTSECVRSTSCTISWRGVWSASSVKEMKELLGLDNSVFGLIPSLVLRGSHTNWTRFNQMTSYITSWTPDRA

SEQ ID NO: 49 - protein, 1033 aa, synthetic construct
MLTDTVVLRPPGLPDPSCPGSCPRVASPKSDAECPKHSCPCGSEFRTKRGLAMHRFHCHPAEVNAERLTTLPTKKLVWTQEEMDGLLRHANGAWKEGMLKTQIFTSLMPHFPGRSAEAIKKRLQSLKWVPARDNPERNSQPLHATSGNVAPATLPTPDPSRVDDQGANIEDPAASWRRALLTAAADHFKEPKLGAGTLKQITQDLLNGKISSRECRVLMGEHAQHWFPHTWKPSSARPEPKARQWKAREIRRANYAAVQKLYKLRRRDAAQAVFSGSWRNAYRGRAPQPDKMDQYWAEVFETASATDVTPPGSPSEIYWSVVSKITPSEVADALKGMRNSAPGLDRITAEELLTWDHPSLAAYYNLMLAAGGPPEHLACSRVTFIPKVENPQTPGDYRPISVASTVLRAFHKILAWRLRDNLQLSPFQHGFLQRDGCLEATALLHTILRKVHNSRKSCAMLFLDVAKAFDTVSHQTLFRVAVELGLPPPLVNYLKCLYSRSTVRLADKATKCGRGVRQGDPLSPLLFIMVMDDIVRKTLPEVGFDLDGQRVDSLAYADDLVLLAEKSPRLQDKLHLLSEALRKAGMSLNARKSRGLTITKVSKRKQMVITPTTYECEGEPIKPMGTDDSVRYLGLHFNWKGRIVPKHTGKLDSLLKELTKAPLKPYQRLQLLKFHAVPKFTHELVLGHAHRNTVKKLDCLTRAAVRKWLRLPQDTPLGYLHANVKDGGLGIPCFSTSIPLLQKKRFEKIATNPATIFQIMQRQDSFRTQGRRLDKPCRLNSTVVTSKTEVREEWGNMLSNSVDGRELRHPEVDKASHEWLLRPDRVFPRLHMRGIQLRGGLLPTKARSGRGNNRQVTSKTCRGACQNVETLNHILQVCDVTHDARCARHNRVLHRLERLLHKKGLRTTLEPVIPAGSSFIKPDLIIETDTQTYVLDVSVVAGYRMTESWDIKVEKYGRPDRVEAIRKWAAIPATKRVEHLPVIVSNRGLCYQPSGDGLRALGLTSRDISDLCLLAISGSLRCYDLYMRGTRAV

SEQ ID NO: 50 - protein, 1423 aa, synthetic construct
MEFGNGLTTHSVLTVECGPNSMVIPDPSFGGGVWGPPVRLSRVSVGTQTSPSDWVDLLSRGLPGARSNSQRVDLVSLFPRHRVDLLSKTQRVDLLSESQRVDLLSKRRRVDLLSDSQRVDLLSKTHRVDLLSKTDRVDLVSQIDLTSKTTNLAENELDLLVNFSLELYRSDLHVHEGVHFSVDWEVLNRLPEVYEELAPQPCVGENLDSSPPQDSEPLVLGEGSGEKAGGGTPEALPPVPGENGGQSPDNIVKVTVPDKNPPCPCCGTRVNSVVSLLEHLKGSHGKKRVRFRCAKCGKENVNYHSVICHFPKCKGAEIEKVPAGEWICEVCGRDFTTKIGLGQHKRLAHPLIRNQERIVASQPKETSNRGAHKRCWTKEEEELLIKLEAQFEGNKNINKLIAEHITTKTAKQISDKRRLLPKKLPEVTDKEPGVCLRTRRAAGSVGTEPGTSQQSQAGILDNGPGKCHLPGRPAAGEKTMEKLRRHPDKDNGRQKTSGQRREGLLQAHYQKTIKEGLSAGAINNFPGAFKQLMDGREMRTMINQTAQDCFGCLESISQIRTAMRGKNSGKKATMEQPARKFQKWMKDRAIKKGNFLRFQRLFHLDRGKLAKIILDDIECLSCDISPSEIYSVFRARWETPGNFAGLGDFKITGKADNNAFKDLITAKEIERNVQEMSKGSAPGPDGITLGDIVKMDPGYSRTAELFNLWLTSGEIPDMVRGCRSVLIPKSTKPERLKDINNWRPITIGSILLRLFSRIVTARLSKACPLNPRQRGFIRAAGCSENLKLLQTIIRTAKSEHKPLGVVFVDIAKAFDTVSHQHILHALQQRGVDPHIIGLVNNMYKDISTYVTTKRDTHTDKIQIRVGVKQGDPLSPLLFNLAMDPLLCKLEESGKGFHRGESSITAMAFADDLVLLSDSWENMQTNIKILETFCDLTGLKTQGEKCHGFYIKPTKDSYTINNCAAWTINGTPLNMINPGESEKYLGLQFDPWTGLAKTNLTTKLEFWLERIDQAPLKPLQKLDILKTYTIPRLTYLADHSEIKAGALEALDQKIRTAVKDWLHLPPCTCDAILYSSTKDGGLGVTKLAGLIPSVQARRLHRIAQSSDETMKEFLEKDQMEQLYRKLWVQAGGEREEIPSIWEALPTSEQSNRGDTTSEWEAPILKPKFPKPCDWRKKEFEKWTKLVSQGRGISSFKNDSISNDWLQYYRRIPHRKLITALQLRANVYPTREFLSRGREENSTKACRHCNVENETCAHIIGQCPVTKDARIKRHNHICDMLSEEARKKDWVVFKEPYIRDTTKELYKPDLIFVKDGHAFVVDVTVRYETTTTSLEEAAAEKVNKYKHLETEVRNLTNAKDVVFMGFPLGARGKWYTGNFELLNTLGLSKSRQVRVAKTLSTVALLSSVDIVHMFASRARRVQDSG

SEQ ID NO: 51 - protein, 1042 aa, synthetic construct
MPPDLPQESGPAFPLSDAQPGTSAGPGLASGWTCVTCGRVFSTKTGLGVHVRRAHPVAANEQSAPPQAKRRWREEELDMLASTEARLVREGSACVNADLLKALPNLGRTLEAIKGVRRKESYKDKVQKLLSGSPPHTSDVSPGDTIGGDVVGELPDGDGAEVRPAGEPEAAQLVAGLRTEEANDRSPCVSIVADLLERATMRMTQGSRRSMKPRGQNRKTRTLHTQTGKPNARQRRRSEYARVQELFRKCPGRAAAEVLDGQYRGVRHQLSELEAYWRPVFEKVSDAPGPTPEVLRGLSHDGRYGGLLDREQLLKLWSPFTEGEVKEVKVDQKTAPGPDGIRPEDWRTVPAKIQAEIFNSWMSQGEIPEPLRQCRTVFVPKKDEPAGPGDYRPISIASVPLRHMHALLARRLEACCPPDARQRGFVRVDGTLENSAVLDAVLGDCRKKLRECHIAVLDFAKAFDTVSHAALVSVLRGKGLPVGFCDYVTRMYSSAKTTFVVGNDRSDPIAVGRGVRQGDPLSPLLFNMVLDVVLGALPKEVGYRLEGERVSALAYADDLVLLSGSTAGMQAAIDAVGETGAMVGLQLNREKSSVLSMVPDGKRKKPHYVTARSFKLGRRWLRQVESEEHWRYLGVDISASGAVTLEQDVKRALENITRAPLKPRQRLEVVRNHLIPRFLHGLVLGRVSDDRLRMLDVQIRQSVRQWLRLPKDVPIGYFHAATRDGGLAVPSLRTCVPDLIVRRFGKLDSSRWSVARAASKSERIVRKVKWAEKQLRKFSIAAPRTEERSVVRFWRSKLHSSVDGAELVESNKVPANTEWLRKGDISGRDFIQCVHTHINALPSRVRNSRGRREGRESELNCRAGCMVRETTAHTIQQCHRTHGGRILRHNCIADLLVRELEQGGWTVEQEPRIETAAGLRKPDIIASRGDGVGVIIDVQVVSGRRPLDDAHRDKRNKYGCDAGLVERVADRLRLAKDHVGVTSCTVSWRGVWSLGSYKEMKRLLGLKEAVYERIALLALRGSHMNWSRFNRMTTMVPCGALD

SEQ ID NO: 52 - protein, 1245 aa, synthetic construct
MGRQLRSLFATIGTQTTPKDWLDVATTDNTRPPDEEIRVCWTGIEFVNSMIAENNIAYNPIWNEKLMILPEAYEGPTSPAREKGIRDQSLERSRNEDVDCPETPSSMSQLSILTASPELENTADTCPGTPLVRMPDDNPACPCCTTRAGNLTRLIDHLKRAHGKRKITFQCSRCERTNENHHSISCHIPKCKGVQEPTQIEGFRCEECNNCFKTKIGLGQHKRLAHPALRNRERIEASRPKETSKRGAHKQVWSQEETEMLQTLSQEFEGRKNINKLIAEKLTTKTAKQISDKRRLLKETLVPEKEKTPERKSRLKKGRTEIPSSGEGELIAHYRKTIKEKISSGKLPILQNTFKQIIEGKKDSLKILNEVAQDKYLHLFEKAIEQKAPKQSHRKPQQRKSKKEWMKKRSKKRGEFLRFQRLFQLDRGKLANIILDEKDSAQCTIPLEEVSNTFRSRWESQNTFQGLGTFESVGRANNSAFKDLITEKEILKNIKELNKKSAPGPDGITLKSLLKMDPDCKEIRELYNLWLLSGKIPDVVRGCRTILIPKSSAPERQSDINNWRPITIGSIILRLYSRILTARLAKACPINPRQRGFIQSSGCAENLKLLQLIIRNAKKQKKPLGVVFVDLAKAFDSVSHTHIIEVLKQKGLDDHIIDLIKDFYHNIHTVIKTKEGETESIGIKTGVKQGDPMSPLLFNLCIDPLLCKLEECGEGFQHGKGKITALAFADDLVLLSSSQSGMEKNIEILEAFCDLTGLKTQGEKCHGFFVRPTKDSYTINDCPPWLVSGTPLNMIHPGQSEKYLGLKIDPWVGLVKPELQNNLEVWLNRIERAPLKPFQKLDILKVYTVPRLIYLADQANVKSTYLEKMDLTIRNRVKNWLHLPTCTCDGILYSSTKDGGLGLTRLAGSIPSIQARRLHRLANSSDDTVKTVVKENDIDKDYEKLWVSAGGDKDKIPSIYNPQTIMVAEYGGSEELSEWEKPAPKKIFPKPCNWRKNEFKKWTTLMSQGIGTAVFENDSTSNHWLVNYKNIPHRKLLTALQLRANVYPTREFLARGRKEDQNTKCRHCQADNESVAHIIGYCPAVQDARIRRHNYICKMISEEAKKKEWLVYEEPQIRDKDLNLYKPDLVFVKENVAYVVDVTVRYESNITKLKHAAEEKVRKYKHLREEIVELTNANEVKFAGFPLGARGKWYEGNYKILSDLGLSKSRQSRVARRLASRALLTSVDIVHMFAGKSRKVTTNPV

SEQ ID NO: 53 - protein, 1097 aa, synthetic construct
MGGLNESNSDSTKSSGFLRLGPFEKPWAKPLAGFAARSKRICIVAKSPKKKNLGRPDLPQESGTTVLSSSAAQPDAPGGSEGRAPDWICPICGRIFGTKTGLGVHKRRAHPVEANAEAAPLQVKRRWSDEELLLLARTEAQLTAETGTCSNVDLLAKLPQLQRTLEAIKGQRRSVKYKGLVKASLEELALAAADLASRGSSAEQAPEALPTETQNAECSEVPSVADDPAAVETPDSNAVKVVENLLREAERRKQCRERGAEAAGRRRRKSQKKRILNTALDTLSRKRQRRIEYARVQELFRKCPSRAAAEVIDGHTRETKHSLEVLEAYWRPILEEVSDAPGPTRQALSALKRGASLYESQGGVDDYSRLWAPVTVDEVKASRIDYRSAPGPDGIRAEDWRAVPAASKAEIFNLWMARGDIPEPLRQCRTIFIPKVESPAGPSDYRPISIASIPLRHMHGILAKRLLACCPPDARQRGFVNADGTLENTAVLDAVLGDSRKKLRECHIAVLDFSKAFDTLSHGALLDLLRARGLPKGFCDYLARLYNTAHTILAVKETYSQPVKVGRGVRQGDPLSPILFNMAMDLILASLPKGVGYGLEGEKISALAYADDLVLLAGSKVGMQEAIDTVVQVGGTMGLLVNPKKSSVLSMVPDGKRKKHHYLTERVFSVGKRRLRQVDCVERWRYLGVDFESSGASLIELEVRKALENITKAPLKPQQRLEIVRGHLIPRFMHGLVLGTISDDRLRMLDIEIRGAVRKWLRLPKDVPKGYYHADVKDGGLAIPSLRTVIPDLVLRRYGGLASSRWSAANAAARSDRIRKKLSWARKQVRKFTRDHPKTQVTSVSQYWREVLHASTDGYELRESARNPASTKWIRERCMQYSGRDYVQFVHCHINALPSRVRASRGRRHGRESELNCRAGCTLRETTAHTIQQCFRTHGGRIERHDCVTRIIAEAMSEQGWKVELEPRFTTSRGVRKPDLIAVRGGAGVILDTQIVSGQRPLDDAHRAKRSKYGSHEELVGLVADKLGLSDAACVRTTSCTISWRGVWSGASSKEMQEVIGLKKAIFEIIPGLVLRGSHMNWTRFNQMTAVSAASSD

SEQ ID NO: 54 - protein, 1012 aa, synthetic construct
MRKHVLQVPAEPEVFPCEIEGCGKRFPTRIGLGQHIRRRHPERMDERVNVARVKERWTVEESKMLARREAEFLVRWERDGRAGFINAVLQAEFPHRTLEAIKGRRRYVEYKRMVEDMCVEIREGEEVEMEEDAGIIPDMVPEEPPDPFLQFFQSLEPPSLENDVFRVLELYRICIDAAVVSRETLLARLGLYLNSILPGIRLPLRGTAVRNRHELPRRKERRRKYGETQDMWVKNRKRCIDMIINDTSNTIQPSREVMETFWTGVLTHESDSKPELHIPDVERTDLWTPITVEEVEKYRLEIQTAAGPDGLTGRDLRNFPKVGLQCLYNLFMWCGELPECLHASRTIFIPKKGFEDAPDKYRPITVPSVIVRGFHKILAGRLAMAFDIDDRQRGFRDIDGSRYNTFELDMILRQHYLQRKPLYMASVDIAKAFPSVSHPSLLECMRSFGVPVKFVDYVAGVYRKGYTILQGQGWTSERIYPKRGVRQGDPLSPPIFNLLTHRLLGCLSGEVGVRVGEKFTNASAFVDDTNLYAMTPKGLQSSIDVVCKFLSSCGMSINIDKSFTLGFRPSGKEKKSTVDITSSFKIGGQMLRKITRLDEWRFLGVRFNWAGRVRIKPLNLLGEDLSLLTKAPLKPQQRLFALRCNLIPKLYHQLALGNVLVGSLNRVDIAVRSAVRDWLKLPPDAPKAYFHSSVADGGLGIPSMRWMAPLLRKRRLEGLRTEEIGAANSFLEREIATCERRLVDRGNRLASLNDVRKLWSGKLHSSVDGCGLRDSIATRGQHHWVSDGTKFLTGRDFINSCRLRISALPTRSRTSRGRVRDRGCRAGCSDPATLHRAPETLNHVLQVCHRTHVARISRHDAVVNYCAQRLRARGCMVHIEPRVKTREGLRKPDLVATKDQVAVVIDAQVVGEQTDLRSAHCNKVRYYQSNSEITEFVSGEYGIPVNNISFTSVTINWKGVWSPCSARDLSGWGFVQNDIKTISSRVLIGGLAAFNIFNANTATGPGIRRGIG

SEQ ID NO: 55 - protein, 1045 aa, synthetic construct
MDPTKKIVDHPDLPQESGSGQNPSSTTQPGATVVNPGSSEGWICPICGRSFRTKAGLGQHKRKAHPVEANVEAAPNEVKRRWREEEVLLLARTEAQLTKENGNCTGRDLHAQLPQLHRTLEAVKGKRRGADYKGLVKTFLEELSLPAPDPIASGCPCLDPHSQPAQLTRVEESSEDVPPAGEPIPVVRAKISPNAERTLSRLLKVAEGRRKRCGLGAKAARRLHRDTLQTEEALETISSRKRRRIEYARVQELFRKSTGRIAAEIITGQSRGVKHSLAELEAYWRPILEEVSEAPGPTAESLRAVRQAAGAESPCFESLWQPISVNEVKAVKVDLETASGPDGIRADDWRSVPIPVKAEIFNSWLMSGEIPEPLRQCRTIFVPKVDTPKHPSDYRPISIASVPLRHLHAILARRLLDCCPPDTRQRGFVHTDGTLENTAVLDAVLGDSRRKLRECHVAVLDFSKAFDTVSHKALVELLGARGLPTKFCEYMARLYNTATTILAVQGKYSQPVRVGRGVRQGDPLSPVLFNLAMDVILASLPKGVGYGLEGERTSALAYADDLVLLAGSRVGMQESIDCVVRTGETLGLYVNRSKSSVLSMVPDGHRKKHHFLTQSTFSVGRKKLRQVSCVERWRYLGVDFDSSGTVTIEHEVSKALTNISKAPLKPQQRLEIVRGHLVPKFLHGLVLGTISDDRLRMLDVQIRGAVKKWLRLPADVPLGYYYASVRDGGLGLPCLRTMVPTVIARRYGRLAESNWPAAAAAARTDRVRRKLKWARKLLSKFGITSPDDTTSVAKFWREKLHTSTDGFELRESSRHPDSTKWIRERCGQLSGRDFVQFVHCHINALPSRMRVSRGRRIGQGADQRCRAGCMVSETTAHVIQQCHRTHGGRIKRHDLVTRVVAEAMTKRGWKVELEPRLVTSLGLRKPDLIGSKGGVGVILDTQVVSGSRPLDDAHRAKRSKYGRNEELVEMVAGRLGLPAGSVRTTSCTISWRGVWSAASLREMQTLLELERNVYGVIPCLVLRGSHMNWSRFNQMTSTITTATPD

SEQ ID NO: 56 - protein, 1261 aa, synthetic construct
MGRPMRISFVSVATQTSPLDLRDNVVSSSIQDHKTGGLLEARFEIRGLEFVNSDLEGLTSQQSPTLTDLLAVLPEVYEVPNSLSLRDKNTCPTPVEDENEEEESSPLGAAAGTSPLPSTPVSRSCEDPPAESTPMVKIPDKNPSCPCCGNVTGKLSGLIEHLKRAHGKKKILFQCARCGRTDVKHHSIACHFPKCKGPVEASPERSWKCEVCEKTFESKIGLGQHKRLVHPVTRNLERITATRPKTGTMRGAHRRCWTEDEIELLRKLDKQYEGQKNINKLIAEHLPTKTTKQISDKRRDLHKSPVKKGKDQGQMKHLELEGEHLVEGEHLVCVGSEMEGGLRSRYRETVSEWLTSGKLSILHGMFEQVIEGEKDPAIVAEKAMKDCLACLASKSLELKAGQDRPRQVARTARPSEPPKGWMKKRAVKRGQFLRFQRLFYLDRGKLAGIILDNVESLKCGIPQDEIHDVFKSRWESPGTFKGLGAFRSKGLADNSAFEAMITAKEISKNIQEMNKNSAPGPDRLSLRDIIKIDPHCVQLMEVFNLWLVTGSVPDVVKECRTVLISKSSLPERQMDINNWRPITIGSIILRLFSRILTARLAKACPINPRQKGFIRSAGCAENLKLLQLLIKNAKKEHRPLGVVFIDLAKAFDTVSHHHLIAVLKQKGVDHHIITLITNMYQNTRTRVTMKNEQSDPIGIQIGVKQGDPMSPLLFNLSVDPLLCKLEEEGSGYQHDSKGVTSMAFADDLVLLSGSWEGMQSNIKILEAFCELTGLRTQGEKCHGFYIQPTKDSYTINDCPPWQINGTPLTMIEPGSSEKYLGLRIDPWTGISKPELLMKIRDWLQRIGEAPLKPFQKVDILKGYTIPRLIYLADQAEVKATSLEELDLTIRTTVKEWLHLPPSTCDAILYSSTRDGGLGITRLSGLIPSIQARRLHRIAQSSDDVVRSIVKREGIEKDFEKLWLTAGGDRNKIPSIWDPSAIRVTSEDPGEDELASEWELPTPKTIFPKPCDWRKQEFINWTKLQSQGRGIVNFEKDKISNNWLERYRGIPHRKLLTALQLRANVYPTREFLARGRQESHIKSCRHCQAENESSAHIIGYCPAVQDARIKRHNQLCDLLIAEVKKKDWIVFQEPILKDKQNEIYKPDLVFVKGDQAMVVDVTIRYESKPSSLADAAAEKVKKYCHLKSQIQELTNAKNIKFKGFPVGARGKWYSGNYEVLKALGISTSRQERIARCMSRKALFTSVDIIHIFVSHSRSLVRT

SEQ ID NO: 57 - protein, 1109 aa, synthetic construct
MVENTNQDGCNPLVLDITLASKNKENKKTPEVCPSCNINLSSRSYSSAAQHVRRCNAPPSQADGYACDICGSKFKTFAGTRQHQRRAHPESYNSKDLQLLENRQCSSRSQYTDGEILAISSIEASLPNRENLLSKNINLHLSEKTGRPVEGIKKLRQRQIYKTHLAKEIQKLSNESIRKICPLTPRVCINKLENTYANAVKNGSNRMEDLSTTSECPASSNPGNINTPCEHIASIEIYSLLQELQVKTTPEMSSIIDNILHNAPRIEIIDKLDKYILGLTPQQNKIFKKPKRMPYVKLKGNRAKRRASEYSRLQKLYEKNPGQVAEQILSNSPLDLSEIPDMKEFAQYYSKVFAYRELNWKPAYSTQPGTINLSQPINPVEIHSALRKLKESAPGTDGVKREHLRSMSRTDLQALLNIIWGAKYFPPSLQMNRTTLIPKSGNLKDPKQWRPITISSRVLRLLNKIITERLENEIRLSHSQRGFTKTDGIMANNSILQSVIRTMRNESKPCTILSIDLAKAFDRVSIVSIIDALIKRGLDLHTIQYIEESYRNTSTILECHGKRSESIKIRRGVKQGDPMSGFLFNLILDDLLKKLNSREGVKINGVNIIALAFADDIVIAAPSPSVMQSHIEIIEKFFDHHGFEVNVDKCAAYQLLSVPGTKRLVIQTKPLLRINNQPVPTLGVSNQLKYLGHQYTYRGVISPTPSRMEEMLARLKKSPLKPWQKLNILQRHLIPKLHHGMQTMDINKKKLQYVDSCIVKFVKATLHLPKTTPTAYIHAPVKSGGLGIPSLCIHISSVYLRRLERLATRGDNETQHIMQAQAMQQLFTKLKRILKDINSNTKSAVQNHWSEQLHTTGLGAGLRAMTSGISSWIYGPPKFWSGRDYIGAISLRIGLLPTKGAPYMNDTDCRNPSCAGTRETLYHILQRCPVTHYTRINRHDNINKTLKDSLQKNGLKCEEVPRINTQSDRYAPDLITVNDDTAYILETTVAYESKTNSLDNAYKIKKEKYSTPEVIAKVKTTYRVSNVKVMPLVVGARGSWMASNDIILSAFNLPRGLRTVICNLSMQWGVSIHRTFMKTVWRLTNRQFDPGGRKHRNKNYAKRNAYSNI

SEQ ID NO: 58 - protein, 1090 aa, synthetic construct
MSPPQQYEGSSSVATPSGGTIVNVVRETGAGASTPRSWNFTAGTPTNENYAGTSTTSQEMEGSSRNSIPAPQSGQSGSDCASSITHQCQMCGRGFNTKTGLGVHKKRAHPLEMNAESAPREAKRRWRDEEVSLMAKTEARLTGEFGICGNMELMRELPNFGRTLEAIKGQRRKPAYKALVLRYMEQLSSPGQEEISEHQEMTADPQISPVPVPRIDGRSSDPPVANVGVEHIYVPSSETQGSQIVAQLLERAAARRGSSGFARRRRDPQDAAESASLSKRKLRRREYAKVQELYRKCRRRAATEIIDGSFREVRHELEELDAYWRPILEGVSQAPGPTPEALRAFNKRKEQDGGEHNRNLSELWKPFTEDEVKACQIDLQTAPGPDGIRAEDWRAVPAAQKAEIYNAWIKRGEIPPELRECRTVFVPKKDDPAGPGDYRPISIASIPLRHMHTILARRLLACRPPDARQRGFICADGTLENTAVLDAVLGDSRRKLRECHTAVLDFSKAFDTVSHAALVQLLRGGGLPVEFCDYIARLYTTSTTVFDVRGRTSPSVKVGCGVRQGDPLSPLLFNMAMDVILASLPKEIGYGLNAERVSALAYADDLVLFAGSKVGMQEAIDTVDRVGRMMGLYINRAKSSVLSMIPDGKRKKIHYITGHTFKVHGKWISQVNCVERWRYLGVDFDSSGCAILEQNIATALENIKKAPLKPQQRVELVRGHLLPRFLHGFVLGNISADRLRQLDIQIRKAVRGWLRLPKDVPIAYFHASIRDGGLGVPALSSMVPDLVVRRFGCLSTSSWGAARAAARTERIRRKLDWARKQVDRFSKEDPSTKAMSITQYWRETLHASTDGYALRETSRVPASTKWVRERCEQVSGRDYVQFVRTHINALPSRVRSSRGCRIGGETQLRCRAGCMVKETTAHTIQQCHRTHGGRIKRHDRVVEFIAEQMGKGKWSTTKEPNIRTELGLRKPDIIAKRDGVGIILDVQVVSGQRPLDDDHRDKRNKYGKHDELVEKVADILGLQKSDVRTTTCTISWRGVWSRNSHRDLRTHIQLTKAALELVPIMVLRGSHTNWSRFNQMTCMKGYERPE

SEQ ID NO: 59 - protein, 1108 aa, synthetic construct
MEKTNKRMYPRPGSRMARALRVSESECVVPGSSGPANAQVEEVPTSAHSVGRTTANELFRCEHCDREFNTKRGLGVHVRFAHKEAANDLIDVERVRPRWPEEEVRLMARAEATATLAGVRYMNQHLSVLFPNRSLEGIKGKRRNAAYQAMVLEFRQQELDSNSDSDDGLVADATMTRVARTPDRPSVAIEQQTPQSPILHDQDGLGRVRLAIATLLESTARYRIYEAGTLREVARDALAGERVVDRLNDWLAAIFPAGRVNPPRPNRNRSVRPPANENREQRRKREFAAMQNLYRTNLRACLGNILDGDITRERPSGISFAQYWQPLIETVSAADGHMEDLRRSATGSRQSLIRARLSNGPSTSGYGVGAGVSRAADKSSLWDPITTTEVVRIPVKTGSAPGLDKITPRAWKAVPADLRALLFNLMLLAREVPSSISSSRTVFLEKGSMPEHPAPSDYRPLSIGSVVLRQFHKILGRRLAALDIFDERQRGFRPVDGVCENVEVLSAVLADARRRCRTVHMACVDLSKAFDTVSHGAIHLALEEAGLPLSFREYIRACYSNATTALQLPDLCETPRIQLGRGVKQGDPLSPLLFNLVVDRALGVLSEDVGYRLNGELIGALAYADDIVLLASTRNGLQENLNRLQAAVRLNGLDVNPHKTTVLSLVASGRDKKVKVDKTPCFTLGATLLRQCSPVEAWRYLGMTYIGARERIEQPPLGEAIEQLSRAPLKPQQRLRLLRDCLLPRYYHRWIVGSVTAKTLRAADVQVRNAVRRWLRFPHDVPVGYFHAPIQSGGLGIPQLRSIIPILKHDRLQRLCRSSLPAAKAAADSTYVARQLVWCERLMRVGNVRVASTVELRQQLASMLHSSCDGGGLKEVASSTLSSHWVSGGAASIPGADYVHYHHVRANCLPSRARTARGRDGREVQCRGGCPERETPAHCVQRCFRTHGGRILRHNDLCRRVCSFLRQTGWQVDEELSYSTSAGRCRPDLTISRDGTAVVVDAQVVSSEGSLAVAHRRKVEKYGTNNDLVDRVADHTGVPRNNVRFTAITISWRGVWSPDSERELCELGLTRNQLRTLTTRVLWGSWLNWKRFNSITSRYEAYARRRPI

SEQ ID NO: 60 - protein, 1135 aa, synthetic construct
MGIQNNIKASEAWHGGFIDHTVCVEGSLPNRLDETGGSITKRSGLLSAKKRQKLIRNSEVVIIKASKKDLTACPACQKEGKNIGLLGAKTLSKHLEQFHADISPQWKCPGCERLFSKIHSWACHFPKCKGKIEKVVGEFKCSVENCEYSTATQMGLSQHERFRHPDLRNKKRRGNIVIEEEEEAAYESSNTNHTAEKDDTICPDSDEESSSDNESFKSIIFDDPIQLTMDQESVENNLQITTEWKVKLIDFIKETRIEDPKYDAWKKKIDNEVSNIMNHELINDLYTQLVDIIMTEEVMTEEEQLPYEKSRKRNKRSHSGKNPRNRKRRSRYHRKSNFQERKKYAYARCQDMFHNCPRKLADAVMNNDLSVTQKSTVLPPKADVENLYKKLWETEGPKKVKFECEPENNKIEIDQVFTAISTTDVIKKIRKVDKETAPGLDGIKKVNINNTSAAAALSVYYNTLLINEYYPKEWRNNRTTLIPKVGKDHQDIKNWRPLTISSLVSRIFSNMIDQRIKRTIQQNPRQKGFVSENGCSNNVMILGEAIKSAKVNGGTFSILDVSKAFDTVPRAAIKEALRVKGITQPIINYIMQMYEDCETTIKTNEGPIQVKLIRGVKQGDPLSSLLFNLVMEPMIIKLQKETEGISFNKNNVTVLAFADDIITMAQNKEQAKMQVKIVHTYLKKLEMELSISKSTSMQYVPLNKSWYLEDPNIIIDGVKLPEAKSEEIITYLGAKIGPWEGLISNFEADIFREIILRTKKLQLKPYQKLQLLTIYLIPRYTYGLIVSPPSQTTLRRIDEEIRNQAKKILHLPESISTDFIYTPKALGGLGLSELEKIVYIAAVRNIIKARDTDDEALRAVAGSENINKKLEKYAEALELPLPCTIKELDKYKCQYKVNRKLQWKKQLCQGKGSEYFEDQVSNAWLYRPELLKASRYTDAIKLRTNTYPVKAILKRAKLSDDDFCRKCHSKRETLGHVLGECTLTKPSRINRHDEVKHLLIDRIARQKDWMVSPEQDLKNINGRYKPDIIAINNKSKKGYIIDVTVRFENGPYLEKARVEKIDKYNTIKKLVEKVYNIKTRDIEIVPIVVGSRGALPSSTRNSLEKLKIQKKDRLTISLIALRSSIEILNQFIDYK

SEQ ID NO: 61 - protein, 1020 aa, synthetic construct
MGYQPIWLKTIGSNTNNFPRITRSRATARDNQFRRRSISNPPVPTLSCEYCDRVFTTKTGMGVHKRRAHPDELDRQRENEHTIVKMRWSQEEISLLALQEVKLIREGKRHINRILATMCTHRSVEAIKKVRQKADYKASVSQLLNVRGGNGESSNTITRASNTTPSAFLDHIKNTVIPTADLHSNELLVIANEAHSASKELTLCKLVACLHNFLPHRVVRPRRQVREEPPSNRRNARRMDYARTQRNFYKHQGRCIKSIIEGSIMVPVPNKELMIPFWRATMEQEEYVSPSDEEELSDYSDIWAPITLDEIKQHLPPISSSSGPDGISPRDLRNTSETSLMIVYNLLIWCEQCPQQFLEAKTIFLPKKCGPSEPGNFRPITIPSVCAKHLHSIIANRINNKVNFDPRQRAFRPTNGCADNTTLMDMVLRHYRKKSTSCYMASLDISKAFDSVSHNTIFGTLKTYGMPEGFVAYMKFVYGNSTTRLTGDGWVSDVVHPKRGVKQGDPLSPVLFNMVINRLLRKLPDEVGVSLGGTNINAIAFADDIMLTAATPAGLQLLLDRTHEFLTECGMKTNRSKCFSISIEASVKKKHTYVGQRTFSIGGHNIPALKRTDEWCYLGINFTADGRCKIFPQSLLEPRLANLKKAPLKPQQRLHALRTVLIPQLYHRLTLGSVMIGSLNKTDAIIRGAVRNWLNLPKDVPNAFFHAPISDGGLGIPAVRWIAPWLRMHCLEGLELPNQQATLTANTFLANEIDIAKRRLLVDGQFLSTRADIDSFWSNKLHTSVDGSGLKEASHAPYAHRWIREPSRLLSGRDFVNCLKLRINALPSKSRTTRGRARLDRQCRAGCMTSETTNHILQGCHRTHGSRVERHNDVVKFIKRSLDEQGWVTSLEPRFETSTGIKKPDLISVIDNVAIVIDAQVVTDGRALDDAHNAKVRKYNVDEIKRLVKERFNEPGVVVTAATLNWRGIWAKQSINKLLSLGIIKKNSAALISTRVAIGGVQSFRIFNRSTLVTPRTGVG

SEQ ID NO: 62 - protein, 1019 aa, synthetic construct
MTNINGGPSGSTIPTLNKGNDDLTSQAETPAQNVPCRFAGCPRVFGKPAGRSIHERSAHPDWYDNYTKREPTKARWNAEENALLARKEAELTTSGVKFMNQALAKAFPERSLESIKSRRKQAQYRQLVEQFVSEGKRGEPPTPDRISMDTEVVSPRRDYDGPIISHLDSLPAPDTTDFCAAELVDICNSAKRSSKQAILERLTLYLRDVFPCNKTRTRQVPKTPAVELPRRKLRRMEYARTQNLWKRNPGRCLKSLLKDHNTTEQPPIEAMVPYWEGLLTKSSEACPEMEDLKTEIYELWAPVTSEEIKSSFPGSSSAPGPDGVPIRVLRRMPKGLLERILNLLMWCGKLPQHLRESRTTMIPKKTNATDPADFRPITVSSGIVRILHKVLARRIAAKIRIDSRQRSFRDSDGCSENVFLLDLALRYHHAHFKPFFMASIDVAKAFDSVSQKALLQTLQLCGAPEPLVQYIQEVYATSVTRLSCGNWTSHLIQPRQGVKQGDPMSPVLFNLVIDRLWRRLPSAVGIQIGDLDINGAAFADDLVLFASTKRGLQLTIDETSDFLSQCGMHINAAKSMTVSLKPVPHDKKTVVDAANVFSCAGKQLTALKRTDEWKYLGIPFTPEGRVVADPVGQLTASLSTITSAPLKPQQRLFGLRTVILPGLYHLLGLGGMRLGVLNKIDRVVRRAIKQWLCLPHDTTSAYFHASVKDGGLGIPSVRWEIPRQRLGRLLALPLSEGAKRTIPGVFLTKEIVNAQNRLKDGGNDLLTPDLIKKRWAEKLYTSVDGIALKESHKVPQQHRWVAEGNRFLTGRDFINLCKMRINALPTRSRTTRGRVEDRYCRGGCNTAETLNHILQQCHRTHSARIQRHNAVATYVARSLKTKSSVVDVEPRIETDEGLRKPDIIAKIGDEVIVIDAQVVGEQTDLNRAHRMKADYYRKNESLVERIKNKYSADMVAFETITITNRGLWSSHSAEGLVKRGLIKKSELKLLSTRAMVGGLNAYRMFNATTNMAWRRVGVG

SEQ ID NO: 63 - protein, 1389 aa, synthetic construct
MVPPRGGAGHCRRERCKASPTVTKPCGPSRLPREPRPSGRGERPQRPLHARTLCFRRVRVTSPNPMPFTTGPNVKRSLGREGSTGPDSSGPSGTLDADQACTGSPAPTTCAPKTGAIPKVRPGGGPQPARAGTTPGPVRTSKNDGAKKFSGSQQAPPPIQSGPASLGCASCGKTYTTRANLARHEKDCGKRDRQKCLFCPFSASTFAGVRQHERRAHPQEYAADLEGKVGAPESQIMSKLASIEARCKGGVVSIKELTKATGLTIHQVRSRREKPLYQKYLEREKRCLREGGIGTLFSTGAPQPRPSAPSVSVRPSPTRPKDAKPSPTTEMPLPRRDTAIVTAPPHPPQAALPPKRLSDVPTPGKRRPSASPQLPHPAKVKRGITHAIQPPTTSRADPIETSLPELTIPPTPPHPPSIPVLTPVPKTPSPPSTAPSINLVTPPPPHTDMTHSSNRGNTIQPKRKRETDSFSPSNIRKRTVTFHESPPKPEEEVRDLPVGPPPRRSLSASDILALGEPLLEIPTELQELPFLDHLLQSRNGADDNTKALISAGLTASLDALDAAITTWLETAFPTATRRRMRGYRDRKPRPAPPANNQQKRVAAFKKAQDLYSKDPKRLASLIIKGEPLMQEEVLPDLPSIEAYYAGLFEVTPPCARFCVHNTKDAGEVFFPIKEEEVLGSMRGWSNSAPGPDGITIAQVKRCPASLLEALFNILLYRRYTPTAFRASRTVLLPKTGNPRELANWRPITISSAVQRLFHRILAKRFATAVDLHPLQRGFTELDGTLANTVLLDTYIKSRRVQGKTFHVVSLDVRKAFDTVSHQAIRHALGRCGIPSALQDYIASTYDGAFTNIEAGGNRTRRIRFMRGVKQGDPLSPILFNMVLDEVLEELNAQPHGGTLPSGDKVAAVAFADDVLLLQDEDVHVPEALDTVLRFFRARGMAINSNKCSAMAATSTQGKTVPRTKSIFKINNSFIPSVQHMNTLKYLGLHFGVAGAEKPSIHNLVGWISNLHSAPLKPDQKLTMLKRHIIPRLLYGLQSPAITSHHLQEADRVIRRAAKRILHLSSRTGSQFLHAAIGDGGLGIPQLRYQVPDILKRRLDGLISKDEYMGRLFRAKGTAVDFYKRVHRLAAQGPPRAYWREQVTTRPLSQGLQACEEDAASRQWLQRIPPGWTGKDFVRAVQLRTGNLPTIGLPYNPPEMSRCRAGCPKRETLSHVLQGCPATHFERIKRHDEVVNKVAAHCRRKGWTTEVEPRIRHPDGQLFIPDLAVHLPGSSIVVCDVQVCWEGQRTLAESWHRKKLVYDHPRFHDAAKRRWTGSTIAVVPLLLGARGVWPRCNQETERLLQLSKHLKGSCVQSCLKWGSTIHRIFMASVWRQATRPALNHQPRPPE

SEQ ID NO: 64 - protein, 1040 aa, synthetic construct
MLPLRLTVLLGCQPARSYLVGPNYRNQASDEIYSAGGPSGSTGPTSATEEADLHETFSCRKPGCDRHFPTSRGRGVHEQRAHKNWHDERQVDLIDQKKAPWCAEEQALLARQEAHLILNGTRFINQALMPFFPKRTLESIKGQRRYQRHKDLVLSILQDLANEYNEPTQAPRAATSQDSQDGSLVLSIKSLFDQFEPLTGSQFNADQLGRICENITVWSKDKVYEELEIYLLQTFPVTTRNRPKASNAAKRPLSRRKARRVDYARTQKAWRKNPCNCARVILKCKTTAKPPAKQDMINFWRNTMTNGTTESPDKEPKRPVISSLWRPVVPDEVSKSFPALTTCPGPDGLTTRLLKAVPTLILCRIFNLIMLCGKLPKHLLLARTTLIPKKDSALNPGDYRPITVQSILTRTFHKIMARRLTELIPLDNRQKAFIPTDGCASNIFDLDMILRYHRQHFKPLYMASIDIAKAFDSVSHNTIRDTLEVMGVPEPMKDYVLDSYKRSSTRLTCDGWESDEIVPTCGVKQGDPVSPIIFNMVIDRLLRRLPSEIGIKIDHAFYNALVFADDMIFLASTPQGLQIIIDKASDFLAKCGLFINASKSFTVALRNVPHLKKSVVDTKTQFMCRGSKLPSLKRESEWKYLGVPFTPEGRTVIKPEEQLLEALNKLTKAPLKPQQRLFALRVLVLPGLYHLLTLGSTTLSRLKKIDAMSRAAVRKWVSLPHDVPNAYFHANVKDGGLSIPSMRWLMPLHRRLRLQKFANPDQPSSVYITSELQRTIRRLTENRLDIDTTDKLQDRWAKLLHDSVDGKGLRESRKVPTQHQWVTEGNRFLSGKDYLNSMKLRINAMPTRSRISRGRINDRHCRAGCNRTETLNHVLQQCHRTHKARIERHNAIAAYIKRALKKSSEKVEEEPHFNIDEGLRKPDLISVSGATALVVDVQILSDHVDLNEAHKIKCDKYSNLESAIKYRYEIDHVSFTTATLSYRGVWSDSSANDLISRNILKKKELKIISTRVIVGGLNCFWRFNRTTSVRSNTLPRTGVG

SEQ ID NO: 65 - protein, 1031 aa, synthetic construct
MQNYNLRNRQASSPAMPASAEEDNSLPHICQGCGNRYRTEVGMKQHIRRYCRGPPNTQHTCATCNITFRTYSGLRQHMRHRHSETYNAELQTESVVAKRRWTEDEQNEMAKAEAEFEGREPTAIINYLASISQRSRDAIKKRRQRRSYAEKVRNYRENRHSSGNSSSEYESANEQTEEHEANEEALVAYLTTLQLNEEDRRLRNSLITNMSSSSVELNEWTSTLLRRYGRVETNHRPTRNNRLIAQLREATGRIKRAIEYRLTQQQYQKDLSRLAHRIIEGKGWSEDSQSLPTAEEIRRYFQEHENVNSREDDSQITDKFPEGVTLSTPVTIEEITTTIKTMKSNSQGVDGITLRRLKNIPKNKLSLLFNAMILLEHTPPELKKSRTIFIPKNAKTNSLNDWRPITISSILIRLLHKILAYRMEVIQLHKNQRGFSRIDGCFANAFTLFSLIKYHRRKGIPICIVSLDIRKAFDTVSHASIIRSLKRCNIDDKTINYIKDNLTNAETKIKTANGDVTAHVRKGVKQGDPMSPALFNILVDEMLCGLNKFEGVTIGTNQKLRCMAYADDILIFAKTRENAEIMLDYVNEFLSDRGMKINAQKSIMLNLKVVPSKKKVYADEQPAKIGDDLIRALRIGEQFKYLGEYFTPSGSTTCSSESLFNELQRIKRAPLKPQQKLNITLTYLIPKYVHYFQRPSVTRKCLKEADRKIRTCIKNILHIPQHSKNAILYAECKEGGLGVFELLSRIPVIMRERIENLKSNDEAFANIATLNPGTITKIERMIRQEVDTKSKQKKHQAQLLDSSISGNGFSQIKNHPAAHNIIRHPPIYWTSEDYIKAIHLRYNLLPTVGIPSNPPRARRCRANCAKTESLSHVLQGCPLTHWERIRRHNFVQIRLADMARRKGWQTLAEPHIRDREGILRKPDLILIKQNQLVISDVGIHWEGPESLNLQYHHKLATYGSQNFIEAVKLKFPNRNITIAPLIIGARGIWASLNRTLGQILEMSNAEAMDLITTTMRGSWIIHQHFMRTIWQ

SEQ ID NO: 66 - protein, 1055 aa, synthetic construct
MGDLTVAKSPNKKILGRPDLPQESGSTVSSSPVVRSGVPGGNAGRPLGWTCPMCGRSFGTKTGLGVHKRRAHPVEANAEAAPALVKRRWSEEELLLMARVEAQLTRETGKCPNADLLAKLPQLRRTLEAVKGQRRSAKYKGLVKTCLEELALSEVEPNARRGSRTERTPEVLPTRESESASEDPPDDEPRRTAQTPVSSNAAQVLEDLLREIERRKRRCEPGAKARRRLRRKNNGFDYPSASDFRSSRRRRRAEYAQMQELFRKCPSRAAAEVIDGHTRAVRHSLGELETYWRPILEAVSSAPGPSKEALKAFGKGLPYGGVNDYAKLWAPVTIDEVKASRIDMRTAPGPDGIRAEDWRAIPASSKAEMFNEWMIQGSIPESLRQCRAIFIPKSDAPKGPGDYRPISIASVPLRHLHSILARRLLACCPPDGRQRGFVSADGTLENTAVLDAVLGDSRKKLRECHVAVLDFSKAFDTVSHAALVELLGARGLPDGFCRYMATMYETASTTLAVGNERSHPVKVGRGVRQGDPLSPILFNMAMDLVLASLPKGIGYRLENEQISALAYADDLVLLAGSKVGMQEAIDRVVQVGEMVGLGINIGKSSVLSMVPDGKRKKHHYLTERVFSVGKKKLRQVSCVERWRYLGIDFESSGASMLEQEVRKALDNISKAPLKPQQRLEVVRGHLIPRFMHGLVLGTISDDRLRMLDIEIRGAVRKWLRLPHDVPKAYFHADVKAGGLAIPSLRTVVPNLILRRYGRLASSRWSAANAAARSERIRRKLSWAKKQLRKLTRENPKTHLTSVEQYWRESLYASTDGLELRESFRNPASTKWIRERCMQYSGRDYVQFVHCHINALPSRVRASRGRRCMGESTLNCRAGCMLRETTAHTIQQCFRTHGGRIERHNCITSVIAEALTKKGWTVVVEPRFMTSVGCRKPDIIAARNGAGVIVDTQIVSGQRPLDDAHRAKRSKYGKHAELVQLVADKLGLSGAESVCATSCTVSWRGVWSGASAREMRELLGLNKSIFEQIPGLVLRGSHMNWTRFNQMTAVSTVTPD

SEQ ID NO: 67 - protein, 1161 aa, synthetic construct
MAQANNLSLGLVRSRSNRTRAGTPVSEGTVVDDPGENSDNERGETLEIVVPIDQANTRCSKCARRGSRRIFNNLKDLNEHMAKDHPLLRVVFICHFCGRKCPSKHSWDCHRPKCPGAPVANDRQHKCDHCARSFNTRVGLSQHERHEHPRARNEQREAQANRQRGQGGRPAHVWSQEETALLIELDERFRNARFPNKEIQRFLPNKTFKQIGEKRRRIPRDVPEAIEIVSDSDSDNDEEEEVFHDAEETQEPLDLTFNNLTTQEEEDDQSIDEWQTAMDEFILAIEPNENNKFRESTNRLKQIWQEHKNAQESLEHQINAFVNELNVVLIGVDKAQPNGEQRNRRNAQERRRRREEPRRRRDGRATANQVRKFRYARCQEMYKQCPKRLAEIALSGDFSFAEKRKDPPAIEAINELYGQLWGVTGPNATDFIVEEGMDEKRQINEVFPPIKIDEVKARICRVKNRSAAGLDGIKKGHINRKGVAELLTVLFNILIRHAIFPKAWSKNRTTLIPKDGKDHNDIKNWRPITVGSLVGRTFAGLIDQRLRGFVKQSRRQKGFTAEDGCRANTLILEKAIETMKGLHGGVVMIADVSKAFDTVPHSAIKNALQRKGVPEHVVGLIVKMYNDCSTSIKGPEEERVEITLRRGVKQGDPLSPLLFNLIMDPIIDSLDREGGGIAMGEEDLSVLAFADDIVLLGGNTETAQAKVNKLDDYLERLGMSLSAPKCSSFEIVSGRDTWYTRNPNLNVRQEEIPAVEADEMFKYLGTRFGPWTGLQRGKEIPKIMEAIRNLQSLSLKPYQKIDILATYIIPHFMYELTASPPAATILNVLDDNIRVQVKRILKLPLSTARGFFYTPKRDGGLGIIRIARAVQLLTVKSGITIAESDDPILREIERNAKFQKRVDRGIAGLRVTRPRTKAAYEQIKNLTREREREAWRDLRSQGQGVDDFKGDKYGNCWLKRPSILKSSRYLDALRMRTNTFGTRVVLSRTTANAPTLCRRCNLQPETLGHIIGQCVFTKPARIMRHDKIKNLIADRVSRTYDVMIEPTVNEGNELKKPDMVIKKEEEAIVVDVTVRYEKGAFLAEAEKEKKNKYARTAILMATRMGVPKGKVLPIVVGSRGTLPRKTIKALEELGINKKDRQTIAMIALRSSIEIANAFIDY

SEQ ID NO: 68 - protein, 1029 aa, synthetic construct
MKANNSRSADRAESTQPEAGPSRSERQETYQCRFPRCLREFSSASGRGVHENSAHKNWYDSFKLKNTAAPVKDRWTREEKLMLARKEAELTYAAQGKRIFLNQELLKTFGHERTLESIKGQRRKDEHKADVSMFLDELHRQPRNDSDDTRDSDSDDSVTIDEDPFLAYFDSLHPLTTNEFHAPKLNAICAITPDTIRENVRVDLGMYLKEIFPANVKSCNEHLRPQMVQQPTQSRKKRRRADYARIQDLWKKNPSECGKRILDDLVEQQDSIPKATMEPYWRTVFTQVKKATPILGEQMNTLDHLWQPVTIKEIKLAFPPKNTAVGPDGVNSRMLRSVPLPILARIFNLILWCGQPPEALCASRTVLIPKKKGAVQPGDFRPITVSSVVIRTLHKAIANRLKAVPLDERQRAFRNTDGCADNTILLDLALKYHRNRHKKMFAAILDMAKAFDSVSFEALKTTMSAKGIPHPLISYIMTLYQNSITILQHGNWESEEISPSCGVKQGDPLSPTLFNFLIDEMLKSITPEIGIDLNGLHVNVLAFADDLILVASTETGLQTLIDQVASYLKNVGLEANANKCATIALKSIPRQKRTAFDEKCRFKIDGVTMPALKRTDKFRYLGINFTPEGRAKLDTKENLETQLERLTKAPLKPQQRLWILRTIVVPRVQYKLILGDATFGYLKKIDKVIRATIRKWLNLPNDAPNGYFHAAIKDGGLGITSIRWTAPALRLRRLQGLLKSYYITGPVAGAYLEKEISRSENKIKDSNNVILRSNRSIEAFWANQLYKTYDGAPLKESRKTKYQHNWIGEGTRFLTGRDFLNSCKARINALPTLSRTARGRIKDRNCRAGCGRIENLNHILQHCHRTHKSRIMRHAAICKYVARNLKKLEYVTEEEPKFRTSEGLRKPDIVATLGKTSVILDAQIISDRFDLDKANASKTEYYANNESLINEVKERYGSDTVLVNAITLNWRGVWSKASCSYLLTRGFIRQKDIKVLSSRTIIGSITGFNQFSRATTARPEKEGRRRGKT

SEQ ID NO: 69 - protein, 1010 aa, synthetic construct
MNLDNCAGRPSGRSGPTTQVEMPTPEAFTCKMPGCGRSFSTKKGCGVHEQHMHKNWYDQQQITADRTVKAHWCAEELALMARHEARFTLDGVRFLNQALLPVLPGRSLEGIKGQRRTQRYKCLVTRFIEELSNESRPETSNVSRAGSPDRSAIAGDLNYNILMALSTLEPIDIDGFHAETLNNICRCCTSWDLDRISVELEIYLTKVFPPSTKKHKKAQTNLNSLRTRRQARRAEYARTQQTWTKNPWRCLRSLLTNATTNSIPKREIMVPFWKAVMTGNNDQSSLPEAVSAPISKLWEPILPDEVAKCLPSTSTAPGPDGLTAKLLKATPSSVLARVFNLFMATGRIPKYLLEARTTLIPKKDGAAEPGEFRPITVPSVLVRAFHKVLAKRVMCSVRIDKRQKAFVPVDGCLENVFDLDLVLRHCRQMFKPLYMASMDLAKAFDSVSHQALEITMRAKGFPEPMIQYIMYGYNGATTRIACDSWESEPFHPSRGVRQGDPLSPIIFNLVMDGLIERMPKEVGVDIDGRRFDVLAFADDLVLIASTPQGLQEMIDVVVDYLPRCGLALNVSKSFTVAVKNLPHIKKSAVDAKAKFTCQGKCLPALKREDHWNYLGIPFSPEGRIINKDDRDQLQQALEKLTKAPLKPQQRLFGLRVMVLPGLYHLLTLGSTNLSRLRKLDALVRNAVRKWLGLPHDTPKAYFHASAKDGGLSIPSLRWLMPLRRRERLEKLKKGDQPITSFFSQEIAKAMRRLQDGQVDLASCKLIDDRWANLLHNSVDGKALKESKKVPQQHQWITEGSRLLSGRDFVNCTKLRINAIPTKSRTSRGRRTDRLCRAGCQETETLNHVLQKCHRTHAARIKRHNALVSYVKRNLDITYDRVFEEPHFKTSDGLRKPDLVAIKGTNGLVIDAQVVSEHSDLEAAHTRKMEYYRPLKEAVMKDHKVTDVQFTSITLSARGLWSLSSSRDLLNLKLLKKKELKILSTRTLIGGIYAVRIFNRTTTSVPRTGIG

SEQ ID NO: 70 - protein, 1025 aa, synthetic construct
MNTNNPGSSDPGDQINEVLPESYVCEFPGCTREFGSKRGRSVHYNHAHKNWYEEHKLGCLGNKARWMEEEGRLLARKEAQIIFEAESQDKRVNINQALGPYFPERTVDAIKGYRRRANYKAMVERFLAEIREQHHKSDIQTPENQTTSSDDCSIDIRNFLASLQPLDVGEYPKFNVSRLNMICKSIQGLHVNGLKDLVASYVHDTFPPQRRGAKVVREASEGPSLSKKRQRKINYARCQQLWEKDRGRCWRAIENEQLLDCQKCLTRDVMEPYWRAVFTNEKHCSPELPMNEDTMDALWNPIGIDEIKSSFPSNSSSPGPDGMTIKEAKEIPLEILLRIYNIFLICESIPEHLLESRTIFIPKKKEVLEPGQLRPLTIAPVLVRSFHKVLANRLKKVHIDSRQRAFRDTDGCSDNTCLLDLALRYTRSSVGQLYMASLDVAKAFDSVSYKALEVVMKAKGLPPPMVRYISSVYRRSVTRFDYGGWLSDPIHPSCGVKQGDPLSPLLFNFIIDEMIKSLPEGIGVKISGMYLNILAFADDLIIMATTTAGLQHLLDCVGNYLSNVGLKINAQKSCSIAITSLGKQKKTAIDATQIFRIKDQGIPTLKRTDEFRYLGVPFTPEGRVDVNIRDILKEKIQRLCKAPLKPQQRLWILRICIIPSLYYLLTVGAIRIGLLRSIDRDIRAAVRKWLHLPTDCPNAYFHADKQDGGLSIPSLRFRAPLLRRAKIIKLRRTGYIIGDIAEGFLEKEECVCDKRLQSGDLWISCQEDLKRMWRDKLAGSIDGKALEDSRNVLNQHDWITDGTKFLKGRDYVNSCRLRINALPTKSRTSRGRAKDRTCRAGCGCVETQNHILQQCHRTHDARIRRHDCVSQYMVEKLKRNNFTVEETPKIRTSQGIKIPDIVATKERECFVLDPQIISDKYDLDHADANKSSKYNDAEMIQLIKERYRAKDVNVFGITLNNRGVWSSRSAKNLLDKNLIKKSDLKLISSRVIIGGLHAFRIFNTRTTVSRSWITKTSVKNRSGIG

SEQ ID NO: 71 - protein, 1029 aa, synthetic construct
MVNSSSAAQPGASGESVGSAGRASEWNCPSCGRGFRTKTGLGVHRRRAHPVEANAEAAPSQVKRRWLDEELDLLARTEALLTLEASGPCTNVALAARLPQLHRTLEAIKGQRRAAKYKGMVKTHLAYLVAQRAADDAGASSSVDASVSAQESGEVESADADDQAPVVQTPISVDAARVLSDLLAGAEARIRRGGLGARAARHLRHTKRHGFLNEPIALDSLSNRKRRRMEYARVQELFRKCPGRAAAEVIDGHCRETKHSLEEMEAYWRPILEAVSNAPGPTPESLRELRPPSDGTHDYSKLWAPITEQEVKASRVDPKTAPGPDGIRAADWSRVSVPLKAEIFNHWMTSGEIPEPLRQCRTVFVPKSDAPQNPGEYRPISIASVPLRHMHSILARRLLDCCPPDMRQRGFVCADGTVENTAVLDAVLGDSRKKLRECHMAVLDFSKAFDTVSHAALVELLRARGLPGAFCSYIARLYETAHTTLAVKNKLSEPVKVGRGVRQGDPLSPILFNMAMDLILANLPSGVGYGLESERVSALAYADDLVLLAGSKVGMQESIDCVARFGDMVGLRINFGKGSVLSMVPDGHRKKHHYLTEKTFSIGRRKLRQVSCVERWRYLGVDFEASGSKTIEHDVKIALNNISKAPLKPQQRLEILRGHLVPRFMHGFVLGTISDDRLRRLDVQIRGAVRKWLRLPHDVPVAYYHAAIKDGGLAIPSLRTVVPDLIVRRFGALGSSQWVAARAAARSTRIRNKLRWARRQQEKFTRKASERDTPCVAQYWREGLHASTDGLELRESSRNPASTKWIRERCMEYSGRDFVQFVHCHINGLPSRVRVTRGRREGRESQLICRAGCVVRETTAHTIQQCFRTHGGRIERHNCVARFVANAMTQQGWKVEHEPRFVTSLGVRKPDIIAVRGAAGVILDAQVVSGQRSLDEAHRAKRSKYGSHAELVRLVAGRLGLPSPDCVRTTTCTISWRGVWSHSSVKEMKDILGLPCHVFERVPGLVLRGSHMNWVRFNQMTAVSTASPH

SEQ ID NO: 72 - protein, 992 aa, synthetic construct
MYSAVPGSPLNEAFPCEFPGCERSFSTKTGRGVHHRRAHPDWSDSRANTASIKTRWTEEEVQLLARKEVDLIRQGMRLLNQALLNAFPNRTLEGIKGKRRQESYKAIVRELLESPEVPDDQPTEPNIENDYSNAFAEFLSALPKPVCQTHQIGRLVKIGRSLSSGRQKVFEDLSLYLREVFPLIKKTPQRAHMRPAAPTLSKRQERRANYARTQDLWRKNRSKCIQLLLNDTSDGKYPPMDVMVPFWETTMKNNCDRSPGMEPVRTLIPQIWAPITFQEIKKSLPNNHTAAGPDGLTARLWKSAPPDVLVRVLNLLMWCGKLPTHLLESKTTLIPKKNKPKEPSDFRPITVSSVIIRSMHKVLATRMSQLIKLDDRQRAFRKTDGCSDNIFLLDLVLRYHHQKHKPLFVASLDIAKAFDTVSHNTIYETLTIMGLPPPMVEYIRGVYNNSTTRLSCAGWSSEPIQPTCGVKQGDPMSPMIFNMVIERLLKRIPQEIGARVEGNTINAAAFADDLVLISSTPTGLQRMLNITAEYLTGCGLNISASKCMTVTLRTVPREKKTVVDKDTVFTCQGRIIPSLKRTDEWTYLGVPFTPEGRTKSDVLQRYTTALNIITRAPLKPQQRLFALRTNVIPSICHQLELGRVSLSLLKKIDKQSRQNIRKWLNLPTDTPVAYFHADVKDGGLGVQSLRWNSPLRRWNRLNNLPAAVTPGSSDPGSFLQKEIHTCMKRLIDNGSQIDSIQDIRKRWSSQLYAKVDGVGLKDSNKTAVQHLWVREGTRFLSGRDFLQICKLRINALPTKSRTARGRPKERLCRAGCNRAETLNHILQQCHRTHGIRIKRHNAVASYIGRGLAKQEYLVDSEPVIGLGDTTRKPDLIAKRGVTAIVIDAQVVHDQADLDREHRRKIEYYEGLKDKIKDLYNVKEVVFTSATLSWRGVWSPASARSLVALGIIRRKDLKVISTRALIGGLTAFHIFNRTTTVDWNGGRTRRGMG

SEQ ID NO: 73 - protein, 1059 aa, synthetic construct
MTRAAARLRTGHPVEQELESSGISAAGSSVRRAPPVGGGHVPLPALQHQCPHCPKSFTTATGLGLHKKSAHREAFNAAINVDRVRRGWSEEELHLLAREEVAAVRRRIRYLNQHLLSIVPNRTIDAIKGARRGPEYRALVASLLQDNPEAPRVDPPTEEVSTAEEVTAPPEMPHPPLSAEEPTGAPAVTAPRDAVESLLHAVEDIHGWNTVDHVRLTRKFLCGGCVLADVSQWLEDTIPSGPSIGVAPGRRLQPYASGSKKMLRRQEYSRIQRLFRKDMSRAAREILDGTGQEDEVPQANVMAAYWGPLVSSPSPPVPPSSPAPIRAELASLWAAVTIEEVGKSPPPLNAAPGLDGITPRLWRSVPAAVKALLFNIVLCTGSWPASLLASRTVFIPKKVPNPTPADYRPISITSVIVRHLHGILARRVRSLGLVDLKQRCFDDGCAENIAVLATILQDARRKLREVHIASLDFAKAFDSVSHSALSDVLARLGVPAEFRGYVDTLYRHSTTTFEVRGERTEYFPLGRGIRQGDPLSPVLFCLVVDRVLGSIPQGVGYLVGEERVAGLAFADDVLLFASTKWGLQTSLDRVTAEAAACGLVLNEKKSSVLSIVPSGKEKKTKIVADPTFKGRGGTILPQLASTAAWRYLGVDFDPDGVRSTRHDLGAYLESIRKAPLKPQQRLKILRCFLLPRLYHGLVLGRTTQGRLRALDRAVRASVRKWLRLPADVAVGYFHAPVREGGLGIPAMTTTIPGLMHDRLVRLEGSSSPAVRACAGLPWAVDRLRWARSVLTRGGEDLTTHYRRGKWWGRKLHASTDGCDLRECPGTTLSTHWIDAGSTAVPGRDYVQYVHVHVNCLPTQIRTSRGPRRAHRDVRCRAGCGVAETAAHIVQSCHRTHGGRIQRHNAVARTVAAALRDKGWTVGDEPVFDTEAGRRKPDIVATKGDELSVVDAQIVSGATPLDGAHDRKVNYYAGNIDLRRALQQKYGVAEDKITFRSCTLSWRGVWSAKSARGLQHLGLTKALLRSIVTRVLQGSHTNWSRWNKITSMAPPTRRVREGVG

SEQ ID NO: 74 - protein, 1140 aa, synthetic construct
MDSEIEKASEADDPENASGGADAAYVAKRRGSASGARVPGAPSDGRWDPIPIRVTQPLTIQGSTDAVMDCPKCLENSRSISCIGAKQLNAHFLEQHPDVHVNWECEKCREVFPKIHSWRCHYPKCRGKKVNPAAEYKCENCKLSFESRIGLSQHERFAHPKIRNTKRAAEAEKPAEKSGRKLTVWTADEMERLEQLNLRFQDERNINIRLMEFFPGKTNKQISDARRRLKPAVVPAPQPDVAPRQPELAAVVEVPRTQNESPARSAAPEQLSFEGEDWRESVLAEIEAESELKGVWATMVARLVSLASKRTTSEDIDLLCDEITECLNKNKGVHKDSRKRRNRKTKSRPPNRMEKRRFAFAKCQELMNNCPRKLADAVAANDLSLIQLRQSPEMAKIKELYTNLWGSPGPQQEPIGVADAEVPLRDIFLPIKPKEVQDKIRRIANSSAVGLDKIEKADLKGKGISIVLAKFFNLLLLKEAYPIAWKQNRTTLIPKASKDLSDVKNWRPITISSMLSRIFSSLLDRRIREVIQQTLRQKGFTKENGCFTNTRLLSAAISEAKASGGVFTVLDISKAFDTIPHKAIYWALIRKGIPAKVANYIVNMYSGCKTTIKARSDGVPIELKRGVKQGDPLSPTLFNLVIEPIIEMVQENTVGVKVEERNLAAMAFADDMVLLAQDRQTSIGQINSVYTELRKRGMALSIEKCFAFEYVPRPKTWYIRDPEMVVRGTPIPYGEPDKAFRYLGATATPWRGLIEGSELHTIQGVIERVKALPIKPMQKILLLRTYLLPRFTYGLVMRPPSKETLKNIDIAIRSGVKQILHLHETTNSAFLYTPMKEGGLGLLETLPMVYLAALRNAAKAAASDDILVRKVICNEASQKVHKSYAAALRLSWPASIKQIEERKRQIKNNYRQEWAQLIAQGQGVQDFGQEPLSNAWLSRSNLLRSSRLIDAIKMRTNTYPTRMTMRRAHDNVDTVCRACGEADETLGHILGQCSATKTKRIKRHNEIVNLLKDRLSVNNRVIVEPTIELHGERFKPDLVILNEERALVLDVTVRYENRNFLAEAAREKVDKYKDISNKLKDNFNVPTSRVVPIVVGSRGALPTETVLELKQLKIKKEDWLTMSLIALRSSIEIANAFMDK

SEQ ID NO: 75 - protein, 1039 aa, synthetic construct
MPRMDDTSKFGDHHLAGSPPGETGPVIQGKTSSSVSSTERGFRCECPGCGKNFLTKIGRGVHHQKVHKDWYDANQNTLHVKVKWNSEESSLLARQEARLALLGVRFMNQELLKVFPNRTLESIKGQRKKASHKATVEQMLNSMGSQEAEDKVSSDPTELTPEGDIGDSGDEVSSREIKICVIKYLETLPPSKTKQFNADRLDYICKSLANWDDAQIFEELSLFFKETFPSRPRRGNEGGPSAKPAAPLPRRKARRVEYARVQKAWRNNRSSCLRSILKDKKTACAPQRDVMTPFWERIMTSTDTTTPGVEFKGEVLSDLWSPVLPEEIRAAFPHSGTAAGPDGFSAMELKKIHLGILCRLFNIIIICGKVPAHLLESRTILIPKKEGASDPGEFRPITVSSIFVRTFHKILAGRMARLIKLDTRQKAFRPIDGCSENIFLMDFALRYCRSNYKPLYMASLDVAKAFDSVSHTAILDTLRSSGVPNLMTDYIASIYKRSVTRLMGDGWESQAIHPTCGVKQGDPLSPIIFNLIIDRLLSKLPQEVGLKLGEHNLNAIGYADDLVLFATTPMGLQQLLDTSAEFLQKCGLCINATKCFTVAIKNVPKEKRSVVDASQTFTCLGRVLPALKRSEEWKYLGVPFSPEGRMNGSPLESLKDSLRILSAAPLKPQQRLFALRTTTLPALYHRLVLGSTTISLLNKIDLTVRASVRKWLDLPHDTPNAYFHADVKDGGLSLPSLRWVIPLQRFERLKNLKIVENGEIPDAMIKFAKVEVRRVEQRLKDGLLFVSSLVEYKSRFAALLHKSNDGSPFINSRDVEVQHRWITDGSRLISGRDFVQMNKLRINAVPLKVRMARGQVRDRHCRAGCKFGETLHHVLQGCYRTHKARIERHNACLEYILKRQKAKFSIHREPVFNTATGKLKPDAILVKGKKAIVIDALIAGERAQLDRAHQDKIDKYKNLANDIKQTYSVSEVAFTSLTISARGVWSKRSYEHLISLGLMRSSDAVILSTRSLIGGLYAIEMFSRMTTSRFPLLLGEELE

SEQ ID NO: 76 - protein, 1183 aa, synthetic construct
MDRINNVDNRPGSRMARAKRAHINSADTAGDGLRGRPEGNSNSTQPHLAAGASHAVQGPALQVLPPVQCELCGREFQTNIGLGVHVRRAHPDAANEAVVIDRVKARWSEEETRLMARAEARGTLAGGIRHMNKHLLGLFPQRTLEAIKGKRRQEAYQDMVKTFCQQVICDDSSTNDGIGDGSTPAGAARTGAAVPLAAIEDGVNSRVSKPAIFFCASESNTPLADNENPASNFLSADESNTRPGLRNYEATSPQLVPRELEVPERLREAIQTNIHSLSRLRSFHAESLCNFAQAILSGSKHTNEISKTIDEWFKQVFPDKTREPPRTGQRKPLGRPVKESGKQKRKREFATVQNLFKKNKRSCLNYILDGDVERVRPSAVAFEEYWRPLMETVSPANGDMSDLLDAYPECRQVRGSRATTARGIHLEGVGASVPGQGGDEPRIPDMSSLWWPITCSEVKRFAVKAGSSPGVDGVSPRMWSSVPIQLRALVYNLLLLAREVPEALSLTRTIFLEKAGMPEVPAPSDYRPLSIGSVALRQFHKILAYRLTHVRNHDERQRGFRPVDGTCENITILSTVLREARRRCRTLHMACVDLSKAFDTVSHEAIYKAIEEAGLPTSFLNYVKSAYCNAKTLLRLRGHTGLPIRLGRGVRQGDPLSPLLFNLVVERALGILSVDVGFRLGNTLVNALAYADDIVLLSSTTQGLQENLCRLSSAFAKLGLAINAKKTGVLSMVASGREKKVRVLTNPTFRLGDVMLPQRSPTAVWSYLGAVFEGATDNLSVPPLGEAIDKISRAALKPQQRIHLLRDYLLPRYYNQWVVQPVTAKTLRAMDVKVRTAVRTWLRLPHDVPTGYYHAPILSGGLGVPYLRSFIPIIKHTRLQRLCRSSLPPARAAVETQFVARQLVWCENQMRVRGVRVTTTAELRQQLSAMLHESCDGKGLRESSAAPSSLSTNWVKDHMLAIPGADYVHYHHVRANCLPSRARVSRGREGRDVRCRAGCPDIETPAHCVQSCVRTHGGRILRHNDLCRQVAGFLKQKGWQVEQELAYSTSAGRRQPDLTIQRGDVAFVVDAQVVSSEGALNVAHERKVAKYGTDEDLVERVAEHTGVPRNNIGFTSITISWRGVWSSASARELRHLGLTVSQLRTLTTRVLWGSWMNWRRFNSITSRYRENGRRPYTSMGSAT

SEQ ID NO: 77 - protein, 1157 aa, synthetic construct
MENIGEASEAAGFENVPGGPVAACVANRRGDVNGARVPGAPPDMRWEPLPLRVTQSYDIQGSVETAIICPKCSENGLNISCLGSGQLNKHVLEEHPSVSLNWSCDKCNKSFPKMHAWRCHYAKCKGRKLANPLATFTCEKCQEKFTSQIGLSQHERHAHPNLRNAKRAAEANAATGQGGRRPTVWTDDELDELREREREFANERFINIKLMEFFPGRTNKQISDARRRLRAMELASQSPQPEDAAIAGPSQDAAHSQSQKSMESQSGSVAVLNSPLREIAPELLEDQWKPRISAELRVEYEVPRKWSAWSTELSQLHESEVSQRDLDTLYERLVADLSDTDPGRNERAKRPKVSERRRRRRKAGTRPNRAQRRRYAFAKCQELMNKCPKRLADVVAANDLSLMQIRETPDTNDTKDLYLGLWGTAGPEQAPLTQVVPTVEVGRILSPITPGEIKSKIRKIPNSSAAGVDGLTKAHIKYDGLSVVLAKLFNLLLVNEAYPSAWKINRTTLIPKPGKDTRNIKNWRPITIASMISRIYSSLLDRRIRGVISQTERQKGFTEENGCFANVHLLGLAITEAKAQGGIFTVLDVSKAFDTVPHAAIKVALERKGIPPLVADYIAKSYIDCHTTIKTQDGVVAVQLKRGVKQGDPLSPLLFNLVVEPIIEAVQDTTLGIKVEGHNLAAMAFADDIVLLASNKEAANTQLRMVHEQLRNRGMSLSIDKCSAFEYVPRHKTWYTRDPGLHLRDEPVPHNEPGETFKYLGAKLTPWKGLMEGFEVETFQDIINRVKTLPIKPMQKIELLKTYLLPRFTYGLIVRPPSNETLKAIDVVIRDGAKRILHLHETTSSAFLYTPKREGGLGLLEVRKQVLVAALRNGVKAMQSADKIVRDSFENDRTRKKYAGYAAALRLQWPLSIQQIDQYKVRMRREYREQLSQQVAQGQGSVDFAYEPICNAWLSRSDLMKSSRIIDAIKLRTNTYPTRVVMSRAHDNVDTTCRACGDHRETLGHILGQCRTTKPKRIKRHNEIVHLLKERLAANDQVMVEPTIEVSGERFKPDLVIMNEKGIIVLDVTVRYENQDFLQIAAREKVAKYTPVANKLKREFKAKSARVAAIVVGSRGALPQSTVHELKQLKIKRTDWLTMSLIALRSSIEMINAFMDA

SEQ ID NO: 78 - protein, 1030 aa, synthetic construct
MVSEQWSLGLPALPTSTTAGPLRTRIASDEICSGPPSGDSLPALPVEPGETHQCDFPGCTRVFNSTRGLGVHRRRAHPTYTDQGMPAPIRKRWTEEESMMLAKEEARLTISGERFLNQALERTFRPRTVEAIKGHRKNPAYRSMVQTFIEEMNNPQQPSSATQEPPADQGNDDIIQEYLRKMPPISGDEYRASELQNIINRMGMQNTQETLLDLTLYLKKIYPPPKTSSGRGKKRMNAKSLTKKQQRRRDYAMTQIQWRRSRKRCIQNILGGATGAKQPQRGIMEPYWAEILRSTSDASPVPEEPPNVHVGIWAPINGAEIVQATLPIDTATGPDGLTARQLKATPPAILQRIFNIVLLCERLPEHLRVARTVFIPKVKNATEPGDFRPITIPSVLTRCLHRILAKRLSTTIQIDDRQRAFRDTDGCRDNIFLLDVILKYHHKRFKDLYLASMDVRKAFDSVSHMALRTIMGALGCPRPFIRYLEAVYRDGFTVLEGEEWKSPPILPQRGVKQGDPLSPVLFNMVMHQLLKKLSPEIGCRVGSECVNAAAFADDLILFASTPQGLQRLIDTATQHLGECGMTINPAKSLTIGIRANGKLKKTAVDVGTRFHCDGKELRPLSRGDEIKYLGVKFTPEGRSRFTPQEVICPGLEALTRAPLKPQQRLFALRFGLIPRVYYHLALGDVTIGVLNKVDVQIRQAVRKWLNLPHDVPTAYFHSDIKDGGLGIPSLRWTAPMQRLGRLKSLRGNHEQLPNESMDHYLEEEIEKCLRRLTDQNILTCNEDIKKRWARLLYTAIDGKGLKESRNVPQQHQWISDGTRLLQGKDFVNCIKARIGALPTRSRTSRGRYTDRQCRAGCAAMETLNHVVQHCHRTHGARVRRHDAIVSYIARNMRHQGYNVQVEPHIHTEEGLRKPDIVATMGRTTMVIDAQVVSEQTDLNAAHKRKTEYYSKNRSLSNDIRKTSASLDVEYHSVTLSWRGVWSSDAAQGLLRRGVIKSSELKVLATRALVGTLAGFNMFSKCTSVRRVGVG

SEQ ID NO: 79 - protein, 1161 aa, synthetic construct
MIRCVCGKDFGSEKGIKQHLRRMYCRGANPPANVCTYCRREFETYSGLRQHIRLGHPVEYNNELIEQVEHPIRDGSRWTDEELIAMGKAEVAFTGRFINRHLSEIFPNRTRDAIQSRRLQHDYKDLVRDLKREAADGASPTVDIIEEVFGTGIGGNNEDSVNNDGDNDEIIEFSINEIARRRSPSHESSQAEENEQQETQTASSESPTKEQSPHEAISPAVTPTAVNEHRETEVDLQQNEGNESLPRAEQSVTDLLNESVLHPGAPEFIPRVRPTSPDPGGGDDPIDAANNIDPIIRFLREYVDRINSWDENDQRLINIFQGPHQAREVQTEAVEGWLESQFTQRNNTRARMPRNGGRNNQTRHRVENERDERPLSRKAQRAIIYKKTQQLFRKNKAELASCIIDDKPCGEATPEPPFASVVEKFGGIFSRPSPPDEHQILDIKESPDGIYAPILPTEIDWAVKSTKSRAAGPDGLRIDQVRRLAPQKLCLLFNLMVLWGTIPTKLKSSRTILIPKGGDTTSADNWRPITVSSILLRLVNKIIGQRLGKLPLHPLQRGFQKIDGVMLNNLTLDCLIRQRRQKRQPYNLISIDLRKAFDSVSHGSIKRAMNRFSVDERIQRFILEGYQNFSTILQLGDKSSDPIAVSRGVKQGDPLSSYLFNMILDELICSLQAKDLGIPLNNTKVSCMGYADDIILMGATVKEAQTVLKHAGKFFEQRGLELNAAKCTATSVGIVPGKKILYTITRNIFHVGGSVIPQHAPGEFWKYLGARVDALGISKPYVDNLKLQLLRIEKSPLKPHQKLCIIKTFLIPRILHLLQTPRITLKLLKESDKLVRMSVRRILHLNRTCADALIHGPIRYGGLGVLSMRTLIPSVMKGRMEKLAAANDTYAAHLATLDDFQKLLSKLTNWCEPAGDSPSVAARWWGQKLEQSYSGGGLIQGQANYHSGSWIDNPPPFWSGGDFVKAIQLRGNLLPTQGIPSNPREQRACRGGCERVESLSHVLQRCPVTHWPRVQRHDRVANLIKRSAIKRGWNVESEPHIRCSNGILKKPDLVCKKADRVLVIEVTVGWESPTSLNVAFQNKKAIYSDALTLEALHTRYPNSRIEVEAIVVGARGTWCALNDRTINKLGCASADIRNIICSTIIGSILVHKEFMKSTYFR

SEQ ID NO: 80 - protein, 1031 aa, synthetic construct
MDGAPTFGGHQRGAPTGDAAPVLNEPRPDPEVPLRGAFACECPGCDRVFRTKTGRGVHYCKAHKDWYDERQNVVYVKARWSLEESTLMARQEARLTMQGVRFMNQELLATMPHRTLEAIKGQRRRVEYKAMVAQALEEMRIPEAAAPPLDPEPVDEDRNPHSPTVQTGVATYEAARERVQEHLRRLPVCEGADFNSERLDTIVESLSEWSRARVFEEMSLYLHETFRRPARARGVRPPPGNRANQSRRKSRRADYARVQKAWKRNRTTCIRDILRDKRTESAPPEDVMVPFWDRIMTSGGTATPGVATHREEIGELWEPITTKEVKEALPPLGTAPGPDSLTVEEFRQVPAGIWTRIFNILMLCGKLPEYLLESKTTLIPKKNGAAEPGEFRPITVSSAIVRAFHKVLANRMSKHIRLDPRQKAFQPIDGCSENIFLIDFVLKHARQNHQPLFMASLDVAKAFDSVTHNAILDILRSSGVPVPMIEYVGEIYRGSSTRLQCGSWTSHAIHPNCGVKQGDPLSPMLFNMVIDRLFSLLPRETGIRIGGTVVNAMGYADDLVLLATTKVGLQQLLDITAEYLQNCGLLVNASKCFTVALKTVPKEKKTVIDARERFRCLGRLIPALKRSDDWKYLGVPFTPEGRLKCNPLDRLKTEIEQLRAAPLKPQQRLFALRTMVIPGLYHLLVLGATNISLLNKLDVTIRGTVRKYLDLPHDVPNAYFHADVKDGGLSIPSFRWTAPLQRFHRLKGLANTAGETGPDAMKCFLEMEIKRAERRLNDHGARIITRAACKARFAKLLHQSNDGNPLKRSRGVEQQHTWLTDGTLLVSGRDFIHMNKLRINAVPLRSRMARGRVRDEQCRAGCNNAETLHHVLQQCHRTHNARIQRHDACLKYLLDRQRTQALVDKEPHFRTASGLLKPDAVIKKGNSAIVVDALIAGERANLDQEHKGKIDKYAALADAVKVRYSVEEVVFTSLTISSRGVWSKRSFSSLTGLKLLNKADAKILSTRALVGGLHAINIFNRRTDVRRRARV

SEQ ID NO: 81 - protein, 1197 aa, synthetic construct
MANLRTMQILLPFESVLCPVCLEADRANYRALSLNDAIRHYSEKHTALEIIYICRLCEKPYKSKHGALCHIPKCPGKKTVTDALQCAECAEAFPTQRGLSQHKRHRHPNKRNEERTAQAQMEKADVPRIGISFTPEDILLMYQLERIYKGDPKITQKMTEHLKTKTLKQISDKRKEPAYCRRRDKYLVEMAKTLPAVKRRSVSLSSASGNVNKTPRDKITRRRGLSVTETDNSNKEIVDIELNDKETGMEPASAVSDTPLDCYRSESEMAENQCQDLTGNNGGHKTRLDITKIILKECRVNLSRCDHMVGTFGSDDEMENDLSESDSWQAGLVEALGKISYAKWPRHLTEIGRSLKEARNKLDIDSVETIYNKIVELLTKSQKSDSHDDRPPRGKHRTKRSHKRYLYARTQDLFKKEPGLLARHVRLGTDHTAQNNASIPRGEVIDLYTALWGSKVPTRVTDCNEAEAMGFDSFGPILAAEIQKRLTRTKKATAPGPDKIKKADLLGPYKCELLAGMFNLMLAAGALPSAWKNNRTTLIPKEGKDGTKVTNYRPLTIGSLLSRLFWGILDERLRSVIQMNPRQKGFVSEAGCYANVRILHELTHQMKKSGGGVGIQLDVSKAFDTVPHEAIPIALKRKGVPAPIVDLIRCSYEGAATTISHPEGEIKITLRRGVKQGDPLSPLLFNLVLEPLLERLEHMPGLPLPGESGISCLAFADDLFLFATDPSKAQDLLDCTVEELGSLGMTIAANKSCAYRIKSSKDSWFMEDPGVERIGEKIPVCTPENKITYLGCSYSVWSGFDLKSVSEHMEQIIKRVKFLKLKPMQKVKLIQVYLIPHFLHQLTIAEPSKAFLIAMDQRLRVAIKQMLHLPLSICDGIIYCGKKDGGLGFPKLQELIPRVSLNAGLKFSESRDPAIRALFGSSVTAVRLRKLANSVRINYPYTKEDVKKYKETCYRTELKRWKELKAQGKAVEAFTDDKIGNMFLQNPQLLKPCRFTTALQLRTNTAGNKTSLNRIAPQNDIACRKCKLQKETLAHILGQCIHTKTARIRRHNEIRDFIENTVSEEDITATISKEPLIDPADGKCLQPDLIIKNQKGVFVVDITVRHEDNDYLEKAKEEKENKYKKLLPQVKQIFEVEHGEVLPIVVGTRGAMPKNTIKALAVLGVKQISKLKTITLMALRSSIEMYHAFIDYDGPLL

SEQ ID NO: 82 - protein, 1210 aa, synthetic construct
MTLCQGYHGLAAHVYPGALLHSIIATENSKKPRNPDCSSVDDPATSHGYPVGSRMDSDLELDLNIDINLDLDLDSNVNMALNDVIEYPNCPSCCINLTSRTLAQRKQHVRRCNRPPPRSDGTYLCETCSSSYKSYAGLRQHIKRAHPEIYNAEDVALIEKRAPSSRAKWTLAEDKQLARRELQLPPGLPQREINIRLASLHPGRSAEAIRKQRLKLSYKTCLQALTTETSSMPVASSALPSTSREINIRLRSPRLRLQRIDSIAPWNNILNKQPITICDSNPDLFPIETSTDSRCNYIQNSDICTYLKELQPSCTPEVQDLVNAVLGDQTFSVIDTMFSMIIHKISSTRHINNSDNVSNPNSNNNSNSNSNSNSNSNSNSNINNVHSKHNNKRIKYNKYNKYKNNDRIVKNNIKKRRQNRASIRAATFSNYQRMFVRNPTQLAHAILDQKDLNAATFPSMEEIEREYHSIFEECPPYSGPLSSRPNDITLLSYPISFRETELTLRRLKQSACGPDGIKREDLRSADMTDLVGLLNLVFGLCRTPSILKRNRTVLIPKKGDLSKVSNWRPITISSLFSRLLHKILAARLSENVKLHYAQRGFTPGDGVMTSYTILDTIVREHRNNGKPLYVVSIDLSKAFDKIHPLAIEQALIAKGVDDHTISYIMSTYREVDTIIECHKQRSSPIKMKRGVRQGDTDSPILFNIVMDKLISSICPAEGVTLGLAKIGCLLFADDIVLFSNTVHGMREHLHKLQTFLHETNMEVNPAKCRALQLVRVIGSKRVAVDTRSRFGISGKAIPTLKVQEQLKYLGHNYHQHGMIAPSTSNLEDMLERLHRAALRPWQKLFILNRYLIPRLLHCSQCSDITAGKLTFIDRLIKRFVKATLHLPITTPTAFLYAKIRDGGLSVPCMRHQIGTIYRRRLQRISTQRDPDFEAAFQTPSFQRLMRRLDIICQGLPPTRSAVSRHWATTLHQSALGNGLKFGSRAANSWILDPPPFWSGRDYIRAIQLRIGLLPTGGAPYINAQAAQCRYPTCVGRRETLSHILQWCPVTHYQRIQRHDRVTQDIAKYLRKMGFNVQEAPRVHTRDNRYIPDLLVIDNKNRIGSIIETTIVWESKNSLLEAATIKRDKYNLPEILDNVRLKYHLLQRPQVFPLVVGARGCWPPSNDIIWRTFKLPNYLQRIITCNVLRYGSSIHRYFMAHVWRSRKKRGT

SEQ ID NO: 83 - protein, 1236 aa, synthetic construct
MKPFVVQTNEILNSIYNIKDVLLTPHRREDRLSRLWLEEGMGFDEDEGVYESSPSQETSISEGTPTLHEPAKYVVSLPNDDPECPVCGDHIGKPTALSIHLKRTHGDKEVEYLCLLCGKSEQRVHSILCHIPKCKGETTTEPEGDWPCPECEKQFQSKSGLSQHKRSAHPALRNAERIQASQPKSDSQRGNHKRCWTDEEVALLLELEKTYLGHKHVNKMIATEIKTKTAKQVGEKRRLLAQSKKKVTVENTNPGPGAERHSNEKGNFKLKETPLSTLEVRPIQPASKLGLNGAYQRKLKVYLENQKKMKQDNGFPDFAKDIFSAWVKGDHNIQEMVEQTSSDILSNFHPTLPKPKRTDRQNNEKGNFKLKEAKSWMRKRAVKRGTFLRYQHLFDRDRCKLASIILDDTERLKCEVSPDEILKVYKEKWETRTPFNGLGHFTSHGKTDNSAFELLISAKEVIKNVKEMNKKSAPGPDGLTIEDLLGEDPEGERLAGLFNIWLVTGYIPNKIKDCRSILIPKTMDPAALRELGNWRPITIGSIVLRLYSRILTTRLAKACPLNARQRGFIAAPGCSENLKILHLLTRDAKRSKKPLGVVFVDIAKAFDSVSHDHIMWVLRERGLDQHMAKIIADSYTNVHTRIEIDKKSTEPISLNVGVKQGDPMSPLLFNLALDPLISQLETTGCGFSHGNKTITALAFADDLVMLSESWDGMTKNIEILEAFCSLSGLKVQAKKCHGFFLSPTHDSYTVNNCKPWRISDDALDMIAPGESEKYLGLRVDPWIGFSKPALSDKLDNWLQRITRAPLKPPQKLEILNTFTIPRLIYLADHTDIKKTMLSSLDKNIRRAVKAWLHLPADACDNFLYSRTRDGGLGVARLSSLIPSIQARRLHRIAHSQDETIRSIALANGITDDYHKLWITAGGDENKIPVITAPVYIDYKLPQGVLDELTEWEKPAPKAIYPIPCSWREAELSEWKTKQCQGSGIQHFKNDPISNTWLRNQRGFPARHYITALKLRSNVYPSREYQGRGNKNMNVNCRHCPAKYESLSHILGQCPAVQGARIQRHNKLCNILKQRARKLKWTIYQEPQLRTAENELRKPDLIFVKDQKALVVDVTVRFEYKDKVFQDAADEKIQHYCSLTPQIKDLTGAEEVEYFGFPLGARGKWPEINYKVLTALGLSDTEQGRTARFFSRRTLLYSIDVINTFASLGKNKPSTVNLYPVNKDHNQEGAAGKSCND

SEQ ID NO: 84 - protein, 1149 aa, synthetic construct
MESINDETLPGSRMARVLRARVRGRAVVAADPDGVVAGSSRQTAGEAMAEGSSQDTAEHGSQVVEQGNQNNMFRCEHCPGEFRTKRGLGVHVVRAHPLVANSAVDVERVKARWPAEEVSLMARAEATATSAGEVLFMNQHLQRYFPNRSLEAIKGKRRQEAYREMVRGFCQEILSRRTTVIDEGDGAGAAMIGADAAPATIETGQEENAPRPAPEQSSQRLRDVITNIIEHSPASRRAYQADTLVAIAREALLSGVVNVDAIERWIGAIFPPTAVRPPQRASAKRRSPPGTHDKREPRENRKQRRRREFAHVQTLYKKNLKTCLSHVLGGGSVRERPSAVEFRDYWRPIMATPSEADGDMTALRESYGTSTQVAACASRSLPPRARGIHLEGLGEGAAADPVPAVDKSSLWGPITPAEVKGTSVKTGTAPGLDQITPRMWNAVPPDLRALLFNLLLLAGTVPWSLTATRTVFLEKGGISERTGPAEYRPLSIGSVIARHLHKILAKRLAALDIFDNRQRGFRPVDGVCENVTVLSAVLDDARRHCRTLHLACVDVSKAFDTVAHDAIHATLQELGLPREFREYVRAVYSSARTVLHGIGSGTDEGAAIKIGRGVRQGDPLSPLLFNLVVDRALGILSEDVGYRMGARLISALGYADDIVLLASTRNGLQENLTRLDNAFKVNGLNINAKKTGVLSMVASGRDKKVKIDITPNFTLGGAWITQRSPVDVWAYLGCMYQGAREYANDPPLAQAIEQLTRAPLKPQQRLRLLRDCLLPRYYHRWVVGTVSAKTLKGMDIVVRTAVRRWLRFPHDVPAGYFHAPIQSGGLGMPLLKTLIPILKYNRLRRLCRSTLPAACAAAETTYVARQLVWCENQMRVRGSRVTTTAELRQHCTAWLRESCDGNGLREAENSAKLSSYWVSGGADSIPGADYVHYHHIRAQCIPSRARVSRGREGRNVHCRAGCLEIETPAHCVQRCFRTHGGRILRHNDLCRQVGNFLQQKGWQVDAELAYSTSAGLRRPDLTIAKGGEAVVVDAQVVSSETALNVSHERKVHKYRSSDDLANLVANRTGVPRENVRFTALTISWRGVWCSRSEEEMRGLGLTTGQLRTLTTRVLWGSWLNWKRFNGITTRYMAGRSIAPAASGAWTEGT

SEQ ID NO: 85 - protein, 1231 aa, synthetic construct
MVQSLALRLVSTGTQTDPLHLWVSRAEIPPPPVVLCEESYDLVTLEFVNSNVCFEGGFHLGNARECLARFPEPYDILPSGLVREERFECEISESGGPVTPVVPPVCSGAGSPPKTPVVSVPDKNPSCPRCSTRFTRMLGLIDHLKRVHGQSKIFFRCSKCGKQHLKYHSIACHVPRCKGMVSVVPTGEWSCEVCHRGFATKVGLGQHKRLKHPLVRNEERIAASRPKESSLRGAHKKVWTEEEEALLKQLVERFAGSRNINQLIMEHIPSKTAKQISDKRRLLSKSPSKKHLGEGEKVGSEHPPQSAKGPQEGQLKLHYIGTIRRRLSAGGFTSCKEAFLKILDGQDPQKVVNEEYEMFRDSLSRLGPRTPRGPRLRRLHGPDASKITAKWMRKRAEKRGKYLRYQRLFQVHRGRLARVILDDTEALQCQIPSGTVHSAFKARWESSVCFKGLETFPVYRDADNIKFQDLITEKEIGKNLDEMKTTSAPGPDGITLGHLSKLDPKFSRLTEIFNLWLVTGTVPDALRECRTVLIPKSSREERLGDINNWRPITIASVVLRLFSRIATARLSRACPINPRQRGFISASGCSENLKLLQLMVKCAKQERKNLGVMFVDIAKAFDTVCHGHILAGLAQRGVDPHIVGLVGEMYNNIKTYIVTRKGRTDPITISSGVNQGDPMSPVLFNLALDPLLCKLETEGKGFHHGGFKITAMAFADDLVLLSDSWEGMCYNIKILETFCELTGLRTQGEKCHGFYIKPTRDSYTINDCPVWQINGTPLNMIDPGTSEKYLGVQIDPWVGFADSGLSAKLEEWLDRIGQAPLKPLQKLDILRTYTIPRLIYLADHTEVRSGLLESLDLAIRGKVKEWLHLPPSTCDAILYSRFKDGGLGIIKLAALIPNIQARRLFKLAQSSDVALVSFLKGEQLERTFEKLWLRAGGTRESIPPIWEPIPMDEGVAEGDKALSEWEAPRSRFHAPRPCDWRKLEFKRWIQLVSQGHGVGNFENDKISNWWMVQYRGIPHRKLITALQLRANVYPTREFLARGRQDVCVRSCRHCGAQLETTAHIIGTCPITQEARIKRHNSICETLSGEAKREGWTVFEEPHLRDEQNELFKPDLIFVKGEKALVVDVTVRYEHSSSSLKEAAAEKAKKYQRLLPQIRDLTNAAAVQFVGFPLGARGKWYERNAELLLALGLSKARLGRIARALASKALLSSVDIVHIFTSKARALYHEIN

SEQ ID NO: 86 - protein, 1175 aa, synthetic construct
MNSNHRSLGPTREGSNDPSSCLNPPEEGGAPTPPHPPAHENLPMMTEEYACPFCPRRFPKKTGLGVHKRRAHPEEHNAALVVDRTKKRYTDEELGIWIDAEAQLVFRGVTKFINVELAKIRPDLERSGDAIRSMRRQPQYKEGVTRRLAELKVLAETAARTPTPDVTPEAPNTPNVPTLPSVTGFEYSELAAWARAEASIISSGGTRNINLVLARQFPRFGRTGADLRRLRVSPLYKRELAAILSEGTSADDRVDVAAPITMNDINRSDVWLYLDTLRADLAAHEGFESQKLNNIISKALGGEDIESEFEEWLKMIFPPPELNPARRRGDNRCSLEGMNAKARRFERRRSYRRIQANWRKNPGTIARQIIKGQPINGDFQGTDFDNNNVEPNLDTFVGAWAPIFGEPSLPFRDVQESPPIRESLSSLMTARISETEASNARVRVSTAPGPDGITARQWGKIPPCIAQAVLNLLLILEKPPKCLTSARTVFIPKTDKPSPRPLEYRPITIQSVILRQFHKVLASRLERADIIDPRQRAFRRADGLAENTYIMQTVLWSTRTQIKSLHMAVLDLSKAFDSVSHSALLAALVQVGCPTSFVRYISSLYEGASTELEVKGQRSGRLPVRRGVRQGDPLSPILFNLIIDIALRSIPAHIGYRMGGNNVNALAFADDVCLLATTRSGLQEALQAFEEALAQRGLFLNPTKSLTVSMEAAGREKRVKMIEGRFTTSGGTLLPSTSVVSTWRYLGLQYGLGGVTGTEKTQTVLGERLERITVGPLKPQQRLRILVTYLLPAFTHGLVFGRVTSDLLRRLDRMVRSTVRRWLDLPADTPLGYFHGPIKEGGMGIPSFSFSIPVLRAERIRNLQRSGLPLGQECWRTEFIQNQLAWCQRRFRTLGVKNSQETKAHFARALHGSNDGRELTESRGCPYSTDWILPNSEGIPSADYRNYHKVHINALPTRVRTTRGRVSRVRDDGVGSDWTARMCCRAGCVNITETAAHVVQNCPRTHGGRIKRHDAMCAYLGGALKDRGWEVKREVRYKARGINLQPDITAIKGDQAIMLDAQVVSASLGLDRAHNNKVEKYGGEILREVVAAKYLVNSDKVKSYAATISWRGVWCARSAKELKDIGIYRGTMRGITTRVLWGSFMNFRAFNRDTTHASTWTRTRRNDVSQRVGVG

SEQ ID NO: 87 - protein, 1017 aa, synthetic construct
MNNQSVGGPSGLTGPTSDSEAAGPSNAETFKCRNPECDRSFPTTRGRGVHEQRAHKNWYDEKQIDNIDSKKAPWSAEEAALLARQEARLVLEGNTKFINLGLKPYFPNRTLESIKGQRRYPRHKDKVLQILREMENDSQGQDQPDLPESNEEPLALNVTSQISPINEYFAKLEPLNITEFQADHLNRICSSTSAWPLCRIFEELEIYLLETFPPTTKQRISGSEPEPVLTKRKARRAEYAKTQRAWTKNPSNCLRFILKGKSMANPPAKEDMVNFWRTTMTNGTNESPENETRRPVIWELWRPISPGEIKGSFPELSTSPGPDGLTSRQLRAVPLPILARVFNLFILSGKLPKHLLKARTTLIPKKDGTLKPEDYRPITVQSTMTRTFHKILARRLTQMVDLDQRQKAFVPTDGCAENIFDLDMILKYHRQNFKPLYMASTDIAKAFDSVSHKTIRDTLHIMGLPNPMVEYIMDSYERSSTILTCGDWESDEIRPTCGVKQGDPLSPILFNMVIDRLLSLIPPEIGVDVGGDHYNALAFADDLIFMATTPNGLQITINTMADFLSKCGLQINAAKSFTVSIRNVPHVKKSVVDGKVKFTCLGRTLPALKREDEWRYLGVPFTPEGRTIASPEIQLKEALDKLTKAPLKPQQRLFGLRTMVLPGLYHLLTLGGTTLSRLKKVDIMTRTAVRKWLNLPHDVVNAYFHANVKDGGLSMPAMRWLMPLHRRKRLLKRVREGQPTDPYLSLEIQRTIRRLTEHGNDMDTLDKLEKRWAELLHSSIDGKGLRESRKVPQQHKWVTEGNRFFSGKDFINAIKLRANALPTRSRTSRGRRSNRQCRAGCNAVETTYHVLQQCYRTHKARIERHNAVVSYLKRALGKTWKRVEEEPRFETPEGIRKPDLVAIKGTAAMVIDAQVVSEHLDLAVAHENKVEKYRPLESAIKDRYVVQDVFFTSATLSYRGVWSVESVNDLIGKKVLKKSELKIVSTRVLVGGLSSFWRFNRMTTAVRRTALQRTGVG

SEQ ID NO: 88 - protein, 1014 aa, synthetic construct
MTRTNVGGPSGFSGPTPALELEGRSEAEEFACRHPDCERTFPTSRGRGVHEQRAHKNWHDERQVDNIVNKKAPWSAEESALLARQEARLTLDGHKFMNQALQPLFPNRTLESLKGQRKYQRHKDQVAQFLRELEEERQRLDHPTDPVSQEIPQDTDSMASFKELFARLEPLDIVEFQADHLNRICQGVATWSVKKISEELEMYLLNTFPLKTDRKEASPRGPERTLTNRQARRAEYARTQRSWTRNPCKCLKEILDHKAPEQTPPKEDMVQFWRTAMTAGTDESPESEPRRPVIQELWKPIDPVEIKKSFPELSTAPGPDGLTSRQLRAVPLQVTARVLNLFMLCGRLPRHLLRARTTLIPKKDGILGPQDYRPITVQSVLTRTFHKVLAKRLTDMVLLDPRQKAFLPKDGCAENIFDLDMILKYHRQQFKPLFMASTDIAKAFDSVSHNSIRDTLLTMGVPDPMVEYVMNAYQRSVTTLASGDWESDDIRPTCGVKQGDPLSPVMFNMVIDRLLRRIPPEIGVNAGGNHFNAFAFADDLVLVATTPNGLQQTINVATDFLASCGLKINAAKSFTVAIRNVPHEKKSVVDGSQKFTCAGRILQALRREDEWRYLGVPFSPEGRTNVQPEIQLKKAVEILTKAPLKPQQRLFALRVMVLPGLYHLLALGNTTLSKLKKVDLIARAAVRKWLCLPHDVLNAYIHANVKDGGLSVPSMRWLMPLHRRKRLQKLVREGQPTDPYLSREIQLTIRRLTDHNLDLDNARKIEQRWAKLLHTSYDGKGLRESRNVPQQHQWITEGNRLLSGKDYINATKLRINAMPTRSRTTRGRGQSRQCRAGCNAVETLNHVLQQCDRTHKARIDRHNAVASYIKRALEKSYQQVDEEPRFVTPEGVRKPDLIAKRGEMALVVDAQVVSEHTNLDEAHAAKVGKYQPLTQAIRERYDVVGVHITSATLNYRGVWSVSSANELNTKGVLKRTELKIISTRVLVGGLNAVWRFNRTTRTVRRNVAQRTGVG

SEQ ID NO: 89 - protein, 1302 aa, synthetic construct
MVQSVSLRYISMATQTDPLDLRVATAAISSIPPTRHDEGFRLMCLESIPPTRHDKGFQLKCLESIPPTRHDKGFQLKCLESIPPTRHDKSFQLKCLESIPPTRHDEGFKLKCLEFVNSSYNEEIGYHLGGIWEGLADLPEPYETLPFRLVQGDVSECVRTVVEEPKSPEALPGLSSEGTPSGTPIVSVPDKNPFCPCCATRFTRMLGLIDHLKRNHGQKKIWFKCSKCGKQNLTYHSIACHLPRCKGEVCVVPTGDWACEVCQKSFTTKIGLGQHKRLAHPLVRNLQRIEASHPKVPSLRGAHKKVWTEEEEALLSQLVMIYEGKKNINQRIMEHLPSKTAKQISDKRRLLMKVPSKGVLSQEEGVGSEDLPQSVDLPQEGLPQEGRLKLQYSVAIKRMFATYGFTSCQEAFAKILDGQDPQMVVNEVYEKFFGSPLELSQDGKSTRRRKSQKGQKASKTTTSWMRKRAKKKGKYLRYQRLFHLHRRRLARIILDDVEDLQCQLPSNIVHAAFKARWETPVGFKGLQSFPPHGRADNTAFQALITPKEVEKNLKEIAKTSAPGPDGITLGQLLKKDPKFSLLTEIFNLWLVAGTIPDLLRDCRTVLIPKSTDLEKVGDINNWRPITIASTVLRLFSRIVTARLTRACPINPRQRGFIRASGCSENLKLLQLVVRHAKREHCDLGVVFVDIAKAFDTISHEHIIMGLRQRGVDSHIVQLAVDLYKNINTFIAMKSGRTDPIKILRGVKQGDPMSPILFNLALDPLLCKLEKNGYGFQRGNLRITAMAFADDLVLLSDSWDGMCGNIKILETFCELTGLQTKGEKCHGFYIRPTRDAYTINDCPAWTINGTPLNMIDPGNSERYLGIQIDPWTGFADSGISKKLEEWLHRIRDAPLKPLQKVDILRTYTIPRLIYLADHTEVRGGPLEFLDLVVRNEVKGWLHLPPSTCDAILYSKCKDGGLGTIKLSALVPSIQAGRLQRLAQSSDETLVQFLKGEHIEVLYEKLWLRAGGVKESIPTIWEPMPVSVSVVSGGGDVSEWEVPVPRPCYPRPCNWRKTEFNRWKHLVAQGHGIENFENDNISNGWLTQYRGIPHRKLITALQLRANVYPTREFLARGKKDVYVTTCRHCGADYETTAHIIGSCPITKDARIKRHNSICETLSNEAKREGWSVFQEPHIRDENQELFKPDLIFIKESKALVVDATVRYEHNNSSLKEAAAEKVKKYQCLLQQVKDLTNAEDIEFIGFPIGARGKWYEENFDLLLALGLSKTRLGKTARALASRALLSSVDIVHIFASKARTVVSN

SEQ ID NO: 90 - protein, 1162 aa, synthetic construct
MSGPAPPAALRQRGPTAPSVGGDPHQSPPALPPSPPPAQFQCVFCQRQFRSLRGLGVHKRSSHPVERDVELAALPDRVKVRWTDEEVRLMARDEAALTAAGEKFLNLALLRSRPERTLEGVKGQRRKAEYRALVAQATADLAAARASPCRPGPAGSPQRSPGHQHAIRRLLGRRVRELMGVYEQVDAQAPVDLPRRADQTPPRLQIPARPCQLSTPELAELAPTSPPPQARLPIQYVLPDARANELQGNVVPFTSLPPYDTFFPSIRLLLPLVMRDEEWQSLQLVEVARSALCGRAKPSETLSALSEWFHSLFPAPPAPSAPEAPRAPAKPKKPKKPKKPLSKRKIRRKEYTLTQRLFKKDMSRAVRSILDGDSLADPPAMPEQGGMFEYWRELMGRESVPLPPRASYPRQAQLADVWGPVSVAEVVRERVALSSAPGPDGVTPKQWSSVPVPLQCLFFNLVLSQGGFPMSLLGSRTVFIPKKGGEGRPSDFRPISVSSVAVRHLHKVLAGRLYKHGLASLGQRCFGDGCAENVFILGSLLRQVRQDPRKGLHVASLDVAKAYDSVSHAAITHALRVKGLPEELCIYIARLYAASHTSLELRGSRSLPVPVNRGVRQGDPLSPLLFAYVVDMILSECPVDVSVRLGDVSVSSLAYADDVLLFGETRRGLTLALNAFEATARRFGLEINTKKSFTLSYVPMGHEKTFFIQMDNPFRLTSGPLPQLDANSRWRYLGVGFGVAGPSNVDPQLATFLERLTRAPLKPQQRLLALRAFLLPRLFHSLVLGRCGKGSLKRADIQVRAAVRKWLNLPPDIPVAFFHAAIRDGGLGIPNLSLDVPAMMDKRLRSLAESSHPAARVVSRSHWADYHRRLSATSLVLEDGTLLNGSRYHRRTYWRQQLASSVDGRELREARRERASTAWLGARSLFIPGRDFIQFVRTWINALPSRIRTSRGRRVRGSASVMCRAGCQLDETGAHIIQGCYRTHGGRIKRHDAIAACLSDELRKRGCLVEREHSYNTAVGPRKPDIFWARGESAGVLDVQVVSGSRPLNRTAREKEEKYGTIPDLQMGIRRRHHLAGNQRIRIDTATLSWRGIWSRRSARQLQEIGLPRGLLTSITTRVLQGSHTNFNRFMQMTAIDTGRARRRRNRRGGPRPPGVVPEGIG

SEQ ID NO: 91 - protein, 1163 aa, synthetic construct
MVTTKQHKDKDPGPSGHGANLYTDENVSGAVSDKEPKCPECERTFPTERSLGMHRHHQHPQEVNTERLAMKAGRRKIWSEHEDTLLIRLANQTWQPKMPKMALYDHLQGAHPLRTAEAIKRRLRTLGWQPEATAHDQRPKTPDDIPQPLRHTDNPADPHTSIPAAAGRRWTESEEAQLRINADSIWRDGMTKKELAIRIGSHMMHRSADAILKRLAAVKWTASASDRTPKLPSTGPPTSTPDREGTPHPATTTLALSSQPRSRPQTPTPATATTPETSSPTQDPDSQPQTPTPAITTTPETSPPIPDPYNAWRRRMMETILTEMKELPLLNTLRETARALMEEEITTEDGARAVEEQIAMMFPVTWRYKEPRPKEQRPCGKPRELRRKQYAAIQRLLHTSRKDAASAVLDGSWREAWRDTAQGPEGLDDFWTEVIGQSGPPAKTPGTASDGEHWALLDPISVEELRGALRSLTNKAVGIDKITATNLLSWHLPSLASLLNLILVTESLPAPMARARITLIPKIQTPTSPNDYRPLAISPILTRALHKVIARRMRDQLEFSPTQYAFLQRDGCLEASMVLHAVLRNAHDHHRPLAMALLDLSKAFDTVTHEALGEAAMKAGLPMPLLRYVNSVLQNSESIIGSNTIKAGRGVRQGDPLSPILFILAMERPISEAHPELGVELESHHLHSILYADDMVLLANSSQDLQTKMDGLTSSLARCGMSLNARKSAALTIEKDGRTKSMLLAPTQYNTGKDQIEPMVIGGTQKYLGLEFDWKGKVTPKRTLDLERMLGELKAAPLKPQQRLALLKDFLIPRLIHGLVLGHAHRNTLRRMDLMVRRAIRLWLRLPKDTSLGLIHAPNNRGGLGIPSLETHIPMAQKSRLSKLLDGNDTIIQAVTKTTAFQVILRRLAIPIQAGGTMVLSPTEARSAWTDRLISSVDGRELAEVDVDGASHLWLKNPDRVFPRLYIRSLQLRGGTLTTQARATRGRVSPGDERRCRGGCQEVETQHHILQRCAKTHDVRCARHNRIVRLVCKKLKRKELSFSVEPIIPRAKTHIKPDIIVHRTERLTVMDVTVVSGYRLHESWDIKTRKYGGEESKEAMGAWWGSKVEVDHLPIVISSRGLIFGPTGRGLRRLGFTSTDIMDLALCTIQGSLNTYDTYMRGT

SEQ ID NO: 92 - protein, 1035 aa, synthetic construct
MSSPPSPAQHPPDVTPQTQFPCPYCTDVFGSKIALGVHRSHRHKAEANEEVDVERKKKRWSAEEKLLMARQEAIAVAAGCKFINAELLTKVPGRTLEAIKGARRDPRYKALVAAALEELLTRTTGGAPTTDQPVSGPPRPSPPRNTPPHVKTPERPTSDEPDAYHTRIKLLIPEVERIEGWHTNTLVSLSNQFLEGLDPHQLITEWVEAVFPPAQTPPRRTNRRTPAVEPISNKARKRLAYARIQRMFRVNMAAAARDILDATPEDEGGPDVKLMSEHWGPFVSIPSAPAPDIPPTPVDPELEWVWSPVSCMEVANVTMPLNSAAGPDGVSVRLWRAVPPPIKALLYNVVLASGGFPASMLASRTVFVPKKGDPNSPGDYRPISVTSVIVRHLHKLLAGRLRASNIIDWRQRCFGDGCNDNVAVLATILQHSRRHLRECHVASLDVAKAYDSVSHGAITAVLRSSGLPHDLARYIDRLYANSTTCFEVRGERSKPFGINRGVRQGDPLSSILFALVVDKILGSLPAEIGYNVGGESVCAIAYADDILLFASTKWGLQSALEAAEQEANSMGLQFNPSKCSVLSLVPSGKEKKIKIITTPGFHLGRGEYHLPQVGPEAMWKYLGVEISPGGPRRVGLEIITYLSRLTRAPLKPQQRMKILRSFLIPRLYHTLVLGRVTLGKLRALDLQIRASVRRWLRLPKDVPVAFIHATIKEGGLGIPSLSTAIPGFMLHRLGTLEHSNAPQIRAVYREEWVQRRLQWARNALTVEGQCLSQKDQRDRWWAQRLHSSVDGWELRECPETSNISSWWVDAGSLAIPGRDFVQYVHVRANTIPTRIRTSREARRGTYPTRCRAGCNVTETAAHVIQGCHRTHGGRIQRHNAVAKTLAAGLRDKGWVVDEEPIFVTTEGKRKPDLVCLRGETAQVIDVQIVSGATSLGDAHERKTSYYKNNSSLVEALSNKLRVGQDSIKFSSCTISWRGIWSSDSADCLMGMGIRKGLLRGITTRVLQGSHTNWTRWNRMTTIHPDQGIRERQGVG

SEQ ID NO: 93 - protein, 1060 aa, synthetic construct
MRTHRPLTPATGQPPTPAAISPVASTSAAKADFACPNCTQSFNTRIGLGVHRRSKHPDAVNAEIVVDRARKGLSQEELRMAASEEAYVLKFKHDKEGNPVPFINKFLAPLFPGRTIDGIKYARRTAAYRQLLEHACAALGEDGSSEEDISSESDEVSDFSGSLDGGPVASPVAVVEPPAEDVGAVPPCHRDSYAEAIRCLVLEVSKSGLETGRSLASLAEQLLEGADVTQKVAEWILALRLPAARRNRRPRRPHRSDNEKGTSRQKIRRREYAKMQSLFVTNMSRAAKLVLDGEVEAVIPSLEEMQEYWCPILEHPSIPVEQLPNLPVDRQRGALAVPISVKEVATVNVPRNSSPGVDGITPDVWGQCPVNVRALLYNMVLYMGSFPGQTLASRTVFVPKKEGASKPPDFRPISITSVVVRQMHKILAQRMMAADMVDERQRCLDDGCAENIAVLASAISHARSQRRELHIVSLDVAKAFDSVSHQAITSALTEAGVPEVIVRYVETTYRLSTTSLEVPGRRSGSISVRRGVRQGDPMSSIIFSLVTDRILRSVPGTVGYSMGHGHLINAVAYADDICLLSSSTTGMSAALAAAESTAVRQGLAFNASKCVATSILPSGRDKKVKVLTVPQFSLRDKVPIQQLGPTEEWRYLGVNLSPSGLRRVSPSMEVELRRITRAPLKPQQRLTILRCFLVTRYYHRLVLAGCTLKVLRELDLQVRAAVRKWLRFPKDVPVAYFHTACRDGGLGVPSFLTTIPGLMRSRLMSMKYSSFRSVRIAHESEWVQNKIRWTERALTLEGEVLSDRASRAKHWADRLYGSVDGKELRECGRSAASTRWIGAAVSGVPGRDYVQYHHTRINCLPTLVRTSRGLRRLRTEIKCRAGCSSTETAAHVIQSCFRTHGGRIKRHDAVCRTLAAFLRKRGMEVHEEPRLRTEVGLRKPDLLIKDRGKGFIVDAQVVSGVPPLDGSHRRKVEKYDSPTVKIAAAAALGLLPASVSVTSCTLSWRGVWSGASVESLRSRLGVPLGLLTGMSTRVLQGSHTNWTRWGQITTRVPGVRVGVG

SEQ ID NO: 94 - protein, 1128 aa, synthetic construct
MITNNNNDASDAASLGPGDAVADSSAREGTSGDDLAISTDTSYDITVPIVGSQVACPVCEKREIHLFFLSLSDLDRHLDQHHTGALLKWNCLYCGKSFSKLHGARCHTPKCRGPIQDPIKLYTCEACPMSFYTQRGLSTHERHAHPAVRNIKRRGADPQDPNKWTEEEIALLKNLWDAYKYHRHPNKMINQVLTNKTIDQIKYQRKKLNLVSGDPTLQEAEAAQATEGGCDPVDPGNAYFKEPGIDGNQNMESIQQWRLQLEKVIMTSTDVPLVLKGFYQRLQDTWVIYKDFNEVFKERVNTLISTSLYPLIKELDKGKIEPKNQRTSKPKKKNNRNSRKRYSYARCQELFHECPKRLADAVINDNKTYLEPPRQLPEAAEVRRLYEDLWGRAGPSEAPIPGSRCSELSLNEIFPPITAEEVAEKITKIRKKAAAGPDGLTKEHLLIPGLPVILARLYNILCWSSYFPPIWKVNRTTLIPKPNKPSSLVENWRPITIGPILGRIFSSILDGRIRRGVVLSLRQKGFTSGNGCKANIDLLSTALSYSKKDKGGVFTIVDISKAFDTIPHSAIKPCLARKGVPDPLIKLVNDMYADNYSKIKASGGSEVDIKILRGVKQGDPLSPLLFNLCLEPLLEQIEEQTSGININDTRKIPVLAFADDVVLLGANVREAQRQVDVLHEYLDGLGMTISRDKSQTFQVVAKKDTWFIKEPNIKLEGANIPSVDPEQAFRYLGAKMGPWKGVHCGIIVPEILSVVRRVRKLSLKPCQKIELLTKYIFPKYIYNLLMNPPADCVLKLLDGEVRQEIKAILHLVPSTATGFFYAPKSCGGLGLPRFEHIVKLGTLKNAINMKNSIDPAVSGLIDMPTDQKLKKIANSLRINWPASIEDIEKARKRLKAAHIQQWSELKSQGQGVFDFARNRLGNVWLDQYNLLKPSRYIDALRLRTNTFGTRAVLARADKKIEVTCRRCRAQPETLGHVLGLCQYTKCLRIKRHDEVTSLLADRLRQKNEVFVEPTVRVEGNLFKPDLVVKNEERVLVVDVTVRYENKDYLSKAAKEKVDKYQECLEHLKRKFNVGWGEVVPVVLGSRGAITNNTERALLAMGIAPHDIKTIIINILRSSIEMCNLFIDD

SEQ ID NO: 95 - DNA, 409 bp, synthetic construct
  source 1..409: mol_type = other DNA
ggcgggagtaactatgactctcttaaggtggccaagtggcggcggtgtggctgcatccggacttggcttctcgaagtgcggtcttgatgtagtcgtgctgctgctgcgaggtgccttccttgggttatagttacagggagagtgacgcgcaatacatgggcctagccctcttagcctctcctctcctgcggtcttctccccttctcgtgggcccgctgatttcgcagagtggaagaccgcaccaggggcttgggggggttaccagccccccctcgcactatccctttttttagttgggggcagtaagcagagaataagtggtggcccttccgctgaaggtgccaccccaactcccccagcacctagccggccaaccggccgtgccgagaatcttgtaacttttgcagct

SEQ ID NO: 96 - DNA, 181 bp, synthetic construct
  source 1..181: mol_type = other DNA
agaacaggattctgacaacgcagaaggggctcactaccgtaattcgttagtcgttgtcagtttcaagtcgactcgaaacctcctcgtggtgttctctgggtgctttctatatactaggttctagaatatagaagtagctgattcgagcggcgaatggtccttggggttgccatccccttgg

SEQ ID NO: 97 - DNA, 198 bp, synthetic construct
  source 1..198: mol_type = other DNA
agtcttcgttcgaccttgagcgggtaaacatcgtggcgcgcgcgctccggtgtgaagtcaaccgtgaccttcggcgagagacaatgcaacgcggatcctaacggtccccgaggatctgtggcaggttttcctgcatcggttggggggccaaacgatgatgaccatggcgagctactggacaatgagctggacaatgga

SEQ ID NO: 98 - DNA, 654 bp, synthetic construct
  source 1..654: mol_type = other DNA
aggccctaacgggtagaggtgcgactggaagctcctggcaggttgtaactgcgtggatgggttgactggtcaatagccagccattttgtccagtcaattcaatacgcgaacacaacttgccatcaaggggatctcgggtgtgcagtactgctttagtacgctggcactcgagattcaactcctacccctagcctgcacgcggcgacgctgctcatgactaatacgggaggaggggaagtacctaaagcagtgaccctggagttggctgatgcggggttcgaagtcccgttggtacattcctttaattcgcggggcgaaacgcgtgatttccatcactccgacccgttaataccgacacttgggcgccgttctggcgcttgggtgagtgggagggcggggtggtccggcttgtgagcgggtttgctgggtcttcggattcggtatgcgcaacgttccttgtctaggggacggaacccgtgcgaggttcgagggtcaaacctcgaatagtcccaaagggtctcaccgaatcggcttttgtcgtgtggtggccgacctaagtctagtcgcttgcttcggttgggacgaacgaaagcgacgggcgctcccggcggaagggcttgcaccccgaccgtcaatgggtactttattcaagca

SEQ ID NO: 99 - DNA, 241 bp, synthetic construct
  source 1..241: mol_type = other DNA
aggcggtcgtttgaggtgtcgggccggttcgccggtactagtatccgagatcagtccttaagagcgattcttcggaatctgtcatagtttagcaagcggtcgcctccaagttggttcctgccgggcaatgtcttcttcggaagatagctaagaaccgcctctgcgactaacggctagtggggttgcctccccctctagtttgtattgaactggcccaaataaaaggaacaaaaagcctcgg

SEQ ID NO: 100 - DNA, 512 bp, synthetic construct
  source 1..512: mol_type = other DNA
ctctggagtagcgacatgccgcttattaacggtgtgtcgttcccgggattggttgtaagttgctcgggggcagggggacttcggtctcgcctgctcaagtctcacttctacctccccgtggtaccgccggtaacatactccgctcacatcagcgtggggtgtagatccccccggatgcgatgagtatggctaaatggagtgaggcgccgaaggagcgaaagacaaaatccacctatcggcttgccgatcaaatgatggcttgccgtcatgaaagaagggagcgattccccagctaaacgttcctttggacgttagtgcaaacggtaacgaattggcttacgcacggtcacgatttggaatgatgggaacctgaaacatagatttcaaataaaacactgagaacattggataatgacaatgtagcctcggacgcggcgtctctcagtcccggaggcgccgtggcggattcttccgctctggaggggactatagttgacgaaccggctgtgccc

SEQ ID NO: 101 - DNA, 603 bp, synthetic construct
  source 1..603: mol_type = other DNA
agggcgggagtaactatgactctcttaagcggtcatgtccctccggcggcgccacccctccagctcagccgcccagttgtttccaactggacgttgcccagctcccacctcgtggaggtgtggagggaagggagccacttttcatcatgtggcgccgtcggattgacatgctcgcttttggagagtcatggttacacgggacggtgatgcgcaatacaggcctctggcccttagcctctcaccgtccaagttgcacggcctacctcctcgaggttcccgctggtcaacgtggttgttgcttccgtaaaattgagcgctatttgcggaagcatcaatcacggctaaatggtgtgcagcgaatactctttcgatgaaagaggaataatccatccgatggaaataatcaaggatctcctcaaggaacactactgaactctcaggataacaccaaggtggtgtgaatattaagtactgatatttaacaacgagtttagcgtacttgtacgcggatggatattgtatacgaaaattggcaagactacggacacgatttcgatatatatttggaaactactctaaggaatatattatttaaaacaaata

SEQ ID NO: 102 - DNA, 490 bp, synthetic construct
  source 1..490: mol_type = other DNA
atcgggagccacatgtcatcaagtggcgccgatggagcgatacggacgctgtcgagagccttagttacatgggatggtgatgcgcaatacaggccttcgggcccctagcctctcaccatccaagttgcacgacctacctccacgaggttcccgctggtcaacgtggttgatgcgtccgcgaagttgagcgctattcgcgggggcaacaaccacggctaaatggcgtgcagccacaaccctttcttaggaaagaggaaagtaaggacgtataaaacattaccatcaatacctggttttccagtcaacactatcaagtgtgaaatgtgagtcttgtgagctcttgtgagctccgaaattaagagcttaaaaaaagagagaaaccacgtatcaatacgttgattgattattatgcgactatggcttgaaatacggaactagatttggataatattactgatctctatgctcggaccaatcactttaaaagaaa

SEQ ID NO: 103 - DNA, 104 bp, synthetic construct
  source 1..104: mol_type = other DNA
tggcgggagtaactatgactctcttagggtaaccaaatgccctaggaagcatagatgtgcctgtatactacctccacgtggttcctgccggtaacaattgggtg

SEQ ID NO: 104 - DNA, 857 bp, synthetic construct
  source 1..857: mol_type = other DNA
taactatgactctcttaaccacatgagagagagagcacacactaactgaaaaccgaaaacggatacgcgaaccagggcagttctcaccaaactgttttggggcaataggcacaaaattatcaagtcctagagctcgcggattccgtggcagttacgtgtgtttgttctgtgactcagtgaatgatgcaaacgtgttgcggttttgagagcttagttactcatgacatgccatacgcacccctaaagttgagctatcgatggatggggaagaatgcgatggatcatgtcattaaggttgctgtacagctgcgattggcatgtgtgtattcctttgtggggttgttctaactctgtgaaggtaaagcacatgaaacatgattttccaccttacatcttttggagcgagagatgaaaccaacgcaagctgttagcttcaactcctagcctctgcctgctcgtggcaccgctgcataaaccaaagaggagggggggagatctaccggcactaaacgctgacttgggtatagctgcctgggccatgcagactcataggtatgatctcttcgaacgcgaatgagtcgctcgtactgtttccagaacctccctcggcaaccgggcgtttcgtccggcggtcggagggggttctcaggtcttgttcttggattccctaaggggtggtacatcgcacaacatctccaatgtgaaatccaagaggtttggggggaagttccccaaacagccccgatgagtctctaatagactaagtgtagcagtttccttcgggtggggccctacaaaaactgtgcacgaacctggcggttgggtttgcagcccgccgctaagggaatttaagcgca

SEQ ID NO: 105 - DNA, 513 bp, synthetic construct
  source 1..513: mol_type = other DNA
cgcaaatcggagagcgggggagtcatggactccacccagtcgcaagactggggccaaggccggtaaacccttcaggtttactagaccgggggaagggaggtgtcctcacttatgtatgtgacgatacccccttctcaaggtccgcatggtacctcctcgtggtggcgccggacactgtcggagggagcgttgtaaggcacctcccacagtgactaaaccatgcggcccgtcccatccccacgaagatccaggaccagtggagagggtaggtccatgcgctcgcccagatcgcaagagcgcccgctatagcttccggcggattggggttaccagccccttccaacagccgttctatttggtcctcaacgaattagaaaaagcctcggacgaggggaatccacgggtccggattccccgggaccttcagggacccttgaagaaggctcggctgcccaaggaggcacagcgcagactatcggtgctacaacgggagctatcccaaaatcgaagcca

SEQ ID NO: 106 - DNA, 814 bp, synthetic construct
  source 1..814: mol_type = other DNA
cggttagtgacgtttattaaggcgatatgttgctgaggcttggttgcgcttgcttaaatagtaagacgcggctggtctcggtggcatatcgccttttttttatgcctttttttatgatttttgttgtatgaaaaggaaggtgaacaactgccccgtcattttactagtgacgtcacagagaaagcaatacgccgctggcacttggccgcgtttgctcagaaagcaatacgccgctgggacttagtagcggacgctggaaagcaatacgccgctggcccttagccgcgttcgtctaaaaacgatacgccgctgggacttagtcggattttgctggaaagggtcgctgcttgcccatgcgtgagcttgatgggccgcgtgagcggggccagtggcgtgttgtaatctcatgagttcgttaggtagtgggacgttgttaagcgacctgctcctaataaagaagaatcatagttgctcaggggcagggggaccttggtctcgtctgctcaagtcacactcctacctcctcgtggtaccgccggtaacatactcgttcgcattagcgtggggtgtctattcccccgagtgtgacgagtatggcgaaatggagtgtggcgaagaaggggcgtaagacataacatcggtctatcggctcaccgattaacgatttttatcgtgaaaaatagaaaaatttaatacctataataggaacttgttcctattttgcttatgacgactggcttgaataagggatacgatttggtatttgtttggatttgatgggcgagaacacagttaattcaattaattgggttaa

SEQ ID NO: 107 - DNA, 94 bp, synthetic construct
  source 1..94: mol_type = other DNA
aagtactgcttcggcagtactcagtagggatcatagtttagcaagcagtctcctcttagtgggtcccgccataaactgtcctcggtggaaagca

SEQ ID NO: 108 - DNA, 323 bp, synthetic construct
  source 1..323: mol_type = other DNA
cggtcctcaaagcaacacgccgcgatggtacttagagagagtcgtggttggttcgtgatgcgccaacgaagtcctctagtaaggaccttcagcctctcacgtccaagttgtacctccacctcctcgtggtgctcccggaaacggcctactttggaagtttagactcggtaggtgtcgactaagcgagtgtacagcgaagtgcgcccctaagggcttcggcccatgcgcctacggtcatggtgagttgtggggccaagaaagaccggggtttcttggggggctctcggttgtcaatccgtttgcccccctacacgcagtaaatt

SEQ ID NO: 109 - DNA, 704 bp, synthetic construct
  source 1..704: mol_type = other DNA
acccctcatgcttttgatacataaacaaacggtgatttggtggagcacttacgtggacctgagtctcgtgggttgtgtattgctgtgctacggccatttggccggaggggttgcaacttgggggttatctctactcctcggggccctaacgggcaggacatagtcatagttactcccctctcgggcctctgcgtacagtcctttcggcattgattgatgcggtgggacggaaaacctttcgtagggtccgagggggcaatggggcttttggttcagtctttctgccatctgctcaacacctatcctgagccttctcgaggcgccgctgacgaagctaatcaggggggtggactacacggctaccagatctaaaaagggcaacaagcgttgtccaagtctcccaggagctgtggttgataaggggaggcgattgaagcgctccgtcatctgtgtgcggttcggtatgtaggggccggaccgtgcacgggtggcgctcgaatactccaaaggagttgcgcgcaccccaatcacggtaaggggtgaagttcgaggggcccaagaattgggctaggtccgaatggacgcgtcacgccttcagggtggcgcgacaagtctcagtgttatcttcggttggaccattacgaaacctgaggcgagccctcggatggtagattagcattccgccgtccggggatatgtcatga

SEQ ID NO: 110 - DNA, 197 bp, synthetic construct
  source 1..197: mol_type = other DNA
acatcaatacctggttttccagtcaacaccactaagtgtgaagtatgagtctttggggctccgaagctaagaactagattaaaaatcacgtattaatacgttgattgattattatgtgaatatggcttagtataaggacttcgatttggataagactactgatttcttatgttcggaaacaaattacctgaaacaaa

SEQ ID NO: 111 - DNA, 812 bp, synthetic construct
  source 1..812: mol_type = other DNA
ggtttgtgacgtttattaaggcgatgtgttgctggggctggttgcgtttgctttaatagtaatgcgcggctggtctcggtggcatatcgcctttttttttttttttatgcctttttttatgattttttgttgtataaaaaggaaggtgaacaactgccccgtcattttactagtgacgtcatggagaaagcaatacgccgctggcacttggccgcgtttgctcagaaagcaatacgccgctgggacttagtagctgacgctggaaagcaatacgccgctggaccttagccgcgttcgtctaaaaacgatacgccgctggggctttgcggggtcgccggaaggggtcgctgctggcccatgcgtgagctggaagagccgtgagagccgggcttgctgagtgttgtaatctctttaaattgttaggtagtgggatgttataagcttgctcctaataaagaagaatcatagttactcaggggcagggggacctcggtctcgtctgctcaagtcgcactcctacctcctcgtggtaccgccggtaacatacttgttcgcattggcgtggggtgtatatcccccgggtgtggcgagtatggctaaatggagtgcggcgaagaaggggcgaatacctgtgtcatcaatcggcttgccgatttaacggtgtttattaccgtaaagaagtgggaataattccccagataaggaactcgttccttggacagtttaatggtacaacttggcttgtcaaaggactaagattgggtatcataaggaaagaaataaagaggaaattaaataaactaagtaaataa

SEQ ID NO: 112 - DNA, 173 bp, synthetic construct
  source 1..173: mol_type = other DNA
actctctgggtatccgccagataccgccgccgcatagttatagctgtgattggttttagcgaattttgattatcacagttggtcgtgcctccccgttggcctcgccggtaacctccgtcaaatgtgacggagggctaaacgaccgtataaattcgcgttggtgtttttgcctt

SEQ ID NO: 113 - DNA, 685 bp, synthetic construct
  source 1..685: mol_type = other DNA
gttttagcgacgtgtatatatggctcttaaagggggagcggcgcgttggcgcggtctatgccgcctatatgcctggaaggtggtgcatgctttgccagttcgcggaaagcaatacgcggctggacttagctgcatttcaccggaaagcaatacgccgctggacttagcagtgggtcgtctacggtccatgcggcctatatgtctggaaggtggtgcatgcttttgccagtccgctaaaagcaatacgccgctggacttagctgcatttcaccggaaagcaatacgccgctggacttagccgcgggtcgcttcctctcgggatcccaagcagcacgccgcctttaaacggtgtgtcgccggaccccggagagtcttggctactcgggggtagacgaggctcaggtcttgtctgcccaagtcacactcctacctcccagtggtaccgccggtaacacgcccgccgcattagcgtggggtgtatatccccccggatgcggcgtgcgtggctaaacggagtgtggcgacgaaggagcgggagacaatattcgaataatttaatttgtttatcggcttgccgataacggtacttggtaccgtgaaatatggaggtatctaaattaaattaaattaaatatggaactcgttcctttggcccctatgacgactggcttgaaaaggatatgag

SEQ ID NO: 114 - DNA, 193 bp, synthetic construct
  source 1..193: mol_type = other DNA
ggcgggagtaactatgactctcgatcgccattaggtgtcatagttagcaagcagccccctctaagttgggtcccgtcacgagtaactccttcgggagcgaaacagagagctgcctctgcgaatccgggtcctgggggttgccagcccactcgacctacgacctgttggcccaaagaacaaaaacaagcctcgg

SEQ ID NO: 115 - DNA, 366 bp, synthetic construct
  source 1..366: mol_type = other DNA
atgaggtcatgctcccaggagacgcgtggacttcgcagtgcattgacgggacatgaaattgtaaagtccgactgtgttgcggagcgcgctcctgcccaaatgggggatgctggggtcagggaatagagagccaacgaacaccttccctctattccgtctggaaaccctccccaaggccgacctccacgtccgacccttaggcaaggtcacgtggcgccgggaaaaggaccatttactgtgcctggtccgagctaacggaggcggccgaactcggcgaaagccaagtggattatttgggtttgctccccacttaattcacgccttcgggcaaatccatatgcaacgaataatgaaaacaagcctcgg

SEQ ID NO: 116 - DNA, 276 bp, synthetic construct
  source 1..276: mol_type = other DNA
aggcagggcgcttcggcgacgggtttgacgatcccgacttacccgagttggctcactctaagggtcttcggactcatacgcctgccccccgccttagggccggagtgagtcggcgtcctcggacgtccgggggaggctggagcccataatgggctcccccggtgcgtagggttgccagccctgcgcgtcgggttattatctgtaggcccaaagattaaagagaatagatataatctccggcgagggcctcggctcgccggacctacagaccaggcc

SEQ ID NO: 117 - DNA, 331 bp, synthetic construct
  source 1..331: mol_type = other DNA
taagatgagggcatagttactaactgggctcagccgcggaagtcgcacctccacgtggtcccgccggatacccttgtggtaaccaagtcgacttctgccccagtgtgagtgagggcctttagcagccctccctattgttgggctcgccacccagtggtagtatcacgatttgctgaaacaaagagtttgcctcagcttgtccgctgttgcatagtgcctagtccacgggccgttgttgttggacaaaagttggtagctcgagagagggtcgactactgactattccttccctcacttggactggtcgtttctaatttactggactgacata

SEQ ID NO: 118 - DNA, 511 bp, synthetic construct
  source 1..511: mol_type = other DNA
cctccgggtagctcaatcggcgcctttcctccagctcagccgtccaactgtttccagttggccgttgcccagctcccacctcgtggaggtgtggaggattcgggagccacatttcatctagtggcgctgattgggctgcgcggacgttgacgagagccacgattacacgggatggtgatgcgcaatacaggctttcaggcccttagcctctcaccatccaagttgcacgacctacctcctcgaggttcccgccggtcaacgtggtcgatgcgtccgcaaagtccagcactttttgcggaggtatcggccacggctaaatggcgtgcagccaccagcaataggctcgcctattagcaaaggctcgaaccctacagggactaacatcagatcccatatttcactcaaggtgaagaacgtacgacttcgtaccgttaagacataaattggccaaacatctggcagtcatgccctaaggacggcattgtccttaatacaattaccaatactaaat

SEQ ID NO: 119 - DNA, 329 bp, synthetic construct
  source 1..329: mol_type = other DNA
taagatgagggcatagttactaactgggctcagccgcggaagccgcacctcctcgtggtcccgccggataccctttatggggaactaagtcggcttccgccccagtgtgagtgagggaacactatccaccctccctatcgtctggactcgccacccagcggtagtctcacgatcctgcagaaacaaaattgtttggttcagcctggtcgctgttcctgaagccttgtccccaggctaagttgggcaaagttggtagtctggagagggccagctactgactgaaactaccctcacttggactgtcctaaatttgaatcctagatttgaca

SEQ ID NO: 120 - DNA, 352 bp, synthetic construct
  source 1..352: mol_type = other DNA
taaggcgacttaagaaggtctcgttacaactgggcatagctgcagagatcgcgcctccacgtggccccgctggtaagcccttaacagggtgactaagtcgatctctgccccagtccaggagccgctgggactcaccagcccagcgattccttccaaattcggtgaaacaaatttctcggtgtaagtcgcaaggcttatcacctgaaacctggccccacggtctgacaggggcaccgggtcaggaagtgggctgaccacccaccccgaacccgagagcgaatctggtcatgactcaactgtcccgaatcctggtccggtccttggaacgggggacggtgcgcggccgatacgt

SEQ ID NO: 121 - DNA, 694 bp, synthetic construct
  source 1..694: mol_type = other DNA
agtctttagttctcctgcattgaacgcgaagtttcccgctcgggtgtgacgaccccgctttagcccagttggtcgaccagaaccggcgacgtggtccgagtctgcaaagaggaggtctgccgagccaatctggaggagtgatgtcccgccacatgggctcctccgattatatagatgcattaaggcatcagaacttcgaggaaagtagtatggggcgaatagttcatttttatcgcgcccaaatgaagcaatgcgcgtcgaatttcatttcttcaaagacaagcaatgcgcgtcaatccatcggcgactacgctaagtcccaggaaaccagggagatactcagaacatcgagcatcgagaaagccgcataagatcgcgtgctatttgccaagtggctgcaggccaggtgaaacctagcccgtacacgcagtgttatacacttgggggggacaaaatactcggagtagaataaaacccaataactttggttatcgtgcgaaacttgtttcgcccatcgactcagatcggtgataaaggggaaagcaggcgtggagaagaggaaggatgcacttgtgcatcggaagcaagatgctttaccaagcatgattgctctatgacaactggctggaacaaggttcatgttaagcaataggttatgccattaggcgaccgacaaataaaataaagaatcaat

SEQ ID NO: 122 - DNA, 466 bp, synthetic construct
  source 1..466: mol_type = other DNA
agtaggtaaggtgtcatagtgctcgcgtatgtggacatgtaccctttggctgccgctatcagttaattgggagaacgatgcagtccacatgcgtcaagggttttactggtccgtattcacaaccaccgaaccataaaatcaactcctacccttggcctgcacgtggcgccgctgccaagccaacaagggggggggggggctactcatgttcaaacagactggttagcaaggacagtctgttcaaatcatctctcgcccaaccatccagtgagtagacccttcgaacgatagagagatgtagattgcgcataccaccccgcgttgaccagaagaatttattccgatggtccaggggtatggtgagcctggagaccttgatcattacacctgatcaaggtgcgcaaacacgcaagttgagccgtgtatatccaggttggctctgggtgagattcccagagattccccg

SEQ ID NO: 123 - DNA, 781 bp, synthetic construct
  source 1..781: mol_type = other DNA
aggtttccacaggcctcgtcattagattagtgacgttacaagggcggtgtgtcgcttggacttggtcgcgcttgctatatagcgaggtgcggccggtcttggcggcgcatcgccatatctttttttaagccgaattcgtgctacacgaaacggaaaaggtgaacagcagcctcgtcatctgactagtgacgttataaaagaaagcaatacgccgctggcactttgccgcgcgtgctcgaaaagcaatacgccgctgggacttggctgtgcgcgccagaaggtaatatgccgctggcccttagccgcgttcgcccaaaaacgatacgccgctgggacttaggcggatgagctggaaggtggcatggcttgcctacgtgtgtgctggaagagccgcgcgagggttgctggttgagcgaagcgatcttgctcgttgggtggcgggacgttggaaaacggcttgtctccaacgatggaagagtcatagttgcttgggggcaggcggggctttggccccgtttgctcaagtcgcactcctacctcctcgtggtaccgccggtaacatactcatttgtgtcagcgcggggcgcatccccccggacaccgagtatggccaaatggagtgcggcgatgaaggagcgagagacgaaaccaaaaaaaaaaaaatcggcttgccgatctaacgatgaatttcatcgtgaagtatggaattatctccctgaaaggaactcgttccttgattagctatggcacgacttggcagtaataaggttaacgattgggc

SEQ ID NO: 124 - DNA, 224 bp, synthetic construct
  source 1..224: mol_type = other DNA
cggagatctggttagcaagcagcctccttcttgtgggtcccgccgattagtacccccctagggggggaagctaagagctgcctctgcgttcaccggtcttgctgggttgccagcccgctcgaccgaattaagtcggccctgatataaatagtaaagcctcggatgaaaataatactatagccggggggtacacagactcacccggtccgactacgcaaaccgac

SEQ ID NO: 125 - DNA, 186 bp, synthetic construct
  source 1..186: mol_type = other DNA
aggactccttggtgaacgtctttagcctcggagcccaagtgccaaccgtacacctcctcgtggtgagagtcgggcaacgtcagtaaaattactggcggccaagtacggtggctatctcagggtcttgggggttgccaaccccctcgaccctcaactgttggcgtgaaacccaaaaacaagcctcgg

SEQ ID NO: 126 - DNA, 434 bp, synthetic construct
  source 1..434: mol_type = other DNA
aggataggactagcagaagggatataatatcgcaattcgtcagcctagtcaagtcgactcgtaacctccacgtggtatcctctgggtgcctggtacataatattattggataatatatgcaaatctattttgcataatatattcataatatgtttgttttaaaatttttcggaattttaaagcatatggtatattattgggtaatatatgcaaatttatttgcataatatattcataatatgtttttgccaggtagcagatacgagcggcgaaatggaccttggggctaccaaccccctggtctataacctgttggtgcaaatttaaataacgagcctcggatatattataataactctagcaatactcctgcggttttacgcccaggagcccaacagggggatgctgaagttttgccacaaaacggatcatcc

SEQ ID NO: 127 - DNA, 247 bp, synthetic construct
  source 1..247: mol_type = other DNA
tcctgcaggtaacctggacctgcaggaggggcctagtgctatactccataagtcataggtcaagtcgactcagaacctcctcgtggtggccgctggatgccagttgctctccgttctggaggcttctggtaactgatctgagcggcgaatgcgggttttaggggttaccagcccctcttacctttaacatgttggtaccagaaatgataataacgagcctcggataaataatcgaattaacatacct

SEQ ID NO: 128 - DNA, 486 bp, synthetic construct
  source 1..486: mol_type = other DNA
acgtccgggtcgctcagtcggcgcattcctccagctcagccgtccaactgttccagttggacgttgcccagctcccacctcgtggaggtgtggaggatacgggagccacatttcatcatgaggcgccgacggggcgacgtggacgttttcgagagccatagttacacgggacggcgatgcgtaatacaggccttctggcccttagcctctcaccgtccaagttgcacgacctacctcctcgaggttcccgctggtcaacgtggtcgatgcgtccgcaaaatacaagcgctatttgcggaggcatcggctacggctaaatggcgtgcagcgaaaacactctttctatgaaagaggaagaaaaggaagtatctaacaccaatacctggttttccggttaacactatagagtgtgaagtatgaagctcccaagagctcggaataaaaaaaataacggaaaaactacgtatttatacgacgactgatcat

SEQ ID NO: 129 - DNA, 176 bp, synthetic construct
  source 1..176: mol_type = other DNA
gggcggtcgtttgaggtgtcgggccggttcgccggtactagtatccgagaccagtccctaagagcgattcttcggaatccgtcatagtttagcaagcagcctccttcacgagggtcccgctgactagtacccccttagggggggaagctaagagctgcctctgcgattttgacatt

SEQ ID NO: 130 - DNA, 237 bp, synthetic construct
  source 1..237: mol_type = other DNA
aggcggtcgtttgaggtgtcgggccggttcgccggtactagtatccgagatcagtccttaagagcgattcttcggaatctgtcatagtttagcaagcggtcgcctccaagttggttcctgccgggcaatgtcttcttcggaagatagctaagaaccgcctctgcgccacacggtcctgggggttgccagcccactcgactgtatacagttggcccaaagaataataaattgcctcgg

SEQ ID NO: 131 - DNA, 252 bp, synthetic construct
  source 1..252: mol_type = other DNA
aggatcaatgggtctgcagaaggggaataatggttcatttatcagcaatggcaagtggactaccgtccttcgttagtcagatcaagtcgactcataggcaacatgtgccccacttaacttgtggcctcctcgtggtgtcctccgggtgctggaccgatgtagtttcggcagcagcgaatatgagcggcgaaaggggccattgggttaccaccccgatggtcctgaaaaactgatggtgcaaacttaaataac

SEQ ID NO: 132 - DNA, 234 bp, synthetic construct
  source 1..234: mol_type = other DNA
tagggaattaatgggttaatattgcacagaaggggagcaatctgcaatcgaagtaattcgagcgcagatccgtatttcgttagttgcaatcttaaccaagtcgactcgaaacctcctcgtggtgttcctggtaatgttgatcgatttttgattgacagctaattcgagcggcgagtctcctcagggttaccttccctgttgagacttattcatgttggtacaaacttaaacaat

SEQ ID NO: 133 - DNA, 206 bp, synthetic construct
  source 1..206: mol_type = other DNA
actatgactctcttaatgctgtgtcaggagtctggttgctgggccgcagtattgagatctcaacctccccgtggtcccgctggacaactctctgggaacctgtgatatgggagttgactgactgagactcagctgcattcgatcccctgccctactgtcctggtgcaccggctaggcagtagaacactacggtgaaaaacaaaacg

SEQ ID NO: 134 - DNA, 188 bp, synthetic construct
  source 1..188: mol_type = other DNA
taagctgtgtcaagagtttggttactgggccgcagtattgagatctcaacctcctcgtggtcccgctggacgactctctgggatccctgtttgggagttgactgactgagactcagctgctttcgttcccctgccctactgttctggtacaccggctagacagtagaacgctacggtgaaaaacacta

SEQ ID NO: 135 - DNA, 454 bp, synthetic construct
  source 1..454: mol_type = other DNA
acgacttggcgtcgggtttggtggtcccatcgcccatcttcaaggggccggcccagtttgacctgaaaaggtccggggctgcgctcccgcgaggatgcaatacgtgaacacttatcaattccctgccactttgaccctagaaggtccaggctacggtgagacttaagtgatttacgcactattccgtcggatgtgttgcgggagtagaggttagttgcttgcccgcccggcgggcacggcggaccggtggggcgagcgactaattctctacagacggaatcaaatcgaagcaggatggctccccccgcaggagccatcagccccgtaaggtcggcttgccggccaagcctatgtgttactgtttggggcttcgaaaaacacagggcgacggggtggattcgggttagcggcccgcttccgcccattaaagttcattgctcaagacaattcaaag

SEQ ID NO: 136 - DNA, 518 bp, synthetic construct
  source 1..518: mol_type = other DNA
agggtttagttgctctccttgccacaaagggatttagggaaccaactctgctgtgcagagagggagacccctaattcccaagtgcctagtgtcacctcctcgtggtgtgggccgggccccattggacgtgcgccaatgggtggctaacgacacaggctattcgggctccaggagtgtgcagcccacacaaacggccagccggttttcgcgttttctggagacccctcaacaaagccgggggcatagaggcctgataagcctcgaacagctttcaggcttgctacctggaagttgtcttttcgctagcaccaaagaagaaaagaagtgtgtcggggctattccccgactgccggtcgctgcctcggacaaccggcctagcgagccaagggagggctgtgtctaactgccctatcccgcccgtccgaaataatgataattcccctgaggagaacgatcgtcagttctcttgccgacaatgttcgcgaacattcacatcttcccgcgggttgtccaatcac

SEQ ID NO: 137 - DNA, 310 bp, synthetic construct
  source 1..310: mol_type = other DNA
accatcgcccttctgctggggttggtggggcctaacggtcggagggaagtaggaggctcgcagtctcgggtaaaaccggggctgtcaatggggtcctgaactttaaccggaacggggctcaactcctaccctcagcctgcacgtggcgccgctgccaagctaatgaggggggaggggggggcatccctcgcgagtaccccggatgtacctcctgtaatggggttctgcgcgtttccatcgcatcatcttgcttcgggtccttgggcccgaggccggatgcggtcaggcaagtccttgggtttcctacggg

SEQ ID NO: 138 - DNA, 230 bp, synthetic construct
  source 1..230: mol_type = other DNA
agtcgtagttctcccaggagatgcgtgaatcaaccttttgatgaacgctctcctgccaaatgggggtgtggggtcaggaagaggtggtacttaagttctaacaccttcttcgctggaaacccaccccaagtccacttccacgtccagtccttaggcaagactacgtggcgccggagaaagagccctaagaacgcctggctcaagctaacggaagtggtcgaactggcttt

SEQ ID NO: 139 - DNA, 569 bp, synthetic construct
  source 1..569: mol_type = other DNA
acggtctctaggggccgggcctaacggctggagctgaattgttgctcacagtctctggtaactggtaaagatccgaacttaaagttcactggcataaaagggtggagtatgagaaggcaaacacccgaccaaagactgtcaatggaaccctatgttgagctggggttcaactcctacccttggcctgcacgtggcgccgctgcgatcgccaacaaggggggggggaggttgcatctaaaatgctaaatgatctccaggatgcttcctctggtcaggagatgcacaattccgttacggtcgccgctgtcagggttcgacagtcaggcaacggcggacgaaatcaggcttgtcccaaggtctctgcggatgtgcaacgtctttaccacctctgacgtgaccttggtgaagctcagggggtggtacctgagtagccccgatgggcgtcgatacgtcggcgtcaagcctatctgctttcttcggaagggggcatatgaacagtagagggcgaacctcggtggtgggttaacgccccgtcacatgaggaaagattatttgttcaggacgctaaa

SEQ ID NO: 140 - DNA, 268 bp, synthetic construct
  source 1..268: mol_type = other DNA
taaggtctcctgggtcctggtgacacggagcgtccactgggcagctcgcatccggggttacgggggggccgttcagtaccgctcccgagggagcgggaaaatgcggcgctcccggaccaggggtttccttgggggtcctcggccaagtaaaagggggtagtgggatgggctcgctgccccctgaggtcgcggagctgtagttacatggttggacctcctcgtggtcccgctggaaacggcttgccggctaactcaaccacgcccaagg

SEQ ID NO: 141 - DNA, 302 bp, synthetic construct
  source 1..302: mol_type = other DNA
ctaaccggccggctcagcccgcgactgccgccgtcgtcggtctacgggccggcggcggcaggcgaggtcaggcttgtacctaggtccccttcggggtgcgcaacgtctctggcgtgacctaaggcggagttcggagggaggctcctcgaacagccccgtgggtcggtgagtacgcccgacggcaagtctatcagctttcttcggttggggcacgataaaagcggagggcgaacctcagtggggggccagcaaccctccactaagaggattttcaaaacgctaaatatcggaacataagaaat

SEQ ID NO: 142 - DNA, 627 bp, synthetic construct
  source 1..627: mol_type = other DNA
actatgactctcttaacttgaactgtaaaaacccctaacggggtattaagatgacatggtcactcgcgacccggtagtagcacacatttcactggcgcgccagctagtcagagggatgtgaatactgcgcgatcgggctcgtcaacggggaagaatgcctgtgccggcatgacgcccaactcctatcctcggcctgcacgtggcgccgctgcgaaagctaacgaggggggggggacagcacctaaataggcaaccctttagcagctgatggggcgttgcaagtgtctcaatggtgttgtctggatctcattttgggaccgcgcacatttccactgttatctccataatcatcggttctcactggtggcagtggagagaacagtcaggcttgtccctgggtcctcttcggagtgtgcagcgcctttccatctaaggcgtgatccaggtggtggctgtggggtgagattccctgcagcagtcccgttgggtgggatcttcggaacccgccaagtctaatggcctacttcggttgggaccttgcgaaggccgtgggcgaacctgggatttgggcttgcaccccatttcccaaagggtaaaactgacgcaatagagaaatatagagaatat

SEQ ID NO: 143 - DNA, 200 bp, synthetic construct
  source 1..200: mol_type = other DNA
cgaccatttggtgttgatattgatactggcgactggctagttggtgttggttgaaagtgctggcgaggttgagctgataattggttgcacgggttgggcacctcctagtggtgaccgctggatagggcttcggcctgagccaagatccaacccaccctccgttgctggcagaccccagtaactgagattaaacagctggt

SEQ ID NO: 144 - DNA, 235 bp, synthetic construct
  source 1..235: mol_type = other DNA
taaggcggcttttgagagtatagttgcaaactgggcttcgttgcagagatcgcacctcctcgtggtcccgctggtagccctgcgtagggtgaccaagtcgatctctgccccaggtacggaaccgttggggctcaccagtccaacggattcctaccaaattcggtgaaacaaattcctcggtaaaagccgcgtggcttattgcctgaaacctggccccccggtttcagacaggggc

SEQ ID NO: 145 - DNA, 622 bp, synthetic construct
  source 1..622: mol_type = other DNA
gggcgggtggagggactgccacccgtctggtctaacggctggagaagatggttactcacaggttctcagaggtgggccgaactgggactcaccagcgtttgagagtcagaagggaggcggtacagaccatgggaacctgtcaacggggcttgaaagcttgggcccaactcctacccttggcctgcacgtggcgccgctgccagagctaacaaggggggggggaggacgcatcggacactaaacggggactaggcaagctggactggtcccaagtctcccagatgcgacctcttagagtgtgggaggcgtgcgcatttccatcgagttcgtcactgtcgttcggggtaactcgggcggcagtggcggattcggtcaggcttgtccctaggttccatacgtggtgcgcagcgtcttaatttcttaaaggcgtaacctaggtgaagttcgggggggaagtaccccgaatagccccgatgggtgttgggcttgcccgacgccaagcctagccgctttcttcggttggggcactataaaagcggtgggcgaacctctgtggtgggcctgctaccccccacaatgaggaactaaaaatgcaaaaggtaagaataatgaaaataattct

SEQ ID NO: 146 - DNA, 313 bp, synthetic construct
  source 1..313: mol_type = other DNA
aagaagtcttatgactctcttgagaaatcatagttactaactgggcttagctgcagaggccacacctcctcgtggtcccgctggataccctcctgggtaactaagttggtctctgccccagtctccggagctttgatgctccaaactatcagtgagctcgccactgactggtagtattttggcgaaacaaactttagtggcaatttaattgccacacgctagcctagcccacggcgtagttagttgggcaaagtcatttgctctttgaggggagagcttctgactgattcctccctcacattgacttgacaaa

SEQ ID NO: 147 - DNA, 460 bp, synthetic construct
  source 1..460: mol_type = other DNA
gctaacaccttgagccgttttactccggggccggtcgcatgtaagagactacgcgatttcccagtcaaccggctagctgggggaagcgtaagtatcgtgaccggtcttgcaaacggggaagagtggtctgtaacaagccacgacgcccaactcctactcacagcctgcccgtggcgccgctgcgtaagccgatgtgagggggggggaacagcaccagaattaagcagcccggaagcagccgttgcggcgctgcgtgcctcgttggtgcaatttccttcgaacggtagacgaggccatgagcctgccttccgctgctgtctccgccgctgccggtcgcacggcaatggcggagacgcagtcaggcttgaccctggattctctacggagtgtgggcgatgtccctccatctttaggggcgtaatccaggtgaggctgtggctgagatgccacagcagtcccg

SEQ ID NO: 148 - DNA, 373 bp, synthetic construct
  source 1..373: mol_type = other DNA
cccgtcggggagttgcgaccccgcttaaagctgagcaattacttgggtgactacagttcttgttgaagtgtagatgccatttggttcgccatttggacagccgttctcctcctagcggaattctgctaggcgcccaagctcccctgcgggggggaaacggagccggtggagtgccaggattgggcttccgccccaccatggtactataaagatagttggaaacgatatagaaaaagcctcggaaaatgatagtggtgggtccactgatccaccgcccccaactatcgataatggcctcgttccagggagattcatctgcgagatcgaaggatgcgcgaaggtgtgtttgtcccagggcggtcttaagcttcat

SEQ ID NO: 149 - DNA, 599 bp, synthetic construct
  source 1..599: mol_type = other DNA
cggaggacctatccttaacgggatactggaaagagcatagttgctctcgacccggtggtagcacacagttctctgacgtgccagctagtcggagggaagtgaatactgcgcgatcgggtcggcaatgggtgaagcgtccttcggggtgcgacgccaacccctatccacagcctgcacgtggcgccgctgcgacagccaatagtggggggggggacttcaccataaagcaactctatagtagctgttgagagatgcgaggcctcttggtgaaagtctcataacgcggaaggaggctagcatgagtccgctatattgttcgtcaccacttcgagccttgggcaacttgaggtggggaacgataaagtggagcccggttcctctacggagtgtgcaagcatccacgaccatctcgggtgcgagccggaagggtccttgcagagcaagggcctggctgtgtgtagggatacgcacagtagtctgatgggggggaaacctccaagtctatcggcacgcttcggcgagacatacgagtgctgagggcgaaccttgtcgaccgggcttgccccccggtcgcgcaagaagggtaaaactgacaagca

SEQ ID NO: 150 - DNA, 298 bp, synthetic construct
  source 1..298: mol_type = other DNA
atgggatcatagttactaactgggctgagctgcagaggttacacctcctcgtggtcccgctggatgcccttcggggtaaccaagttaacctctgccccagtgggggaagtataactacttcctccctatcgtctggcctcgccaaccattcgatagattcgaaccggtgatataaactcctggcaacgactttcagtcgctgcccaaagcctcgcctacgggcacagttgggctcagtcacacagcacttgagggggtgttgtttggcttatactactcctctatctggactggcaaa

SEQ ID NO: 151 - DNA, 396 bp, synthetic construct
  source 1..396: mol_type = other DNA
atgaggtcatgctcccaggagacgcgtggacttcgcagtgctttgacggtacaagaaattgtaaagtccgactgtgttgtgaagcgcgctcctacccaaacgggggatgctggggtcagggaatagagagccaacaaacaccttgcctctattccgtctggaaaccctccccaaggccgacctccacgtccgacccttaggcaaggtcacgtggcgccgggaaaaggaccatacaatgtgcctggtccgagctaacggaggcggccgaactaggcgaaagccgagtggaactactgggtttgcagcccgctaatttcacgccttcgggcaaacgcaattgcagtgaaaagtaaaacaagcctcggatgctgtcgatcccaagtagtggcgacagaa

SEQ ID NO: 152 - DNA, 605 bp, synthetic construct
  source 1..605: mol_type = other DNA
agggactctcttagggaggtgtggacctgctggctccctcccgtaaaactggcttgacggctcccgattccagagttcttctgggtgaaagggttcagtcagtcagaaaggccacagcaggtagagatggagcctgtgcatctcaacggatacctatcaaacgagttatatcgaaaacgttataacgacataggtacacgcacagcgtggacatgggaagtcgttaacctactggcgctctcccataaaactggctcgatggctcccgataccagatttagtctgggggaaagggttccgtcagtcagtaagaccacagtaggtagagatgatgcttgtacatctcgacggtgacctatcaacgtgttatatcggaaacggtataacgacataggtaacgcatcgtactgccgatgtgtgagatgcgagcgagtcggcgtaagccgacagacactggcatctcagacgtcggtgtgttggtccccttgctccgtgtgcggggttgctccgtattgcggtgtctggtgcgtgcggcgagattgtctagtcagactctcgtagcaatggggtgaagcctggtcttgaacctcgcagtcccgtaaagg

SEQ ID NO: 153 - DNA, 619 bp, synthetic construct
  source 1..619: mol_type = other DNA
agtaactatggctctcttaagccaacactgtgagtgctcggaccattggtcccttcctttcaggtcatctgggcagggaccgccgatccctgtaccaagcgaacaatgccgacaccaccgtgcatttcgcggctgcaaattatatggggggaccttggtctttctatcgtcgggccacagaagctttttcttccttttgggcttactgttaatatacatatatatatatacaaacatattgtatcatgtttattattgatgatattgttttcatctccgtttccttcgggagcggatgcgtgtacgagtgggcgtcacgctgggccgcagtgccttaagagcttagttacctaaaggatcgatgatgcgaacacacagcctatctcgatcaagtctggcaccaccttctcgtggtttccgctgacaatctcaaccgaggcagcccggtgagagcaaacggtgtccagtcgaacgtgcgtgcaccccaatggggtagcgtcggtgcttttgtacggctactcaaaacctatgtcctacggtgagttgtggggtcaagaaaggacgggttgcttcgggggctctcggttgtcaatccgttagtccccaacacgcagcaaat

SEQ ID NO: 154 - DNA, 344 bp, synthetic construct
  source 1..344: mol_type = other DNA
aatttgggctttgtcttgtccggcaaaggtcaccatctttccgtaaggatcgacggtgccgctagctttactcgacacgagacctcattttgagaaaggttactccttaatcctaaaggattaccacaaaggaattagaggaaccaactttaatagcaaagggggagaccctctatttccgagtgcctagtaccacctcctcgcggtgtgggctggaaacaattacatctatacagaagtaattggccaacggtacaggttaatgattaatggctaacgatcttgtgctctgagatcgtaacaaaagcccaaaaagagaggagcggagtgtctattcgaaaatc

SEQ ID NO: 155 - DNA, 112 bp, synthetic construct
  source 1..112: mol_type = other DNA
actgcactattaatttaagagatcatagacgtgcctatcttgctccttctcgtggtgcccgccgagcacagtgattcatttcactgtggcaaatgcagaaggcttagaccat

SEQ ID NO: 156 - DNA, 227 bp, synthetic construct
  source 1..227: mol_type = other DNA
tcagggggtcgtagttacagggacggtgatgcgtaacacagacctctagcaaggtccttcagcctctcccgttcaaggtgtactccaaaacctcctagtggttcctgccggtaacgtggtgtgcttcggtacccacggctaattggagacatcgccctcggctcttgcgggttgccagccctctcgagcctaaatgtgttggcccaaataatataaaaaagcctcgg

SEQ ID NO: 157 - DNA, 144 bp, synthetic construct
  source 1..144: mol_type = other DNA
gcaaatcggagagcgggggagtcatggactccacccagtcgcaagactggggccaaggccggtgaacccttcaggtttactaggccgggggaagggaggtgtcctcacttatgtatgtgacgacacccccttctcaaggtccgc

SEQ ID NO: 158 - DNA, 227 bp, synthetic construct
  source 1..227: mol_type = other DNA
agcatccgtgtcgctcctacgacgcctgccctccagcttagccgtccgactgttgccagccagacgttgcccagctcccacctcgtggaggtgtggaaggcacgggagccacgttccatcaagtggcgtcgtcggagcgatacggatgctgtcgagagtcatagctagcaagcagctacctcctcgttggttcccgctgtgagtaccttcctcggaagggaaaccga

SEQ ID NO: 159 - DNA, 617 bp, synthetic construct
  source 1..617: mol_type = other DNA
acgacagttgaatagtgcagacgtgtttacacaacgctccgtggaaagagagtttagttcgggtaattgacggaaaaaaattataataaaaactttgtcggatagcgcgaaaaatttcccacaccatagtttttatttcgcgaaaatcccccaagggatggcaaagatattggcggattaaatttataggttgggaaattcgtaatacattcgcgtggactccaaccggcaagaacctgaagctgaagcacgtggcagagtgagagcacaacgaaatcgtcaaactatcgttcagaggttcgtgcactagatcaaacgaaagcacacctaggtgtgaagggtaaaactaacatactagggtggtggtattcggtgtgccaagacacaaaccgatttctgcattaccgacgagggtacgcggcttggatagcgtccgtagacacacaccacacgcaataccgacgaggtgagtggatttgatagggtgtcctaggacataaaccagcaggttaaattctgtggctagccgataccacagcgcgaacacagatctaggcaccgaaccgaagcacattccatctgaatagagagttagcttccaaggaagtaagataacg

SEQ ID NO: 160 - DNA, 682 bp, synthetic construct
  source 1..682: mol_type = other DNA
agtaagcaaacctgcggtattggcccatggcggttgtgtctgcacacatatgaaaatatgtgggcaaccttgactgatcaatgtggcattgtcttggtggaatgctcacacccctaacggggaagatgttgtcttagatactcacgggttggtcgtagcacgaatttccgagcccgccggctagcttcggggatgcgaagaatgcgcggccagcccgtcaaaggggaagagtgacttgtcacgacgcccaactcctatccacagcctgcccgtggcgccgctgccaagctgatgtgggagggggggatccgcacctaaattaggcagcccgatagtagccgttgcggtgctgcatgcctcgttggtgcaaatatcctaatccactcgtcgacgaggccacaagcgctattccattgcggtctccaccatcgccgatacggtcctcacggatacgtctgcggtggtggagaccggaatcaggcttgaccctggattctctttgagtggtgcgacagccttccatcttaaggttgtaatccaggtggtgactgcgaggtgagattcctcgcagtagtcccgatggttggtctctacggagaccttcaagcctagcggttttcttcggttgggaccttgcaaaaaccgtgggcgaatcccgtggttgggtttgcggcccgaccat

SEQ ID NO: 161 - DNA, 706 bp, synthetic construct
  source 1..706: mol_type = other DNA
agagtcttagttctcctgcattgaacgcgaagtttcccgctcgggtgtgacgaccccgctttagcccagttggtcgaccggaaccggcgacgtggtccgagtctgcaaagaggagggctgccgaaccaatccggaggagtgatgtcccgccacatgggctcctccgattttatagatgcataaagcatcagtacttcgagataagtagtatggggcgaatagatcattttatcgtgcccgaaggaagcaatgcgcgtcgaatctaaattcttctaagacaagcaatgcgcgtcaatccatcgactactacgctaagtcccaatgaactgaggagatactcaagacatcgagcatcgaagaggccgcataagatcgcgtgctatttaccgagtggctgcaggccaagggcactatcgacggtatatgcagtgttttacacttggggggatattaatctcgaaggggaatgaagaccactagtctcgtactgagaggtcaagcggaaagtccgctcaccgggatagtccgatgaaaactatacttgtataaggcgaataacatatcgtagcgacattacgacgcctcataagtcacacttgagcagtgtacgacttacttatgacgttctatggcaatctaggcaaacgactcatcatttggtacattagtgtctaagcttagcaaaacaccccaaaggaacaaaata

SEQ ID NO: 162 - DNA, 176 bp, synthetic construct
  source 1..176: mol_type = other DNA
actcccaggtgaacgcctttagccttggagcccaagtgccaaccgtacacctcctcgtggtgagagtcgggcaacgtcaccggcggccgagtacggtggctatctacgggtcttggtgggttgccagccccctcgacccaaaccgtgttggtccaaatttaagaaacaagcctcgg

SEQ ID NO: 163 - DNA, 325 bp, synthetic construct
  source 1..325: mol_type = other DNA
agaaccacggcgtgatctgaagtgggcgcagcctcgtttttgtctctcgtcaaaaggtatacgacgctggtacttggaagaatttctcccaagtgctggctgagtgtgccggcgggaggcaggctcgtttttgtgtcccgccaaaggtatacgccgctggggactttgaggtctagaagtgcctctcctccacctcctcgtggtgcccgccggtaacagactggacccctagtctggctaacggaggtaggctaatcggatcctgtgggttgccagcccactcgatcctcaacaagtcggcccaaacaatataaatcagcctcgg

SEQ ID NO: 164 - DNA, 152 bp, synthetic construct
  source 1..152: mol_type = other DNA
agtccaacccccccgcctcgtggtgagagttggtaacatacactgctaattatagtgtatggctaacggggctctggcagggtgtttttggggttaccagcccctcaaactccaaaaatttaattggtccaaattgcaaaagagtgcctcgg

SEQ ID NO: 165 - DNA, 646 bp, synthetic construct
  source 1..646: mol_type = other DNA
ggttgctcacgggtcggtcgacgtgcgtgtttccggcgacagcggctactcgctggggatacgacgtggagcgcgatcggctcgtcaacggggaagagtggtctcggccacgacgcccaactcctatccacagcctgctcgtggcgccgctgcgatcgctaatgtgggggggggggacacgcgtctttaacatcatgcgaccctgcactggccgttaaggtgtccatcgcctcggtggcgcgtgtccttcgaacgactgcgaggccaagcacgtttccactatggtctgcgccgccggtgtgcctttcctttttggatcgtacgccggtggcgcaggacgtagtcaggcttgtcactgggttctctacggagtgtgcggcgcccccccttccatctttgggggcgtaatccaggtgaggttggagctgagacgctgcaacagtccttcagggaggttttttcagccgttttcacggctttttcctccaagtccaatagcttccttcgggtgggacctttgcgaaagctatgggcgaatcccagtggtagggtttgcggcccgccacaattggggaaattgattattgcaaaaagtccgatcagaaaatgtgagggccaccctgacctcccacaggagtcggggagc

SEQ ID NO: 166 - DNA, 650 bp, synthetic construct
  source 1..650: mol_type = other DNA
aggagagtgcgaaaacgcgtcctcgattaaagaagaatgataagtggcacttactatcgtaggccacaataaaaagggataagctatatgcagacatacaaattcaaggctaacaatatctgagcctaaactgaggattgcgccattagcactggcctatcgcttgaaggtgtcgggtcaggaaactgacctaggatccgagacgggccgacctggaagcagttgtttgaggtgtcggggggcatttgctccctagtatccgagactagtctaccatggtactcctttagaggcttagtttagcaagtagccacctccgcgttggttcccgccgctagtaccctcgaagactgtcgttgaggaagggagcatagtcttccctgtgtccgacagtcacggtggatatttgatttaatatctgaatccacccacaaggcagtcgtttgaggtgtcgggtcttaatggcctagtatccgagactagccttttcgaggagaagctaagagctaccgctgcgcctaaaaggggtcggacgggttgccagccctcgaccccaccgaactggcccgaatatcagaaataaagcctcggatggatatagtaaccccgggcccactggctctaggggtccagctcctcgaagccgaggt

SEQ ID NO: 167 - DNA, 313 bp, synthetic construct
  source 1..313: mol_type = other DNA
tttggcgggagtaactatgactctcttaagggtcatagtgcagggtcgttgatgcgtctcttatagcctctcacgaccaagtcgcgcgtcatgcttccaagtagccccgccggacaacataggtgtggtctgttggattacacctatggctgaatgacgcgttgcgaaccgggcccggtgggctgccaccccatcggaatccctttcagtaggccctaacagtaaagatggaaatattactagccggcggaggcctcggaccgccggccctactgacacattacctcatacgcccaatccccccgctggaccc

SEQ ID NO: 168 - DNA, 655 bp, synthetic construct
  source 1..655: mol_type = other DNA
cgggggagtaactatgactctctcgggggagtaactatgactctctcggtcctcaaagcaacacgccgcgatggtacttagagagagtcgtagttgggtcgtgatgcgccaacgaagtcctctagtaaggaccttcagcctctcacgtccaagttgtaccccacctcctcgtggtcactcccgggcaacgtccccaccatagcaagacggtacggtgggcgacggctaacgggtgtacagcgccgagcaaattagcacgctcggaactcgccaagctggcgcatcagggataacttgttaggcctgtcatcggccgaaaaggcgaagcgaagaaaggaatcaaatggcgtaagccatcatgggggaacagattattctgtgaaaaagggaaattgctgtcccagaattgggaaactaacccccagctcgccttttctcttcttccaagccggctctttgtagtccgggtataccttgtgcaatcagacaccaaagaaaccatcggggtgaggagcagcctagagtgcccagagtttgtttgcggaaatcaaaagcaacacgccgcaaactaatttataaccgtcggggtgaggtgcagcccacgattgcccgaccgtcgtcttggctccaggcgacggtctattcaggagcaaat

SEQ ID NO: 169 - DNA, 336 bp, synthetic construct
  source 1..336: mol_type = other DNA
agcgatgattccactgttactatgtaacactaacgtcatgtgtaatacatgcaattggggtgtcccgtggttaccatgccacgggcgccccttttcacgttaagcaatttttaagttctgtaaaagtacagagagtgctgttggttcgcccatacacttgagttcttttacacaggaaaagcttaaagagagaagtagttgagcaagtagccccctcttcgtgggtcccgctagcaactagtcattgactaggctaagagctacctctgcgcctatcggcttttcgggtcgccagcccgtttagcttaaagtataacaggcccaaataatgaagct

SEQ ID NO: 170 - DNA, 827 bp, synthetic construct
  source 1..827: mol_type = other DNA
ggcgggaggacctatgactctcttaaggtgaagtgtaaagcctttgaacaaatgagtgctaggggatggacctcggtcctactccagaccggaaggcgtacaacgctctctctgctgagttcgaataggccaatgatcatttgattaagccccggtcaatgccatcctgcctcgttctgacacaaacgtgggggcaaccccatgcgaagtcgcgactgtatccctgggttctgcctggggatatacagggtgttctccccaatctccttgcgtcaaggggagtaaggtaatagccctgcgaagcggtcggcgccggtaaccaaatttgcgaaggcgggccggtgggcgaggatgtggatgttgagggaaaccttagtcggttgggatataccgacgcaggccaggacgaggtagttcgtagagtggggtacgttgtgatgggctgggggatcctagtcactcatacaaaatgcatcaactccaactcaatgcaatagatcgaatgtatacgaaaagagaccatagttataatgggaagatgcagcgcaagtcacagcttcccttcttccaatggttgtactctacctcctcgtggtaccgctgggtctctctcgaaatgagagtcggactgagtacagcgaaaccgaaggtatcaccacttcgggccggttctcggtgccagcgggctttttccgtaggtggttagcgccccgaaggttcgccatgcgccacgttctatgggaagttgtgggggccgataaaagaactggcttttagggggctctcggctgtcaatccgtttgccccctagcacgcttagaattttc

SEQ ID NO: 171 - DNA, 647 bp, synthetic construct
  source 1..647: mol_type = other DNA
ccaaccccacctcctcgtggtgagagtcgggcaacgtctggggctcaccccccggcggccaacggggctctggcataaggacaccgaaaggctctttccttgaagtaactatgcgacaacatcttagcacataaactagtggcgaactggccttgccagataattaagaattgctaatatctaggaaggggtagtgctgtcattggcaggactcccctttttgctttaaaaaaattaaagcaacacgccgcgaggagtcttagtcactctggtgtcctacccgtgagttatctagtctaataaccggctagctgaatagcccgtaaacccacggctttaattagtagatatgggcgatccgagtcgccaacagcgtcgaaagagctctgcgaagtatattgaacctataagttggctgaagtcgactattcatggggaaacagagataacactctgtgaaatgctgatcagcaaataagtgcctttggcattattctgccccttagcttggctcgaactgacgcatgttataagtattgcgtctcgagcgattaagtccatcgtgtggcgaggtgctgccgaatgcccggacgtcgcgtcagctctgcgtgacgatcctattaaaggagcgaatatatatttatgat

SEQ ID NO: 172 - DNA, 263 bp, synthetic construct
  source 1..263: mol_type = other DNA
cgggtcagtggctcaggtggtagcgtgtctgactcgtagcaggaaggccagggttcgaatcccgggtgtggcaagaatttccaaggtggttttttccctcgctgcgagtttgatacctactatcggagtcacggggtgaccaactgcaatgatttcccctttttccagggagtcggcgatttgttggaagcccaagttgaccaccccgccccataagggtacccgccgctctgtctccccagtttgggaacagagaaaggggg

SEQ ID NO: 173 - DNA, 617 bp, synthetic construct
  source 1..617: mol_type = other DNA
agattgttgtacggctccggccgtacaacaatcgagattgcttacatatgttattgaatgcgacggactgtgaataaaaagagtgataaagcagccctctgtggcgcccaaatccacaaacgagcagcaatctcctaacaaagaacgaagtacggtggtcttgcctccaaggggcggagctggggaccaggcaagtagaaatactacctggaagctaaaaggaccattctcacctaaagtcagacggagcttatgccgttctgactcaacatccttggtgaattttaacaaaagtgtttcatcgaaaaattgaaggctagagtatacaattttatccattttacatattgacataattttatttccaattgacattatatctaattttaactattgaccgaccacatctaatttgccgtcggcgtagattcttttaggataacgaccactcgatagagtgacaaaaactctctctgagagtaatttagaatagggaagggcgaagtaacccaaattcaatcgaatatcgggcgagaaaaatattccgctcggtccccttacccaccgaaagataaaaagcgttgcgttgatattttctaaaaggtcgaatcatcaag

SEQ ID NO: 174 - DNA, 160 bp, synthetic construct
  source 1..160: mol_type = other DNA
gagttgtagttagcaagcgaccccctctcacttcggttcgggtcccgccacgagtagccttcgggtgaaaccaagagtcgcctctgtgtctttcagccctaggggctaccaccccctagggcgcagtcaaactggtccaaacgcaagaaatcagcctcgg

SEQ ID NO: 175 - DNA, 351 bp, synthetic construct
  source 1..351: mol_type = other DNA
gttactttggcctggaaatttttcacaacatcgagaatatttccaggtcaagccgagcagctacctcctcgtggtcccgctggaaacacgacttatgtcgtggctaaacgctgctcggattgatggttaagtgcagccgataaagcacgttacgcgaagatctgcgtatggtgaggagcagcctgttataaatgcccaaacgcctacctcgggcttccgggggaaggagcggacaggttagccccctgtcttctcgtcaaataactggctttcagtgcgcagaatgaagcctcggacctgaacgcacatggtcccgtgcgtcaggagaatcaggggactactgttgacaaa

SEQ ID NO: 176 - DNA, 385 bp, synthetic construct
  source 1..385: mol_type = other DNA
agacatagttacttgggtaggtgatgcgtcttacagaccacttaggtccgttagcctctccctaccaagtggttgatgcccacctcctataggtgcctgccggtaacggggtcgatggaggaatccggaaaccccggctaagaggctcaaccgcgaatggggagcaactggatttcgatctctccggttaccccccgagccaaatgggggatgctggggtcaggggacgaaggatcaatccaacccgcttctatcccgtctggaaaccctccccaaggccgtacttcacgtccagcccttaggcaaggcgacgtggctctggaaaagggacccccttctgtgcctggtcccaattaacgaagccggccgaaacgggcgcaagccc

SEQ ID NO: 177 - DNA, 206 bp, synthetic construct
  source 1..206: mol_type = other DNA
tactggacaagaagattgtaccttggggcaggtgcatcttcagaccttctgagcccctagcccgatatcaggctaaagtctgtacagtggctactcagacggagtgccctgacacaaccataagtgaggtgcaatgcacaagaacctcacagacaaccaatattaaggacaaatacggctactcttatgtagctgagaaaagaaat

SEQ ID NO: 178 - DNA, 359 bp, synthetic construct
  source 1..359: mol_type = other DNA
aggggctggcgatccgggtggcgtgttacgctcggttctgccgacctaagagggtcaggctctcttaggtccttgattctcattgcgagtctctttggacctggggttagagttattggttacttgggtcgagagagcaatctctcgatcaagtcattaacgcccacctcctcgtggtcccgctggaacgctgcatctttgcagtaaccaagtggcgttatggcgaaacatgtgcgccccaaagggctatcagcccatgtgcctaggtctacggtgagctgtggggccaagaaagaccgggcttcgggggactttcggctgttaatccgtctgctcccccaacacgcagttaattatga

SEQ ID NO: 179 - DNA, 342 bp, synthetic construct
  source 1..342: mol_type = other DNA
tggggtagccggagagagctagttactaactgggcatagctgcagaagtcacacctccgcgtggtcccgctggatgcccgtaaagggtaactgagttgacttctgccccagtgtgagtgagggtgcactagttgccccctccctatcggttggactcgccacccagcggtagtttcacaatttgcagaaaaaccggtttggcgcagccgttcgctgttgcccgaagccttgtccacgggctaagttggacacagtctgcagctggtggagggttctggctgtggacttggacaaccctcaattgattgtgacagtgaccaattaccatctttcggtcttaca

SEQ ID NO: 180 - DNA, 637 bp, synthetic construct
  source 1..637: mol_type = other DNA
ctcttgggtttcgcagtgcaaccacctcctcgtggtgaatcctgggtagcggggcttttgcccccgcgagctaagagttgcacgttcccgcacgacatgagccgggacattaaacaatagcatgtcaccgtcccgttaaggttgttagccgggacgttaaaacaatagcacttcaccaatcctgaggtcgtaccgggagtacatactagcactggcacacggagggtcatctagatcgaaaaatcttttgtgggaggttttttcttatctatacaacacgacaaaattttcttagtaatgtggaatactaataatttcggcctttatttcttcggttcgacccctcggtttctaaattatttttatcgctatcgatctgcttacttcaaccgagggggcttttcgggtttgcatcgcccaacttttctgactctccggctgtgatgctacacagttatcgaaaaagcaccctgaataacatgagcagggggtcctcaaggcccaggtcgtcccttctggacttcttgtgccaatagccgagcacaaccttgtgtgtggaccttaaataatgtgaccaggctccgcgggggggtgtgggtggtgccaaccacttacatgacccccaaataaatcaagc

SEQ ID NO: 181 - DNA, 173 bp, synthetic construct
  source 1..173: mol_type = other DNA
gatcgccattaggtgtcatagtttagcaagcagccccctctatgttgggtcccgccacgagtaactccttcgggagtgaaaccgagagctgcctctgtgaatccgggtcctgggggttgccagccctctcgacctgagacctagtcggcccaagaaatacagaactgcctcgg

SEQ ID NO: 182 - DNA, 171 bp, synthetic construct
  source 1..171: mol_type = other DNA
gatcgccattaggtgtcatagtttagcaagcagccccctctatgttgggtcccgccacgagtaactccttcgggagtgaaaccgagagctgcctctgcgaaatcgggtcctgggggttgccgtcccccccgaccttgacgtgttggcccaaacgacaatacacagcctcgg

SEQ ID NO: 183 - DNA, 340 bp, synthetic construct
  source 1..340: mol_type = other DNA
taaggcagcttgagagggttggttactaactgggcttagctgcggaagtcacacctcctagtggtcccgctggacacccgtaaagggtgactgagttgacttctgccccagtgtgagtgagggggtttactctccctccctgttgtctggactcgccacccagcgacagtttcacgaattcgctgaaacaaataataattggcacagctattagctgtcggccagagcctggtccacgggtctaagttggacgcagttagcagcagtgggggagggccccgctgttgactttgacaaccctcacttggacgtgtctcaattttttcctcctaatttgacc

SEQ ID NO: 184 - DNA, 310 bp, synthetic construct
  source 1..310: mol_type = other DNA
tttggggggacaaacctccgtatgcctagtggtgtgacaggctctaacaccgagtctcgagcaccgcaagtctaggtcgccagaagttgccctgtgggttgctgtctggcctaagtatggaggatgtgccaaagatgctcgctcctcgtgcgtccccctgggtgctacactgtgtgtccgggttcgtcctgttgagtaaagtgtagcagctgaagcatcgcggcttagggcgtgtcgggttaccagcccgacacatgccctttcagttggtcaggacacggtagaaaatgcctcggatggcggaggccaa

SEQ ID NO: 185 - DNA, 133 bp, synthetic construct
  source 1..133: mol_type = other DNA
gcgggggtcatggttgcatggtccgggcgcctcctcgtggcgaccgctggagaaggaggggcttgcgcccctcccgagctaagatccggaccaccctcccggcggggtaaccgccctccgggagggtaaagtg

SEQ ID NO: 186 - DNA, 252 bp, synthetic construct
  source 1..252: mol_type = other DNA
actgggtgtcatagttagcaagcagcctccttctagtgggtcccgccgcctagtacctccttcgggggggaagctaagagctgcctctgcgatcttcgggcctgttcgggttgccagcccgttcaggccgtcaaatgtgttggtcaggacaatcaaaatagacaaagagaaatccgccggcgagggcttcggccctctcggatcgccggcccaacgcatccctcgtcgtcgtcccccaatatacccccttca

SEQ ID NO: 187 - DNA, 135 bp, synthetic construct
  source 1..135: mol_type = other DNA
gaggggggttcttcggaaccgccttcccctcgcgcgcctgaggcgccttccccctcagcttctgctgggggggtcgcatgccgaatttgccagcctgtacggacatttactacctcccgtgggctggcgaatcat

SEQ ID NO: 188 - DNA, 339 bp, synthetic construct
  source 1..339: mol_type = other DNA
tcatagttgctcgggggcggacgagactcaggtcttatccgcccaagtcgcactcctacctccccgtggtaccgccggtaacacgcccgccgcattagcgtggggtgcatatccccccggatgcggcgtgcgtggctaaacggagtgtggcgatgaaggagcgaaagacattctaactaacaatcggcttaccgattaacggcttgctcgcagccgtgaaatataatatcttaaggctttatggaactcgttccttgacttccttttgatgacgacatggcaagtcaacggacatgagacggatgcttttggatcagttaactcagatcacaaacaaca

SEQ ID NO: 189 - DNA, 282 bp, synthetic construct
  source 1..282: mol_type = other DNA
gctgctggttatttttcttcgccttgactcctggggcctatccacaggaggaataaaccgtctttgttcttccttctttgtgtctttattttgtgctttttgttgttgttcttccgcactgatatatatgtatatgtgtatgttaattttatacttgtattttgtggtaccgccctgtaagtccccacctcggtggcggacatggcgtcaaacacctgccacgtactatatatgtatatattttgtgttattcaaattattttgaataaagacggctgttga

SEQ ID NO: 190 - DNA, 199 bp, synthetic construct
  source 1..199: mol_type = other DNA
ccaaatacctggttcattatttgcttgctaccttggcataacatcaaaggtacaaacatcgcgcaagtggaaaacacgaggagggttttagtacgtaggcgctgtagaatttccaccgtgaggattttctgaagaagcagtgaatcgtgcatgctaggagaacctagtatctttcgaagatttccctaccttggcgatc

SEQ ID NO: 191 - DNA, 469 bp, synthetic construct
  source 1..469: mol_type = other DNA
tcgttttacactagtacgtattaaagtacgtataagaattgactaatcttgacaaagcggctgcccgctgatataaaaaaatgtatagcccgtttcggcattatacaccgaacggtgcgagtcgtactatacgaaagtatagtacggcgcggggcgggttctgtgtatgtggcgctatatatagttatatatgtggagccacatgcatagagcccggggcgacacctccacgtggtttccctggacacatgcctctggcaagtgactaagttcgcctcccgaaagggtccccgttactaatttttccaagcccgtagggcagctcccttggcagtggttttagggacatggaatctcgttaatcataatagaaataggcgtagttgttaaatttattgttattgttatacacctcacggggaggttataccccggaagtctgctatcacctacggtgatagcacaggtt

SEQ ID NO: 192 - DNA, 231 bp, synthetic construct
  source 1..231: mol_type = other DNA
gtgtccggggtcctgtggagtttcgggccgctgccgagcccttgggggcttagtaccaaagttttgtgagtactctttccctgctggtcatccatgagctcccaattcccaacaatcggcacaatgctacgcccaagatggaagacagggactagtggtatcttccgggcatccatgatgattataaatcgcgtatactaaagacgaatagcatcatgtgatggcggaaaa

SEQ ID NO: 193 - DNA, 151 bp, synthetic construct
  source 1..151: mol_type = other DNA
attcggatttgtcttcagttcatactgtgctacctgcagtctcaaggtacaaggagagaggggttttagtgagtaccaccaacatctgagcctcacactactggtaatacaacatctttactagtgtgccataatcgcatttcccctcctt

SEQ ID NO: 194 - DNA, 216 bp, synthetic construct
  source 1..216: mol_type = other DNA
cccagactccataatttatacaatatgtgttatttatttatttatttttattaaattgtacatatggaattatttgcaatatttatcttatacaatgtgtcgtctaatttattgaatttgccgtaatttaattacaaggcaaatgtttaagttttaatttccctacgagggggtacctcggaagtctgaagcttcttttgaagtttcacagaatta

SEQ ID NO: 195 - DNA, 408 bp, synthetic construct
  source 1..408: mol_type = other DNA
aacaggtatagctctcctcaattaactatttgaatattacctgatagtaccaggattttataaaaaaattcccccatttatatatattttttaatatatatttttacttcttgcacattcaatcgtttttatttactcctacttatctgtttttatattttatctatttcaccgttcccatgcatgtattttaatccttcagaaactcgtttatttgaggcatatattttacccattactttatattttattcctgtagcttatttttattatatttattaaatttagtacataagcaatcagaagtataaaccgacttagtattgagtatcaatacttttttatacttcttatcctacgaggggacacctcggaagtctgctggagttatattttagcacaggaaat

SEQ ID NO: 196 - DNA, 443 bp, synthetic construct
  source 1..443: mol_type = other DNA
aatagatcaggttacttactataggtaaggttctgacaccttcaaacttcaattgttaaaattctccaattttatatatacacgattttattttgcctacttcatgtttgttttgttatcgtagttttaatttgttattatagtctagtcattttatcccataagcttacagctattttcataaattgactattgtcaatattttaacccttgttatgacatagcatgcctatattttatcactgcatgcgataatacaggctcaccctgtattacatgttgacctatcgaaatgttaatttattaatttattatcttcccccctaacttaatcttatttgtttcctatttttttctctttcagctttttagttattttaccctacgagggggcacctcggaagtgtgttgctttgaataaaagcttaaaaattacataggaa

SEQ ID NO: 197 - DNA, 172 bp, synthetic construct
  source 1..172: mol_type = other DNA
ggattcattggctttgaaggacaataccttgagtgccgcctgctcttccatctcaggcgtaaacaaggaagcgcagactcgaagtggttttagtgagtagtgtcttttttaagatgagtctcacactacccgtatcttcccgggtgtccataaatggatttccacttccttc

SEQ ID NO: 198 - DNA, 168 bp, synthetic construct
  source 1..168: mol_type = other DNA
ggactcgggatccagtggataggggcgataccgggcctaacatgaccatctcaccggtacaatgttagcgtctggcgggcataacatctggcccgccatctctttggtccatgatgattacattatatgtcacaatggacgaacaagcatgatgtgcgctaattcaaa

SEQ ID NO: 199 - DNA, 202 bp, synthetic construct
  source 1..202: mol_type = other DNA
ccctgcggaagggcaccccccttcgatcgaccgcgaccttccccctcttggcggaccgaccaacaactcgaggatctgcagagaaagaagatatatgtattattatatatgtgtatcatgtattattatattattgtaaaccctacgggtgggtaataactaaacccggaatgtgttaattagaatcaataaacattttaaa

SEQ ID NO: 200 - DNA, 179 bp, synthetic construct
  source 1..179: mol_type = other DNA
tttaacgaaattcaatttattttattttattttaattatttatttatttattgaattgtacatatgaaatttattagtatttatttatttattattgactttgtcaattttagtttatattttattccctacgagggggtacctcggaagtttggagcctttagtgaggttccataggt

SEQ ID NO: 201 - DNA, 144 bp, synthetic construct
  source 1..144: mol_type = other DNA
agctatcgaacaccgtgctgcctacggactaggcacaatgagaaggagtgggttttttagtgggtatgtccaaaaggaagagccccacacaaacagcaattgaacagcggctgagtgcgtaatgcatttttccctctccttctt

SEQ ID NO: 202 - DNA, 225 bp, synthetic construct
  source 1..225: mol_type = other DNA
tggatggcgcgtagagttgggggcctccgcatcaatcgcgcgagtttgaatgattgcgtgaatggtcgaggtgaagtagtacgagctgcctgtggctagctccgaccttcacgctccagggtgttgactgcgaacaccaacagtaaacagctctgcaatatcgttgtcggtggcgacttggcgactgtgattttgtagcagtcgtcgcgcaccgtagacgaaaca

SEQ ID NO: 203 - DNA, 334 bp, synthetic construct
  source 1..334: mol_type = other DNA
gcgcccgcgcggtttagttcgtgcaggctgccgccgtgcctatcagggggggtcatccttgtgggaagtacagcaatgtgcttctcgcagcttgacatcccgtttgaacgagtacctgtacctcccctgagtttacatccagaacggcgtaattccgtgcgagatggttcagggactgggtcgcacgaacgggtgccgatggggacaccagtctctgggaaccttgttaatccataatgattacaagttcccgttgaatgatcggttttgatttgattctagggatgtcagtaattattatggatgaagcatgtgtcagtggctcggattaaaa

SEQ ID NO: 204 - DNA, 401 bp, synthetic construct
  source 1..401: mol_type = other DNA
aatagattaggttactttgattgtaaattaggttttacattctaaattttaactttcatttttggtcaattctacatgcatgattttattataattgcctccaacagggagtatatcgttttcgtttctgtttttgttattttaaacactgttatcaatctagtcatttttcccacacgactatagctatttctataaattgactattgtcaatattttaaccccttttatgacttagcatgccaataatttattactgcatgcgctatattaatacatgaccatcacgtattacattgttgacccacgatagattttagtttttagttttaacttttaccctacgagggggtacctcggaagtgtgtgttgcttcgaaagaagccattttacataggaaa

SEQ ID NO: 205 - DNA, 214 bp, synthetic construct
  source 1..214: mol_type = other DNA
cccaggcttttaagtttaataatattcataatttatttatttatttatttattaagtaattcattaatttgtatatatatatctatttatctagttatttatttatttattaatttgtcgtaatcttttgattacaaggcaaatgctttaattttatttccctacgagggggtttacctcggaagtatggtcctcgtttgaggttccacagatt

SEQ ID NO: 206 - DNA, 368 bp, synthetic construct
  source 1..368: mol_type = other DNA
gcattctctgtgaggtcgggaatgcgattggttgtgcctctgaaggctggaataaagtaggtaagggatatgagacaaggcacagcgagggaattcgaaatagtatgggtaaacggcaatgatatctatgactctctaagggtcttaacttacctactatctagcgaaaccaacgccaaggatacgggcttggccgattagtagggaacagaagcgaactaaccacaccgatgattctgcccggaaagtccggggacgccaaggttgtggatagagcggataaggggacgcgtccatgccatcgacgctattcgtgtcgacgagcgacattaaaactcgaaatgtgcactctgatgagtgttgttaaa

SEQ ID NO: 207 - DNA, 289 bp, synthetic construct
  source 1..289: mol_type = other DNA
cctaaaatattttaacttaaatgttatttatttaaattatttaatttatattataaaatcaattgtatatatgaaataaagacgacaaactaattatttagtcgtctattttaacactgtatcgtcctatttattattattttatattatgactgtatattgtctcttgaattgacttatcaattttgaggcaaataaccgagcaatcggttattatatactattattttaatatccctacgagggggtacctcggaagtgtagacctcgcttagaggtctataggata

SEQ ID NO: 208 - DNA, 167 bp, synthetic construct
  source 1..167: mol_type = other DNA
gaggaacaacgactatcggttaattgggtgctgcctgccctaacatctcaggcataaggagaaggtggggttttagtgggtattcacgttttttagtgctcgagtcccacactaccggctgaacaacatcacagccggtgtgcgtaaaagcatttccccacctttgc

SEQ ID NO: 209 - DNA, 156 bp, synthetic construct
  source 1..156: mol_type = other DNA
aggaacatgaactttcatgccataagaacggcactacctatagaattaggataatattaataacttatatttaattttcgcctaccagcgggcaaactataattaccatcggaatacactggtcaaattgtatgaaggcagaaaaatacaaggttc

SEQ ID NO: 210 - DNA, 196 bp, synthetic construct
  source 1..196: mol_type = other DNA
gcgtatggcatggcgtttttgcgcctctactggtagggttacccgtccccacaccatcctcgacgggcaatatgccgtttttgcagccatcgcagctgtactggaggggttttagtcggtagtctgcttcacggcaggagtccgacactaccccctgacaccagggggtgtgcataaagcatttcccctccaaggc

SEQ ID NO: 211 - DNA, 191 bp, synthetic construct
  source 1..191: mol_type = other DNA
ataaacaaggcccctccccctggctgggtccgctggacagtggaatcggttttgggtgggaggggttcatccttcataaccgcgtctttagcccatatttgggctataaattccgatttggacatcaggtggacgggccaccttacttaacccggaaaaggaacatatacagtttgtatgtgtttaataaa

SEQ ID NO: 212 - DNA, 301 bp, synthetic construct
  source 1..301: mol_type = other DNA
accagagcgaacttatttatttatttaacaatctactctaattcatcttatcctatttcaattttattttatactatttacgatttgatcttattttacttacatgacatcgtatttacaagacactcatacgcatttttatctatgacaattttatctattaaatctaaaatattttattactacccttaccattgttattgttgttattatttatagttatatagttataagttgttaagtttgtaaccaccggggatataggcccggaaagtacgtccgttttaaaatgtcgacgaag

SEQ ID NO: 213 - DNA, 194 bp, synthetic construct
  source 1..194: mol_type = other DNA
attgttaaataaataaatggcatgtgacacttcgcatggattaggaggggtggacaccggtggggcttgtcattccgctcataccgtgtcacccgcccatatttgggcttattaaattccgatctggacaaggtggacgggccaccttcacttaacccggacaagaagcatacgttcttagtatgtttattaga

SEQ ID NO: 214 - DNA, 354 bp, synthetic construct
  source 1..354: mol_type = other DNA
gtagtcactctgcactttctgtaacttgcacggggtgtgggacgtgggtttggggtgtgggctatgggtgtttttatatggggggatcctttgtgggaatgtctatttattgtatgtctatcttttcaataaaaagacggtagctaggttcgcaaggcagccacaagccattagccagttaggtagcttcatagtgggtaggtgacaggaacttttgactcagaacgcgtccattaacatctattacggaccactgtaaccggacatgaaccgattaaccggatttgtccagggtggacgggccacctgtacttaacccggaaagggaacatatattattatgtgttcgaaata

SEQ ID NO: 215 - DNA, 421 bp, synthetic construct
  source 1..421: mol_type = other DNA
ttaattggtctatgatacctggccaatgtgcgcctcaaattttaactaatgacatcgacttaagatctaagtcgaagctgcttcttaacaagcccgtgacccgtagggacgcggtgcgagtcgtacggcagtgcaaaccatgagcacttgccacggcggcggagccgggagctaatactcctggacggacacctccacgaggtctttcccgggcaacgctcagaacatcgagcggctaagttccgtaaatcatgtttatgaactccgttaagcgctaatttttccaagtctggcagtctatgacgtgttagaggcgaagctcttgatcgccctaacgatataccgtacgaggaggtataacctcggaagtatacggtaacaagcagcgacgaacaccggtcgccgcaaccgtacaggaata

SEQ ID NO: 216 - DNA, 270 bp, synthetic construct
  source 1..270: mol_type = other DNA
gtcggggagcgagtgaccggattcctgtggtcgcgggccgccgccgggcccaggagtcaaatctcccccagaggcttctttccccctgttgttaacagaacaactttttgacttcgcccaacatccttccggcgtaatctttgtagtgcggtagcagtagaggattctggggagtcctgctacgacgggaacatgggaatcaatgcgttaatccgtaatgatttacagtagttttcgctaatggacgtaggcatcactactgagctaaaa

SEQ ID NO: 217 - DNA, 198 bp, synthetic construct
  source 1..198: mol_type = other DNA
gtcagacccagttttttaaattattatttatttctttatttatttatttatttatttattttgttgttacgtccatgcatgatagtgaataaatttgctgtaacctttgggttacatagcaaatatgttatattttaatgtccctacgagggggataaacctcggaagtacggtcctcgtatcgaggatcgcaggttt

SEQ ID NO: 218 - DNA, 148 bp, synthetic construct
  source 1..148: mol_type = other DNA
atgtcaaattttgtcgtactaactgtgctgcctgcagataatcaggcataatacaggggggtttttagtgagtataacaacacctgagtctcacactaccggtataacaacatctgtaccggtgtgcgtaatagcattttcccctctc

SEQ ID NO: 219 - DNA, 76 bp, synthetic construct
  source 1..76: mol_type = other DNA
agtgcgaaagaaggagaaaatatagtgctgcctcacaggcataatgttattgttacattgctgtaaacaaataatt

SEQ ID NO: 220 - DNA, 193 bp, synthetic construct
  source 1..193: mol_type = other DNA
cgaagcatcccctttgtctcggttgctatctggccaaacacctttcaggtacaaaatcgtcaacatatgctaatacgctaaggaggttttagtgcgtaggcgtctagggcttgctctagatgaatcgtacataactcttacactgtgaatgctttgtaagagtatcttttgaagatttccttccttcgacgac

SEQ ID NO: 221 - DNA, 189 bp, synthetic construct
  source 1..189: mol_type = other DNA
gtcctacataaaatcggttgctatctgacctaacatctttcaggtataaactcgtcaaatatttgctaatatgctaaggaggttttagtgcgtaggcgtctaggacttgttctagatgaatcgtacataactcttacactgtgaatgctttgtaagagtatcttttgaagatttccttccttcgacgac

SEQ ID NO: 222 - DNA, 393 bp, synthetic construct
  source 1..393: mol_type = other DNA
gaatgatggaattttatccaataaattttaacctttttttaaggaaattttatcaatatacttttatctacacgagggaaatttcttttatttttacactgagtatgtattttaacgtttttatatacaatcatattattagtgaaaaccattttacatacttaattactaggcatgcaattgcgtaaacgtgaatcaaaattttacataattaactacttagcatgctaatgttttatcgaggacaaatatctactaactttaatacttaatgactgacattcttatctattaataacaggctattgacaagctcaatacacttattaatatttaaatcctacgaggggattcctcggaagtatgcaattccaattgaattagcacaggaaa

SEQ ID NO: 223 - DNA, 295 bp, synthetic construct
  source 1..295: mol_type = other DNA
caattaattctctcgtagtttattccttattttcattctcctcgtacatcttcgctattctcactgcttctcactactctatatatctactctttcttcggctattctttactgcatatttgtctctcgttctccttcatgcgcgatttcccctccttctacatttcccctttcttctatggattcttccctcttaacggttggacactctgaatctattactagcctaccagcgggcctctgatcaaatacactggactactaaacttattcctagaatatttatagagtgtca

SEQ ID NO: 224 - DNA, 160 bp, synthetic construct
  source 1..160: mol_type = other DNA
gatgacgacgaaaccggaacatcttgtgctgcctgcaatatcaggcacaatacaaaaagggggagtgtttagtgggtatgcccttagggacaggtgagtcccacactaccagctaccgccgtatagatggtgtgcgtaaccgcatttcaacccccttttc

SEQ ID NO: 225 - DNA, 155 bp, synthetic construct
  source 1..155: mol_type = other DNA
caccgaggtttactttcctcattgaaagtcaactacactggccaacatctattgtgtacaaagtaaagggaaaacagtcatatgacgtgccattcatatatggcattgttgtgtgggcaaattcatttcctacaaaaaaaaaaaataaaaaaaaa

SEQ ID NO: 226 - DNA, 225 bp, synthetic construct
  source 1..225: mol_type = other DNA
ccaaagcgtctggttcattatttgcctgctaccttggcataacatcaaaaaggtataaaaatcgcataaagtattgcataaaatggctataaggtttttttagtacgtatgggctgtggatcgttgaattgtgctcattgatccgtatgcagcgaatcgtgcatgcttgatgtgaactttgcctagcatcaagtatcttttgaagattttcttacctttgcgatc

SEQ ID NO: 227 - DNA, 123 bp, synthetic construct
  source 1..123: mol_type = other DNA
tgattacaaaaacaataattcatgaccgcgatcacaggatgggacaaacagggttggacgggccacccggggtttggtacaccccaaataactcgaaacagaaacccgcgtgggtttattaaa

SEQ ID NO: 228 - DNA, 125 bp, synthetic construct
  source 1..125: mol_type = other DNA
atgattacaaaaacaataattcatgaccgtgttcactggatgggacaatctgggttggatgagccacccggggtttggaacaccccaaataactcaaaacagaaacccgcgtgggtttgtaaaaa

SEQ ID NO: 229 - DNA, 249 bp, synthetic construct
  source 1..249: mol_type = other DNA
tcctcaaagttgagggagatccagtgggggttgctgccgtacccatcggtggtactggtcccacatttctctgaaagtccctaatgttcgaccaagcccggtaccaccatcgaacacctaaacggcaaaatgcctcatggcacatgagccaagtaggggggtagggacacgggaacctccggctcgtctgtgatgattaagctccctcacgggaggtcactagacgaaggcatgtgttgtgggctcaaa

SEQ ID NO: 230 - DNA, 195 bp, synthetic construct
  source 1..195: mol_type = other DNA
ggagatttggcccctctctatcaacctgggtaacctgcaccccaccatctaatacggcagcaggcaattaagccgtcccactcaacatctggtggaagagtgaggagttttagtgagtatgcccgttaaatagggagattctcacactgttcggactttttgatccgttcgtctttaaaagatttctcctcactg

SEQ ID NO: 231 - DNA, 221 bp, synthetic construct
  source 1..221: mol_type = other DNA
gggtctgggatcctgtggttctcctgggttactgccgtaccctagtggttccaaattcgaacgcaatacgactgacattggtcccataacatcttggagccacgcctacatcaaacggcaaaatgtcagcccgacgatggttcttagggacagtcgtcctcgttgtctgtaatgattagactagtcgttgctaacgacgaggacatgatgggcggctaaaa

SEQ ID NO: 232 - DNA, 193 bp, synthetic construct
  source 1..193: mol_type = other DNA
tgcaaaagcaaaaatcccccttccaaaaattcccctatccttccccactcccatccttaccccctcccaatcctatccctctccctaaatctcgttaggaccattcaatgagtattacatatatctcaagtgcctaccagcgggcacggtcatcaaaatacactggaaaaattgcacaaactgcaatctgtcc

SEQ ID NO: 233 - DNA, 215 bp, synthetic construct
  source 1..215: mol_type = other DNA
gtgtccgggtcccgtggttctcctgggttcaggtctgaccctgtggttccacttcgaacgcaatacagctgcattcggtcccgcacaacatctgggaaccacgccaacaccaacaggccaaatgtagctcgcgacgataccgattgggacagtcgcctcgttatccgtaatgattttagccctttgttactaatagcgagtgcgcgctgcataaa

SEQ ID NO: 234 - DNA, 119 bp, synthetic construct
  source 1..119: mol_type = other DNA
aattgcgtgggttcagtgggaagctcgtgatgatttgaatagtcttgtgcttcacccacgatgacacgtgacggccacggttcaagccggaaagcgggttgcggaaaagcctgccacag

SEQ ID NO: 235 - DNA, 234 bp, synthetic construct
  source 1..234: mol_type = other DNA
gggcccggaatcctgtggattctcctgggtcgctgccgaacccttgtggcttcgcgtcaagtttgaatactgacatcttatccccgagtgccaaccggagccgccgcaccagcaatcggcaaaatgtcagccttgacgatagttagatggggaatgtcgtcctcgttcttccataatgattagaaaaactgattgttgtgggagagacgaaagcatcagtaagggcggcgaaaa

SEQ ID NO: 236 - DNA, 223 bp, synthetic construct
  source 1..223: mol_type = other DNA
ggggtcctgtggattgggtccctgcgaaacccagtgatcccggaccccacgcggtagcttcaatcgtggtgcctcagttgcaaaatctcgcggatcacagcccaacaatttgcaaaatgccgtacccaatcgctgtgggagttagagggcaataggtcactcccgggtatgcccgttatggggcgtcaatcactaattcggagacgaagtagtgcggccgaaa

SEQ ID NO: 237 - DNA, 190 bp, synthetic construct
  source 1..190: mol_type = other DNA
gactaccgagcgccaccggaggggacccagaggaccgtctcggtgattaggcggtgaccgccctggcctctacctgaaatagcgatcaaatcgccttagagggcctgatttggggggttgggcagacactctgtgtcattactgccctttacctttccacttcttgcctgtctaataataaatgttaaca

SEQ ID NO: 238 - DNA, 324 bp, synthetic construct
  source 1..324: mol_type = other DNA
tgaatttagattgtactgtttgtagatttttgtaggactgtttttatatattgttttcttgtatgtaacttttgtattttttattacttgtactttattttataaataaactagacggtagctaggttcgtagggcagccacaagccagttaggtagctcatagtaggtagggacagagactttaattctaaacgcgtcaagtgccacctgatttggactgaattaccggacctgaaccaatttaccggatatgtccaaggtggacgggccacctttacttaacccggaaaaggagcatatattatttatatgtgttcgaaata

SEQ ID NO: 239 - DNA, 224 bp, synthetic construct
  source 1..224: mol_type = other DNA
gggtccgagatcctgtgggttctcccgggtacatgccggacccagtggtaccaccttcgaacgcactacggctgcatttcgtcccgcacaccatcagggtaccacgccgacaccaaccggcaaaatgccagcccgcgacgatactgatgggacagtcgcacgttggtccatgatgattatgacactgtcattaatgatcggggcaatgtaccgtgcggcgtaaa

SEQ ID NO: 240 - DNA, 266 bp, synthetic construct
  source 1..266: mol_type = other DNA
gacaccctggccgtaagactcctttgaaaccatatgaaggtgcacatggacctattccggtctatgtaacaaacaccagatggtggattaggagcagccttaacttatcagtggaatctcgtaatgattactagcctcatgacgccgtcacataccgatgaacgacatctaatcggtaatgccgacttcccccggctatgaccaaatgggtggacgggccaccctcacttaacccggaaagtaacatacctcggtatgttcgatac

SEQ ID NO: 241 - DNA, 253 bp, synthetic construct
  source 1..253: mol_type = other DNA
gcagccaaggtcctgtggttttgggtcgctgcaggacccatgcggtaccggactctgcatcctagtgtgcatcttggatgttacacaggtccccaatacgaacaactgtgtgccgctccgaacaccaacctgcacaatgccggcctattgagcctctggagacgtgggtggggacataaggtcgctcacgtggccgactatctcttagtcgggcgctatcgcgaagttgggtagacgtaggcaagcggaataa

SEQ ID NO: 242 - DNA, 126 bp, synthetic construct
  source 1..126: mol_type = other DNA
cagaagaggaggccgttgtcgattcatggtaccgcctgccatacaggcgtaatattttttagggaccagccttcaccctggcttaattttattatttttgtttatgggtgatgagctggcagttag

SEQ ID NO: 243 - DNA, 223 bp, synthetic construct
  source 1..223: mol_type = other DNA
gtaatagaggtcctgtggaaacgggtgctgcactcccgtcgttccttggacctctcgcggtaactttttgcgaggccctcgttgccaagaacttaaggaacaagcccaccaagtgcacaatgccatacccaaacgctgaggggagctagagggcgatttggtactcccgggtatgcccgttgaaggggtgttgctcaattcgaggacgaagcgggcggcgtaa

SEQ ID NO: 244 - DNA, 371 bp, synthetic construct
  source 1..371: mol_type = other DNA
ttattgttattgtattctggttgtattgttttttgctgtattactgttgattgttcttattgtatctattgctctgtctcctattgttcttgttttattgttatttgttgccttgctgtttgaatattgatttttcaataaaactacccctggtactcctgccctgttaccctggccctggaaaggttgggagagtaagctggaactcgtattcatgacgtgtcacatcaagccaaattttaaagacaaaatatgaattggaattgacaaattttaccaatttggaccacttactggactagacacaggtggacgggccacctttacttaactcggaaaagaaacacatatacttttatgtgtttgttaaa

SEQ ID NO: 245 - DNA, 148 bp, synthetic construct
  source 1..148: mol_type = other DNA
acatataagttcttatctttgtttcttttatttttattttatatatattattattgaatatttttagcctaccagcgggctcagctaaatagcgtgtcatctagaatacactggaaaaattgtttacaggcagaaataacaacggttc

SEQ ID NO: 246 - DNA, 255 bp, synthetic construct
  source 1..255: mol_type = other DNA
ggagtcggggtcctgcgactcaaggatcgacgccgggtcctgctggtgccgggcggcagtgcagctacactgccgcaaggacatgcccctgatactatctaggcaccgtttccaccaccggcgcaataaacatggcaaattgctagggcgtgagatgggggccattgagggtctttcgctccgttaatctgtaatgattacagtcgtcggagcgtgtcagcgcaaagatgaagcatgcacgttgtttttgaaaaa

SEQ ID NO: 247 - DNA, 231 bp, synthetic construct
  source 1..231: mol_type = other DNA
gcagaggtggagatgcctttatctggcttgtgaggtctgcctactgggcactctgcatgagtgagcgagtgatcgaggcgcaattaggtttcaccttaaaggtgttgttgtgtctttccagggcggtcacgccaaacagcaaaacgtagacgcgccctgcaataacgtacagtgggtagattgggtttcatttagcctatatttgtttccctctgccgctggaacgtacaa

SEQ ID NO: 248 - DNA, 154 bp, synthetic construct
  source 1..154: mol_type = other DNA
agctataaaaagcttaaagtcttaaattacaggacaagaaagggtacaaacgtagctgtaccaatgggaaagaataaaaatccgttaattccattcatgtcatacaagagagaagcctcttgaaagtctgaaaccaaaagagtttcacagggaa

SEQ ID NO: 249 - DNA, 153 bp, synthetic construct
  source 1..153: mol_type = other DNA
aataattgatatcgatcttctacatgcgtgctgccggcgcaaccatcatacaggcacaataacttgagtgttttttagtgagtaagagtctcacacttctatgactagtagttacagtcatagagtccatgatggatttttctctcaagtcat

SEQ ID NO: 250 - DNA, 155 bp, synthetic construct
  source 1..155: mol_type = other DNA
agacgtggtcgtttccctttgcacgtgctgcctgcgtactcaggcataataaaaggtggcggttttagtgggtattcacgaatattgtgagagccccacactaccggcgaacaagacccgagccggtgtgcgcaaactgcatttccccacctttc

SEQ ID NO: 251 - DNA, 274 bp, synthetic construct
  source 1..274: mol_type = other DNA
gcccaccaatcagcgacataccttatatatacttacctggcgtagaaatggatttgacaataccgaatgtgactcacccggacatgcttgttatacctggatgtgaccttcctacccaattggacctaaaggcaccttcaccgctgctcctggacctgaaacaaaatatatatttatttatgaaatatgtgtatatatatatatcagtactgtaagccccccggccgggcaacacctaaggccgaaatgtattttatgaaataaaatatttgaa

SEQ ID NO: 252 - DNA, 139 bp, synthetic construct
  source 1..139: mol_type = other DNA
atgtgaaaggtggatccagctttttggtgctgcctgccatattctcaggcataagacgatggagttttagtgggtatgccctatgaacgggagagtcccacagtaccgcctgcggtctgcgtaacgcatttctccatct

SEQ ID NO: 253 - DNA, 205 bp, synthetic construct
  source 1..205: mol_type = other DNA
cgagaagaaagaaaaaaaaatggtacagctgccaaaaacctcagctgaaaaatgtaaagcaaaaataaacattatgttagtgttgtttttaatatataaaagctacaatttttataataggataaattaagctatcaatattgtaagaatagtaagaacttaattaaataaaaaacactccaagcgttttttgctatcggagaca

SEQ ID NO: 254 - DNA, 260 bp, synthetic construct
  source 1..260: mol_type = other DNA
gcacccggggtcctgtggtcttttgggtttctacaggacccacgcggtaccggacccgcaatcttaccgtataggtagaacgctacgcaggtctcctccccagcatcaactgcgtaccgcttccaacaccaacctgtacaatgcagccacggccgcggtcactagacgggagtaggagacagcatggtcactcccggggccgtcgactctcaacagtcgggtgctcagcacaaagttgggggacgaaggcatgcggaaaa

SEQ ID NO: 255 - DNA, 499 bp, synthetic construct
  source 1..499: mol_type = other DNA
ctacgttgacaaagtccgagagacgggcactctatcgatcaatacctgtgctctctctagtggacaacgctgcatgtttaaattattctgggagacgtcccatttatatgtctatttatttatttatttaacttatatatctgttcgtgttgtaacctttgacgaacattgacgcagcgtataagataggaagcccactgctaatacatgaataagcagggtgcgagtcgtaaggcactgaacaactatcagttgccacggcggcggagccaggcttaggtctgggcggacacctccacgcggtctttcccggtaacattcagaggaatggccaagatccgcaaagtcgtactttgatacgaactccgttcgggctaatttttccaagacccaaagctcgaataaaaatgtggtgatatcttttgaacgcaaggaaccctacgatgaggatcatcatcggaagtatacggtgtgggtataagtcccccgtacaggaaca

SEQ ID NO: 256 - DNA, 145 bp, synthetic construct
  source 1..145: mol_type = other DNA
cgacaaacagcaacaccgggaagtcagggaaggaggaatggaggggtcagcttcgcaacgtagtaatagttctatcgcttagacataagttcgaacgatataatgttatttgtacttacctgttaggatacctgcaaataattgt

SEQ ID NO: 257 - DNA, 151 bp, synthetic construct
  source 1..151: mol_type = other DNA
aagcatcgacgcctcttcatagtgtgctgcctgcactctcaggcataataggaggtggagttttagtgggtataccctacaagacgggagagtcccacactaccgggtaccgtcgcatacccggtgtgcgtaacgcatttctccaccttcc

SEQ ID NO: 258 - DNA, 160 bp, synthetic construct
  source 1..160: mol_type = other DNA
catcttcaactctccaggggtacccgttccccacaagatccaaacccgcaacgggcaagaaagcggtgacaaggaggttttagtgggtagcatctattgtagtcccacataaccggaagacctagtctgatggtatgcgtaatgcatttcctccttgtat

SEQ ID NO: 259 - DNA, 239 bp, synthetic construct
  source 1..239: mol_type = other DNA
gcagccgaggtcctgtggatatctgggtttctacagggcccattgtgatacgggaccacaccgcactctgttgtacagagggcgcgatacagccgtatatgttaatgaacacctagtatcacacccaccatcaacctgtacaatactgacagccataaacaagtgagactgtatgtggtcaatctcgaacttggctttacatagaacaccttatgtaagcgatggcatgtcggcggaaa

SEQ ID NO: 260 - DNA, 75 bp, synthetic construct
  source 1..75: mol_type = other DNA
ccgagtagttaccaaagcttagatagtacctacgccttagcgtataatgtcatagttacgatatgtattatgatt

SEQ ID NO: 261 - DNA, 137 bp, synthetic construct
  source 1..137: mol_type = other DNA
gcgcatgaggtaaacatatagttcactgggaacccgccccatattatcttctttctatgtttgtgggcactcccgctaggtcgagagcccccctactgtgcttctttgtcctttcgtcattttgcgggcaactcatt

SEQ ID NO: 262 - DNA, 310 bp, synthetic construct
  source 1..310: mol_type = other DNA
cctgtctcaatgtgctcacagtacaaactaatttaaaatttaacttataacgaagatgctaacctggggcatcggagttggccggccttgcaacgtgggagaagcggaagtgattcccgccaccgcggcgagccgagccctggcacgtttattgctgtattaacttataataataatttatttgcttatttatttatttatctaatttaataatgttttacccatttacactcatctaagcgggtgaatgatccgacacggttactggagacctactccgggaagtccgcccgctacgcgggcgcaggtt

SEQ ID NO: 263 - DNA, 152 bp, synthetic construct
  source 1..152: mol_type = other DNA
gccttgtattgttgattatattgaaatgtgctgcctgcattctcaggcataataaagagtgggttttagtgagtatgacggggtgaaacgtcgagtctcacactaccatttggagaaattccaatggtgtgcataaagcatttcccctctcc

SEQ ID NO: 264 - DNA, 214 bp, synthetic construct
  source 1..214: mol_type = other DNA
accgacatttgctttatccgtactcacgataataatagaggtacaatattcgaggaatgtagtctcccaagaggctctttatgtaccgaagtcctgggcctggcgtagccgtataacaacttgcccagcaataccaagtggagtatctgcgccgctgaagtgtgcttctaacaagcattatgcttcgggggtaagatagtatccacttggcaca

SEQ ID NO: 265 - DNA, 243 bp, synthetic construct
  source 1..243: mol_type = other DNA
agacatacctcgctcctcgcaccgggtccaattggccagtggtaggcagggctctgcttgatgagctagctatgcgattaatccatatgacatatgctgaatgtacatttatatatttaattttgttagtgacactgccgattttatcgatatagattattttaagttatttatcaactaaggacacgcacactggggagctaccccgggaagtccgtccgcccaagggcgttgacgcaggaa

SEQ ID NO: 266 - DNA, 93 bp, synthetic construct
  source 1..93: mol_type = other DNA
ccaggtccacttacaggtgctacctacccataaggtataagtatttctcacttgtaatatatatgtaatgtatatataataaagagtagaaat

SEQ ID NO: 267 - DNA, 136 bp, synthetic construct
  source 1..136: mol_type = other DNA
gatcttacacattgacgaaaatattttatcagcatcaacgcaacgcaagacaaacaaattataggccactgggcggccattccatcatgcccaaaatagttaagcacagtccgtctgaggcttacacacattatcc

SEQ ID NO: 268 - DNA, 146 bp, synthetic construct
  source 1..146: mol_type = other DNA
cggacccgacctacgctcatcgttcaaatagtgacgccttctctaaggcgcaatgcagagggtttttagtgggtaaacacagcaatgtggagtcccacactagtcaatatcacctgactgaagtgcgtaatgcattttccctctat

SEQ ID NO: 269 - DNA, 216 bp, synthetic construct
  source 1..216: mol_type = other DNA
ctaaaaatgcgactccaaaattgtacataaaaactatataattgtaaataaaattaaattaaatatcaatttacttattcattgaataacaaaaagtactaaaatgtacagcacgcaggggcgaaatagagtgagtaagctcaggtgtgaaaattttctcacaaaatacaagaataccacacttaagtgggaagtttgcgtgaaaatgcatagaaa

SEQ ID NO: 270 - DNA, 446 bp, synthetic construct
  source 1..446: mol_type = other DNA
tggaatggtgtgtggctggggatctaatcgcgtggtgtgtgcgctaatggtggtgtgtgcgccccaaattctattgatccccccagtcgcacgcgcgtgtccttctttaatcgttccatcttatttactctactcgagcagccatgcgccatgaacacgaccggcgggcctagattatacacatatttgactgtttacatttatatttacatttaatactatacacttatactgactatttatatctacgtttacaattttaatatttgactatttacatttatatttacattttaatagattttattaatataactttccagtaatttacgacaaatgacaataaatgaatctactccagtaaaagcctaccagtgggccttctagtaattagtcgacatgtacactggactaacattgtatctttaacatacaataaagtaaca

SEQ ID NO: 271 - DNA, 104 bp, synthetic construct
  source 1..104: mol_type = other DNA
attatcatgaccgcgatcaccagattgacgtaggttggacgggccacctggggtaccagttactccgaataacccgtaaaagaaacccgcaagggtttgttaaa

SEQ ID NO: 272 - DNA, 198 bp, synthetic construct
  source 1..198: mol_type = other DNA
agttgggtgccgccgtgacagcggagtatgattcatgtatgaatgtcaaggaggaaagccgctagtcggcttcgttcatatattacctccagggtgttgcctgcgaacaccgacagcaaacagccctgcaatatcgatatcggggaggtggacctctgcgcatatatatagcggaggtactgccctggcatcgaaaca

SEQ ID NO: 273 - DNA, 176 bp, synthetic construct
  source 1..176: mol_type = other DNA
ggccccttccctgcatggtgagatctggatagtgggaattggttttgggagggaggggttcatcagttcataacgtgtcttgccctttgtgggctacaaattccaatctggtcaggtggacgggccacctttacttaacccggaaaagaaacatataattttatatgtttattaaa

SEQ ID NO: 274 - DNA, 222 bp, synthetic construct
  source 1..222: mol_type = other DNA
aaacgagccccccccagcaggcgcgtgcttgcacgcaaaagtgcactttgcacaaatttaacttcaacttttaaaatttttctttcaatttaacttctaactctgagcgccccggagtctaatactttttctttctgaacacaaaatttatatctattgtactcgcaaaattctctttcgcattatcaaaatttaaataaggggtagttatatttcgaaaat

SEQ ID NO: 275 - DNA, 170 bp, synthetic construct
  source 1..170: mol_type = other DNA
gctatggaatgccaattcagaagtttgggtgctgcctgccccattattcaggcataacgaaaaggtggagttttagtgggtattcgcgttttttagtgagagtcccacactaccggctgtacaacatctcagccggtgtaagtgcgtaaacagcatttctccacctttgc

SEQ ID NO: 276 - DNA, 173 bp, synthetic construct
  source 1..173: mol_type = other DNA
gtgacggaaatatagttcggaatatttgggtgctgcctgccctaagacctcaggcataacgagaaggtggttttttagtgggtatgcgcgttttttttagtgcgagtcccacactaccggctgaacaacatctcagctggtgtcagtgcgtaaatagcatttttccatcttcg

SEQ ID NO: 277 - DNA, 205 bp, synthetic construct
  source 1..205: mol_type = other DNA
ataaacttgtttaaacttgttggcccctacccagctgtcggttacctagacagtggaattggttttgggtgggagaggttcatccttctatcgcgtcttgtaccatcacccagatttgggtccaaatttctgatctggaccaggtggacgggccaccttcacttaacccggaaaaggaacatataatttttatatgttcattaaa

SEQ ID NO: 278 - DNA, 366 bp, synthetic construct
  source 1..366: mol_type = other DNA
gcgggaacctccgacttgaaccagccctgctcactcgggcggacatcgaggcggtgcggctgtcttccctgcgataccggtggtagaggacggcggccgcctccaagacagcaggacctgcacactggtcacatgtaaaactgaccggctggatgcgaaccctacatgctctgttatattcaagttttgtttgcttttgcgctcaaatattgtttgtattcctagtagtagaggttaaacattgtaacaactgttttaaaccagtcctgttgttttgtaaaggatgtggtttgtagagtgaaaataaataaaaagttggctcgccctcgcacagcattttgtgtgtgagggggggccttttttttt

SEQ ID NO: 279 - DNA, 116 bp, synthetic construct
  source 1..116: mol_type = other DNA
aagaaacacgcattatgaaactgttcaagacggcgataaccggatactgatcgtctacaagcgcgtccaggcagcgcctcaggtggacacccgtccacctgaaaactagataaaac

SEQ ID NO: 280 - DNA, 114 bp, synthetic construct
  source 1..114: mol_type = other DNA
ggcggacgacggcgacataaaccggggcacccgtccccttccatcaaagcgggcaaaatataacccccctcggccctttttggctagggtcaccgacgggggagtatgaaaaaa

SEQ ID NO: 281 - DNA, 230 bp, synthetic construct
  source 1..230: mol_type = other DNA
gcttcgcctcccccaatatatttagggtacctgcacctgtcccgatagacatctagggaatatgaattcggcagcaggaaataaagccgtcgccctctggctgtagccagagggtaagcaagaaatggttttttagtgggtatgccacagggagtggtgattcccacactaccttggcgatgcgttggatcgacaaggtgcctataaaaggtttttagccgttaacttgt

SEQ ID NO: 282 - DNA, 246 bp, synthetic construct
  source 1..246: mol_type = other DNA
acgacccagtgtctattttaaactttatctatttatttaactatttattatttatttatttatttattatatttaattacttcactattcaaaaacttttattattgctatgcaatagtattatatcaacgaataaattacaactggtcgccgggaattgttcacaatttgaggcgaatccagtattttaatgcctacgaggggcttcctcggaagtatgggtcacgtcggtgactcacagcaaaa

SEQ ID NO: 283 - protein, 1144 aa, synthetic construct
MVDTPPQRANSSLGYNDRAMTMDSDLINDTTCPSCRTDLSNRTAASAAQHRRRCNAPPVQPGGYVCEICEKAFKTYAGLRQHQRRAHIEQYNNADLELREKRQVSSRAQYTDDEIKMIAYDEVAIPDRAKLSGKRIIEILAQKSGRPVEGLKKLRQKDVYKKYYADAIDESRTHDLRKRCPLSPRVHLIRCEDIPTSRNINIQHTPPCPNITTNHQRDDGCNHIDSERVCDLLRGIKNKTTDDMSKIIDSVLSNTPSEDVMHMLDQYIKSLIKYQTVPDEEGNETTQNKANRNNNSKYDNKTKKCKNRNSNQADKNCMNNNRNNNKNSNKYRCKNNNGNSKDKSTNNQYIKRKGNRAKTKSSEYARMQKLYQRNPTQLAEHILTGNSLNVRESPTMGEFAEYYKKLFADIDLGWQPRRRSSNSDEGVDLSYPILAIEIHEQLRKLKESAPGADGINRQMLRQMPRIDLLALLNVIWGMKTLPPILRLNRTTLLPKAGDLLDPKQWRPITISSRILRLLNKIIVSRLENEICLFHTQRGFTRTDGTMANSTILQTLIKTNRNNAKSFTILSIDLAKAFDSVNIVSIVDALIEKNIDPHTIDYIINTYKDVTTILECHGSRSEPIEIKRGVKQGDPMSGFLFNLVIDRLLLQLNECEGVEVNGVRVKALAFADDIVLVAPTPKAMRNNIRIIEGFFKKHGLQVNASKCAAFQNLRVPGTKRLVVDTRPQLKINQNPIPTLGVASQLKYLGYNFNYCGTITPSPSKLEGMLSNLKSSPLKPWQKLRILQRYLIPRLHHSLQSMDINKKKLKYVDNCIIKFVKTILHLPVTTPTPYIHAPLRSGGLGIPSLTVHIAAVYLNRLERLIARGDDETRSVLKSQVVQHTMDRLSRILPNIDVPTKQAVKKYWSEQLHSTALGAGLCMMTPGLSKWVYEPPRFWKGRDYIGAIKLRIGLLPTKGAPYMKDTDCRNPSCAGTRESLYHVLQRCPITHHIRINRHDNINKIIKTSLGNANIRTEQTPRFSTKSDRYVPDIVAIEKDTAYIIETTVAYETKAMSMSNAYSIKKHKYDTPDLISKVKDTYRVPQVRIMPLVVGARGCWLNTNDGIVSKFQLSRKLKYIICTTALQWGVSIHRYFMNTVWRHPIRNK

SEQ ID NO: 284 - protein, 1300 aa, synthetic construct
MSTVNLPLVDQSGWEASRINNLPAAAVVTTRSVKFNNCSYRDPRRSTSTQRDEGTAKPEAYLSPSEDPGMATRINEPAGQKEADTINVMESAERTANLPAEGSIITKTFYNKAFQKTKKGTVSSTPMNSNQQGEEDNINVLAYNTETIAAPPSDDRTTNKPETIVVPPSHDRTSIKEVVIHYPVDGLLCKTCNTLFPNVSLFAKHLRRSHQTKIIRTKCSRCQKTGEFHAIACHYPKCKGAKQTVMGEFQCEACEANFTTKSGLGQHERHMHPELRNTKRIEAAKPTTKKRKGAWTEEEEILLEHLEISLAGAKFINVEIAKVLTTKTAKQISDKRRLLQKKRQTQIATPATPKTFEVNEQEPIQKEENGSETSVDQQIPYKIPAVLPKHREVDEQLKQAEELLKKAKDPICMAQARGLVDCITEQLNGGHNEKHPRQRPKDNKKKAENKNNNNGAKRRYATKKAAYKEAQDLYKSNRRQLARKIMGAPDTLQCPISREELEARFREKLSKPNNKADIKNFTGYAGRADEELLLGPIGEEEVDEALSGIDKHSAPGPDGIKLKHLEAIQETDASRLPRLFTLWLNTRSIPESMKKSRTVLIPKCEDQDRLKNLDNWRPITIGPMLLRLFTKIIAKRLSKAVNINARQKGFLASTPGCNENIAILQNIIKGSKKNKKELAVVFVDLAKAFDSVGHKLLIGSLRRMKAPAAFVDLIRDLYTNNTTVIEGKGKPTEQIKVERGVKQGDPLSPLLFNIAIDPLISTLEQTGQGITMPYGNNTLNCTALAFADDIALLSNSHAGMQENLKILQSYCEKTGLDINVNKTKGFHFTCKRKTFMYNHAAKWRIRNETIKYIPPGDTERYLGARIDPWAGVKESEWEEKLKTWVEGLRTAPLKPTQRLELLRVHVIPRLYFHLILTEASQNILMKLDQIIRNAVKEFLHLPQHVADGLLYSKNKDGGLAIPKLEIQIPSAIIRKRESLERSTDPIIRASFQFREEDNKETIAEFRSLKALREIKDFLVEKKIPVGRNNEVLAMIIEGGQLSEKQRQRLLKPPNEYGAWRDWEFEKWCKMVCQGDGIKYFQEDKISNSWLKPHINTRTRNFISSLLLRSNLYPTRTTLSRGNPQANKRCRRCNSQLETLAHISGHCPYVKNKRIKRHNVVLEHLRKHVGKYGWTSYLEPRLVTSDGNLWKPDIIFKKDDSSKVAVVDVTVRFENNDNSLEKAWIEKTQKYKHLSEEVERLTGGTKPQFFGFVIGARGKWFGKNDSLMNFLGIKRFKTFASKVSRETIFMTLELLQIFNDY

SEQ ID NO: 285 - protein, 1094 aa, synthetic construct
MSPGYSVDVHLDLDYSSDYLVCPSCHINLSSRILAQRKQHIRRCNQPPPNVDGTYACDVCGTSYKTYGGLRQHIKRSHPVEYNVQDTELTEGRAPNSRAKWSTAEEKQLARLESQLPTGLLQTEVNARLASMHTGRSAEAIKKHRQKPAYKTILAEILSELTITTASTPPRSQSSYRNICPLTPRLHLQRIDFIAPRKSIQPPPSPTIITDNRTVSPTSENVVCKLIKHPGICTYLKELLPSCTAQIQTLISQLFNINSFAQLDPAISSAIRSLAQNARTPGSKNRSSRKHNHKKQKHKGKQNRAAMRSTTFSNYQRLFAKNPSQLAHAILDDREITTNTFPPMAEIEEEYRQIFEQCPEYTSSIPHSPTVVAQLDHPITLRETELALRRLKQSASGPDGISREDLRKANTADLVGLLNIVFGLCNTPSILRHNRTVLIPKKGDPTKVSNWRPITISSLFTRLLHKILASRLSDSIKLHHAQRGFSPGDGVMTSFTILDTIVREHRNGGKPLFVLSIDLTKAFDKIHPSAIENALMSKGVDQHTVKYIMSTYSDVDTIMECHGQRSSPIKMCRGVRQGDTDSPILFNIVMDDLVSSINPAEGVSLGEAKIGCLLFADDIVLLSNTQHGIREHLHKLHTFLERTHMEVNPSKCRALQLARVPGTKRVAVDTKPLFTISGKTVPTLKVLEQLKYLGHNYSQRGMIAPSTSNLEIMLERLRHAALRPWQKLHILNRYLIPRLMHCNQSSDVTVGKLVHIDRLIRRFVKATLHLPATTPTSYLYAKIRDGGLSIPCLRHQIGIIYRRRLQRISLQRDPDFDAAFQTPSINRLMRKLDIICQGIPTARSAVARYWAMNLHQSALGNGLKYNTRATNSWILNPPPFWTGRDYIRAIQLRIGLLPTGGAPYINSDAAQCRYPTCVGRRETLSHILQWCPVTHYQRIQRHDRATQDLANHLKNIGFNVQEAPRVNTRDNRYIPDLLVIDNNNHTGSIIETTVVWEHGSSLLDSAQLKKDKYNIPEILENIQKKYNLLQQPNVLPFVIGARGGWPPSNDTIWRQYHLPDYLQRTIVCNVLRYGSSIHKFFMTHTWRKRKKKRN

SEQ ID NO: 286 - protein, 1043 aa, synthetic construct
MKNQGHPDLPQESGSMVISSPLARTGALGGNVGSGGRPIEWTCPVCARSFTTKTGLGVHKRRAHPVEANIEAAPNMVKRRWGEEELLLLARTEAQLTKEKGKCSNVDLLAELPQLHRTLEAIKGQRRGAKYKLLVQSLLAPPTLVPLARRGSGAEPTPTEARPSDVTGEVATEATTPADEPSAVQPPISSNAAHVIEELIKRAETRKQRGGLGGRAARLLRKRTRNGIDDTASVSKRLLRRKEYARVQELYKKCPSRAAAEVIDGVCRETPHSLEEMEAFWRPILEAASDAPGPRPEALRALERAEQYGGARDYTKLWVPFTSDEVKASKLTGNTAPGPDGIRAEEWRTIPAIVKAEIFNQWMTTGIPEQLRQCKTIFVPKSDDPANPSEYRPITIASVPLRHMHSILARRLLDCCPPDTRQRGFICADGTLENTAVLDAVLGDSRRKLRECHVAVLDFSRAFDTVSHGALVKILENRGLPKAFCNYIATMYGAASTTLAVKGQSSTSVKVGKGVRQGDPLSPILFNMVMDLILASLPSGVGYRLENDLVSALAYADDLVLLAGSKVGMQESIDTVVQIGETMGLKVNLGKSSVLSMVPDGHRKKHHYLTGKTFKVGRYKLRQVSCVERWRYLGIDFDASGSANIEHQLKRALDNISRAPLKPQQRLGVARGHLIPRFMHGLVLGTVSDDRLRMLDVQIRGAVRKWLRLPGDIPTAYFHADTKDGGLAIPSLRAVVPELISRRFGLLASSRWSAARAAARSDRIRKKLKWASKMLKKFTRNDVVDETRPAKRYWREALHASTDGYELRESARNPSSTKWLRERSGQFSGRDFVQFVHCHINALPSRVRNSRGRRHGRESELNCRAGCTVRETTAHTIQQCFRTHGGRIERHNCVAKVIAEAMNNKGWITTIEPKIITSQGVRKPDIIASRDGIGVIVDAQVVSGQRPLDDAHRAKRNKYGKHEELVELVAGRLGLPDTGCVSATSCTLSWRGVWSGASARELRLLLGLDNSILDLIPGLVLRGSHMNWTRFNQLTAISSATPG

SEQ ID NO: 287 - protein, 1238 aa, synthetic construct
MATTNTASDTALPGPGSAVADLSALEGTITDDTALPPVHEQNGAQIGYPQSEDAIDCPFCVMEKERHFSFMNLSDLDKHLKTHHLSCEVEWICLKCIKKFTKLHGVRCHYPKCSGLRVDIPKPFKCDMCMMSFDSIIGRSQHERHEHPALRNKKRQMQSKDERQECDEQEEEAYQNNYRRVWTRAETKLLMELDATYEQYTQPNVEIRKFIPNKSLKQISDKRREIRELKNKAINCTTADTNENPKETANKYADKNLDETVNKYADKTTDKTTDKYADKITDKYADKGKTENSGKSNAGNEPADKIVCITLSSSDDEFVDAEEGSLPTNSENAADVLNDPWHLMLRTAIEADGDTPIMASGDTPELISTIYDQLMDIWSTSSNNLPEIKEKLDEFISITLTKFLIDNTNEIDKNGRNMNKNKQDKSKKGRGKNNNNNNAGNKNRNTRKRYLYARLQDMYNNCPKKLADMAVNDDQSLINPVKFELPLAKDIEYLYNRLWQTPGPTNLQGRKHIAPFEVTETFYPITHDEIAERIKLLKNKTAGGPDGLQKRHMQKKGIPKLLSTLYNILLYANYYPNSWKENRTTLIPKIGKDLNNAANWRPITISSILSRSFSAIIDKRLRKFIVQNFRQKGFTSEDGCKYNIALFNEAISTAKRQGGGVFVVLDISKAFDTIPHSMLRISLERRGVPEQIITLITKMYDNCSTVIRARGDEKVPIKILRGVKQGDPLSPLLFNLTTEPLLEELEEKTQGMKVNDTNLPVLAFADDIVLLAKDVEEAQKQLEYVARYLEDLGMSISVNKSACFQIKRLKDTWYIKQMQLKIKNEDIPNVDPGEIFKYLGANMGPWKGLYRGVVVPEIIKIIKRVRKFALKPCQKIDLIENYILPRFIYGLLVSPPSDAVLKLLDSEIRLQIKEILHLVQSTATGFFYTPKASGGLGLPRFEHIIKLSILRNSIKMKKSLDPSVSGLFNEKIEDKLKSIANSLRINWPATIEDIENAKRRLKKENINQWSNLSSQGQGVYDFRRSKIGNAWLIEQNLLKPSRFIDALRLRTNTLGTRVVLARADKNISTTCRRCKAQPETLGHILGLCIHTKSLRIKRHDEVKNFLADKLADKYEVSVEPTIKAGGNLYKPDLIIKGEKGTFIIDVTVRYENRDYLQKAEEEKENKYKPCLDYITRIRHSNTNKVLPIVLGSRGAVPPSTVRNLRYLGIKSRDIKTIALIVIRCSIEIANIFIDYDKV

SEQ ID NO: 288 - protein, 1329 aa, synthetic construct
MTSYRILGQDHRSAVSKAVTAGLEAGSGAVWRSCLTPGLEIGQRVYWRINLPSGRWDQPTGERVIPKRSVGVNTTAPVVGTWQPLGAAIRKTSSVGTQTATADHCRLDLRRTSGSAVVIPQVTIESKTFVNSFIEAERARVVLMSQPVNAEGVVTEGAGTEESRGEPDPGGSRRSGNTLEDQGMRVIPTNEEASAPRGDDQDGGNHEGADDQAENAETTPEAEIEEEQSSPRRGNIDTAVFLEVLLPDPDPLCSLCGVHIGTVGRFGKHFANRHAAVKVVYKCRGCGRSNHNSHSISCHVPKCAGRGERVAQGEFACDLCQARFQTGVGLTQHRRHAHPVERNRERVAEAGASGKLKGTKLWSVAELEILIELSRRHSDNPKVNKIIATALGNGKTAEQVRSKRRNMAAELAHNSSHEAAQDGLTDSILELAAPVVSQPPPNLVHTELRRALEEGGGEEGENEVRAIVDLLREVDQDPGLMDSTASELIRMLGREGGAPGQRRGEGSGRVLKGWERRLSQKRRDYRVHQRLFTSDPARLAALVLDGGQGAECAIPISLIHETFRDRWEGGVAFGGLGGFKSRYTADNGEFAFPILAAEVKENLNRMKNGSAPGPDRISKQALLNWDPRGEQLARLFTTWLVHGVIPDVFKECRTRLLPKSGDAGELGDVSGWRPITIGSLVLRLFSRILTMRLARACPLNPRQRGFLASSGGCADNLMILNGMIRHSRGSGTPLAVVFVDFAKAFDSISHEHILCALEQRQLDQHVIELIRNSYVNCVTRVGSAGDFTPPINMKVGVKQGDPMSPLLFNLALDPLIQALDDLGGGFGWGDHTIAALAFADDLVLVSGSDAGMRRSLKIVESFCQLTGLKVQPRKCHGFYIDKGVVNGCDPWVMGGTPIHQVGPGEAVRYLGVDVDPGQGIVTPDLIPKLQEWIERIKKAPLKPSQRVRVLNSFAVPRLLYQADHCDVPVATLAAMDLTIRGAVKAWLHLAPSTTNGLLYSRNRDGGMGIMRLERMVPAMQARRIFRLSQSEDAWTRRVTERTVAPVEWQRKWRLAGGSIDECPRLGREEGTDARGSSLSLPDWRQDENRAWAALQVQGVGACQFRGDKVSNSWLADPAKVGFRQRHFIAALALRAGTYPTREFLARGRNKAGAACRRCGARLESCSHILGQCPFVKGSRVRRHHKICDLLAIEAERVGWSVTREFRLTTPEGELRIPDLVCTKGNVVLVLDVTIRYEMNSETLAHAYTEKVVHYAPCARQIGGMVGGSEVHVRGFPVGARGKWFAPNDRVLSELGLSVGRRAFFARLISRRALLYSLDVLRDFLCDSVW

SEQ ID NO: 289 - DNA, 379 bp, synthetic construct
  source 1..379: mol_type = other DNA
agagtcagctcccagaagcgcgtgaatgcggaacaatttgaccagaattacctggtccggttcttgctgaagcgtgcttctgccaaaacgggggacgggggtcagggcatgatggatccaacatctacatcatgtcgtctggaaaccctccccaaggccattttcacgtccgatccttaggcaagatcacgtggcgccgggaaaaggaccaaagtagtgcctggtccgagctaacgagaatggccgagccctggcgaaagccaaatgggtctttcaggtttgcagcctgccagactcatgccttcgggcaacttatattgcaccaaaaaatacggaaacaagcctcggaagctgccggtccctaacagtggcggcagca

SEQ ID NO: 290 - DNA, 195 bp, synthetic construct
  source 1..195: mol_type = other DNA
ttccggtgcccgccccggacactatcaccaccgcatagttacagctgtgattggtttttttcgaattttgattttcacagcttagtcgtgcctcctcgctggcctcgccggtaacctgggtcaactttgaccgagggctaaacgactgctaaaattcgagtgtttttgctgtatggtgaaagcgattgttaaaaa

SEQ ID NO: 291 - DNA, 418 bp, synthetic construct
  source 1..418: mol_type = other DNA
atgaggtcatgctcccaggagacgcgtggacttcgcagtgctttgacggtacaagctattgtaaagtccgactgtgttgcgaagcgtgctcctgcccaaatgggggatgctggggtcagggaatagagagctatccaacaccttgcctctattccgtctggaaaccctccccaaggccgacctccacgtccgacccttaggcaaggtcacgtggcgccgggaaaaggaccatacacaatgcctggtccgagctaacggaggcggccgaaccgggcgtaagcccaaacgactttatgtcagaggcacccctgggtcagctgccctgggacgcctcggagcctagctccacttttgaaatgctactaaacgtgtaaagaagcctcggaaccccgactgcctttttgatgacgactcggcg

SEQ ID NO: 292 - DNA, 669 bp, synthetic construct
  source 1..669: mol_type = other DNA
agtaattcgtcgaatgtaactatgactggaaatcgtagtaggattcacgtgacccttaacgggggaagtgtatgaatggctgctcccgggtcgggtgtcgtgcacgtttctgagcccatcggcaagcacggggacgtgaaccacacgcattcgatccggcaaaacggggaagagtagactgggggtcgtggtttcgcgccctttagtcacaaagcccaactcctatccaaggcctgcacgtggcgccgctgctctagctaacatggaggggggggattcgtgcctaaataagcaacccggttggccgttccggcgttgcatgtctcgttggtgcagtatatcctaaagacatttggcgaggccaagagcatcattttccactatgctctgcgcctgcctctatacagagagcgggcgcagagcgcagtcaggcttgtccctggatcccccgaggggtgatgcggcactcaaacatctgtacgagtgtgatccaggtgttggctgcgggttgagatacctgcagcagtcccgatggctggtctctacggattccggcaagcctagcgactctcttcggttgggacctttcgaaaagtcgtgggcgaaccctggtggcggggtttgcggccctaccacaacaggaaattacaaattgcaaaaagtccagat

SEQ ID NO: 293 - DNA, 485 bp, synthetic construct
  source 1..485: mol_type = other DNA
gttgctcctaggggcgggcgtgtgttacatattgactgctagtagtggtccggtaactacacgcactgctcctaccaaatggggaagtggctctggatacaacgcttggcgaacatctcccactgtatcaaaagagcccttctcaaggcctatcccacgtccgacccttaggcaaggtcacgtggcgccgggaaaaggatcattgcaatgcctgatccgagctaacgggataggccgcaatccaccaacagcgcagctgttggaaagaactggagtggcagtatattataacaaaaaatcggttttccgttaacaggggactcctgtgaagtattattcgaaacctgaaaacttatatatcatcgcgtaatatacgcagatttgcactcccttgacgacctaggcatgtatatggacacgattttcggtaatacctaatgaattaactgtttagatttagatttaggacaaaacacggactagaa

SEQ ID NO: 294 - DNA, 204 bp, synthetic construct
  source 1..204: mol_type = other DNA
taagggctctagctcttttggggggctgtgcccattgacagtagctccgtgggaaagagctgctggggtgctgcagaaacatagtggccctcacctcctcgtggtcccgccggtaacacaccctagggtgtggctaagagggtatccttcctatgtggcagacctgatcacctgccacattacctgatcacaaacagacatggt

SEQ ID NO: 295 - DNA, 408 bp, synthetic construct
  source 1..408: mol_type = other DNA
atcacagtacattcttattagctctagttaccctttttaatacatccattttattcctatatatgttaaacccaactgttttaaaatataagtaattaataaatctgttctgtattcggacatgcttttaaagccaattcaacttaaatccacattcaaattttcctactattattattatggttttaatcacctttaccctccattaaataaaattctactagttttcttgctcgcgttcccgtatatatatgtgtatgtatgcgtgcgaatacatatatatatatatatatatatatatatattagtcccttttaatagcgttaatccattctttcggcctaccagcgggccataataatacactggcaatattgtgaagagctccagctcaaataacaaatgttc

SEQ ID NO: 296 - DNA, 273 bp, synthetic construct
  source 1..273: mol_type = other DNA
tgtattatatttattactttgcccttctgctccgcctgcgttgcggtgtggcaggtgtaccgccccagtgaaatttattttgaaacaaaaaaaaaaaaaaaaaaaaaaaaaaaaaaaaaaaaaaaaaaaaaaaaaaaaaaaaaaaaaaaaaaaaaaatttagtaacacgggacgcgtccataacatcgacgctcgcgtcagtaacatcgacgcctgggctgtcgacgagcgacatgaaaactcgaaatgtgcactcgtttgagtgttgttaaa

SEQ ID NO: 297 - DNA, 355 bp, synthetic construct
  source 1..355: mol_type = other DNA
attagcttgaattggtgttatattacattatattatattatattcaattatattatgttctattatattatattaaattattttatgttctattatattatattaaattatattacgttatattactttctaattgttaatcgtttatttatcgaatttcttatgtaagttttctttttatttatctatttaatttaatctacaatgtaaattctatgtatacttgtaatttaagataagtaatcttaatccgcgctacgcctaccagtgggccgactagtgtcccttcgacctagttgacaaaaaaatacactgtacaacttatgtattcttatgattacaataaagagtttta

SEQ ID NO: 298 - DNA, 261 bp, synthetic construct
  source 1..261: mol_type = other DNA
gtcattggggtcctgtgggtaagggtttctacaggacccgagtggtagcaaccctaacaaccgatctgtcatacgatagtttcgtgcgttgagcaaatcccatgtaacaccttgcgtaccacatctgacatcaacctgtacaatgtcgcaccacctgagccctccagatggcacttaacggtagattgggaatgaaaaagggtctaacgttaggaggggcgctaatcacttcattggttggtagaaggcaggcggctgaaa

SEQ ID NO: 299 - DNA, 292 bp, synthetic construct
  source 1..292: mol_type = other DNA
ccaaaatgttttacttcatcactaataaattttaaataaagaaacataactagcagcgtccccgctgcaatacgattttggattttatccaattttaaacgtttacttatttatttattgttgagtttaattttaattttaattctaattttaataacgtaactggttgtagaacatcaacgactagatttaaaattgaccaaggtcaatacattgataaattttaatatttaccttacgagggggttcctcggaagtatgccactttttctttaagaataggcacaggaaa

SEQ ID NO: 300 - DNA, 169 bp, synthetic construct
  source 1..169: mol_type = other DNA
ggttccctcaccctctggttggtggtcagccagtaaaaggggggacagtggggctcgtaatgattacaagcactgggagttacatggatgactcccggatgcgtatcatgcgtagcaccacgatgatatggacggaccgcttaatccggaaaagctacggctctaaatg

SEQ ID NO: 301 - DNA, 23 bp, synthetic construct
  source 1..23: mol_type = other DNA
tgtgccgaggctcaggcacgctc

SEQ ID NO: 302 - DNA, 22 bp, synthetic construct
  source 1..22: mol_type = other DNA
ggcctcccacttattctacacc

SEQ ID NO: 303 - DNA, 23 bp, synthetic construct
  source 1..23: mol_type = other DNA
cagcaagtgggaaggtgtaatcc

SEQ ID NO: 304 - DNA, 25 bp, synthetic construct
  source 1..25: mol_type = other DNA
cccattctatcatcaacgggtacaa

SEQ ID NO: 305 - DNA, 24 bp, synthetic construct
  source 1..24: mol_type = other DNA
gcctagcagccgacttagaactgg

SEQ ID NO: 306 - DNA, 22 bp, synthetic construct
  source 1..22: mol_type = other DNA
tttctctcgcacacagggcatg

SEQ ID NO: 307 - DNA, 22 bp, synthetic construct
  source 1..22: mol_type = other DNA
cgcccaagtgtcggtattaacg

SEQ ID NO: 308 - DNA, 20 bp, synthetic construct
  source 1..20: mol_type = other DNA
tgaccagtcaacccatccac

SEQ ID NO: 309 - DNA, 20 bp, synthetic construct
  source 1..20: mol_type = other DNA
tcacgctgctcaggcttgtc

SEQ ID NO: 310 - DNA, 25 bp, synthetic construct
  source 1..25: mol_type = other DNA
ctcgctctgctcgttgatgatgcac

SEQ ID NO: 311 - DNA, 22 bp, synthetic construct
  source 1..22: mol_type = other DNA
gttgcattgtctctcgccgaag

SEQ ID NO: 312 - DNA, 24 bp, synthetic construct
  source 1..24: mol_type = other DNA
aaggctttgcaggtactcgatcac

SEQ ID NO: 313 - DNA, 25 bp, synthetic construct
  source 1..25: mol_type = other DNA
cgtaccaatccttgtgggctctctg

SEQ ID NO: 314 - DNA, 21 bp, synthetic construct
  source 1..21: mol_type = other DNA
aagaagacattgcccggcagg

SEQ ID NO: 315 - DNA, 25 bp, synthetic construct
  source 1..25: mol_type = other DNA
cttctcttcacgtttctcacggcag

SEQ ID NO: 316 - DNA, 24 bp, synthetic construct
  source 1..24: mol_type = other DNA
acgatgggcacggtgatctcgaag

SEQ ID NO: 317 - DNA, 25 bp, synthetic construct
  source 1..25: mol_type = other DNA
tagaagtgagacttgagcaggcgag

SEQ ID NO: 318 - DNA, 25 bp, synthetic construct
  source 1..25: mol_type = other DNA
ctttcagattccagaggcaggccag

SEQ ID NO: 319 - DNA, 23 bp, synthetic construct
  source 1..23: mol_type = other DNA
ccaggcagctatacccaagtcag

SEQ ID NO: 320 - DNA, 20 bp, synthetic construct
  source 1..20: mol_type = other DNA
tttggtgagaactgccctgg

SEQ ID NO: 321 - DNA, 24 bp, synthetic construct
  source 1..24: mol_type = other DNA
gtgttatctccctgtgttccttcg

SEQ ID NO: 322 - DNA, 24 bp, synthetic construct
  source 1..24: mol_type = other DNA
ccacactccatttcgccatactcg

SEQ ID NO: 323 - DNA, 22 bp, synthetic construct
  source 1..22: mol_type = other DNA
gcggcgtattgctttctctgtg

SEQ ID NO: 324 - DNA, 22 bp, synthetic construct
  source 1..22: mol_type = other DNA
atctgcttggcgctcttgttgg

SEQ ID NO: 325 - DNA, 24 bp, synthetic construct
  source 1..24: mol_type = other DNA
gtcaggtggtggttgatgaagtgg

SEQ ID NO: 326 - DNA, 21 bp, synthetic construct
  source 1..21: mol_type = other DNA
cgtcgctatttgtggtgctgg

SEQ ID NO: 327 - DNA, 21 bp, synthetic construct
  source 1..21: mol_type = other DNA
gccgattgttccttccagagc

SEQ ID NO: 328 - DNA, 23 bp, synthetic construct
  source 1..23: mol_type = other DNA
ttcttcgccgcactccatttagc

SEQ ID NO: 329 - DNA, 22 bp, synthetic construct
  source 1..22: mol_type = other DNA
aaaggcgatatgccaccgagac

SEQ ID NO: 330 - DNA, 23 bp, synthetic construct
  source 1..23: mol_type = other DNA
gaatggaactcgccggtcttgtg

SEQ ID NO: 331 - DNA, 24 bp, synthetic construct
  source 1..24: mol_type = other DNA
tggccctcgggaactgtaattctg

SEQ ID NO: 332 - DNA, 23 bp, synthetic construct
  source 1..23: mol_type = other DNA
cgtttagccctccgtcacatttg

SEQ ID NO: 333 - DNA, 23 bp, synthetic construct
  source 1..23: mol_type = other DNA
acttgatcgcccaccagaggaac

SEQ ID NO: 334 - DNA, 22 bp, synthetic construct
  source 1..22: mol_type = other DNA
ttcgtcgccacactccgtttag

SEQ ID NO: 335 - DNA, 23 bp, synthetic construct
  source 1..23: mol_type = other DNA
ccagacatataggccgcatggac

SEQ ID NO: 336 - DNA, 22 bp, synthetic construct
  source 1..22: mol_type = other DNA
actcggtgccgcttaaaggttc

SEQ ID NO: 337 - DNA, 25 bp, synthetic construct
  source 1..25: mol_type = other DNA
gagcagcttcttcatcgctccaagg

SEQ ID NO: 338 - DNA, 23 bp, synthetic construct
  source 1..23: mol_type = other DNA
agctctctgtttcgctcccgaag

SEQ ID NO: 339 - DNA, 24 bp, synthetic construct
  source 1..24: mol_type = other DNA
aacaggatctttctctggccgtgc

SEQ ID NO: 340 - DNA, 23 bp, synthetic construct
  source 1..23: mol_type = other DNA
ctaggcacagaaggcacgatcac

SEQ ID NO: 341 - DNA, 25 bp, synthetic construct
  source 1..25: mol_type = other DNA
ccaacaatagggagggctgctaaag

SEQ ID NO: 342 - DNA, 23 bp, synthetic construct
  source 1..23: mol_type = other DNA
ttcttctggccgtgggctctttg

SEQ ID NO: 343 - DNA, 24 bp, synthetic construct
  source 1..24: mol_type = other DNA
accaccttggcgctttcaactctc

SEQ ID NO: 344 - DNA, 24 bp, synthetic construct
  source 1..24: mol_type = other DNA
agacgatagggagggtggatagtg

SEQ ID NO: 345 - DNA, 22 bp, synthetic construct
  source 1..22: mol_type = other DNA
tcagggtcagcacagagttcag

SEQ ID NO: 346 - DNA, 25 bp, synthetic construct
  source 1..25: mol_type = other DNA
tctgctggtgatgaagtcctccttg

SEQ ID NO: 347 - DNA, 23 bp, synthetic construct
  source 1..23: mol_type = other DNA
tttggaaggaatcgctgggctgg

SEQ ID NO: 348 - DNA, 25 bp, synthetic construct
  source 1..25: mol_type = other DNA
gcaccttctcctgggctcttgtttc

SEQ ID NO: 349 - DNA, 24 bp, synthetic construct
  source 1..24: mol_type = other DNA
tccatcggttgttccagggctatc

SEQ ID NO: 350 - DNA, 23 bp, synthetic construct
  source 1..23: mol_type = other DNA
aggttcccagagagttgtccagc

SEQ ID NO: 351 - DNA, 24 bp, synthetic construct
  source 1..24: mol_type = other DNA
agcagattgttcgttagcctccac

SEQ ID NO: 352 - DNA, 25 bp, synthetic construct
  source 1..25: mol_type = other DNA
ttggcgttcaggtaatcttcggtgg

SEQ ID NO: 353 - DNA, 24 bp, synthetic construct
  source 1..24: mol_type = other DNA
cccgttccggttaaagttcaggac

SEQ ID NO: 354 - DNA, 25 bp, synthetic construct
  source 1..25: mol_type = other DNA
tctgcctgtctcgtcgctgatgatg

SEQ ID NO: 355 - DNA, 24 bp, synthetic construct
  source 1..24: mol_type = other DNA
ttctatcggtcagtctcacgccac

SEQ ID NO: 356 - DNA, 22 bp, synthetic construct
  source 1..22: mol_type = other DNA
tgggtttccagcgaagaaggtg

SEQ ID NO: 357 - DNA, 22 bp, synthetic construct
  source 1..22: mol_type = other DNA
tttgagcgttagcatcggcagg

SEQ ID NO: 358 - DNA, 24 bp, synthetic construct
  source 1..24: mol_type = other DNA
cgtcagaggtggtaaagacgttgc

SEQ ID NO: 359 - DNA, 23 bp, synthetic construct
  source 1..23: mol_type = other DNA
tttggtcgggtgtttgccttctc

SEQ ID NO: 360 - DNA, 23 bp, synthetic construct
  source 1..23: mol_type = other DNA
tctcacttcggtcagggcatcag

SEQ ID NO: 361 - DNA, 24 bp, synthetic construct
  source 1..24: mol_type = other DNA
gctgcttcccatttccagtctcac

SEQ ID NO: 362 - DNA, 23 bp, synthetic construct
  source 1..23: mol_type = other DNA
taactacagctccgcgacctcag

SEQ ID NO: 363 - DNA, 23 bp, synthetic construct
  source 1..23: mol_type = other DNA
agcttcgctcaccacttccacag

SEQ ID NO: 364 - DNA, 24 bp, synthetic construct
  source 1..24: mol_type = other DNA
cgtcacaggtccattcgctagatc

SEQ ID NO: 365 - DNA, 21 bp, synthetic construct
  source 1..21: mol_type = other DNA
aagctgatagacttgccgtcg

SEQ ID NO: 366 - DNA, 22 bp, synthetic construct
  source 1..22: mol_type = other DNA
tgctttcgcccacgttcaggtc

SEQ ID NO: 367 - DNA, 24 bp, synthetic construct
  source 1..24: mol_type = other DNA
aagcctgactgttctctccactgc

SEQ ID NO: 368 - DNA, 25 bp, synthetic construct
  source 1..25: mol_type = other DNA
gcgccagtgaaatgtgtgctactac

SEQ ID NO: 369 - DNA, 20 bp, synthetic construct
  source 1..20: mol_type = other DNA
ttgaaatggtcggcagcagc

SEQ ID NO: 370 - DNA, 23 bp, synthetic construct
  source 1..23: mol_type = other DNA
gtgtggtcagtctttcagcgttc

SEQ ID NO: 371 - DNA, 25 bp, synthetic construct
  source 1..25: mol_type = other DNA
gccagcactttcaaccaacaccaac

SEQ ID NO: 372 - DNA, 23 bp, synthetic construct
  source 1..23: mol_type = other DNA
ttcgttctcggccaggttggttg

SEQ ID NO: 373 - DNA, 24 bp, synthetic construct
  source 1..24: mol_type = other DNA
tcagcagatcaacccagtcgctag

SEQ ID NO: 374 - DNA, 23 bp, synthetic construct
  source 1..23: mol_type = other DNA
atcgacttggtcaccctacgcag

SEQ ID NO: 375 - DNA, 22 bp, synthetic construct
  source 1..22: mol_type = other DNA
aacactcttccgcaggtcacac

SEQ ID NO: 376 - DNA, 22 bp, synthetic construct
  source 1..22: mol_type = other DNA
acgacagtgacgaactcgatgg

SEQ ID NO: 377 - DNA, 25 bp, synthetic construct
  source 1..25: mol_type = other DNA
accgcctcccttctgactctcaaac

SEQ ID NO: 378 - DNA, 23 bp, synthetic construct
  source 1..23: mol_type = other DNA
gggcattctcaccagaggtgttc

SEQ ID NO: 379 - DNA, 22 bp, synthetic construct
  source 1..22: mol_type = other DNA
tccttgggtgtggtttgtgtgc

SEQ ID NO: 380 - DNA, 23 bp, synthetic construct
  source 1..23: mol_type = other DNA
aatactaccagtcagtggcgagc

SEQ ID NO: 381 - DNA, 21 bp, synthetic construct
  source 1..21: mol_type = other DNA
agctcttccttcgcttcctcc

SEQ ID NO: 382 - DNA, 23 bp, synthetic construct
  source 1..23: mol_type = other DNA
agccacagcctcacctggattac

SEQ ID NO: 383 - DNA, 23 bp, synthetic construct
  source 1..23: mol_type = other DNA
agttgggcgtcgtggcttgttac

SEQ ID NO: 384 - DNA, 21 bp, synthetic construct
  source 1..21: mol_type = other DNA
tggctccaggttgtgttgtgc

SEQ ID NO: 385 - DNA, 25 bp, synthetic construct
  source 1..25: mol_type = other DNA
tggatgcttgcacactccgtagagg

SEQ ID NO: 386 - DNA, 23 bp, synthetic construct
  source 1..23: mol_type = other DNA
attcacttccctccgactagctg

SEQ ID NO: 387 - DNA, 21 bp, synthetic construct
  source 1..21: mol_type = other DNA
ttcacagcttctgctcacagg

SEQ ID NO: 388 - DNA, 24 bp, synthetic construct
  source 1..24: mol_type = other DNA
tcttgtgatcctggatgctgctgc

SEQ ID NO: 389 - DNA, 25 bp, synthetic construct
  source 1..25: mol_type = other DNA
aggccagacgatagggaggaagtag

SEQ ID NO: 390 - DNA, 24 bp, synthetic construct
  source 1..24: mol_type = other DNA
acacactcgctttcgctcactctc

SEQ ID NO: 391 - DNA, 23 bp, synthetic construct
  source 1..23: mol_type = other DNA
tgtcagcggaaaccacgagaagg

SEQ ID NO: 392 - DNA, 24 bp, synthetic construct
  source 1..24: mol_type = other DNA
ccagatgacctgaaaggaagggac

SEQ ID NO: 393 - DNA, 24 bp, synthetic construct
  source 1..24: mol_type = other DNA
acagttccactcgcttgctcttcc

SEQ ID NO: 394 - DNA, 24 bp, synthetic construct
  source 1..24: mol_type = other DNA
tccgtagagaacccagtgacaagc

SEQ ID NO: 395 - DNA, 23 bp, synthetic construct
  source 1..23: mol_type = other DNA
agcaggctgtggataggagttgg

SEQ ID NO: 396 - DNA, 23 bp, synthetic construct
  source 1..23: mol_type = other DNA
gccgctgtatgtctcgaactctc

SEQ ID NO: 397 - DNA, 24 bp, synthetic construct
  source 1..24: mol_type = other DNA
gggttacttcgcccttccctattc

SEQ ID NO: 398 - DNA, 23 bp, synthetic construct
  source 1..23: mol_type = other DNA
aggagattgctgctcgtttgtgg

SEQ ID NO: 399 - DNA, 24 bp, synthetic construct
  source 1..24: mol_type = other DNA
tggcttggattctctcggcgtttc

SEQ ID NO: 400 - DNA, 25 bp, synthetic construct
  source 1..25: mol_type = other DNA
ttccctcgctgatgctggtttcttg

SEQ ID NO: 401 - DNA, 25 bp, synthetic construct
  source 1..25: mol_type = other DNA
cacttatggttgtgtcagggcactc

SEQ ID NO: 402 - DNA, 23 bp, synthetic construct
  source 1..23: mol_type = other DNA
tccgctaacgggctgatctgttg

SEQ ID NO: 403 - DNA, 25 bp, synthetic construct
  source 1..25: mol_type = other DNA
tctttctctccacgtccacttcctc

SEQ ID NO: 404 - DNA, 21 bp, synthetic construct
  source 1..21: mol_type = other DNA
acacatttgacggcctgaacg

(19) United States
(12) Patent Application Publication
(10) Pub. No.: US 2025/0236890 A1
(43) Pub. Date: Jul. 24, 2025

(54) NON-LTR RETROTRANSPOSON SYSTEM AND USE THEREOF

(71) Applicant: Beijing AstraGenomics Technology Co., Ltd., Beijing (CN)

(72) Inventors: Chen ZHAO, Beijing (CN); Daqi YU, Beijing (CN); Ting WEI, Beijing (CN); Chengxi SHI, Beijing (CN)

(21) Appl. No.: 18/871,762

(22) PCT Filed: Mar. 12, 2024
(86) PCT No.: PCT/CN2024/081116

§ 371 Date: Dec. 4, 2024

(30) Foreign Application Priority Data
Apr. 11, 2023 (CN) 202310380978.4

(51) Int. Cl. C12N 15/90; C12N 9/22; C12N 15/85

(52) U.S. Cl. CPC C12N 15/907; C12N 9/22; C12N 15/85; C12N 2800/107; C12N 2800/90

(57) ABSTRACT

Provided is a non-LTR retrotransposon system. An isolated functional protein encoded by a retrotransposon, a nucleic acid encoding the functional protein, a nucleic acid set, a nucleic acid construct, a composition, a recombinant vector, a recombinant host cell and a kit are provided. A method for introducing an exogenous nucleic acid fragment into the genome of a host cell, a method for editing the genome of a host cell, and a method for obtaining a host cell containing an exogenous nucleic acid fragment in the genome are also provided.

### CROSS-REFERENCE TO RELATED APPLICATIONS

This application claims priority to Chinese Patent Application No. 202310380978.4, filed with the China National Intellectual Property Administration on Apr. 11, 2023, the entire contents of which are hereby incorporated by reference in their entirety for all purpose.

### TECHNICAL FIELD

The present application relates to the field of molecular biology, and specifically to a non-LTR retrotransposon system and use thereof. The present application further specifically relates to: an isolated functional protein encoded by a retrotransposon, a nucleic acid encoding the functional protein, a nucleic acid set and a nucleic acid construct, and a composition, a recombinant vector, a recombinant host cell and a kit comprising the functional protein. The present application further specifically relates to: a method for introducing an exogenous nucleic acid fragment into the genome of a host cell, a method for editing the genome of a host cell, and a method for obtaining a host cell containing an exogenous nucleic acid fragment in the genome. The present application further specifically relates to use of the functional protein, the nucleic acid, the nucleic acid set, the nucleic acid construct, the composition, the recombinant vector, or the recombinant host cell for introducing an exogenous nucleic acid fragment gene into the genome of a host cell or preparing a drug or a preparation for gene therapy, cell therapy, genome research, or stem cell induction and post-induction differentiation.

### BACKGROUND

A transposon is a DNA sequence that can be inserted or removed within the genome to transfer its own sequence or a complete copy of its own sequence within or between genomes (Mobile DNA III). Transposons are mainly divided into two categories. Among them, type I retrotransposons use RNA as an intermediate. In the process of moving from a donor site to a new insertion site, type I retrotransposons require the host's RNA polymerase to transcribe the transposable element into RNA, and then the reverse transcriptase domain encoded by the retrotransposon reverse-transcribes the RNA copy of the transposable element into DNA which is inserted into a new site. During the retrotransposition process of this type of retrotransposons, the sequence of the donor site would not be cleaved from the original site, so a plurality of copies of the retrotransposable element may be present in the genome.

According to different replication and integration mechanisms, retrotransposons can be divided into two categories: LTR (Long Terminal Repeat) and non-LTR. The latter is a type of mobile genetic element widely distributed in eukaryotic cell genomes. According to the characteristics of the nuclease functional domain contained, non-LTR retrotransposons can be further divided into two categories: apurinic/apyrimidinic nuclease (APE-type) and restriction enzyme-like nuclease (RLE-type). Among them, R2 family retrotransposons are a major representative of the RLE type non-LTR retrotransposons. Some R2 retrotransposons have high target site specificity and would specifically integrate into the gene encoding 28s rRNA.

Gene insertion and integration of large fragments have important application value in fields such as gene therapy, molecular breeding of animals and plants, and engineering of industrial microorganisms. Currently, there is a lack of effective tools and systems for insertion and integration of a large fragment gene in the industry. In recent years, the scientific community has developed some tools and methods capable of inserting and integrating a large fragment gene, but these methods still have some problems. For example, in cellular immunotherapy and gene therapy for hereditary diseases, lentivirus or retrovirus are most commonly used to integrate gene sequences, and based on this, there are several therapeutic products for the treatment of tumors and genetic disorders (Aiuti, A., Roncarolo, M. G. and Naldini, L. (2017) Gene therapy for ADA-SCID, the first marketing approval of an ex vivo gene therapy in Europe: paving the road for the next generation of advanced therapy medicinal products. EMBO Mol. Med. 9, 737 740; Aiuti, A. et al. (2009) Gene therapy for immunodeficiency due to adenosine deaminase deficiency. N. Engl. J. Med. 360, 447 458). However, using viruses to integrate a large fragment gene has some potential application limitations: first, the randomness of virus integration in the genome creates the risk of cancer; second, the size of an exogenous gene the virus can carry is also limited, which is not conducive to the transfer of a therapeutic large fragment gene; third, the immunogenicity of the virus may affect the long-term expression of an exogenous therapeutic gene and re-administration; fourth, the production of viruses needs the help of living cells, which makes the quality control and downstream processing of such products more complicated and more expensive, and has certain disadvantages in terms of industrialization. Therefore, non-viral large fragment integration can avoid various disadvantages caused by viral integration and become a valuable tool in gene therapy.

As a non-viral gene integration tool, retrotransposons can achieve the integration in the host genome and stable expression of a large fragment of an exogenous gene through RNA delivery, which can not only reduce negative effects such as immunogenicity, but can also be combined with LNP and other delivery technologies to directly treat target cells in vivo. Due to the lack of relevant retrotransposon tools, there are few applications of retrotransposon technology in gene therapy or other fields. There are few companies in the world that are mining a large number of active retrotransposons and trying to develop them into therapeutic tools, therefore, it is necessary to quickly discover and obtain more active retrotransposon products, and verify and detect their functions to provide more options for the development of gene therapy strategies.

It should be noted that methods described in this section are not necessarily methods that have been previously conceived or employed. It should not be assumed that any of the methods described in this section is considered to be the prior art just because they are included in this section, unless otherwise indicated expressly. Similarly, the problem mentioned in this section should not be considered to be universally recognized in any prior art, unless otherwise indicated expressly.

### SUMMARY

Based on this, in order to seek more advanced and more effective non-viral gene integration tools, the present application provides a non-LTR retrotransposon system and use thereof. The present application further specifically provides an isolated functional protein encoded by a retrotransposon, wherein, the functional protein has a functional protein sequence selected from the following (i) or a variant sequence of the aforementioned functional protein with functional protein activity in (ii)-(iv): (i) at least one amino acid sequence as shown in any one of SEQ ID NO: 1-94; (ii) at least one sequence having deletion, substitution, insertion, mutation of 1, 2, 3, 4, 5, 6, 7, 8, 9 or 10 amino acids of the amino acid sequence as shown in any one of SEQ ID NO: 1-94; (iii) at least one amino acid sequence having at least 70%, 80%, 90%, 95% or 99% identity with the amino acid sequence as shown in any one of SEQ ID NO: 1-94; and (iv) at least one sequence obtained by further fusing the amino acid sequence as shown in any one of SEQ ID NO: 1-94 with other sequences. The functional protein encoded by the retrotransposon provided in the present application has high retrotransposition activity and can provide more options for the development of gene integration tools.

According to an embodiment of the present application, an isolated functional protein encoded by a retrotransposon can be provided, wherein, the functional protein includes the amino acid sequence as shown in formula (I):

C(X₁)ₐC(X₂)_{b}H(X₃)_{c}H (I).
among them, a, b and c are the number of amino acids; C is cysteine; H is histidine; (X₁) is any amino acid, and a is 1, 2, 3 or 4; (X₂) is any amino acid, and b is 11, 12, 13, 14, 15, 16 or 17; and (X₃) is any amino acid, and c is 4.

According to an embodiment of the present application, an isolated functional protein encoded by a retrotransposon can be provided, wherein, the functional protein includes the amino acid sequence as shown in formula (II):

G(X₄)_{d}QGD(X₅)ₑS(X₆)_{f}F(X₇)_{g}D (II),
among them, d, e, f and g are the number of amino acids; D is aspartic acid; F is phenylalanine; G is glycine; Q is glutamine; S is serine; (X₄) is any amino acid, and d is 2; (X₅) is any amino acid, and e is 2; (X₆) is any amino acid, and f is 3; and (X₇) is any amino acid, and g is 30, 31, 32, 33, 34, 35 or 36.

According to an embodiment of the present application, an isolated functional protein can be provided, wherein, the functional protein includes the amino acid sequence as shown in formula (III):

C(X₈)ₕC(X₉)ᵢE(X₁₀)ⱼH(X₁₁)ₖC(X₁₂)ₗRH(X₁₃)ₘPD(X₁₄)ₙ(X₁₅)(X₁₆)ₒK(X₁₇)ₚY (III),
among them, h, i, j, k, l, m, n, o and p are the number of amino acids; C is cysteine; D is aspartic acid; E is glutamic acid; H is histidine; K is lysine; R is arginine; P is proline; Y is tyrosine; (X₈) is any amino acid, and h is 2, 3 or 4; (X₉) is any amino acid, and i is 3, 4, 5, 6, 7, 8, 9 or 10; (X₁₀) is any amino acid, and j is 3; (X₁₁) is any amino acid, and k is 4; (X₁₂) is any amino acid, and 1 is 9; (X₁₃) is any amino acid, and m is 31, 32, 33, 34, 35 or 36; (X₁₄) is any amino acid, and n is 11, 12, 13, 14, 15, 16, 17, 18 or 19; (X₁₅) is aspartic acid, or glutamic acid; (X₁₆) is any amino acid, and o is 15, 16, 17, 18 or 19; and (X₁₇) is any amino acid, and p is 3.

According to an embodiment of the present application, an isolated functional protein encoded by a retrotransposon is provided, wherein, the functional protein includes at least two of the amino acid sequences as shown in formula (I), formula (II), and formula (III).

According to an embodiment of the present application, an isolated functional protein encoded by a retrotransposon is provided, wherein, the functional protein includes the amino acid sequences as shown in formula (I), formula (II), and formula (III).

According to an embodiment of the present application, a nucleic acid can be provided, wherein, the nucleic acid encodes the functional protein described in the present application.

According to an embodiment of the present application, a nucleic acid set can be provided, the nucleic acid set includes a 5′-untranslated region, wherein, the 5′-untranslated region includes at least one of the nucleotide sequences as shown in SEQ ID NO: 95-188.

According to an embodiment of the present application, a nucleic acid set can be provided, the nucleic acid set includes a 3′-untranslated region, wherein, the 3′-untranslated region includes at least one of the nucleotide sequences as shown in SEQ ID NO: 189-282.

According to an embodiment of the present application, a nucleic acid set can be provided, the nucleic acid set includes a 5′-untranslated region and a 3′-untranslated region, wherein, the 5′-untranslated region includes the nucleotide sequence as shown in any one of SEQ ID NO: 95-188 or a variant thereof, and the 3′-untranslated region includes the nucleotide sequence as shown in any one of SEQ ID NO: 189-282 or a variant thereof, the RNA transcribed from the nucleic acid set can bind to the functional protein encoded by a specific retrotransposon.

According to an embodiment of the present application, a nucleic acid construct can be provided, the nucleic acid construct includes the nucleic acid described in the present application and/or the nucleic acid set described in the present application.

According to an embodiment of the present application, a composition may be provided, wherein, the composition includes: a functional protein or a functional fragment thereof encoded by a R2 family retrotransposon, or a nucleic acid encoding the functional protein or the functional fragment thereof, the functional protein or the functional fragment thereof has the function of catalyzing the insertion of an exogenous nucleic acid fragment into the genome of a cell; and a nucleic acid set, the nucleic acid set can be recognized by a functional protein or a functional fragment thereof encoded by a specific retrotransposon.

According to an embodiment of the present application, a recombinant vector can be provided, wherein, the recombinant vector includes the nucleic acid encoding the functional protein described in the present application, the nucleic acid described in the present application, and the nucleic acid set described in the present application, the nucleic acid construct described in the present application, or the composition described in the present application.

According to an embodiment of the present application, a recombinant host cell can be provided, wherein, the recombinant host cell comprises the functional protein described in the present application, the nucleic acid encoding the functional protein described in the present application, the nucleic acid described in the present application, and the nucleic acid set described in the present application, the nucleic acid construct described in the present application, the composition described in the present application, or the recombinant vector described in the present application.

According to an embodiment of the present application, a method for introducing an exogenous nucleic acid fragment into the genome of a host cell can be provided, wherein, the method comprises: delivering the functional protein described in the present application, the nucleic acid encoding the functional protein described in the present application, the nucleic acid described in the present application, and the nucleic acid set described in the present application, the nucleic acid construct described in the present application, the composition described in the present application, or the recombinant vector described in the present application into the host cell.

According to an embodiment of the present application, a method for editing the genome of a host cell can be provided, wherein, the method comprises: delivering the functional protein described in the present application, the nucleic acid encoding the functional protein described in the present application, the nucleic acid described in the present application, and the nucleic acid set described in the present application, the nucleic acid construct described in the present application, the composition described in the present application, or the recombinant vector described in the present application into the host cell.

According to an embodiment of the present application, a method for obtaining a host cell containing an exogenous nucleic acid fragment in the genome can be provided, wherein, the method comprises: delivering the functional protein described in the present application, the nucleic acid encoding the functional protein described in the present application, the nucleic acid described in the present application, the nucleic acid set described in the present application, the nucleic acid construct described in the present application, the composition described in the present application, or the recombinant vector described in the present application into the host cell.

According to an embodiment of the present application, use of the functional protein described in the present application, the nucleic acid encoding the functional protein described in the present application, the nucleic acid described in the present application, the nucleic acid construct described in the present application, the nucleic acid construct described in the present application, the composition described in the present application, the recombinant vector described in the present application, or the recombinant host cell described in the present application for introducing an exogenous nucleic acid fragment into the genome of a host cell can be provided.

According to an embodiment of the present application, use of the functional protein described in the present application, the nucleic acid encoding the functional protein described in the present application, the nucleic acid described in the present application, the nucleic acid set described in the present application, the nucleic acid construct described in the present application, the composition described in the present application, the recombinant vector described in the present application, or the recombinant host cell described in the present application for gene therapy, cell therapy, genomic research, or stem cell induction and post-induction differentiation can be provided.

According to an embodiment of the present application, a kit can be provided, wherein, the kit includes the functional protein described in the present application, the nucleic acid encoding the functional protein described in the present application, the nucleic acid described in the present application, the nucleic acid set described in the present application, the nucleic acid construct described in the present application, the composition described in the present application, the recombinant vector described in the present application, or the recombinant host cell described in the present application.

It should be understood that the content described in this section is not intended to identify critical or important features of the examples of the present application, and is not used to limit the scope of the present application. Other features of the present application will be easily understood through the following description.

### BRIEF DESCRIPTION OF THE DRAWINGS

The accompanying drawings exemplarily show embodiments and form a part of the specification, and are used to explain exemplary implementations of the embodiments together with a written description of the specification. The embodiments shown are merely for illustrative purposes and do not limit the scope of the claims. Throughout the accompanying drawings, the same reference numerals denote similar but not necessarily same elements.

FIG. 1 shows a schematic diagram of the order of the elements in the vector in example 1.

FIG. 2 shows the detection results of the retrotransposition activity by amplifying the 3′ junction of 28s rRNA gene with retrotransposons. Different lanes in FIG. 2 correspond to different retrotransposons in example 2 (sample codes 1-100 in Table 1).

FIG. 3 shows the detection results of the retrotransposition activity by utilizing a single copy gene (36b4) as reference. Different lanes in FIG. 3 correspond to different retrotransposons in example 3 (sample codes 3, 4, 5, 7, 11, 14, 19, 20, 21, 23, 26, 28, 29, 43, 47, 48, 49, 50, 51, 52 53, 54, 55, 56, 57, 59, 60, 63, 75, 83, 87, and 98 in Table 1).

FIG. 4 shows the results of quantitative analysis of the retrotransposition activity detected in FIG. 3.

FIG. 5 shows the detection results of the integrity of retrotransposons by amplifying the 5′ junction of 28s rRNA gene with different loci within retrotransposons. Different lanes in FIG. 5 correspond to different retrotransposons in example 4 (sample codes 3, 4, 5, 7, 11, 14, 19, 20, 21, 22, 23, 26, 28, 29, 43, 47, 48, 49, 50, 51, 52, 53, 54, 55, 56, 57, 59, 60, 63, 75, 83, 87, and 98 in Table 1).

### DETAILED DESCRIPTION OF EMBODIMENTS

Unless otherwise indicated or contradicts the context, the terms or expressions used herein should be read in conjunction with the entire content of the present disclosure and as understood by those of ordinary skill in the art. All technical and scientific terms used herein have the same meanings as commonly understood by those of ordinary skill in the art, unless otherwise defined.

In the present application, the terms “nucleic acid” and “polynucleotide” are used interchangeably, and refer to polymerization forms of nucleotides of any length, including deoxyribonucleotides, ribonucleotides, combinations thereof, and analogs thereof.

In the present application, the terms “polypeptide” and “peptide” are used interchangeably, and refer to polymers of amino acids of any length. Therefore, polypeptides, oligopeptides, proteins, antibodies and enzymes are all included in the definition of polypeptide.

As described in the present application, the “fragment” of a sequence refers to a portion of a sequence. For example, the fragment of a nucleic acid sequence refers to a portion of the nucleic acid sequence, and the fragment of an amino acid sequence refers to a portion of the amino acid sequence.

As described in the present application, a “variant” of a sequence is a polynucleotide or polypeptide that differs from a reference polynucleotide or polypeptide, respectively, but retains essential properties. A typical variant of a polynucleotide differs in nucleic acid sequence from another reference polynucleotide, and the differences in nucleic acid sequence may or may not alter the amino acid sequence of the polypeptide encoded by the reference polynucleotide. A typical variant of a polypeptide differs in amino acid sequence from another reference polypeptide. Generally, the differences are limited so that the sequences of the reference polypeptide and the variant are generally very similar, and are identical in many regions. A variant polypeptide and a reference polypeptide may differ in amino acid sequence by one or more substitutions, additions, deletions in any combination. The substituted or inserted amino acid residue may or may not be a residue encoded by the genetic code. Variants of polynucleotides or polypeptides may be naturally occurring, such as allelic variations, or they may be unknown naturally occurring variants. Non-naturally occurring polynucleotide and polypeptide variants can be produced by mutagenesis techniques, direct synthesis, and other recombinant methods known to the skilled artisan.

The expression “functional protein encoded by a retrotransposon” as used in the present application refers to a polypeptide that catalyzes the integration of an exogenous nucleic acid fragment into a target site (such as genome or extrachromosomal DNA).

The term “exogenous nucleic acid fragment” used in the present application includes any gene of interest or any gene or fragment thereof that is transposable. In some non-limiting embodiments, the exogenous nucleic acid fragment is of a different origin than the host cell, for example, a nucleic acid sequence isolated from an organism different from the host cell, i.e., the exogenous nucleic acid fragment is exogenous to the host cell.

The terms “domain” and “functional domain” as used in the present application are used interchangeably and refer to the structure of a biomolecule that contributes to a specific function of the biomolecule, and may include a contiguous region of the biomolecule (such as a contiguous sequence) or different non-contiguous regions (e.g., non-contiguous sequences). Examples of protein functional domains include, but are not limited to, DNA binding domains, RNA binding domains, reverse transcriptase functional domains, and nuclease functional domains.

The term “Untranslated region” as used in the present application refer to a nucleic acid sequence located at both ends of a transposable element and flanking a transposable nucleic acid sequence. Among them, the untranslated region located at the 5′ end of the transposable nucleic acid sequence is called the 5′-untranslated region, and the untranslated region located at the 3′ end of the transposable nucleic acid sequence is called the 3′-untranslated region. In some embodiments, the RNA transcribed from the untranslated region can bind to the functional protein encoded by a specific retrotransposon.

The term “nucleic acid construct” as used in the present application is defined as a single-stranded or double-stranded nucleic acid molecule herein, and preferably refers to an artificially constructed nucleic acid molecule. Optionally, the nucleic acid construct further includes one or more operably linked regulatory sequences, which can direct the expression of a coding sequence in a suitable host cell under compatible conditions. The term “expression” is understood to include any step involved in the production of a protein or polypeptide, including, but not limited to, transcription, post-transcriptional modification, translation, post-translational modification and secretion. The term “regulatory sequence” includes all components necessary or advantageous for expression of the polypeptide/protein of the present application. Each regulatory sequence may be naturally present or exogenous to the nucleic acid sequence encoding the protein or polypeptide. These regulatory sequences include, but are not limited to, leader sequences, polyadenylation sequences, propeptide sequences, promoters, signal sequences, and transcription terminators. At a minimum, the regulatory sequences should include promoters and initiation and termination signals for transcription and translation. Regulatory sequences with linkers can be provided for the purpose of introduction into specific restriction sites for linking the regulatory sequences to the coding region of a nucleic acid sequence encoding a protein or polypeptide.

The term “promoter” as used in the present application refers to a polynucleotide sequence that can control the transcription of a coding sequence. Promoter sequences include specific sequences sufficient to enable RNA polymerase to recognize, bind, and initiate transcription. In addition, promoter sequences may include sequences that optionally modulate the recognition, binding and transcription initiation activities of RNA polymerase in the nucleic acid construct provided in the present application. A promoter can affect the transcription of a gene located on the same nucleic acid molecule as the promoter or a gene located on a different nucleic acid molecule from the promoter.

The term “host cell” as used in the present application include, but are not limited to, an animal cell, a plant cell, an algal cell, a fungal cell, a yeast cell, or a bacterial cell. This term includes a progeny of an original cell into which an exogenous nucleic acid fragment has been introduced. Exemplary host cell includes human embryonic kidney cell HEK293T. It is understood that, due to natural, accidental or intentional mutations, the progeny of a single parent cell may not necessarily be identical to the original parent morphologically or in terms of genome or total DNA complement.

The term “vector” as used in the present application refers to a nucleic acid molecule capable of transporting another nucleic acid molecule connected to it. Examples of vectors include, but are not limited to, plasmids, viruses, bacteria, phages, and insertable DNA fragments. The term “plasmid” refers to a circular double-stranded DNA capable of accepting an exogenous nucleic acid fragment and replicating in prokaryotic or eukaryotic cells.

### A Functional Protein Encoded by a Retrotransposon

The present application provides a non-LTR retrotransposon system and use thereof. According to an embodiment of the present application, an isolated functional protein encoded by a retrotransposon can be provided, wherein, the functional protein has a functional protein sequence selected from the following (i) or a variant sequence of the aforementioned functional protein with functional protein activity in (ii)-(iv): (i) at least one amino acid sequence as shown in any one of SEQ ID NO: 1-94; (ii) at least one sequence having deletion, substitution, insertion, mutation of 1, 2, 3, 4, 5, 6, 7, 8, 9 or 10 amino acids of the amino acid sequence as shown in any one of SEQ ID NO: 1-94; (iii) at least one amino acid sequence having at least 70%, 80%, 90%, 95% or 99% identity with the amino acid sequence as shown in any one of SEQ ID NO: 1-94; and (iv) at least one sequence obtained by further fusing the amino acid sequence as shown in any one of SEQ ID NO: 1-94 with other sequences.

According to an embodiment of the present application, an isolated functional protein encoded by a retrotransposon can be provided, wherein, the functional protein includes the amino acid sequence as shown in formula (I):

C(X₁)ₐC(X₂)_{b}H(X₃)_{c}H (I).
among them, a, b and c are the number of amino acids; C is cysteine; H is histidine; (X₁) is any amino acid, and a is 1, 2, 3 or 4: (X₂) is any amino acid, and b is 11, 12, 13, 14, 15, 16 or 17; and (X₃) is any amino acid, and c is 4.

According to an embodiment of the present application, an isolated functional protein encoded by a retrotransposon can be provided, wherein, the functional protein includes the amino acid sequence as shown in formula (II):

G(X₄)_{d}QGD(X₅)ₑS(X₆)_{f}F(X₇)_{g}D (II),
among them, d, e, f and g are the number of amino acids; D is aspartic acid; F is phenylalanine: G is glycine; Q is glutamine; S is serine: (X₄) is any amino acid, and d is 2; (X₅) is any amino acid, and e is 2: (X₆) is any amino acid, and f is 3; and (X₇) is any amino acid, and g is 30, 31, 32, 33, 34, 35 or 36.

According to an embodiment of the present application, an isolated functional protein can be provided, wherein, the functional protein includes the amino acid sequence as shown in formula (III):

C(X₈)ₕC(X₉)ᵢE(X₁₀)ⱼH(X₁₁)ₖC(X₁₂)ₗRH(X₁₃)ₘPD(X₁₄)ₙ(X₁₅)(X₁₆)ₒK(X₁₇)ₚY (III),
among them, h, i, j, k, l, m, n, o and p are the number of amino acids; C is cysteine; D is aspartic acid; E is glutamic acid; H is histidine; K is lysine; R is arginine; P is proline; Y is tyrosine; (Xx) is any amino acid, and h is 2, 3 or 4; (X₉) is any amino acid, and i is 3, 4, 5, 6, 7, 8, 9 or 10; (X₁₀) is any amino acid, and j is 3; (X₁₁) is any amino acid, and k is 4; (X₁₂) is any amino acid, and 1 is 9; (X₁₃) is any amino acid, and m is 31, 32, 33, 34, 35 or 36; (X₁₄) is any amino acid, and n is 11, 12, 13, 14, 15, 16, 17, 18 or 19; (X₁₅) is aspartic acid, or glutamic acid; (X₁₆) is any amino acid, and o is 15, 16, 17, 18 or 19; and (X₁₇) is any amino acid, and p is 3.

According to an embodiment of the present application, an isolated functional protein encoded by a retrotransposon is provided, wherein, the functional protein includes at least two of the amino acid sequences as shown in formula (I), formula (II), and formula (III).

According to an embodiment of the present application, an isolated functional protein encoded by a retrotransposon is provided, wherein, the functional protein includes the amino acid sequences as shown in formula (I), formula (II), and formula (III).

In some embodiments, the retrotransposon is a non-LTR (Non-Long Terminal Repeat) retrotransposon. In some embodiments, the non-LTR retrotransposon is a restriction enzyme-like nuclease (RLE) retrotransposon. In some embodiments, the retrotransposon is a R2 family retrotransposon.

In some embodiments, the functional protein includes a DNA binding domain that can bind to a specific region in the genome of a cell, preferably, the DNA binding domain can bind to a gene encoding 28s rRNA. In some non-limiting examples, the functional protein takes the 28s rRNA gene as a target site. In some non-limiting examples, the functional protein takes the non-28s rRNA gene as a target site.

In some embodiments, the functional protein further includes an RNA binding domain, a reverse transcriptase functional domain, and/or a nuclease functional domain. In some non-limiting examples, the RNA binding domain of the functional protein associates with the RNA transcribed by the retrotransposon to form RNP (Ribonucleoprotein). In some non-limiting embodiments, the functional protein includes a nuclease functional domain and/or a reverse transcriptase functional domain. The nuclease functional domain creates a single-stranded nick at the DNA target site, and then uses the 3′ end of the DNA exposed by this cleavage to prime reverse transcription of retrotransposons, in which transcribed RNA serves as template for reverse transcription. Finally, the insertion at the target site is achieved by “copy-paste”.

In some embodiments, the species sources of the functional protein include Arthropoda or Chordata. In some embodiments, the species sources of the functional protein include Insecta, Actinopteri, Chondrichthyes, Testudines, Lepidosauria or Aves. In some embodiments, the species sources of the functional protein include Accipiter gentilis, Actinemys marmorata, Agelaius tricolor, Agrochola macilenta, Anagrus nilaparvatae, Andrena haemorrhoa, Anoplius nigerrimus, Artemisiospiza belli, Asobara japonica, Athalia rosae, Blastobasis lacticolella, Bombus hortorum, Bombus hypnorum, Bombus pratorum, Bombus vancouverensis, Brenthis ino, Cardiocondyla obscurior, Cerceris rybyensis, Clusia tigrina, Colaptes auratus, Crematogaster levior, Crotalus tigris, Cuora mccordi, Dinocampus coccinellae, Dolichovespula saxonica, Drosophila albomicans, Drosophila saltans, Ennomos quercinarius, Eopsaltria australis, Eristalis pertinax, Erithacus rubecula, Euphyes dion, Heliconius hecale, Hesperophylax magnus, Ichneumon xanthorius, Jera tricuspidata, Junonia litoralis, Lasioglossum baleicum, Lasioglossum lativentre, Lasioglossum morio, Leptopilina heterotoma, Lysandra coridon, Marasmarcha lunaedactyla, Mimumesa dahlbomi, Muschampia plurimacula, Neodiprion fabricii, Neodiprion pinetum, Nephrotoma flavescens, Nomada fabriciana, Nylanderia fulva, Nymphalis io, Papilio machaon, Pararge aegeria, Podocnemis expansa, Poecilia wingei, Prinia subflava, Scaptomyza hsui, Schistocerca americana, Seladonia tumulorum, Sphecodes monilicornis, Triplophysa tibetana, Trypoxylus dichotomus, Urbanus tucuti, Venturia canescens, Vespa crabro, Zaprionus camerounensis, or Zaprionus kolodkinae.

According to an embodiment of the present application, a nucleic acid can be provided, wherein, the nucleic acid encodes the functional protein described in the present application.

### Nucleic Acid Construct

According to an embodiment of the present application, a nucleic acid set can be provided, the nucleic acid set includes a 5′-untranslated region, wherein, the 5′-untranslated region includes at least one of the nucleotide sequences as shown in SEQ ID NO: 95-188.

According to an embodiment of the present application, a nucleic acid set can be provided, the nucleic acid set includes a 3′-untranslated region, wherein, the 3′-untranslated region includes at least one nucleotide sequence as shown in SEQ ID NO: 189-282.

According to an embodiment of the present application, a nucleic acid set can be provided, the nucleic acid set includes a 5′-untranslated region and a 3′-untranslated region, wherein, the 5′-untranslated region includes the nucleotide sequence as shown in any one of SEQ ID NO: 95-188 or a variant thereof, and the 3′-untranslated region includes the nucleotide sequence as shown in any one of SEQ ID NO: 189-282 or a variant thereof, the RNA transcribed from the nucleic acid set can bind to the functional protein encoded by a specific retrotransposon. In some embodiments, the RNA transcribed from the untranslated region can recognize and bind to the RNA binding domain of the functional protein encoded by the specific retrotransposon.

According to an embodiment of the present application, a nucleic acid construct can be provided, the nucleic acid construct includes the nucleic acid encodes the functional protein described in the present application and/or the nucleic acid set described in the present application. In some embodiments, the nucleic acid construct further includes an exogenous nucleic acid fragment. In some embodiments, the exogenous nucleic acid fragment is operably inserted into the nucleic acid construct through a polyclonal insertion site, the exogenous nucleic acid fragment may be one or more, and may be the same or different. In some embodiments, the exogenous nucleic acid fragment includes any gene of interest or any gene that is transposable, preferably, the exogenous nucleic acid fragment includes a gene of a natural functional protein, an artificial chimeric gene, and/or a gene of a non-coding RNA. In some embodiments, the gene of a non-coding RNA includes a variety of RNAs with known functions and RNAs with unknown functions, such as rRNA, tRNA, small interfering RNA (siRNA), small nuclear RNA (snRNA), small nucleolar RNA (snoRNA), microRNA (miRNA), and/or long non-coding RNA (lncRNA). In some embodiments, the artificial chimeric gene includes a gene of a chimeric antigen receptor. In some embodiments, the gene of a natural functional protein includes a fluorescence-based reporter gene, a luciferase gene, and/or an antibiotic resistance gene. In some non-limiting embodiments, the exogenous nucleic acid fragment may further include a primer series for polymerase chain reaction to facilitate screening for retrotransposition activity.

In some embodiments, the nucleic acid construct further optionally includes a homologous sequence having 100% identity with at least 10 nucleotides of a specific region in the cell genome, preferably, further optionally includes a homologous sequence having 100% identity with at least 10 nucleotides of the gene encoding 28s rRNA. In some non-limiting embodiments, a part of the homologous sequence is identity to the target site to facilitate the initiation of the reverse transcription process. In some non-limiting embodiments, the nucleic acid construct includes 5′-untranslated region and 3′-untranslated region described in the present application, and the homologous sequences are upstream of 5′-untranslated region and/or downstream of 3′-untranslated region. In some non-limiting embodiments, the homologous sequence is upstream of 5′-untranslated region. In some non-limiting embodiments, the homologous sequence is downstream of 3′-untranslated region. In some non-limiting embodiments, the homologous sequences are upstream of 5′-untranslated region and downstream of 3′-untranslated region.

In some embodiments, the nucleic acid construct further includes a promoter and a poly (A) sequence. The promoter can be any suitable promoter sequence, that is, a nucleic acid sequence that can be recognized by a host cell expressing the exogenous nucleic acid fragment. The promoter sequence contains a transcriptional regulatory sequence that mediates the expression of the protein or polypeptide. The promoter can be any nucleic acid sequence having transcriptional activity in a selected host cell, including mutant, truncated and heterozygous promoters, and can be derived from genes encoding extracellular or intracellular proteins or polypeptides homologous or heterologous to the host cell. In some embodiments, the promoter includes CMV, EF1a, SV40, PGK, UbC, human beta actin, CAG, TRE, UAS, Ac5, GFAP, Polyhedrin promotor, TBG, ALB, ApoEHCR-hAAT, CaMKIIa, GAL1, TEF1, GDS, ADH1, CaMV35S, Ubi, H1, U6, T7, T7lac, Sp6, araBAD, trp, lac, Ptac, or pL. Poly (A) tailing signal sequences well known in the art, as well as various truncated forms of poly (A) tailing signals, can be used in the present application.

In some embodiments, the nucleic acid construct further includes any transcription termination sequence (i.e., a sequence that is recognized by the host cell to terminate transcription) to control the expression of the exogenous nucleic acid fragment. Any terminator that is functional in the host cell of choice can be used in the present application.

Optionally, the nucleic acid construct may further include a suitable leader sequence (i.e., an untranslated region in the mRNA that is important for translation in the host cell) to control the expression of the exogenous nucleic acid fragment. The leader sequence is operably linked to the 5′-terminus of the nucleic acid sequence encoding the polypeptide. Any leader sequence that is functional in the host cell of choice can be used in the present application.

Optionally, the nucleic acid construct may further include a propeptide coding region to control the expression of the exogenous nucleic acid fragment, the propeptide coding region encodes an amino acid sequence located at the amino terminus of the polypeptide. The resulting polypeptide is called a zymogen or propolypeptide. The propolypeptide is usually inactive and can be converted into a mature active polypeptide by catalytic or autocatalytic cleavage of the propeptide from the propolypeptide.

Optionally, the nucleic acid construct may further include a regulatory sequence that can regulate the expression of the exogenous nucleic acid fragment according to the growth conditions of the host cell. Examples of the regulatory sequence are systems that turn gene expression on or off in response to chemical or physical stimuli, including in the presence of regulatory compounds. Other examples of the regulatory sequence are those that enable gene amplification. In these instances, the exogenous nucleic acid fragment should be operably linked to the regulatory sequence.

### Retrotransposition Composition

According to an embodiment of the present application, a composition may be provided, wherein, the composition includes: a functional protein or a functional fragment thereof encoded by a R2 family retrotransposon, or a nucleic acid encoding the functional protein or the functional fragment thereof, the functional protein or the functional fragment thereof has the function of catalyzing the insertion of an exogenous nucleic acid fragment into the genome of a cell; and a nucleic acid set, the RNA transcribed by the nucleic acid set can be recognized by a functional protein or a functional fragment thereof encoded by a specific retrotransposon.

In some embodiments, the composition is selected from at least one of the following groups (1) to (95), and any one of the following groups (1) to (94) includes: a functional protein-related sequence and a nucleic acid set,
(1) the functional protein-related sequence is an amino acid sequence comprising the sequence as shown in SEQ ID NO: 1 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 95; and the 3′-untranslated region is a nucleotide sequence comprising the sequence as shown in SEQ ID NO: 189;
(2) the functional protein-related sequence is an amino acid sequence comprising the sequence as shown in SEQ ID NO: 2 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 96; and the 3′-untranslated region is a nucleotide sequence comprising the sequence as shown in SEQ ID NO: 190;
(3) the functional protein-related sequence is an amino acid sequence comprising the sequence as shown in SEQ ID NO: 3 or a nucleic acid encoding the amino acid sequence: the nucleic acid set includes a 5′-untranslated region and a 3′-untranslated region, and the 5′-untranslated region is a nucleotide sequence comprising the sequence as shown in SEQ ID NO: 97; and the 3′-untranslated region is a nucleotide sequence comprising the sequence as shown in SEQ ID NO: 191;
(4) the functional protein-related sequence is an amino acid sequence comprising the sequence as shown in SEQ ID NO: 4 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 98; and the 3′-untranslated region is a nucleotide sequence comprising the sequence as shown in SEQ ID NO: 192;
(5) the functional protein-related sequence is an amino acid sequence comprising the sequence as shown in SEQ ID NO: 5 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 99; and the 3′-untranslated region is a nucleotide sequence comprising the sequence as shown in SEQ ID NO: 193;
(6) the functional protein-related sequence is an amino acid sequence comprising the sequence as shown in SEQ ID NO: 6 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 100; and the 3′-untranslated region is a nucleotide sequence comprising the sequence as shown in SEQ ID NO: 194;
(7) the functional protein-related sequence is an amino acid sequence comprising the sequence as shown in SEQ ID NO: 7 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 101; and the 3′-untranslated region is a nucleotide sequence comprising the sequence as shown in SEQ ID NO: 195;
(8) the functional protein-related sequence is an amino acid sequence comprising the sequence as shown in SEQ ID NO: 8 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 102; and the 3′-untranslated region is a nucleotide sequence comprising the sequence as shown in SEQ ID NO: 196;
(9) the functional protein-related sequence is an amino acid sequence comprising the sequence as shown in SEQ ID NO: 9 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 103; and the 3′-untranslated region is a nucleotide sequence comprising the sequence as shown in SEQ ID NO: 197;
(10) the functional protein-related sequence is an amino acid sequence comprising the sequence as shown in SEQ ID NO: 10 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 104; and the 3′-untranslated region is a nucleotide sequence comprising the sequence as shown in SEQ ID NO: 198;
(11) the functional protein-related sequence is an amino acid sequence comprising the sequence as shown in SEQ ID NO: 11 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 105; and the 3′-untranslated region is a nucleotide sequence comprising the sequence as shown in SEQ ID NO: 199;
(12) the functional protein-related sequence is an amino acid sequence comprising the sequence as shown in SEQ ID NO: 12 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 106; and the 3′-untranslated region is a nucleotide sequence comprising the sequence as shown in SEQ ID NO: 200;
(13) the functional protein-related sequence is an amino acid sequence comprising the sequence as shown in SEQ ID NO: 13 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 107; and the 3′-untranslated region is a nucleotide sequence comprising the sequence as shown in SEQ ID NO: 201;
(14) the functional protein-related sequence is an amino acid sequence comprising the sequence as shown in SEQ ID NO: 14 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 108; and the 3′-untranslated region is a nucleotide sequence comprising the sequence as shown in SEQ ID NO: 202;
(15) the functional protein-related sequence is an amino acid sequence comprising the sequence as shown in SEQ ID NO: 15 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 109; and the 3′-untranslated region is a nucleotide sequence comprising the sequence as shown in SEQ ID NO: 203;
(16) the functional protein-related sequence is an amino acid sequence comprising the sequence as shown in SEQ ID NO: 16 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 110; and the 3′-untranslated region is a nucleotide sequence comprising the sequence as shown in SEQ ID NO: 204;
(17) the functional protein-related sequence is an amino acid sequence comprising the sequence as shown in SEQ ID NO: 17 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 111; and the 3′-untranslated region is a nucleotide sequence comprising the sequence as shown in SEQ ID NO: 205;
(18) the functional protein-related sequence is an amino acid sequence comprising the sequence as shown in SEQ ID NO: 18 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 112; and the 3′-untranslated region is a nucleotide sequence comprising the sequence as shown in SEQ ID NO: 206;
(19) the functional protein-related sequence is an amino acid sequence comprising the sequence as shown in SEQ ID NO: 19 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 113; and the 3′-untranslated region is a nucleotide sequence comprising the sequence as shown in SEQ ID NO: 207;
(20) the functional protein-related sequence is an amino acid sequence comprising the sequence as shown in SEQ ID NO: 20 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 114; and the 3′-untranslated region is a nucleotide sequence comprising the sequence as shown in SEQ ID NO: 208;
(21) the functional protein-related sequence is an amino acid sequence comprising the sequence as shown in SEQ ID NO: 21 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 115; and the 3′-untranslated region is a nucleotide sequence comprising the sequence as shown in SEQ ID NO: 209;
(22) the functional protein-related sequence is an amino acid sequence comprising the sequence as shown in SEQ ID NO: 22 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 116; and the 3′-untranslated region is a nucleotide sequence comprising the sequence as shown in SEQ ID NO: 210;
(23) the functional protein-related sequence is an amino acid sequence comprising the sequence as shown in SEQ ID NO: 23 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 117; and the 3′-untranslated region is a nucleotide sequence comprising the sequence as shown in SEQ ID NO: 211;
(24) the functional protein-related sequence is an amino acid sequence comprising the sequence as shown in SEQ ID NO: 24 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 118; and the 3′-untranslated region is a nucleotide sequence comprising the sequence as shown in SEQ ID NO: 212;
(25) the functional protein-related sequence is an amino acid sequence comprising the sequence as shown in SEQ ID NO: 25 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 119; and the 3′-untranslated region is a nucleotide sequence comprising the sequence as shown in SEQ ID NO: 213;
(26) the functional protein-related sequence is an amino acid sequence comprising the sequence as shown in SEQ ID NO: 26 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 120; and the 3′-untranslated region is a nucleotide sequence comprising the sequence as shown in SEQ ID NO: 214;
(27) the functional protein-related sequence is an amino acid sequence comprising the sequence as shown in SEQ ID NO: 27 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 121; and the 3′-untranslated region is a nucleotide sequence comprising the sequence as shown in SEQ ID NO: 215;
(28) the functional protein-related sequence is an amino acid sequence comprising the sequence as shown in SEQ ID NO: 28 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 122; and the 3′-untranslated region is a nucleotide sequence comprising the sequence as shown in SEQ ID NO: 216;
(29) the functional protein-related sequence is an amino acid sequence comprising the sequence as shown in SEQ ID NO: 29 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 123; and the 3′-untranslated region is a nucleotide sequence comprising the sequence as shown in SEQ ID NO: 217;
(30) the functional protein-related sequence is an amino acid sequence comprising the sequence as shown in SEQ ID NO: 30 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 124; and the 3′-untranslated region is a nucleotide sequence comprising the sequence as shown in SEQ ID NO: 218;
(31) the functional protein-related sequence is an amino acid sequence comprising the sequence as shown in SEQ ID NO: 31 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 125; and the 3′-untranslated region is a nucleotide sequence comprising the sequence as shown in SEQ ID NO: 219;
(32) the functional protein-related sequence is an amino acid sequence comprising the sequence as shown in SEQ ID NO: 32 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 126; and the 3′-untranslated region is a nucleotide sequence comprising the sequence as shown in SEQ ID NO: 220;
(33) the functional protein-related sequence is an amino acid sequence comprising the sequence as shown in SEQ ID NO: 33 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 127; and the 3′-untranslated region is a nucleotide sequence comprising the sequence as shown in SEQ ID NO: 221;
(34) the functional protein-related sequence is an amino acid sequence comprising the sequence as shown in SEQ ID NO: 34 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 128; and the 3′-untranslated region is a nucleotide sequence comprising the sequence as shown in SEQ ID NO: 222;
(35) the functional protein-related sequence is an amino acid sequence comprising the sequence as shown in SEQ ID NO: 35 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 129; and the 3′-untranslated region is a nucleotide sequence comprising the sequence as shown in SEQ ID NO: 223;
(36) the functional protein-related sequence is an amino acid sequence comprising the sequence as shown in SEQ ID NO: 36 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 130; and the 3′-untranslated region is a nucleotide sequence comprising the sequence as shown in SEQ ID NO: 224;
(37) the functional protein-related sequence is an amino acid sequence comprising the sequence as shown in SEQ ID NO: 37 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 131; and the 3′-untranslated region is a nucleotide sequence comprising the sequence as shown in SEQ ID NO: 225;
(38) the functional protein-related sequence is an amino acid sequence comprising the sequence as shown in SEQ ID NO: 38 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 132; and the 3′-untranslated region is a nucleotide sequence comprising the sequence as shown in SEQ ID NO: 226;
(39) the functional protein-related sequence is an amino acid sequence comprising the sequence as shown in SEQ ID NO: 39 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 133; and the 3′-untranslated region is a nucleotide sequence comprising the sequence as shown in SEQ ID NO: 227;
(40) the functional protein-related sequence is an amino acid sequence comprising the sequence as shown in SEQ ID NO: 40 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 134; and the 3′-untranslated region is a nucleotide sequence comprising the sequence as shown in SEQ ID NO: 228;
(41) the functional protein-related sequence is an amino acid sequence comprising the sequence as shown in SEQ ID NO: 41 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 135; and the 3′-untranslated region is a nucleotide sequence comprising the sequence as shown in SEQ ID NO: 229;
(42) the functional protein-related sequence is an amino acid sequence comprising the sequence as shown in SEQ ID NO: 42 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 136; and the 3′-untranslated region is a nucleotide sequence comprising the sequence as shown in SEQ ID NO: 230;
(43) the functional protein-related sequence is an amino acid sequence comprising the sequence as shown in SEQ ID NO: 43 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 137; and the 3′-untranslated region is a nucleotide sequence comprising the sequence as shown in SEQ ID NO: 231;
(44) the functional protein-related sequence is an amino acid sequence comprising the sequence as shown in SEQ ID NO: 44 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 138; and the 3′-untranslated region is a nucleotide sequence comprising the sequence as shown in SEQ ID NO: 232;
(45) the functional protein-related sequence is an amino acid sequence comprising the sequence as shown in SEQ ID NO: 45 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 139; and the 3′-untranslated region is a nucleotide sequence comprising the sequence as shown in SEQ ID NO: 233;
(46) the functional protein-related sequence is an amino acid sequence comprising the sequence as shown in SEQ ID NO: 46 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 140; and the 3′-untranslated region is a nucleotide sequence comprising the sequence as shown in SEQ ID NO: 234;
(47) the functional protein-related sequence is an amino acid sequence comprising the sequence as shown in SEQ ID NO: 47 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 141; and the 3′-untranslated region is a nucleotide sequence comprising the sequence as shown in SEQ ID NO: 235;
(48) the functional protein-related sequence is an amino acid sequence comprising the sequence as shown in SEQ ID NO: 48 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 142; and the 3′-untranslated region is a nucleotide sequence comprising the sequence as shown in SEQ ID NO: 236;
(49) the functional protein-related sequence is an amino acid sequence comprising the sequence as shown in SEQ ID NO: 49 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 143; and the 3′-untranslated region is a nucleotide sequence comprising the sequence as shown in SEQ ID NO: 237;
(50) the functional protein-related sequence is an amino acid sequence comprising the sequence as shown in SEQ ID NO: 50 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 144; and the 3′-untranslated region is a nucleotide sequence comprising the sequence as shown in SEQ ID NO: 238;
(51) the functional protein-related sequence is an amino acid sequence comprising the sequence as shown in SEQ ID NO: 51 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 145; and the 3′-untranslated region is a nucleotide sequence comprising the sequence as shown in SEQ ID NO: 239;
(52) the functional protein-related sequence is an amino acid sequence comprising the sequence as shown in SEQ ID NO: 52 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 146; and the 3′-untranslated region is a nucleotide sequence comprising the sequence as shown in SEQ ID NO: 240;
(53) the functional protein-related sequence is an amino acid sequence comprising the sequence as shown in SEQ ID NO: 53 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 147; and the 3′-untranslated region is a nucleotide sequence comprising the sequence as shown in SEQ ID NO: 241;
(54) the functional protein-related sequence is an amino acid sequence comprising the sequence as shown in SEQ ID NO: 54 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 148; and the 3′-untranslated region is a nucleotide sequence comprising the sequence as shown in SEQ ID NO: 242;
(55) the functional protein-related sequence is an amino acid sequence comprising the sequence as shown in SEQ ID NO: 55 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 149; and the 3′-untranslated region is a nucleotide sequence comprising the sequence as shown in SEQ ID NO: 243;
(56) the functional protein-related sequence is an amino acid sequence comprising the sequence as shown in SEQ ID NO: 56 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 150; and the 3′-untranslated region is a nucleotide sequence comprising the sequence as shown in SEQ ID NO: 244;
(57) the functional protein-related sequence is an amino acid sequence comprising the sequence as shown in SEQ ID NO: 57 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 151; and the 3′-untranslated region is a nucleotide sequence comprising the sequence as shown in SEQ ID NO: 245;
(58) the functional protein-related sequence is an amino acid sequence comprising the sequence as shown in SEQ ID NO: 58 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 152; and the 3′-untranslated region is a nucleotide sequence comprising the sequence as shown in SEQ ID NO: 246;
(59) the functional protein-related sequence is an amino acid sequence comprising the sequence as shown in SEQ ID NO: 59 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 153; and the 3′-untranslated region is a nucleotide sequence comprising the sequence as shown in SEQ ID NO: 247;
(60) the functional protein-related sequence is an amino acid sequence comprising the sequence as shown in SEQ ID NO: 60 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 154; and the 3′-untranslated region is a nucleotide sequence comprising the sequence as shown in SEQ ID NO: 248;
(61) the functional protein-related sequence is an amino acid sequence comprising the sequence as shown in SEQ ID NO: 61 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 155; and the 3′-untranslated region is a nucleotide sequence comprising the sequence as shown in SEQ ID NO: 249;
(62) the functional protein-related sequence is an amino acid sequence comprising the sequence as shown in SEQ ID NO: 62 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 156; and the 3′-untranslated region is a nucleotide sequence comprising the sequence as shown in SEQ ID NO: 250;
(63) the functional protein-related sequence is an amino acid sequence comprising the sequence as shown in SEQ ID NO: 63 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 157; and the 3′-untranslated region is a nucleotide sequence comprising the sequence as shown in SEQ ID NO: 251;
(64) the functional protein-related sequence is an amino acid sequence comprising the sequence as shown in SEQ ID NO: 64 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 158; and the 3′-untranslated region is a nucleotide sequence comprising the sequence as shown in SEQ ID NO: 252;
(65) the functional protein-related sequence is an amino acid sequence comprising the sequence as shown in SEQ ID NO: 65 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 159; and the 3′-untranslated region is a nucleotide sequence comprising the sequence as shown in SEQ ID NO: 253;
(66) the functional protein-related sequence is an amino acid sequence comprising the sequence as shown in SEQ ID NO: 66 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 160; and the 3′-untranslated region is a nucleotide sequence comprising the sequence as shown in SEQ ID NO: 254;
(67) the functional protein-related sequence is an amino acid sequence comprising the sequence as shown in SEQ ID NO: 67 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 161; and the 3′-untranslated region is a nucleotide sequence comprising the sequence as shown in SEQ ID NO: 255;
(68) the functional protein-related sequence is an amino acid sequence comprising the sequence as shown in SEQ ID NO: 68 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 162; and the 3′-untranslated region is a nucleotide sequence comprising the sequence as shown in SEQ ID NO: 256;
(69) the functional protein-related sequence is an amino acid sequence comprising the sequence as shown in SEQ ID NO: 69 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 163; and the 3′-untranslated region is a nucleotide sequence comprising the sequence as shown in SEQ ID NO: 257;
(70) the functional protein-related sequence is an amino acid sequence comprising the sequence as shown in SEQ ID NO: 70 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 164; and the 3′-untranslated region is a nucleotide sequence comprising the sequence as shown in SEQ ID NO: 258;
(71) the functional protein-related sequence is an amino acid sequence comprising the sequence as shown in SEQ ID NO: 71 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 165; and the 3′-untranslated region is a nucleotide sequence comprising the sequence as shown in SEQ ID NO: 259;
(72) the functional protein-related sequence is an amino acid sequence comprising the sequence as shown in SEQ ID NO: 72 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 166; and the 3′-untranslated region is a nucleotide sequence comprising the sequence as shown in SEQ ID NO: 260;
(73) the functional protein-related sequence is an amino acid sequence comprising the sequence as shown in SEQ ID NO: 73 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 167; and the 3′-untranslated region is a nucleotide sequence comprising the sequence as shown in SEQ ID NO: 261;
(74) the functional protein-related sequence is an amino acid sequence comprising the sequence as shown in SEQ ID NO: 74 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 168; and the 3′-untranslated region is a nucleotide sequence comprising the sequence as shown in SEQ ID NO: 262;
(75) the functional protein-related sequence is an amino acid sequence comprising the sequence as shown in SEQ ID NO: 75 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 169; and the 3′-untranslated region is a nucleotide sequence comprising the sequence as shown in SEQ ID NO: 263;
(76) the functional protein-related sequence is an amino acid sequence comprising the sequence as shown in SEQ ID NO: 76 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 170; and the 3′-untranslated region is a nucleotide sequence comprising the sequence as shown in SEQ ID NO: 264;
(77) the functional protein-related sequence is an amino acid sequence comprising the sequence as shown in SEQ ID NO: 77 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 171; and the 3′-untranslated region is a nucleotide sequence comprising the sequence as shown in SEQ ID NO: 265;
(78) the functional protein-related sequence is an amino acid sequence comprising the sequence as shown in SEQ ID NO: 78 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 172; and the 3′-untranslated region is a nucleotide sequence comprising the sequence as shown in SEQ ID NO: 266;
(79) the functional protein-related sequence is an amino acid sequence comprising the sequence as shown in SEQ ID NO: 79 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 173; and the 3′-untranslated region is a nucleotide sequence comprising the sequence as shown in SEQ ID NO: 267;
(80) the functional protein-related sequence is an amino acid sequence comprising the sequence as shown in SEQ ID NO: 80 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 174; and the 3′-untranslated region is a nucleotide sequence comprising the sequence as shown in SEQ ID NO: 268;
(81) the functional protein-related sequence is an amino acid sequence comprising the sequence as shown in SEQ ID NO: 81 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 175; and the 3′-untranslated region is a nucleotide sequence comprising the sequence as shown in SEQ ID NO: 269;
(82) the functional protein-related sequence is an amino acid sequence comprising the sequence as shown in SEQ ID NO: 82 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 176; and the 3′-untranslated region is a nucleotide sequence comprising the sequence as shown in SEQ ID NO: 270;
(83) the functional protein-related sequence is an amino acid sequence comprising the sequence as shown in SEQ ID NO: 83 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 177; and the 3′-untranslated region is a nucleotide sequence comprising the sequence as shown in SEQ ID NO: 271;
(84) the functional protein-related sequence is an amino acid sequence comprising the sequence as shown in SEQ ID NO: 84 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 178; and the 3′-untranslated region is a nucleotide sequence comprising the sequence as shown in SEQ ID NO: 272;
(85) the functional protein-related sequence is an amino acid sequence comprising the sequence as shown in SEQ ID NO: 85 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 179; and the 3′-untranslated region is a nucleotide sequence comprising the sequence as shown in SEQ ID NO: 273;
(86) the functional protein-related sequence is an amino acid sequence comprising the sequence as shown in SEQ ID NO: 86 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 180; and the 3′-untranslated region is a nucleotide sequence comprising the sequence as shown in SEQ ID NO: 274;
(87) the functional protein-related sequence is an amino acid sequence comprising the sequence as shown in SEQ ID NO: 87 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 181; and the 3′-untranslated region is a nucleotide sequence comprising the sequence as shown in SEQ ID NO: 275;
(88) the functional protein-related sequence is an amino acid sequence comprising the sequence as shown in SEQ ID NO: 88 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 182; and the 3′-untranslated region is a nucleotide sequence comprising the sequence as shown in SEQ ID NO: 276;
(89) the functional protein-related sequence is an amino acid sequence comprising the sequence as shown in SEQ ID NO: 89 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 183; and the 3′-untranslated region is a nucleotide sequence comprising the sequence as shown in SEQ ID NO: 277;
(90) the functional protein-related sequence is an amino acid sequence comprising the sequence as shown in SEQ ID NO: 90 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 184; and the 3′-untranslated region is a nucleotide sequence comprising the sequence as shown in SEQ ID NO: 278;
(91) the functional protein-related sequence is an amino acid sequence comprising the sequence as shown in SEQ ID NO: 91 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 185; and the 3′-untranslated region is a nucleotide sequence comprising the sequence as shown in SEQ ID NO: 279;
(92) the functional protein-related sequence is an amino acid sequence comprising the sequence as shown in SEQ ID NO: 92 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 186; and the 3′-untranslated region is a nucleotide sequence comprising the sequence as shown in SEQ ID NO: 280;
(93) the functional protein-related sequence is an amino acid sequence comprising the sequence as shown in SEQ ID NO: 93 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 187; and the 3′-untranslated region is a nucleotide sequence comprising the sequence as shown in SEQ ID NO: 281;
(94) the functional protein-related sequence is an amino acid sequence comprising the sequence as shown in SEQ ID NO: 94 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 188; and the 3′-untranslated region is a nucleotide sequence comprising the sequence as shown in SEQ ID NO: 282; or
(95) a variant of any of the aforementioned group (1)-group (94),
wherein, the functional protein-related sequence is the amino acid sequence of the variant of the functional protein in each group or a nucleic acid sequence encoding the variant, the variant has a variant sequence of the aforementioned functional protein with functional protein activity selected from the following (i)-(iii): (i) at least one sequence having deletion, substitution, insertion, mutation of 1, 2, 3, 4, 5, 6, 7, 8, 9 or 10 amino acids of the amino acid sequence of the functional protein in each group; (ii) at least one amino acid sequence having at least 70%, 80%, 90%, 95% or 99% identity with the amino acid sequence as shown in any one of SEQ ID NO: 1-94; and (iii) at least one sequence obtained by further fusing the amino acid sequence as shown in any one of SEQ ID NO: 1-94 with other sequences.

In some embodiments, the nucleic acid set further includes an exogenous nucleic acid fragment. In some embodiments, the exogenous nucleic acid fragment is operably inserted into the nucleic acid construct through a polyclonal insertion site, the exogenous nucleic acid fragment may be one or more, and may be the same or different. In some embodiments, the exogenous nucleic acid fragment includes any gene of interest or any gene that is transposable, preferably, the exogenous nucleic acid fragment includes a gene of a natural functional protein, an artificial chimeric gene, and/or a gene of a non-coding RNA. In some embodiments, the gene of a non-coding RNA includes a variety of RNAs with known functions and RNAs with unknown functions, such as rRNA, tRNA, small interfering RNA (siRNA), small nuclear RNA (snRNA), small nucleolar RNA (snoRNA), microRNA (miRNA), and/or long non-coding RNA (lncRNA). In some embodiments, the artificial chimeric gene includes a gene of a chimeric antigen receptor. In some embodiments, the gene of a natural functional protein includes a fluorescence-based reporter gene, a luciferase gene, and/or a resistance gene. In some non-limiting embodiments, the exogenous nucleic acid fragment may further include a primer series for polymerase chain reaction to facilitate screening for retrotransposition activity.

In some embodiments, the nucleic acid set further optionally includes a homologous sequence having 100% identity with at least 10 nucleotides of a specific region in the cell genome of, preferably, further optionally includes a homologous sequence having 100% identity with at least 10 nucleotides of the gene encoding 28s rRNA. In some non-limiting embodiments, a part of the homologous sequence are identity to the target site to facilitate the initiation of the reverse transcription process. In some non-limiting embodiments, the nucleic acid construct includes 5′-untranslated region and 3′-untranslated region described in the present application, and the homologous sequences are upstream of 5′-untranslated region and/or downstream of 3′-untranslated region. In some non-limiting embodiments, the homologous sequence is upstream of 5′-untranslated region. In some non-limiting embodiments, the homologous sequence is downstream of 3′-untranslated region. In some non-limiting embodiments, the homologous sequences are upstream of 5′-untranslated region and downstream of 3′-untranslated region.

In some embodiments, the nucleic acid and/or nucleic acid set further includes a promoter and a poly (A) sequence. The promoter can be any suitable promoter sequence, that is, a nucleic acid sequence that can be recognized by a host cell expressing the nucleic acid sequence. The promoter sequence contains a transcriptional regulatory sequence that mediates the expression of the protein or polypeptide. The promoter can be any nucleic acid sequence having transcriptional activity in a selected host cell, including mutant, truncated and heterozygous promoters, and can be derived from genes encoding extracellular or intracellular proteins or polypeptides homologous or heterologous to the host cell. In some embodiments, the promoter includes CMV, EF1a, SV40, PGK, UbC, human beta actin, CAG, TRE, UAS, Ac5, GFAP, Polyhedrin promotor, TBG, ALB, ApoEHCR-hAAT, CaMKIIa, GAL1, TEF1, GDS, ADH1, CaMV35S, Ubi, H1, U6, T7, T7lac, Sp6, araBAD, trp, lac, Ptac, or pL. Poly (A) tailing signal sequences well known in the art, as well as various truncated forms of poly (A) tailing signals, can be used in the present application.

In some embodiments, the nucleic acid encoding the amino acid sequence and/or the nucleic acid set further includes any transcription termination sequence, i.e., a sequence that is recognized by the host cell to terminate transcription. The termination sequence is operably linked to the 3′-terminus of the nucleic acid sequence encoding the protein or polypeptide. Any terminator that is functional in the host cell of choice can be used in the present application.

Optionally, the nucleic acid encoding the amino acid sequence and/or the nucleic acid set may further include a suitable leader sequence, i.e., an untranslated region in the mRNA that is important for translation in the host cell. The leader sequence is operably linked to the 5′-terminus of the nucleic acid sequence encoding the polypeptide. Any leader sequence that is functional in the host cell of choice can be used in the present application.

Optionally, the nucleic acid encoding the amino acid sequence and/or the nucleic acid set may further include a propeptide coding region, which encodes an amino acid sequence located at the amino terminus of the polypeptide. The resulting polypeptide is called a zymogen or propolypeptide. The propolypeptide is usually inactive and can be converted into a mature active polypeptide by catalytic or autocatalytic cleavage of the propeptide from the propolypeptide.

Optionally, the nucleic acid encoding the amino acid sequence and/or the nucleic acid set may further include a regulatory sequence that can regulate the expression of the polypeptide according to the growth conditions of the host cell. Examples of the regulatory sequence are systems that turn gene expression on or off in response to chemical or physical stimuli, including in the presence of regulatory compounds. Other examples of the regulatory sequence are those that enable gene amplification. In these instances, the nucleic acid sequence encoding the protein or polypeptide should be operably linked to the regulatory sequence.

### Recombinant Vector, Recombinant Host Cell and Kit

According to an embodiment of the present application, a recombinant vector can be provided, wherein, the recombinant vector includes the nucleic acid encoding the functional protein described in the present application, the nucleic acid described in the present application, the nucleic acid set described in the present application, the nucleic acid construct described in the present application, or the composition described in the present application. The recombinant vector can be any suitable vector. In some embodiments, the recombinant vector includes, but is not limited to, a recombinant cloning vector, a recombinant eukaryotic expression plasmid, or a recombinant viral vector. In some embodiments, the recombinant eukaryotic expression plasmid includes pcDNA3.1, pCMV, pUC18, pUC19, pUC57, pBAD, pET, pENTR, pGenlenti, or pAAV. In some embodiments, the recombinant virus vector includes a recombinant adenovirus vector, a recombinant adeno-associated virus vector, a recombinant retrovirus vector, a recombinant herpes simplex virus vector, or a recombinant vaccinia virus vector. The recombinant vector of the present application can be constructed using methods well known in the art. For example, depending on the restriction sites contained in the backbone vector used, appropriate restriction sites can be added to both ends of the nucleic acid construct of the present application, and then loaded into the backbone vector.

According to an embodiment of the present application, a recombinant host cell can be provided, wherein, the recombinant host cell comprises the functional protein described in the present application, the nucleic acid encoding the functional protein described in the present application, the nucleic acid described in the present application, the nucleic acid set described in the present application, the nucleic acid construct described in the present application, the composition described in the present application, or the recombinant vector described in the present application. The recombinant host cell can be any host cell in which retrotransposons can be used. In some embodiments, the recombinant host cell includes, but is not limited to, an animal cell, a plant cell, an algal cell, a fungal cell, a yeast cell, or a bacterial cell. In some embodiments, the animal cell includes a mammalian cell. In some embodiments, the mammalian cell includes a primary cell (e.g., a mesenchymal stem cell, an endothelial cell, an epithelial cell, a fibroblast, a keratinocyte, a melanocyte, a smooth muscle cell, and an immune cell), an immortalized cell line (e.g., HEK293, NIH-3T3, RAW-264.7, STO, VERO, CT26, hTERT immortalized human endothelial/epithelial/fibroblast/keratinocyte/ductal/cell lines), a cancer cell line (e.g., Hela, HepG2/3, HL-60, HT-1080, HT-29, A549, SW620, HCT-15, HCT116, MDA-MB-231, MCF7, SK-OV-3, PANC-1, AsPc-1, THP-1, Huh7, KG-1, RAJI, HB-CB, Jurkat, K562, CRL5826, CHO, MDCK, and Renca), an embryonic stem cell line (e.g., H1, H9, WIBR2, WIBR3, G-Olig2, ESF158, RW.4. R1, and D3) and differentiated cells thereof, or an induced pluripotent stem cell line and differentiated cells thereof.

According to an embodiment of the present application, a kit can be provided, wherein, the kit includes the functional protein described in the present application, the nucleic acid encoding the functional protein described in the present application, the nucleic acid described in the present application, the nucleic acid set described in the present application, the nucleic acid construct described in the present application, the composition described in the present application, the recombinant vector described in the present application, or the recombinant host cell described in the present application.

### Method and Use

The retrotransposon-based tools and methods for large fragment gene insertion and integration provided in the present application can be applied to many fields such as gene therapy, crop breeding, model animal engineering, and industrial microorganism engineering. Especially in the field of gene therapy and cell therapy, the tools and methods can be applied to gene writing therapy, which is of great significance for the treatment of genetic diseases that require long fragment gene correction.

According to an embodiment of the present application, a method for introducing an exogenous nucleic acid fragment into the genome of a host cell can be provided, wherein, the method comprises: delivering the functional protein described in the present application, the nucleic acid encoding the functional protein described in the present application, the nucleic acid described in the present application, the nucleic acid set described in the present application, the nucleic acid construct described in the present application, the composition described in the present application, or the recombinant vector described in the present application into the host cell.

According to an embodiment of the present application, a method for editing the genome of a host cell can be provided, wherein, the method comprises: delivering the functional protein described in the present application, the nucleic acid encoding the functional protein described in the present application, the nucleic acid described in the present application, the nucleic acid set described in the present application, the nucleic acid construct described in the present application, the composition described in the present application, or the recombinant vector described in the present application into the host cell.

According to an embodiment of the present application, a method for obtaining a host cell containing an exogenous nucleic acid fragment in the genome can be provided, wherein, the method comprises: delivering the functional protein described in the present application, the nucleic acid encoding the functional protein described in the present application, the nucleic acid described in the present application, t the nucleic acid set described in the present application, the nucleic acid construct described in the present application, the composition described in the present application, or the recombinant vector described in the present application into the host cell.

The method of delivery into the host cell can be any suitable method. In some embodiments, the delivery method includes but is not limited to cationic liposome delivery, lipoid nanoparticle delivery, cationic polymer delivery, vesicle-exosome delivery, gold nanoparticle delivery, polypeptide and protein delivery, retrovirus delivery, lentivirus delivery, adenovirus delivery, adeno-associated virus delivery, electroporation delivery, agrobacterium infection delivery, or gene gun delivery. The methods of cell transfection and culture are routine methods in the art, and appropriate transfection and culture methods can be selected according to different cell types.

The host cell can be any host cell in which retrotransposons can be used. In some embodiments, the host cell includes, but is not limited to, an animal cell, a plant cell, an algal cell, a fungal cell, a yeast cell, or a bacterial cell. In some embodiments, the host cell includes a mammalian cell. In some embodiments, the host cell includes a primary cell (e.g., a mesenchymal stem cell, an endothelial cell, an epithelial cell, a fibroblast, a keratinocyte, a melanocyte, a smooth muscle cell, and an immune cell), an immortalized cell line (e.g., HEK293, NIH-3T3, RAW-264.7, STO, VERO, CT26, hTERT immortalized human endothelial/epithelial/fibroblast/keratinocyte/ductal/cell lines), a cancer cell line (e.g., Hela, HepG2/3, HL-60, HT-1080, HT-29, A549, SW620. HCT-15, HCT116, MDA-MB-231, MCF7, SK-OV-3, PANC-1, AsPc-1, THP-1, Huh7, KG-1, RAJI, HB-CB, Jurkat, K562, CRL5826, CHO, MDCK, and Renca), an embryonic stem cell line (e.g., H1, H9, WIBR2, WIBR3, G-Olig2, ESF158, RW.4, R1, and D3) and differentiated cells thereof, or an induced pluripotent stem cell line and differentiated cells thereof.

According to an embodiment of the present application, use of the functional protein described in the present application, the nucleic acid encoding the functional protein described in the present application, the nucleic acid described in the present application, the nucleic acid set described in the present application, the nucleic acid construct described in the present application, the composition described in the present application, the recombinant vector described in the present application, or the recombinant host cell described in the present application for introducing an exogenous nucleic acid fragment into the genome of a host cell can be provided. The host cell can be any host cell in which retrotransposons can be used. In some embodiments, the host cell includes, but is not limited to, an animal cell, a plant cell, an algal cell, a fungal cell, a yeast cell, or a bacterial cell. In some embodiments, the host cell includes a mammalian cell. In some embodiments, the host cell includes a primary cell (e.g., a mesenchymal stem cell, an endothelial cell, an epithelial cell, a fibroblast, a keratinocyte, a melanocyte, a smooth muscle cell, and an immune cell), an immortalized cell line (e.g., HEK293, NIH-3T3, RAW-264.7, STO, VERO, CT26, hTERT immortalized human endothelial/epithelial/fibroblast/keratinocyte/ductal/cell lines), a cancer cell line (e.g., Hela, HepG2/3, HL-60, HT-1080, HT-29, A549, SW620. HCT-15, HCT116, MDA-MB-231, MCF7, SK-OV-3, PANC-1, AsPc-1, THP-1, Huh7, KG-1, RAJI, HB-CB, Jurkat, K562, CRL5826, CHO, MDCK, and Renca), an embryonic stem cell line (e.g., H1, H9, WIBR2, WIBR3, G-Olig2, ESF158, RW.4, R1, and D3) and differentiated cells thereof, or an induced pluripotent stem cell line and differentiated cells thereof.

According to an embodiment of the present application, use of the functional protein described in the present application, the nucleic acid encoding the functional protein described in the present application, the nucleic acid described in the present application, the nucleic acid set described in the present application, the nucleic acid construct described in the present application, the composition described in the present application, the recombinant vector described in the present application, or the recombinant host cell described in the present application for gene therapy, cell therapy, genomic research, or stem cell induction and post-induction differentiation can be provided.

The above various embodiments and preferences for the present application can be combined with each other (as long as they are not inherently contradictory to each other and are suitable for the use of the present application), and the various embodiments formed by such combinations are considered as a part of the present application.

### EXAMPLES

Exemplary embodiments of the present application are described below in conjunction with the accompanying drawings, where various details of the examples of the present application are included to facilitate understanding. It should be understood that they are considered to be exemplary only and not intended to limit the protection scope of the present application. The protection scope of the present application is only defined by the claims. Therefore, those of ordinary skill in the art should be aware that various changes and modifications can be made to the examples described herein, without departing from the scope of the present application. Likewise, for clarity and conciseness, the description of well-known functions and structures is omitted in the following description.

Unless otherwise stated, the reagents and instruments used in the following examples are conventional products that are commercially available. Unless otherwise stated, experiments are performed under conventional conditions or conditions recommended by the manufacturer.

### Example 1: Establishment of a Screening System for Retrotransposon Activity

We designed a high-throughput screening system to detect the insertion activity of different retrotransposons of the present application at specific sites in the 28s rRNA gene of the human genome.

The order of the elements in the vector is shown in FIG. 1, and the specific description is as follows:
first, BGI TECH SOLUTIONS (BEIJING LIUHE) CO., LIMITED was entrusted to synthesize the corresponding DNA sequence and construct the retrotransposon plasmid. The schematic of an example retrotransposon construct is shown in FIG. 1 and has, from the 5′ end to the 3′ end, 5′-untranslated region (5′ UTR), the DNA sequence (ORF) coding the amino acid sequence of the functional protein optimized by human codons, and 3′-untranslated region (3′ UTR). The synthesized sequence mentioned above was cloned into the plasmid vector pCDNA3.1 that already contains a CMV promoter element and a poly (A) element, and the retrotransposon is transcribed in a eukaryotic cell under the control of the CMV promoter and subsequently translated into a functional protein.

Secondly, based on the functional characteristics of the R2 retrotransposon, we added sequences (R105, R100 in FIG. 1) on both sides of the 5′ UTR and 3′ UTR of the R2 retrotransposon, the added sequences were homologous to the sequences on both sides of the insertion site in human 28s rRNA gene to improve the integrity of retrotransposition products (Eickbush DG, Luan DD, Eickbush TH. Integration of Bombyx mori R2 sequences into the 28S ribosomal RNA genes of Drosophila melanogaster. Mol Cell Biol. 2000; 20 (1): 213-23). These two homologous sequences would be transcribed into RNA together during the retrotransposon transcription process.

In order to use a unified method to detect the retrotransposition activity of all retrotransposons, we inserted a consensus sequence (universal seq) between the ORF region encoding a functional protein and the 3′ UTR, which contained a PCR primer F1 (with a sequence as shown in SEQ ID NO: 301, 5′-TGTGCCGAGGCTCAGGCACGCTC-3′) with high specificity and amplification efficiency in this sequence. After the retrotransposition process was completed and R2 was inserted into the 28s rRNA gene, the genomic DNA was extracted and used as a template to amplify the junction of 28s rRNA gene with retrotransposons using F1 and the reverse primer al (with a sequence as shown in SEQ ID NO: 302, 5 ‘-GGCCTCCCACTTATTCTACACC-3’), about 200 bp downstream of the insertion site. PCR products of different lengths would be amplified for different R2 samples due to differences in the length of their 3′ UTR sequences. The products were further analyzed by Sanger sequencing to verify whether directional insertion at the 28s rRNA gene locus was achieved.

TABLE 1
Functional proteins encoded by retrotransposons
and their corresponding untranslated regions

| Sample | Functional protein | 5′-untranslated | 3′-untranslated |
|---|---|---|---|
| Code | sequence | region | region |
| 1 | SEQ ID NO: 1 | SEQ ID NO: 95 | SEQ ID NO: 189 |
| 2 | SEQ ID NO: 2 | SEQ ID NO: 96 | SEQ ID NO: 190 |
| 3 | SEQ ID NO: 3 | SEQ ID NO: 97 | SEQ ID NO: 191 |
| 4 | SEQ ID NO: 4 | SEQ ID NO: 98 | SEQ ID NO: 192 |
| 5 | SEQ ID NO: 5 | SEQ ID NO: 99 | SEQ ID NO: 193 |
| 6 | SEQ ID NO: 283 | SEQ ID NO: 289 | SEQ ID NO: 295 |
| 7 | SEQ ID NO: 6 | SEQ ID NO: 100 | SEQ ID NO: 194 |
| 8 | SEQ ID NO: 7 | SEQ ID NO: 101 | SEQ ID NO: 195 |
| 9 | SEQ ID NO: 8 | SEQ ID NO: 102 | SEQ ID NO: 196 |
| 10 | SEQ ID NO: 9 | SEQ ID NO: 103 | SEQ ID NO: 197 |
| 11 | SEQ ID NO: 10 | SEQ ID NO: 104 | SEQ ID NO: 198 |
| 12 | SEQ ID NO: 11 | SEQ ID NO: 105 | SEQ ID NO: 199 |
| 13 | SEQ ID NO: 284 | SEQ ID NO: 290 | SEQ ID NO: 296 |
| 14 | SEQ ID NO: 12 | SEQ ID NO: 106 | SEQ ID NO: 200 |
| 15 | SEQ ID NO: 13 | SEQ ID NO: 107 | SEQ ID NO: 201 |
| 16 | SEQ ID NO: 14 | SEQ ID NO: 108 | SEQ ID NO: 202 |
| 17 | SEQ ID NO: 15 | SEQ ID NO: 109 | SEQ ID NO: 203 |
| 18 | SEQ ID NO: 285 | SEQ ID NO: 291 | SEQ ID NO: 297 |
| 19 | SEQ ID NO: 16 | SEQ ID NO: 110 | SEQ ID NO: 204 |
| 20 | SEQ ID NO: 17 | SEQ ID NO: 111 | SEQ ID NO: 205 |
| 21 | SEQ ID NO: 18 | SEQ ID NO: 112 | SEQ ID NO: 206 |
| 22 | SEQ ID NO: 19 | SEQ ID NO: 113 | SEQ ID NO: 207 |
| 23 | SEQ ID NO: 20 | SEQ ID NO: 114 | SEQ ID NO: 208 |
| 24 | SEQ ID NO: 21 | SEQ ID NO: 115 | SEQ ID NO: 209 |
| 25 | SEQ ID NO: 22 | SEQ ID NO: 116 | SEQ ID NO: 210 |
| 26 | SEQ ID NO: 23 | SEQ ID NO: 117 | SEQ ID NO: 211 |
| 27 | SEQ ID NO: 24 | SEQ ID NO: 118 | SEQ ID NO: 212 |
| 28 | SEQ ID NO: 25 | SEQ ID NO: 119 | SEQ ID NO: 213 |
| 29 | SEQ ID NO: 26 | SEQ ID NO: 120 | SEQ ID NO: 214 |
| 30 | SEQ ID NO: 27 | SEQ ID NO: 121 | SEQ ID NO: 215 |
| 31 | SEQ ID NO: 28 | SEQ ID NO: 122 | SEQ ID NO: 216 |
| 32 | SEQ ID NO: 29 | SEQ ID NO: 123 | SEQ ID NO: 217 |
| 33 | SEQ ID NO: 286 | SEQ ID NO: 292 | SEQ ID NO: 298 |
| 34 | SEQ ID NO: 30 | SEQ ID NO: 124 | SEQ ID NO: 218 |
| 35 | SEQ ID NO: 31 | SEQ ID NO: 125 | SEQ ID NO: 219 |
| 36 | SEQ ID NO: 32 | SEQ ID NO: 126 | SEQ ID NO: 220 |
| 37 | SEQ ID NO: 33 | SEQ ID NO: 127 | SEQ ID NO: 221 |
| 38 | SEQ ID NO: 34 | SEQ ID NO: 128 | SEQ ID NO: 222 |
| 39 | SEQ ID NO: 35 | SEQ ID NO: 129 | SEQ ID NO: 223 |
| 40 | SEQ ID NO: 36 | SEQ ID NO: 130 | SEQ ID NO: 224 |
| 41 | SEQ ID NO: 37 | SEQ ID NO: 131 | SEQ ID NO: 225 |
| 42 | SEQ ID NO: 38 | SEQ ID NO: 132 | SEQ ID NO: 226 |
| 43 | SEQ ID NO: 39 | SEQ ID NO: 133 | SEQ ID NO: 227 |
| 44 | SEQ ID NO: 40 | SEQ ID NO: 134 | SEQ ID NO: 228 |
| 45 | SEQ ID NO: 41 | SEQ ID NO: 135 | SEQ ID NO: 229 |
| 46 | SEQ ID NO: 42 | SEQ ID NO: 136 | SEQ ID NO: 230 |
| 47 | SEQ ID NO: 43 | SEQ ID NO: 137 | SEQ ID NO: 231 |
| 48 | SEQ ID NO: 44 | SEQ ID NO: 138 | SEQ ID NO: 232 |
| 49 | SEQ ID NO: 45 | SEQ ID NO: 139 | SEQ ID NO: 233 |
| 50 | SEQ ID NO: 46 | SEQ ID NO: 140 | SEQ ID NO: 234 |
| 51 | SEQ ID NO: 47 | SEQ ID NO: 141 | SEQ ID NO: 235 |
| 52 | SEQ ID NO: 48 | SEQ ID NO: 142 | SEQ ID NO: 236 |
| 53 | SEQ ID NO: 49 | SEQ ID NO: 143 | SEQ ID NO: 237 |
| 54 | SEQ ID NO: 50 | SEQ ID NO: 144 | SEQ ID NO: 238 |
| 55 | SEQ ID NO: 51 | SEQ ID NO: 145 | SEQ ID NO: 239 |
| 56 | SEQ ID NO: 52 | SEQ ID NO: 146 | SEQ ID NO: 240 |
| 57 | SEQ ID NO: 53 | SEQ ID NO: 147 | SEQ ID NO: 241 |
| 58 | SEQ ID NO: 54 | SEQ ID NO: 148 | SEQ ID NO: 242 |
| 59 | SEQ ID NO: 55 | SEQ ID NO: 149 | SEQ ID NO: 243 |
| 60 | SEQ ID NO: 56 | SEQ ID NO: 150 | SEQ ID NO: 244 |
| 61 | SEQ ID NO: 57 | SEQ ID NO: 151 | SEQ ID NO: 245 |
| 62 | SEQ ID NO: 58 | SEQ ID NO: 152 | SEQ ID NO: 246 |
| 63 | SEQ ID NO: 59 | SEQ ID NO: 153 | SEQ ID NO: 247 |
| 64 | SEQ ID NO: 60 | SEQ ID NO: 154 | SEQ ID NO: 248 |
| 65 | SEQ ID NO: 61 | SEQ ID NO: 155 | SEQ ID NO: 249 |
| 66 | SEQ ID NO: 62 | SEQ ID NO: 156 | SEQ ID NO: 250 |
| 67 | SEQ ID NO: 63 | SEQ ID NO: 157 | SEQ ID NO: 251 |
| 68 | SEQ ID NO: 64 | SEQ ID NO: 158 | SEQ ID NO: 252 |
| 69 | SEQ ID NO: 65 | SEQ ID NO: 159 | SEQ ID NO: 253 |
| 70 | SEQ ID NO: 66 | SEQ ID NO: 160 | SEQ ID NO: 254 |
| 71 | SEQ ID NO: 67 | SEQ ID NO: 161 | SEQ ID NO: 255 |
| 72 | SEQ ID NO: 68 | SEQ ID NO: 162 | SEQ ID NO: 256 |
| 73 | SEQ ID NO: 69 | SEQ ID NO: 163 | SEQ ID NO: 257 |
| 74 | SEQ ID NO: 70 | SEQ ID NO: 164 | SEQ ID NO: 258 |
| 75 | SEQ ID NO: 71 | SEQ ID NO: 165 | SEQ ID NO: 259 |
| 76 | SEQ ID NO: 72 | SEQ ID NO: 166 | SEQ ID NO: 260 |
| 77 | SEQ ID NO: 73 | SEQ ID NO: 167 | SEQ ID NO: 261 |
| 78 | SEQ ID NO: 74 | SEQ ID NO: 168 | SEQ ID NO: 262 |
| 79 | SEQ ID NO: 75 | SEQ ID NO: 169 | SEQ ID NO: 263 |
| 80 | SEQ ID NO: 76 | SEQ ID NO: 170 | SEQ ID NO: 264 |
| 81 | SEQ ID NO: 77 | SEQ ID NO: 171 | SEQ ID NO: 265 |
| 82 | SEQ ID NO: 78 | SEQ ID NO: 172 | SEQ ID NO: 266 |
| 83 | SEQ ID NO: 79 | SEQ ID NO: 173 | SEQ ID NO: 267 |
| 84 | SEQ ID NO: 80 | SEQ ID NO: 174 | SEQ ID NO: 268 |
| 85 | SEQ ID NO: 81 | SEQ ID NO: 175 | SEQ ID NO: 269 |
| 86 | SEQ ID NO: 82 | SEQ ID NO: 176 | SEQ ID NO: 270 |
| 87 | SEQ ID NO: 83 | SEQ ID NO: 177 | SEQ ID NO: 271 |
| 88 | SEQ ID NO: 84 | SEQ ID NO: 178 | SEQ ID NO: 272 |
| 89 | SEQ ID NO: 85 | SEQ ID NO: 179 | SEQ ID NO: 273 |
| 90 | SEQ ID NO: 86 | SEQ ID NO: 180 | SEQ ID NO: 274 |
| 91 | SEQ ID NO: 87 | SEQ ID NO: 181 | SEQ ID NO: 275 |
| 92 | SEQ ID NO: 88 | SEQ ID NO: 182 | SEQ ID NO: 276 |
| 93 | SEQ ID NO: 287 | SEQ ID NO: 293 | SEQ ID NO: 299 |
| 94 | SEQ ID NO: 288 | SEQ ID NO: 294 | SEQ ID NO: 300 |
| 95 | SEQ ID NO: 89 | SEQ ID NO: 183 | SEQ ID NO: 277 |
| 96 | SEQ ID NO: 90 | SEQ ID NO: 184 | SEQ ID NO: 278 |
| 97 | SEQ ID NO: 91 | SEQ ID NO: 185 | SEQ ID NO: 279 |
| 98 | SEQ ID NO: 92 | SEQ ID NO: 186 | SEQ ID NO: 280 |
| 99 | SEQ ID NO: 93 | SEQ ID NO: 187 | SEQ ID NO: 281 |
| 100 | SEQ ID NO: 94 | SEQ ID NO: 188 | SEQ ID NO: 282 |

### Example 2: Retrotransposition Activity Assay

### 2.1 Cell Treatment (Day 0):

After HEK293T cells (commercially purchased) were cultured to the logarithmic growth phase, they were digested and dispersed into single cells with 0.25% Trypsin (Thermo), and added to a 96-well cell culture plate pre-coated with PDL (Sigma) at a cell concentration of 1×10⁴ cells/well, and cultured overnight at 37° C. in 5% CO₂.

### 2.2 Cell Transfection (Day 1):

A dosage of 200 ng of each retrotransposon plasmid constructed in example 1 was mixed with the transfection reagent Lipofectamine 2000 (Thermo) according to the ratio of transfection plasmid mass (μg): transfection reagent volume (μL)=1:2, and allowed to stand at room temperature for 15 minutes to form a transfection complex. The transfection complex was transferred to the cell culture plate and incubated with the cells, and two parallel tests were performed for each sample to be screened.

### 2.3 Cell Collection and Genome Extraction (Day 4)

72 hours after transfection, the cells were digested and dispersed into single cells with 0.25% Trypsin (Thermo). The cells were collected by centrifugation and the genomic DNA of the cells was extracted using a genome extraction kit (BEIJING BIOTEKE BIOTECHNOLOGY CO., LTD, DP1202), and the concentration of the genomic DNA was measured using a nanodrop one ultramicrovolume spectrophotometer (Thermo).

### 2.4 Detection

The genomic DNA of all samples was subjected to PCR analysis according to the conditions in Table 2 and Table 3, and the PCR products were detected by 1% agarose gel electrophoresis.

TABLE 2
PCR conditions

| | Amount of each |
|---|---|
| | component added |
| Name of each component | (μL) |
| PrimeSTAR ® HS DNA Polymerase (Takara) | 0.25 |
| dNTP Mixture (Takara) | 2 |
| 5 × PrimeSTAR ® Buffer (Mg²⁺ plus) | 5 |
| Primer F1 | 0.5 |
| Primer a1 | 0.5 |
| Genomic DNA | 200 ng |
| RNase Free Water | Make up to 25 μL |

TABLE 3
PCR program

| | |
|---|---|
| 98° C. 2 min | 1 cycle |
| 98° C. 10 s | 35 cycles |
| 62° C. 5 s | |
| 72° C. 30 s | |
| 72° C. 5 min | 1 cycle |

### 2.5 Detection Results

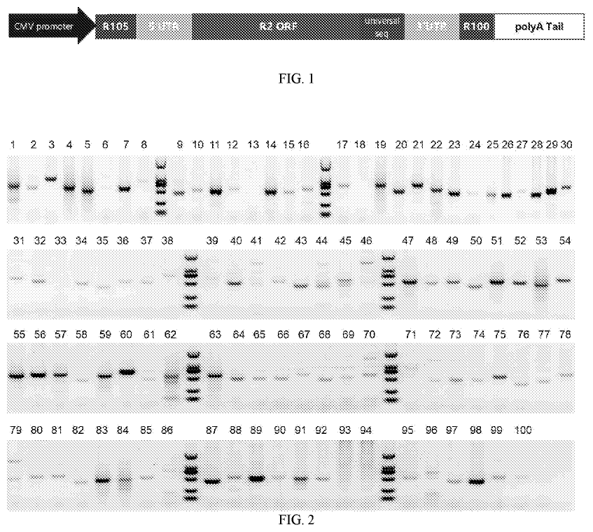

The results of retrotransposition activity are shown in FIG. 2. Different lanes in FIG. 2 correspond to different retrotransposons numbered 1-100 in Table 1. The results show that the lanes corresponding to most retrotransposons show a single band (e.g. the retrotransposons numbered 1-5, 7-12, 14-17, 19-32, 34-92, and 95-100 in Table 1), indicating that these functional proteins encoded by the retrotransposons has certain retrotransposition activity. However, the lanes corresponding to the 6 retrotransposons have no bands (e.g. the retrotransposons numbered 6, 13, 18, 33, 93, and 94 in Table 1), indicating that these functional proteins encoded by the retrotransposons do not have retrotransposition activity. Compared with these inactive retrotransposons (the retrotransposons numbered 6, 13, 18, 33, 93, 94 in Table 1), the other 94 retrotransposons show better retrotransposition activity (the retrotransposons numbered 1-5, 7-12, 14-17, 19-32, 34-92, and 95-100 in Table 1).

### Example 3: Relative Quantitative Analysis of Transposon Activity

Using a human single-copy gene 36b4 as an internal reference gene, a relative quantitative analysis of the retrotransposition activities of some retrotransposons in Table 1 was performed. The method was the same as that described in examples 1 and 2, the only difference was that: during the PCR process, an additional PCR primer pair targeting the single-copy gene 36b4 was added at the same time:

36b4-F:
5′-CAGCAAGTGGGAAGGTGTAATCC-3′ (the sequence is
as shown in SEQ ID NO: 303);
36b4-R:
5′-CCCATTCTATCATCAACGGGTACAA-3′ (the sequence is
as shown in SEQ ID NO: 304).

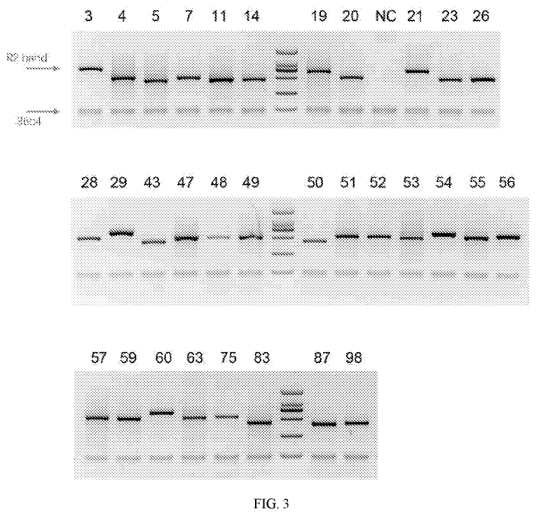

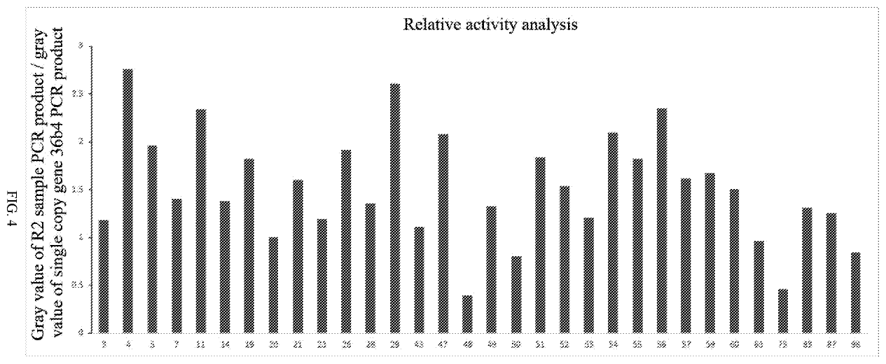

The results of retrotransposition activity are shown in FIG. 3. The numbers on different lanes correspond to the corresponding numbered transposon vectors in Table 1. The detection results in FIG. 3 were quantitatively analyzed using Image Lab version 6.1.0 (Bio-Rad Laboratories Inc) software. The calculation formula=gray value of R2 band/gray value of 36b4 band. The quantitative analysis results are shown in FIG. 4 and Table 4. The above results show that all functional proteins encoded by the retrotransposons in the present application have certain retrotransposition activities, and some functional proteins have relatively high activity.

TABLE 4
Relative quantitative results of retrotransposon activity

| Sample Code | Ratio of gray value |
|---|---|
| 3 | 1.18 |
| 4 | 2.75 |
| 5 | 1.96 |
| 7 | 1.40 |
| 11 | 2.34 |
| 14 | 1.38 |
| 19 | 1.88 |
| 20 | 1.00 |
| 21 | 1.60 |
| 23 | 1.9 |
| 26 | 1.91 |
| 28 | 1.36 |
| 29 | 2.61 |
| 43 | 1.11 |
| 47 | 2.08 |
| 48 | 0.40 |
| 49 | 1.3 |
| 50 | 0.81 |
| 51 | 1.84 |
| 52 | 1.53 |
| 53 | 1.20 |
| 54 | 2.10 |
| 55 | 1.82 |
| 56 | 2.35 |
| 57 | 1.61 |
| 59 | 1.67 |
| 60 | 1.51 |
| 63 | 0.96 |
| 75 | 0.46 |
| 83 | 1.31 |
| 87 | 1.25 |
| 98 | 0.84 |

### Example 4: Detection of 5′ Junction of Retrotransposons

The method used to detect the 5′ junction of 28s rRNA gene with the retrotransposons in Table 1, which showed the integrity of retrotransposition event. The method is the same as that described in examples 1 and 2, the only difference was that: 3 reverse primers were designed for different positions in the transposon ORF region and 5′ UTR sequence (the sequences of the reverse primers are shown in Table 4), and paired with the forward primer (28s-up-F1, with a sequence as shown in SEQ ID NO: 305), about 200 bp upstream of the insertion site in 28s rRNA gene for PCR analysis, respectively. The integrity of the 5′ end sequences of these samples after insertion was detected to preliminarily determine the retrotransposition integrity of different R2 samples.

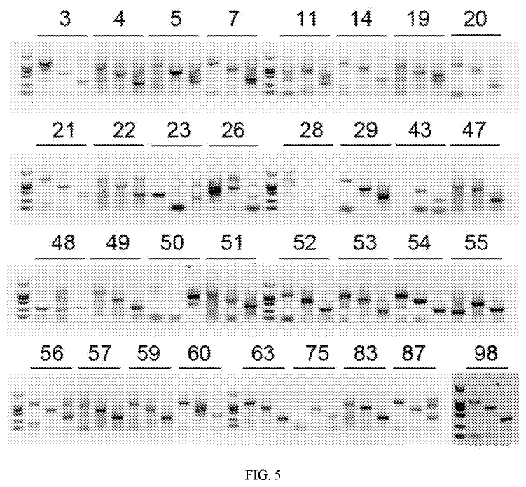

Retrotransposition integrity assay results are shown in FIG. 5. The numbers on different lanes correspond to the corresponding numbered transposon vectors in Table 1. The 3 lanes for each vector correspond to the PCR bands obtained by pairing the 3 reverse primers (with sequences as shown in Table 5) with the forward primer (28s-up-F1, with a sequence as shown in SEQ ID NO: 305), respectively. The results show that the retrotransposition products obtained from the functional proteins encoded by the retrotransposons in the present application have relatively good integrity.

TABLE 5
Primer sequences used for retrotransposition integrity assay

| | Corresponding | | |
|---|---|---|---|
| | transposon numbers in | | PCR product |
| Primer name | Table 1 | Primer sequence | length (bp) |
| 3-ORF-R1 | 3 | SEQ ID NO: 309 | 727 |
| 3-ORF-R2 | | SEQ ID NO: 310 | 507 |
| 3-5′UTR-R3 | | SEQ ID NO: 311 | 276 |
| 4-ORF-R1 | 4 | SEQ ID NO: 306 | 989 |
| 4-5′UTR-R2 | | SEQ ID NO: 307 | 551 |
| 4-5′UTR-R3 | | SEQ ID NO: 308 | 258 |
| 5-ORF-R1 | 5 | SEQ ID NO: 312 | 994 |
| 5-ORF-R2 | | SEQ ID NO: 313 | 635 |
| 5-5′UTR-R3 | | SEQ ID NO: 314 | 323 |
| 7-ORF-R1 | 7 | SEQ ID NO: 315 | 1054 |
| 7-ORF-R2 | | SEQ ID NO: 316 | 739 |
| 7-5′UTR-R3 | | SEQ ID NO: 317 | 296 |
| 11-ORF-R1 | 11 | SEQ ID NO: 318 | 1108 |
| 11-5′UTR-R2 | | SEQ ID NO: 319 | 721 |
| 11-5′UTR-R3 | | SEQ ID NO: 320 | 277 |
| 14-ORF-R1 | 14 | SEQ ID NO: 321 | 1105 |
| 14-5′UTR-R2 | | SEQ ID NO: 322 | 798 |
| 14-5′UTR-R3 | | SEQ ID NO: 323 | 381 |
| 19-ORF-R1 | 19 | SEQ ID NO: 324 | 1045 |
| 19-ORF-R2 | | SEQ ID NO: 325 | 658 |
| 19-ORF-R3 | | SEQ ID NO: 326 | 414 |
| 20-ORF-R1 | 20 | SEQ ID NO: 327 | 1097 |
| 20-5′UTR-R2 | | SEQ ID NO: 328 | 804 |
| 20-5′UTR-R3 | | SEQ ID NO: 329 | 280 |
| 21-ORF-R1 | 21 | SEQ ID NO: 330 | 1030 |
| 21-ORF-R2 | | SEQ ID NO: 331 | 628 |
| 21-5′UTR-R3 | | SEQ ID NO: 332 | 307 |
| 22-ORF-R1 | 22 | SEQ ID NO: 333 | 1071 |
| 22-5′UTR-R2 | | SEQ ID NO: 334 | 712 |
| 22-5′UTR-R3 | | SEQ ID NO: 335 | 394 |
| 23-ORF-R1 | 23 | SEQ ID NO: 336 | 923 |
| 23-ORF-R2 | | SEQ ID NO: 337 | 638 |
| 23-5′UTR-R3 | | SEQ ID NO: 338 | 297 |
| 26-ORF-R1 | 26 | SEQ ID NO: 339 | 1029 |
| 26-ORF-R2 | | SEQ ID NO: 340 | 621 |
| 26-5′UTR-R3 | | SEQ ID NO: 341 | 307 |
| 28-ORF-R1 | 28 | SEQ ID NO: 342 | 979 |
| 28-ORF-R2 | | SEQ ID NO: 343 | 607 |
| 28-5′UTR-R3 | | SEQ ID NO: 344 | 329 |
| 29-ORF-R1 | 29 | SEQ ID NO: 345 | 995 |
| 29-ORF-R2 | | SEQ ID NO: 346 | 595 |
| 29-5′UTR-R3 | | SEQ ID NO: 347 | 343 |
| 43-ORF-R1 | 43 | SEQ ID NO: 348 | 948 |
| 43-ORF-R2 | | SEQ ID NO: 349 | 578 |
| 43-5′UTR-R3 | | SEQ ID NO: 350 | 287 |
| 47-ORF-R1 | 47 | SEQ ID NO: 351 | 922 |
| 47-ORF-R2 | | SEQ ID NO: 352 | 696 |
| 47-5′UTR-R3 | | SEQ ID NO: 353 | 302 |
| 48-ORF-R1 | 48 | SEQ ID NO: 354 | 986 |
| 48-ORF-R2 | | SEQ ID NO: 355 | 675 |
| 48-5′UTR-R3 | | SEQ ID NO: 356 | 309 |
| 49-ORF-R1 | 49 | SEQ ID NO: 357 | 963 |
| 49-5′UTR-R2 | | SEQ ID NO: 358 | 580 |
| 49-5′UTR-R3 | | SEQ ID NO: 359 | 319 |
| 50-ORF-R1 | 50 | SEQ ID NO: 360 | 994 |
| 50-ORF-R2 | | SEQ ID NO: 361 | 598 |
| 50-5′UTR-R3 | | SEQ ID NO: 362 | 389 |
| 51-ORF-R1 | 51 | SEQ ID NO: 363 | 992 |
| 51-ORF-R2 | | SEQ ID NO: 364 | 612 |
| 51-5′UTR-R3 | | SEQ ID NO: 365 | 383 |
| 52-ORF-R1 | 52 | SEQ ID NO: 366 | 916 |
| 52-5′UTR-R2 | | SEQ ID NO: 367 | 576 |
| 52-5′UTR-R3 | | SEQ ID NO: 368 | 284 |
| 53-ORF-R1 | 53 | SEQ ID NO: 369 | 958 |
| 53-ORF-R2 | | SEQ ID NO: 370 | 603 |
| 53-5′UTR-R3 | | SEQ ID NO: 371 | 250 |
| 54-ORF-R1 | 54 | SEQ ID NO: 372 | 895 |
| 54-ORF-R2 | | SEQ ID NO: 373 | 602 |
| 54-5′UTR-R3 | | SEQ ID NO: 374 | 288 |
| 55-ORF-R1 | 55 | SEQ ID NO: 375 | 936 |
| 55-5′UTR-R2 | | SEQ ID NO: 376 | 525 |
| 55-5′UTR-R3 | | SEQ ID NO: 377 | 307 |
| 56-ORF-R1 | 56 | SEQ ID NO: 378 | 910 |
| 56-ORF-R2 | | SEQ ID NO: 379 | 564 |
| 56-5′UTR-R3 | | SEQ ID NO: 380 | 364 |
| 57-ORF-R1 | 57 | SEQ ID NO: 381 | 904 |
| 57-5′UTR-R2 | | SEQ ID NO: 382 | 626 |
| 57-5′UTR-R3 | | SEQ ID NO: 383 | 338 |
| 59-ORF-R1 | 59 | SEQ ID NO: 384 | 885 |
| 59-5′UTR-R2 | | SEQ ID NO: 385 | 587 |
| 59-5′UTR-R3 | | SEQ ID NO: 386 | 290 |
| 60-ORF-R1 | 60 | SEQ ID NO: 387 | 868 |
| 60-ORF-R2 | | SEQ ID NO: 388 | 593 |
| 60-5′UTR-R3 | | SEQ ID NO: 389 | 325 |
| 63-ORF-R1 | 63 | SEQ ID NO: 390 | 894 |
| 63-5′UTR-R2 | | SEQ ID NO: 391 | 612 |
| 63-5′UTR-R3 | | SEQ ID NO: 392 | 258 |
| 75-ORF-R1 | 75 | SEQ ID NO: 393 | 922 |
| 75-5′UTR-R2 | | SEQ ID NO: 394 | 557 |
| 75-5′UTR-R3 | | SEQ ID NO: 395 | 325 |
| 83-ORF-R1 | 83 | SEQ ID NO: 396 | 950 |
| 83-5′UTR-R2 | | SEQ ID NO: 397 | 697 |
| 83-5′UTR-R3 | | SEQ ID NO: 398 | 322 |
| 87-ORF-R1 | 87 | SEQ ID NO: 399 | 939 |
| 87-ORF-R2 | | SEQ ID NO: 400 | 588 |
| 87-5′UTR-R3 | | SEQ ID NO: 401 | 312 |
| 98-ORF-R1 | 98 | SEQ ID NO: 402 | 852 |
| 98-ORF-R2 | | SEQ ID NO: 403 | 613 |
| 98-5′UTR-R3 | | SEQ ID NO: 404 | 326 |

It should be stated that the above are only the preferred examples of the present application and are not intended to limit the present application. For those of ordinary skill in the art, various modifications and changes can be made to the present application. Although the specific embodiments have been described, for the applicant or a person skilled in the art, the substitutions, modifications, changes, improvements, and substantial equivalents of the above embodiments may exist or cannot be foreseen currently. Therefore, the submitted appended claims and claims that may be modified are intended to cover all such substitutions, modifications, changes, improvements, and substantial equivalents. It is important that, as the technology evolves, many elements described herein may be replaced with equivalent elements that appear after the present application.

1. An isolated functional protein encoded by a retrotransposon, wherein, the functional protein has a functional protein sequence selected from the following (i) or a variant sequence of the aforementioned functional protein with functional protein activity in (ii)-(iv):
(i) at least one amino acid sequence as shown in any one of SEQ ID NO: 1-94;
(ii) at least one sequence having deletion, substitution, insertion, mutation of 1, 2, 3, 4, 5, 6, 7, 8, 9 or 10 amino acids of the amino acid sequence as shown in any one of SEQ ID NO: 1-94;
(iii) at least one amino acid sequence having at least 70%, 80%, 90%, 95% or 99% identity with the amino acid sequence as shown in any one of SEQ ID NO: 1-94; and
(iv) at least one sequence obtained by further fusing the amino acid sequence as shown in any one of SEQ ID NO: 1-94 with other sequences.

2. An isolated functional protein encoded by a retrotransposon, wherein, the functional protein includes an amino acid sequence as shown in formula (I):
C(X₁)ₐC(X₂)_{b}H(X₃)_{c}H (I),
wherein,
a, b and c are the number of amino acids;
C is cysteine;
His histidine;
(X₁) is any amino acid, and a is 1, 2, 3 or 4;
(X₂) is any amino acid, and b is 11, 12, 13, 14, 15, 16 or 17; and
(X₃) is any amino acid, and c is 4.

3. An isolated functional protein encoded by a retrotransposon, wherein, the functional protein includes an amino acid sequence as shown in formula (II):
G(X₄)_{d}QGD(X₅)ₑS(X₆)_{f}F(X₇)_{g}D (II),
wherein,
d, e, f and g are the number of amino acids;
D is aspartic acid;
F is phenylalanine;
G is glycine;
Q is glutamine;
S is serine;
(X₄) is any amino acid, and dis 2;
(X₅) is any amino acid, and e is 2;
(X₆) is any amino acid, and f is 3; and
(X₇) is any amino acid, and g is 30, 31, 32, 33, 34, 35, or 36.

4. An isolated functional protein encoded by a retrotransposon, wherein, the functional protein includes an amino acid sequence as shown in formula (III):
C(X₈)ₕC(X₉)ᵢE(X₁₀)ⱼH(X₁₁)ₖC(X₁₂)ₗRH(X₁₃)ₘPD(X₁₄)ₙ(X₁₅)(X₁₆)ₒK(X₁₇)ₚY (III),
wherein,
h, i, j, k, l, m, n, o and p are the number of amino acids;
C is cysteine;
D is aspartic acid;
E is glutamic acid;
H is histidine;
K is lysine;
R is arginine;
P is proline;
Y is tyrosine;
(X₈) is any amino acid, and h is 2, 3 or 4;
(X₉) is any amino acid, and i is 3, 4, 5, 6, 7, 8, 9 or 10;
(X₁₀) is any amino acid, and j is 3;
(X₁₁) is any amino acid, and k is 4;
(X₁₂) is any amino acid, and 1 is 9;
(X₁₃) is any amino acid, and m is 31, 32, 33, 34, 35 or 36;
(X₁₄) is any amino acid, and n is 11, 12, 13, 14, 15, 16, 17, 18 or 19;
(X₁₅) is aspartic acid, or glutamic acid;
(X₁₆) is any amino acid, and o is 15, 16, 17, 18 or 19; and
(X₁₇) is any amino acid, and p is 3.

5. An isolated functional protein encoded by a retrotransposon, wherein, the functional protein includes at least two of the amino acid sequences as shown in formula (I), formula (II), and formula (III).

6. An isolated functional protein encoded by a retrotransposon, wherein, the functional protein includes the amino acid sequences as shown in formula (I), formula (II), and formula (III).

7. The functional protein according to claim 1, wherein the retrotransposon is a non-LTR (non-Long Terminal Repeat) retrotransposon.

8. The functional protein according to claim 7, wherein the non-LTR retrotransposon is a restriction enzyme-like nuclease (RLE) retrotransposon.

9. (canceled)

10. The functional protein according to claim 1, wherein the functional protein includes a DNA binding domain that can bind to a specific region in the genome of a cell, wherein the specific region in the genome of a cell includes a gene encoding 28s rRNA.

11. The functional protein according to claim 10, wherein the functional protein further includes an RNA binding domain, a reverse transcriptase functional domain, and/or a nuclease functional domain.

12.-14. (canceled)

15. A nucleic acid, wherein, the nucleic acid encodes the functional protein according to claim 1.

16. A nucleic acid set, comprising a 5′-untranslated region, wherein, the 5′-untranslated region includes at least one of the nucleotide sequences as shown in SEQ ID NO: 95 188.

17. A nucleic acid set, comprising a 3′-untranslated region, wherein, the 3′-untranslated region includes at least one of the nucleotide sequences as shown in SEQ ID NO: 189-282.

18. A nucleic acid set, comprising a 5′-untranslated region and a 3′-untranslated region, wherein, the 5′-untranslated region includes the nucleotide sequence as shown in any one of SEQ ID NO: 95-188 or a variant thereof, and the 3′-untranslated region includes the nucleotide sequence as shown in any one of SEQ ID NO: 189-282 or a variant thereof, the RNA transcribed from the nucleic acid set can bind to the functional protein encoded by a specific retrotransposon.

19. A nucleic acid construct, comprising the nucleic acid according to claim 15, wherein the nucleic acid construct further comprising an exogenous nucleic acid fragment, the exogenous nucleic acid fragment is operably inserted into the nucleic acid construct through a polyclonal insertion site, and the exogenous nucleic acid fragment may be one or more, and may be the same or different.

20. (canceled)

21. The nucleic acid construct according to claim 19, wherein the exogenous nucleic acid fragment includes any gene of interest or any gene that is transposable.

22. The nucleic acid construct according to claim 58, wherein the gene of a natural functional protein includes a fluorescence-based reporter gene, a luciferase gene, and/or an antibiotic resistance gene.

23. The nucleic acid construct according to claim 58, wherein the artificial chimeric gene includes a gene of a chimeric antigen receptor.

24. The nucleic acid construct according to claim 58, wherein the gene of a non-coding RNA includes rRNA, tRNA, small interfering RNA (siRNA), small nuclear RNA (snRNA), small nucleolar RNA (snoRNA), microRNA (miRNA), and/or long non-coding RNA (lncRNA).

25. The nucleic acid construct according to claim 19, wherein the nucleic acid construct further includes a promoter and a poly (A) sequence.

26. (canceled)

27. A composition, wherein, the composition includes:
a functional protein or a functional fragment thereof encoded by a R2 family retrotransposon, or a nucleic acid encoding the functional protein or the functional fragment thereof, wherein the functional protein or the functional fragment thereof has a function of catalyzing an insertion of an exogenous nucleic acid fragment into the genome of a cell; and
a nucleic acid set, wherein the RNA transcribed by the nucleic acid set can be recognized by a functional protein or a functional fragment thereof encoded by a specific retrotransposon.

28. The composition according to claim 27, wherein the composition is selected from at least one of the following groups (1) to (95), and any one of the following groups (1) to (94) includes: a functional protein-related sequence and a nucleic acid set,
(1) the functional protein-related sequence is an amino acid sequence comprising the sequence as shown in SEQ ID NO: 1 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 95; and the 3′-untranslated region is a nucleotide sequence comprising the sequence as shown in SEQ ID NO: 189;
(2) the functional protein-related sequence is an amino acid sequence comprising the sequence as shown in SEQ ID NO: 2 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 96; and the 3′-untranslated region is a nucleotide sequence comprising the sequence as shown in SEQ ID NO: 190;
(3) the functional protein-related sequence is an amino acid sequence comprising the sequence as shown in SEQ ID NO: 3 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 97; and the 3′-untranslated region is a nucleotide sequence comprising the sequence as shown in SEQ ID NO: 191;
(4) the functional protein-related sequence is an amino acid sequence comprising the sequence as shown in SEQ ID NO: 4 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 98; and the 3′-untranslated region is a nucleotide sequence comprising the sequence as shown in SEQ ID NO: 192;
(5) the functional protein-related sequence is an amino acid sequence comprising the sequence as shown in SEQ ID NO: 5 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 99; and the 3′-untranslated region is a nucleotide sequence comprising the sequence as shown in SEQ ID NO: 193;
(6) the functional protein-related sequence is an amino acid sequence comprising the sequence as shown in SEQ ID NO: 6 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 100; and the 3′-untranslated region is a nucleotide sequence comprising the sequence as shown in SEQ ID NO: 194;
(7) the functional protein-related sequence is an amino acid sequence comprising the sequence as shown in SEQ ID NO: 7 or a nucleic acid encoding the amino acid sequence; the 101; and the 3′-untranslated region is a nucleotide sequence comprising the sequence as shown in SEQ ID NO: 195;
(8) the functional protein-related sequence is an amino acid sequence comprising the sequence as shown in SEQ ID NO: 8 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 102; and the 3′-untranslated region is a nucleotide sequence comprising the sequence as shown in SEQ ID NO: 196;
(9) the functional protein-related sequence is an amino acid sequence comprising the sequence as shown in SEQ ID NO: 9 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 103; and the 3′-untranslated region is a nucleotide sequence comprising the sequence as shown in SEQ ID NO: 197;
(10) the functional protein-related sequence is an amino acid sequence comprising the sequence as shown in SEQ ID NO: 10 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 104; and the 3′-untranslated region is a nucleotide sequence comprising the sequence as shown in SEQ ID NO: 198;
(11) the functional protein-related sequence is an amino acid sequence comprising the sequence as shown in SEQ ID NO: 11 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 105; and the 3′-untranslated region is a nucleotide sequence comprising the sequence as shown in SEQ ID NO: 199;
(12) the functional protein-related sequence is an amino acid sequence comprising the sequence as shown in SEQ ID NO: 12 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 106; and the 3′-untranslated region is a nucleotide sequence comprising the sequence as shown in SEQ ID NO: 200;
(13) the functional protein-related sequence is an amino acid sequence comprising the sequence as shown in SEQ ID NO: 13 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 107; and the 3′-untranslated region is a nucleotide sequence comprising the sequence as shown in SEQ ID NO: 201;
(14) the functional protein-related sequence is an amino acid sequence comprising the sequence as shown in SEQ ID NO: 14 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 108; and the 3′-untranslated region is a nucleotide sequence comprising the sequence as shown in SEQ ID NO: 202;
(15) the functional protein-related sequence is an amino acid sequence comprising the sequence as shown in SEQ ID NO: 15 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 109; and the 3′-untranslated region is a nucleotide sequence comprising the sequence as shown in SEQ ID NO: 203;
(16) the functional protein-related sequence is an amino acid sequence comprising the sequence as shown in SEQ ID NO: 16 or a nucleic acid encoding the amino acid sequence; the 110; and the 3′-untranslated region is a nucleotide sequence comprising the sequence as shown in SEQ ID NO: 204;
(17) the functional protein-related sequence is an amino acid sequence comprising the sequence as shown in SEQ ID NO: 17 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 111; and the 3′-untranslated region is a nucleotide sequence comprising the sequence as shown in SEQ ID NO: 205;
(18) the functional protein-related sequence is an amino acid sequence comprising the sequence as shown in SEQ ID NO: 18 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 112; and the 3′-untranslated region is a nucleotide sequence comprising the sequence as shown in SEQ ID NO: 206;
(19) the functional protein-related sequence is an amino acid sequence comprising the sequence as shown in SEQ ID NO: 19 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 113; and the 3′-untranslated region is a nucleotide sequence comprising the sequence as shown in SEQ ID NO: 207;
(20) the functional protein-related sequence is an amino acid sequence comprising the sequence as shown in SEQ ID NO: 20 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 114; and the 3′-untranslated region is a nucleotide sequence comprising the sequence as shown in SEQ ID NO: 208;
(21) the functional protein-related sequence is an amino acid sequence comprising the sequence as shown in SEQ ID NO: 21 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 115; and the 3′-untranslated region is a nucleotide sequence comprising the sequence as shown in SEQ ID NO: 209;
(22) the functional protein-related sequence is an amino acid sequence comprising the sequence as shown in SEQ ID NO: 22 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 116; and the 3′-untranslated region is a nucleotide sequence comprising the sequence as shown in SEQ ID NO: 210;
(23) the functional protein-related sequence is an amino acid sequence comprising the sequence as shown in SEQ ID NO: 23 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 117; and the 3′-untranslated region is a nucleotide sequence comprising the sequence as shown in SEQ ID NO: 211;
(24) the functional protein-related sequence is an amino acid sequence comprising the sequence as shown in SEQ ID NO: 24 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 118; and the 3′-untranslated region is a nucleotide sequence comprising the sequence as shown in SEQ ID NO: 212;
(25) the functional protein-related sequence is an amino acid sequence comprising the sequence as shown in SEQ ID NO: 25 or a nucleic acid encoding the amino acid sequence; the 119; and the 3′-untranslated region is a nucleotide sequence comprising the sequence as shown in SEQ ID NO: 213;
(26) the functional protein-related sequence is an amino acid sequence comprising the sequence as shown in SEQ ID NO: 26 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 120; and the 3′-untranslated region is a nucleotide sequence comprising the sequence as shown in SEQ ID NO: 214;
(27) the functional protein-related sequence is an amino acid sequence comprising the sequence as shown in SEQ ID NO: 27 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 121; and the 3′-untranslated region is a nucleotide sequence comprising the sequence as shown in SEQ ID NO: 215;
(28) the functional protein-related sequence is an amino acid sequence comprising the sequence as shown in SEQ ID NO: 28 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 122; and the 3′-untranslated region is a nucleotide sequence comprising the sequence as shown in SEQ ID NO: 216;
(29) the functional protein-related sequence is an amino acid sequence comprising the sequence as shown in SEQ ID NO: 29 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 123; and the 3′-untranslated region is a nucleotide sequence comprising the sequence as shown in SEQ ID NO: 217;
(30) the functional protein-related sequence is an amino acid sequence comprising the sequence as shown in SEQ ID NO: 30 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 124; and the 3′-untranslated region is a nucleotide sequence comprising the sequence as shown in SEQ ID NO: 218;
(31) the functional protein-related sequence is an amino acid sequence comprising the sequence as shown in SEQ ID NO: 31 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 125; and the 3′-untranslated region is a nucleotide sequence comprising the sequence as shown in SEQ ID NO: 219;
(32) the functional protein-related sequence is an amino acid sequence comprising the sequence as shown in SEQ ID NO: 32 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 126; and the 3′-untranslated region is a nucleotide sequence comprising the sequence as shown in SEQ ID NO: 220;
(33) the functional protein-related sequence is an amino acid sequence comprising the sequence as shown in SEQ ID NO: 33 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 127; and the 3′-untranslated region is a nucleotide sequence comprising the sequence as shown in SEQ ID NO: 221;
(34) the functional protein-related sequence is an amino acid sequence comprising the sequence as shown in SEQ ID NO: 34 or a nucleic acid encoding the amino acid sequence; the 128; and the 3′-untranslated region is a nucleotide sequence comprising the sequence as shown in SEQ ID NO: 222;
(35) the functional protein-related sequence is an amino acid sequence comprising the sequence as shown in SEQ ID NO: 35 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 129; and the 3′-untranslated region is a nucleotide sequence comprising the sequence as shown in SEQ ID NO: 223;
(36) the functional protein-related sequence is an amino acid sequence comprising the sequence as shown in SEQ ID NO: 36 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 130; and the 3′-untranslated region is a nucleotide sequence comprising the sequence as shown in SEQ ID NO: 224;
(37) the functional protein-related sequence is an amino acid sequence comprising the sequence as shown in SEQ ID NO: 37 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 131; and the 3′-untranslated region is a nucleotide sequence comprising the sequence as shown in SEQ ID NO: 225;
(38) the functional protein-related sequence is an amino acid sequence comprising the sequence as shown in SEQ ID NO: 38 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 132; and the 3′-untranslated region is a nucleotide sequence comprising the sequence as shown in SEQ ID NO: 226;
(39) the functional protein-related sequence is an amino acid sequence comprising the sequence as shown in SEQ ID NO: 39 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 133; and the 3′-untranslated region is a nucleotide sequence comprising the sequence as shown in SEQ ID NO: 227;
(40) the functional protein-related sequence is an amino acid sequence comprising the sequence as shown in SEQ ID NO: 40 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 134; and the 3′-untranslated region is a nucleotide sequence comprising the sequence as shown in SEQ ID NO: 228;
(41) the functional protein-related sequence is an amino acid sequence comprising the sequence as shown in SEQ ID NO: 41 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 135; and the 3′-untranslated region is a nucleotide sequence comprising the sequence as shown in SEQ ID NO: 229;
(42) the functional protein-related sequence is an amino acid sequence comprising the sequence as shown in SEQ ID NO: 42 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 136; and the 3′-untranslated region is a nucleotide sequence comprising the sequence as shown in SEQ ID NO: 230;
(43) the functional protein-related sequence is an amino acid sequence comprising the sequence as shown in SEQ ID NO: 43 or a nucleic acid encoding the amino acid sequence; the 137; and the 3′-untranslated region is a nucleotide sequence comprising the sequence as shown in SEQ ID NO: 231;
(44) the functional protein-related sequence is an amino acid sequence comprising the sequence as shown in SEQ ID NO: 44 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 138; and the 3′-untranslated region is a nucleotide sequence comprising the sequence as shown in SEQ ID NO: 232;
(45) the functional protein-related sequence is an amino acid sequence comprising the sequence as shown in SEQ ID NO: 45 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 139; and the 3′-untranslated region is a nucleotide sequence comprising the sequence as shown in SEQ ID NO: 233;
(46) the functional protein-related sequence is an amino acid sequence comprising the sequence as shown in SEQ ID NO: 46 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 140; and the 3′-untranslated region is a nucleotide sequence comprising the sequence as shown in SEQ ID NO: 234;
(47) the functional protein-related sequence is an amino acid sequence comprising the sequence as shown in SEQ ID NO: 47 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 141; and the 3′-untranslated region is a nucleotide sequence comprising the sequence as shown in SEQ ID NO: 235;
(48) the functional protein-related sequence is an amino acid sequence comprising the sequence as shown in SEQ ID NO: 48 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 142; and the 3′-untranslated region is a nucleotide sequence comprising the sequence as shown in SEQ ID NO: 236;
(49) the functional protein-related sequence is an amino acid sequence comprising the sequence as shown in SEQ ID NO: 49 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 143; and the 3′-untranslated region is a nucleotide sequence comprising the sequence as shown in SEQ ID NO: 237;
(50) the functional protein-related sequence is an amino acid sequence comprising the sequence as shown in SEQ ID NO: 50 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 144; and the 3′-untranslated region is a nucleotide sequence comprising the sequence as shown in SEQ ID NO: 238;
(51) the functional protein-related sequence is an amino acid sequence comprising the sequence as shown in SEQ ID NO: 51 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 145; and the 3′-untranslated region is a nucleotide sequence comprising the sequence as shown in SEQ ID NO: 239;
(52) the functional protein-related sequence is an amino acid sequence comprising the sequence as shown in SEQ ID NO: 52 or a nucleic acid encoding the amino acid sequence; the 146; and the 3′-untranslated region is a nucleotide sequence comprising the sequence as shown in SEQ ID NO: 240;
(53) the functional protein-related sequence is an amino acid sequence comprising the sequence as shown in SEQ ID NO: 53 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 147; and the 3′-untranslated region is a nucleotide sequence comprising the sequence as shown in SEQ ID NO: 241;
(54) the functional protein-related sequence is an amino acid sequence comprising the sequence as shown in SEQ ID NO: 54 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 148; and the 3′-untranslated region is a nucleotide sequence comprising the sequence as shown in SEQ ID NO: 242;
(55) the functional protein-related sequence is an amino acid sequence comprising the sequence as shown in SEQ ID NO: 55 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 149; and the 3′-untranslated region is a nucleotide sequence comprising the sequence as shown in SEQ ID NO: 243;
(56) the functional protein-related sequence is an amino acid sequence comprising the sequence as shown in SEQ ID NO: 56 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 150; and the 3′-untranslated region is a nucleotide sequence comprising the sequence as shown in SEQ ID NO: 244;
(57) the functional protein-related sequence is an amino acid sequence comprising the sequence as shown in SEQ ID NO: 57 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 151; and the 3′-untranslated region is a nucleotide sequence comprising the sequence as shown in SEQ ID NO: 245;
(58) the functional protein-related sequence is an amino acid sequence comprising the sequence as shown in SEQ ID NO: 58 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 152; and the 3′-untranslated region is a nucleotide sequence comprising the sequence as shown in SEQ ID NO: 246;
(59) the functional protein-related sequence is an amino acid sequence comprising the sequence as shown in SEQ ID NO: 59 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 153; and the 3′-untranslated region is a nucleotide sequence comprising the sequence as shown in SEQ ID NO: 247;
(60) the functional protein-related sequence is an amino acid sequence comprising the sequence as shown in SEQ ID NO: 60 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 154; and the 3′-untranslated region is a nucleotide sequence comprising the sequence as shown in SEQ ID NO: 248;
(61) the functional protein-related sequence is an amino acid sequence comprising the sequence as shown in SEQ ID NO: 61 or a nucleic acid encoding the amino acid sequence; the 155; and the 3′-untranslated region is a nucleotide sequence comprising the sequence as shown in SEQ ID NO: 249;
(62) the functional protein-related sequence is an amino acid sequence comprising the sequence as shown in SEQ ID NO: 62 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 156; and the 3′-untranslated region is a nucleotide sequence comprising the sequence as shown in SEQ ID NO: 250;
(63) the functional protein-related sequence is an amino acid sequence comprising the sequence as shown in SEQ ID NO: 63 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 157; and the 3′-untranslated region is a nucleotide sequence comprising the sequence as shown in SEQ ID NO: 251;
(64) the functional protein-related sequence is an amino acid sequence comprising the sequence as shown in SEQ ID NO: 64 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 158; and the 3′-untranslated region is a nucleotide sequence comprising the sequence as shown in SEQ ID NO: 252;
(65) the functional protein-related sequence is an amino acid sequence comprising the sequence as shown in SEQ ID NO: 65 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 159; and the 3′-untranslated region is a nucleotide sequence comprising the sequence as shown in SEQ ID NO: 253;
(66) the functional protein-related sequence is an amino acid sequence comprising the sequence as shown in SEQ ID NO: 66 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 160; and the 3′-untranslated region is a nucleotide sequence comprising the sequence as shown in SEQ ID NO: 254;
(67) the functional protein-related sequence is an amino acid sequence comprising the sequence as shown in SEQ ID NO: 67 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 161; and the 3′-untranslated region is a nucleotide sequence comprising the sequence as shown in SEQ ID NO: 255;
(68) the functional protein-related sequence is an amino acid sequence comprising the sequence as shown in SEQ ID NO: 68 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 162; and the 3′-untranslated region is a nucleotide sequence comprising the sequence as shown in SEQ ID NO: 256;
(69) the functional protein-related sequence is an amino acid sequence comprising the sequence as shown in SEQ ID NO: 69 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 163; and the 3′-untranslated region is a nucleotide sequence comprising the sequence as shown in SEQ ID NO: 257;
(70) the functional protein-related sequence is an amino acid sequence comprising the sequence as shown in SEQ ID NO: 70 or a nucleic acid encoding the amino acid sequence; the 164; and the 3′-untranslated region is a nucleotide sequence comprising the sequence as shown in SEQ ID NO: 258;
(71) the functional protein-related sequence is an amino acid sequence comprising the sequence as shown in SEQ ID NO: 71 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 165; and the 3′-untranslated region is a nucleotide sequence comprising the sequence as shown in SEQ ID NO: 259;
(72) the functional protein-related sequence is an amino acid sequence comprising the sequence as shown in SEQ ID NO: 72 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 166; and the 3′-untranslated region is a nucleotide sequence comprising the sequence as shown in SEQ ID NO: 260;
(73) the functional protein-related sequence is an amino acid sequence comprising the sequence as shown in SEQ ID NO: 73 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 167; and the 3′-untranslated region is a nucleotide sequence comprising the sequence as shown in SEQ ID NO: 261;
(74) the functional protein-related sequence is an amino acid sequence comprising the sequence as shown in SEQ ID NO: 74 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 168; and the 3′-untranslated region is a nucleotide sequence comprising the sequence as shown in SEQ ID NO: 262;
(75) the functional protein-related sequence is an amino acid sequence comprising the sequence as shown in SEQ ID NO: 75 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 169; and the 3′-untranslated region is a nucleotide sequence comprising the sequence as shown in SEQ ID NO: 263;
(76) the functional protein-related sequence is an amino acid sequence comprising the sequence as shown in SEQ ID NO: 76 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 170; and the 3′-untranslated region is a nucleotide sequence comprising the sequence as shown in SEQ ID NO: 264;
(77) the functional protein-related sequence is an amino acid sequence comprising the sequence as shown in SEQ ID NO: 77 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 171; and the 3′-untranslated region is a nucleotide sequence comprising the sequence as shown in SEQ ID NO: 265;
(78) the functional protein-related sequence is an amino acid sequence comprising the sequence as shown in SEQ ID NO: 78 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 172; and the 3′-untranslated region is a nucleotide sequence comprising the sequence as shown in SEQ ID NO: 266;
(79) the functional protein-related sequence is an amino acid sequence comprising the sequence as shown in SEQ ID NO: 79 or a nucleic acid encoding the amino acid sequence; the 173; and the 3′-untranslated region is a nucleotide sequence comprising the sequence as shown in SEQ ID NO: 267;
(80) the functional protein-related sequence is an amino acid sequence comprising the sequence as shown in SEQ ID NO: 80 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 174; and the 3′-untranslated region is a nucleotide sequence comprising the sequence as shown in SEQ ID NO: 268;
(81) the functional protein-related sequence is an amino acid sequence comprising the sequence as shown in SEQ ID NO: 81 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 175; and the 3′-untranslated region is a nucleotide sequence comprising the sequence as shown in SEQ ID NO: 269;
(82) the functional protein-related sequence is an amino acid sequence comprising the sequence as shown in SEQ ID NO: 82 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 176; and the 3′-untranslated region is a nucleotide sequence comprising the sequence as shown in SEQ ID NO: 270;
(83) the functional protein-related sequence is an amino acid sequence comprising the sequence as shown in SEQ ID NO: 83 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 177; and the 3′-untranslated region is a nucleotide sequence comprising the sequence as shown in SEQ ID NO: 271;
(84) the functional protein-related sequence is an amino acid sequence comprising the sequence as shown in SEQ ID NO: 84 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 178; and the 3′-untranslated region is a nucleotide sequence comprising the sequence as shown in SEQ ID NO: 272;
(85) the functional protein-related sequence is an amino acid sequence comprising the sequence as shown in SEQ ID NO: 85 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 179; and the 3′-untranslated region is a nucleotide sequence comprising the sequence as shown in SEQ ID NO: 273;
(86) the functional protein-related sequence is an amino acid sequence comprising the sequence as shown in SEQ ID NO: 86 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 180; and the 3′-untranslated region is a nucleotide sequence comprising the sequence as shown in SEQ ID NO: 274;
(87) the functional protein-related sequence is an amino acid sequence comprising the sequence as shown in SEQ ID NO: 87 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 181; and the 3′-untranslated region is a nucleotide sequence comprising the sequence as shown in SEQ ID NO: 275;
(88) the functional protein-related sequence is an amino acid sequence comprising the sequence as shown in SEQ ID NO: 88 or a nucleic acid encoding the amino acid sequence; the 182; and the 3′-untranslated region is a nucleotide sequence comprising the sequence as shown in SEQ ID NO: 276;
(89) the functional protein-related sequence is an amino acid sequence comprising the sequence as shown in SEQ ID NO: 89 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 183; and the 3′-untranslated region is a nucleotide sequence comprising the sequence as shown in SEQ ID NO: 277;
(90) the functional protein-related sequence is an amino acid sequence comprising the sequence as shown in SEQ ID NO: 90 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 184; and the 3′-untranslated region is a nucleotide sequence comprising the sequence as shown in SEQ ID NO: 278;
(91) the functional protein-related sequence is an amino acid sequence comprising the sequence as shown in SEQ ID NO: 91 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 185; and the 3′-untranslated region is a nucleotide sequence comprising the sequence as shown in SEQ ID NO: 279;
(92) the functional protein-related sequence is an amino acid sequence comprising the sequence as shown in SEQ ID NO: 92 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 186; and the 3′-untranslated region is a nucleotide sequence comprising the sequence as shown in SEQ ID NO: 280;
(93) the functional protein-related sequence is an amino acid sequence comprising the sequence as shown in SEQ ID NO: 93 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 187; and the 3′-untranslated region is a nucleotide sequence comprising the sequence as shown in SEQ ID NO: 281;
(94) the functional protein-related sequence is an amino acid sequence comprising the sequence as shown in SEQ ID NO: 94 or a nucleic acid encoding the amino acid sequence; the nucleic acid set includes a 5′-untranslated region and a 3′-untranslated region, and the 5′-untranslated region is a nucleotide sequence comprising the sequence as shown in SEQ ID NO: 188; and the 3′-untranslated region is a nucleotide sequence comprising the sequence as shown in SEQ ID NO: 282; or
(95) a variant of any of the aforementioned group (1)-group (94),
wherein, the functional protein-related sequence is the amino acid sequence of the variant of the functional protein in each group or a nucleic acid sequence encoding the variant, and the variant has a variant sequence of the aforementioned functional protein with functional protein activity selected from the following (i)-(iii):
(i) at least one sequence having deletion, substitution, insertion, mutation of 1, 2, 3, 4, 5, 6, 7, 8, 9 or 10 amino acids of the amino acid sequence of the functional protein in each group;
(ii) at least one amino acid sequence having at least 70%, 80%, 90%, 95% or 99% identity with the amino acid sequence as shown in any one of SEQ ID NO: 1-94; and
(iii) at least one sequence obtained by further fusing the amino acid sequence as shown in any one of SEQ ID NO: 1-94 with other sequences.

29. The composition according to claim 28, wherein the nucleic acid and/or nucleic acid set further includes a promoter and a poly (A) sequence.

30. (canceled)

31. The composition according to claim 28, wherein the nucleic acid set further includes an exogenous nucleic acid fragment.

32. The composition according to claim 31, wherein the exogenous nucleic acid fragment is operably inserted into the nucleic acid set through a polyclonal insertion site, the exogenous nucleic acid fragment may be one or more, and may be the same or different.

33. The composition according to claim 32, wherein the exogenous nucleic acid fragment includes any gene of interest or any gene that is transposable.

34. The composition according to claim 59, wherein the gene of a natural functional protein includes a fluorescence-based reporter gene, a luciferase gene, and/or a resistance gene.

35. The composition according to claim 59, wherein the artificial chimeric gene includes a gene of a chimeric antigen receptor.

36. The composition according to claim 59, wherein the gene of a non-coding RNA includes rRNA, tRNA, small interfering RNA (siRNA), small nuclear RNA (snRNA), small nucleolar RNA (snoRNA), microRNA (miRNA), and/or long non-coding RNA (lncRNA).

37. A recombinant vector, wherein, the recombinant vector includes the nucleic acid encoding the functional protein according to claim 1.

38. The recombinant vector according to claim 37, wherein the recombinant vector includes a recombinant cloning vector, a recombinant eukaryotic expression plasmid, or a recombinant viral vector.

39.-40. (canceled)

41. A recombinant host cell, wherein, the recombinant host cell comprises the functional protein according to claim 1.

42. The recombinant host cell according to claim 41, wherein the recombinant host cell includes an animal cell, a plant cell, an algal cell, a fungal cell, a yeast cell, or a bacterial cell.

43.-44. (canceled)

45. A method for introducing an exogenous nucleic acid fragment into the genome of a host cell, wherein, the method comprises: delivering the functional protein according to claim 1 or a nucleic acid encoding the functional protein according to claim 1 into the host cell.

46. A method for editing the genome of a host cell, wherein, the method comprises: delivering the functional protein according to claim 1 or a nucleic acid encoding the functional protein according to claim 1 into the host cell.

47. (canceled)

48. The method according to claim 46, wherein a delivery method includes cationic liposome delivery, lipoid nanoparticle delivery, cationic polymer delivery, vesicle-exosome delivery, gold nanoparticle delivery, polypeptide and protein delivery, retrovirus delivery, lentivirus delivery, adenovirus delivery, adeno-associated virus delivery, electroporation delivery, agrobacterium infection delivery, or gene gun delivery.

49.-56. (canceled)

57. A kit, wherein, the kit includes the functional protein according to claim 1.

58. The nucleic acid construct according to claim 21, wherein the exogenous nucleic acid fragment includes a gene of a natural functional protein, an artificial chimeric gene, and/or a gene of a non-coding RNA.

59. The composition according to claim 33, wherein the exogenous nucleic acid fragment includes a gene of a natural functional protein, an artificial chimeric gene, and/or a gene of a non-coding RNA.
